SEQUENCE LISTING (12 sequences)

SEQ ID NO: 1 - DNA, 20 bp, Suncus murinus
  source 1..20: mol_type = genomic DNA
tcgcagtccaaaatgaccga

SEQ ID NO: 2 - DNA, 20 bp, Suncus murinus
  source 1..20: mol_type = genomic DNA
tacctgatcccccattccgt

SEQ ID NO: 3 - DNA, 122 bp, Suncus murinus
  source 1..122: mol_type = genomic DNA
tcgcagtccaaaatgaccgagagcgctggcgacagtttctccgagggcttggcccctcccccacccacctcctccttcttcttcacatcttcagaggagcccacggaatgggggatcaggta

SEQ ID NO: 4 - DNA, 20 bp, Suncus murinus
  source 1..20: mol_type = genomic DNA
ccattgggcaaggtctggat

SEQ ID NO: 5 - DNA, 20 bp, Suncus murinus
  source 1..20: mol_type = genomic DNA
ggagctggagatggagatgc

SEQ ID NO: 6 - DNA, 216 bp, Suncus murinus
  source 1..216: mol_type = genomic DNA
ccattgggcaaggtctggatctcaaagaacttggcaatcctggagtggtctctggcatgcccattcttctctggtttggcggggctatcaggggtgctgtggaggccatgttgagatgggtcagggagggtcagctggtggtggctgggtggaatgggactgtagcgggtcctctcaggcgggctggtgctggacagcatctccatctccagctcc

SEQ ID NO: 7 - DNA, 20 bp, Suncus murinus
  source 1..20: mol_type = genomic DNA
aaaagggcaggaaggactcg

SEQ ID NO: 8 - DNA, 20 bp, Suncus murinus
  source 1..20: mol_type = genomic DNA
ggttgccttcttctcccgaa

SEQ ID NO: 9 - DNA, 156 bp, Suncus murinus
  source 1..156: mol_type = genomic DNA
aaaagggcaggaaggactcgaaactctctcagccccaacttggcacccaagctcagcttagaagttcgaaaactcagtaacggcaggctgtcaacatccctaaagttgggtccactgcaacctagatcagtgccacttcgggagaagaaggcaacc

SEQ ID NO: 10 - DNA, 20 bp, Suncus murinus
  source 1..20: mol_type = genomic DNA
acaggcctctcagtgtctca

SEQ ID NO: 11 - DNA, 20 bp, Suncus murinus
  source 1..20: mol_type = genomic DNA
tctgggcctggttctactgt

SEQ ID NO: 12 - DNA, 169 bp, Suncus murinus
  source 1..169: mol_type = genomic DNA
acaggcctctcagtgtctcagcacagacagacagacagacgtgcctgcatctgtcttgttctcgccccacacccgagtccccctgccagacgtcccctgcaacactaccagccaaggagttactgctgctcgggtggctggaacccacgacagtagaaccaggcccaga

(19) United States
(12) Patent Application Publication
(10) Pub. No.: US 2023/0352136 A1
(43) Pub. Date: Nov. 2, 2023

(54) SYSTEMS AND METHODS TO EVALUATE DRUG-INDUCED GASTROINTESTINAL DYSRHYTHMIA

(71) Applicants: The Chinese University of Hong Kong, Hong Kong (CN); Auckland UniServices Ltd., Auckland (NZ)

(72) Inventors: John Anthony Rudd, Hong Kong (CN); Yuen Hang Julia Liu, Hong Kong (CN); Peng Du, Hong Kong (CN)

(21) Appl. No.: 18/179,583

(22) Filed: Mar. 7, 2023

Related U.S. Application Data
(60) Provisional application No. 63/268,957, filed on Mar. 7, 2022.

(51) Int. Cl. G16H 20/10; G01N 33/50

(52) U.S. Cl. CPC G16H 20/10; G01N 33/502

(57) ABSTRACT

The subject invention pertains to the application of GI slow-wave network analysis to profile GI side effects for applications including high-throughput drug screening on a microelectrode array (MEA) platform. Slow-wave data can be obtained, evaluated, interpreted, and used to build a comprehensive database based on the effects of specified drugs on GI pacemaker activity for predictive and classification purposes. In one example, pacemaker potentials were recorded extracellularly on a 60-channel MEA system using full-thickness GI segments isolated from Suncus murinus. Basic slow-wave parameters, including frequency, amplitude, slope, period, and power partitions, were derived. Signal regularity was also evaluated using detrended fluctuation analysis and sample entropy analysis. Signal propagation, velocity, and activation time patterns were also constructed and compared before and after treatment with dopamine (0.1-100 μM).

### CROSS-REFERENCE TO RELATED APPLICATION

This application claims the benefit of U.S. Provisional Patent Application Ser. No. 63/268,957, filed Mar. 7, 2022, which is hereby incorporated by reference in its entirety including any tables, figures, or drawings.

### REFERENCE TO SEQUENCE LISTING

The Sequence Listing for this application is labeled “CUHK.174XC1.xml” which was created on Feb. 8, 2023 and is 12,043 bytes. The entire content of the sequence listing is incorporated herein by reference in its entirety.

### BACKGROUND OF THE INVENTION

Medicines often unexpectedly disturb gastrointestinal (GI) functions. The process of drug approval by the US Food and Drug Administration do not have requirements regarding the drug potential adverse effects at its non-target sites, such as the GI. Drug discovery and development phase focus only on the drug efficacy to treat a target disease and very often test only using cell-based models or rodents, without evaluating the side effects on GI function. Drug adverse effects, such as vomiting, nausea, diarrhea, and constipation, etc. can remain until entering clinical trials, which wastes a large amount of money and time. Conventional approaches to drug discovery often fail to identify emetic liability and GI complications until reaching clinical trials. Unfortunately, the late identification of GI adverse effects can result in unnecessary experimentation, the termination of clinical investigations, and the loss of projected revenue. Conventional approaches to investigating drugs affecting the GI tract are useful, but they do not usually capture effects on ‘slow waves’, which are known to be disturbed during nausea and emesis. Dopamine is a well-known neurotransmitter and a precursor of noradrenaline and adrenaline biosynthesis. Dopamine receptor agonists are used to treat cardiovascular disorders and Parkinson's disease, but they have adverse side effect profiles that include nausea, vomiting, and constipation.

### BRIEF SUMMARY OF THE INVENTION

Embodiments of the subject invention can provide an index and guidelines on the potential effects of drugs to induce GI dysfunction using a standardized and efficient method for prediction using a learning model based on a novel database built using a standardized methodology.

Embodiments can advantageously employ dopamine as an exemplar drug to introduce the application of GI slow-wave network analysis to profile GI side effects for high-throughput drug screening on a microelectrode array (MEA) platform. In certain embodiments, slow-wave data can be obtained, evaluated, interpreted, and used to build a comprehensive database based on the effects of drugs on GI pacemaker activity for predictive and classification purposes.

Embodiments can provide a technique to record GI pacemaker activity, also named slow waves, in GI tissues isolated from small animals using the microelectrode array (MEA) platform. Embodiments have demonstrated >75% success rate in recording high quality pacemaker signals. Embodiments can provide analytical programs (e.g., automatic programs using MATLAB, The MathWorks, Inc., Natick, MA) for efficient and blind data analysis for slow wave feature extractions.

Embodiments can provide a standardized protocol to evaluate acute drug effects on GI pacemaker activity. Data can be collected and stored in a drug database. In one embodiment, a drug database contains more than 100 exemplar drugs and continues to grow. In this embodiment, the database value is advantageously enhanced since the data was compiled using standardized methodology enabling highly reliable drug-to-drug comparisons. The application of the database includes, but is not limited to, drug research and development, food safety test, research on traditional remedies, and development of personalized drug therapy.

Embodiments can provide drug testing services using a standardized methodology and automatic data analytical pipeline to generate standardized drug testing reports. Such reports can include slow wave features extracted to be compared with the existing database to add value, advance development, or generate revenues. In certain embodiments, machine learning models are provided to beneficially refine features for different purposes, such as to predict nausea potential, or to classify drugs. Such refined features can be referred to as Gastrointestinal Dysrhythmia Indexes. These indexes can provide important insights on the potentials of drugs to cause (or treat) GI dysrhythmia. Embodiments can provide information generated by business models (e.g., drug testing services) that will be useful in decision making in drug discovery and development, and medical prescription.

Embodiments can provide an efficient and standardized method for testing the effects of one or more specified substances on pacemaker activity of gastrointestinal tissues. Certain embodiments can provide test results to clients; advantageously apply a standardized method to build databases relevant to the effects of substances on pacemaker activity of gastrointestinal tissues; and generate machine learning models based on the databases to provide consistent, reliable and reproducible results with minimized bias and minimized errors to clients (e.g., to determine the risk of side effects of a drug; or to determine the agonist or antagonist actions of a drug.)

Embodiments can provide a standardized methodology to record electrical pacemaker signals from the surface of freshly isolated gastrointestinal tissues from a living organism. Embodiments can further provide a set of instructions written in a machine-readable format to allow automatic, efficient, consistent, and non-biased extraction of features from recorded pacemaker signals. Embodiments can further provide a database built using a standardized method to allow generation of machine learning models, with or without the integration of other sources of databases for generating results of classifications, comparisons and predictions related to the tested substances.

Embodiments can provide a novel business model that is unique in its method to generate revenue, optionally attached to the MEA technology, and the provided automatic analytical pipelines, and a provided novel and private database produced under highly standardized protocols.

Embodiments of a business model is accordance with the subject invention can provide advantages including, but not limited to the following. Efficiency, wherein an experiment can be done, for example, within 24 hours tested on 4 segments of the GI at 3 different doses on 6 animal replicates within the limitation of an existing MEA platform. Efficiency and Accuracy, wherein data analysis can be performed using developed automatic and blinded software programs to reduce time spent, bias, and human errors. Consistency and reliability, wherein data can be generated using a standardized method allowing reliable data comparison to additional data stored in a database. Comprehensive results, wherein the method can apply the use of MEA technology over conventional methods such as single microelectrodes, patch clamping, and calcium imaging for recording pacemaker activity. The MEA technology can be advantageously employed to capture pacemaker signals over a larger area including information on propagating and networking behavior, together with a developed program for comprehensive feature extractions to generate comprehensive information on drug effects on GI motility in terms of pacemaker activity. Blinded study, wherein the data collection and data analysis process can be made more effectively blinded, thus avoiding bias. The unique predictive power, wherein there are no known standardized database for evaluating drug effects on pacemaker activity at the GI. The current drug databases generated based on a standardized protocol in accordance with embodiments of the subject invention provides a unique and novel predictive power for evaluating the potential of a candidate cause (or to treat) GI dysrhythmia.

Embodiments can advantageously employ the use of related art elements, including but not limited to existing developed MEA platforms including the machines (Multichannel Systems, Germany) and devices such as the MEA chips (Ayanda Biosystems or Multichannel Systems, Germany); the application of the MEA to record GI pacemaker activity in small animals; the application of the MEA to evaluate drug effects; the application of the data analysis using known formulas and algorithms to extract slow wave features, including continuous wavelet transform, Hilbert transform, detrended fluctuation analysis, sample entropy, and machine learning.

GI motility is highly coordinated by rhythmic electrical signals, called pacemaking activity or slow-waves, controlled by network of interstitial cells of Cajal (ICC). These propagating electrical signals can be recorded using a microelectrode array (MEA) platform, and drug-induced effects on GI pacemaking activity can be tested using a standardized methodology as disclosed herein. It is proposed that these electrical signals are the languages representing healthy bowel movement and reflecting non-conscious health and disease conditions of our body.

Electrical data is a category of big-data which is currently highly-underutilized in biology and medicine. To decode the language of rhythmic GI pacemaking signals, embodiments of the subject invention take in the signals during drug treatment as the signals work to activate various receptors, ion channels, enzymes, and the like. The final pacemaking signals can integrate signals directly coming from ICC, indirect interactions with enteric neurons and smooth muscles, and signals coming beyond the GI. Embodiments of the subject invention decode and translate these signals with the help of artificial intelligence (AI) technology, to identify correlations between GI pacemaker activity and health and disease. The inventors have collected and integrated a large amount of drug testing data on GI pacemaker activity in previously published and unpublished studies, where in certain embodiments a standardized and robust MEA methodology is provided to test different drugs at different effective doses in different GI segments including, stomach, duodenum, ileum, and colon [1-6]. Embodiments provide a new type of electrophysiological drug database based on changes in 24 signal features extracted from recorded GI pacemaker activity before and after acute drug treatment. These extracted signal features are referred to as electrical features (EF). This drug database is referred to as the Gastro-Intestinal Pacemaker Activity Drug Database (GIPADD). While the GIPADD is intended to undergo continued growth and future expansion, certain embodiments reported herein were created using a cut-off database with 89 drugs and 4,867 datasets. Embodiments provide application of this novel EF drug database, GIPADD, to predict drug adverse effects (AEs).

In the emerging field of AI drug discovery, AI had been applied in designing drug molecules and predicting drug targets, responses and AEs. Current methodologies mainly used drugs' physical and chemical properties, AI-predicted or known drug-protein-receptor binding interactions, and genetic or gene expression profiles [7,8]. Embodiments of the subject invention introduce EF drug profile into this research field. Related art drug AEs prediction AI models have significant limitation in data quality, including bias, non-standardized, and non-validated data collection methodologies. Data used in related art systems and methods mainly came from publications which often present data that supports a hypothesis, instead of a pure factual data presentation, where negative data can be unreported. Prediction algorithms based on these data suffered significant biased towards what is discovered or hypothesized by investigators, limiting the ability to discover novel pathways or correlations. Embodiments of the subject invention provide systems and methods for creating and maintaining physiological drug databases produced by standardized and validated methodology, providing numerous advantageous improvements in AI drug discovery. In certain embodiments GIPADD is produced by a highly-standardized methodology, i.e., drug profiles of each categorized drug stored in GIPADD are highly consistent, and therefore, can act as positive and negative controls for each other depending on the testing hypothesis. Additionally, GIPADD stores unique EFs, which potentially integrate further with other drug databases storing physical, chemical, genetic data, and the like. Certain embodiments integrate GIPADD with side-effect resource (SIDER) to test the hypothesis to use drug-induced GI pacemaker EFs to predict drug AEs. Moreover, GI function goes far beyond digestion for absorbing life-supporting nutrients, but also contributes to at least 70 percent of our immunity and 95 percent of serotonin release controlling our emotions [45]. It expresses almost all known receptors or proteins found in the brain, liver, reproductive organs, etc. Without being bound by theory, the inventors hypothesize that AEs prediction using GI pacemaker activity can go beyond predicting only GI-related AEs, to include AEs in immunology, cardiovascular, psychology, and other areas of interest.

In certain embodiments GIPADD provides novel EF big-data resources for training AI models in drug discovery to correlate drug EF profiles for the prediction of drug adverse effects or drug targets. GIPADD is a small but growing database. Problems like lack of datasets and imbalanced datasets for certain AEs can be improved by adding more drug profiles into the database through standardized drug testing methodology. GIPADD is a novel drug database storing EFs providing a novel and advantageous source of big-data learning materials for the emerging field of artificial intelligence drug discovery, as well as a novel and uniquely advantageous solution to challenges of working with biased learning materials.

Certain embodiments provide specified novel and innovative solutions. In data analysis, embodiments provide a novel methodology to combine the use of the extracted slow wave features by known algorithms to evaluate GI dysrhythmia based on comparative change of these features as standards to evaluate potentials of drugs to cause (or to treat) GI dysrhythmia. In data analysis, embodiments can provide a novel fully automatic methodology to apply phase-based spatial analysis to evaluate percentage change of dominant propagation patterns as a major feature for drug functional tests in GI dysrhythmia, the features extracted can also be stored into the database. Embodiments can provide a stated standardized drug testing protocol (including the stated experiment methodology and the specialized data analytical pipeline) to provide drug testing services to generate revenue. Embodiments can provide a private, proprietary, or novel database built using a stated standardized protocol for useful, consistent, and reliable data comparison to generate revenue.

Embodiments can provide a standardized methodology to allow an easy, reliable, and consistent comparison between test agents and compounds in the proprietary database. One existing problem in studying drug effects in physiology is that data are scattered in separate literatures and were conducted in separate laboratories using different experimental protocols. This makes data comparison very difficult, especially if a large-scale data comparison is aimed for building predictive or classification learning models. Additionally, physiology experiments for drug testing are often tedious and require high-level training to maintain consistency, a high success rate, and unbiased analysis of results.

Embodiments can include the use of a MEA platform for high-throughput drug screening and testing using a highly efficient and standardized methodology and analytical pipelines for building a database. The database can systematically store information on drug effects on GI pacemaker activity. Using the database, reliable and systematic drug-to-drug comparison, grouping and application to machine learning for building classification and predictive model can be made more feasible, efficient, and reliable. For example, the classification of drug actions on a specific receptor, or the refined features for predicting the potential of a drug to induce nausea. Certain embodiments can provide the construction of Gastrointestinal Dysrhythmia Indexes for guiding drug discovery processes in terms of the potentials of drugs to induce GI adverse effects such as nausea, vomiting, diarrhea, constipation, and the like. The same or similar model and database in accordance with the subject invention can be used to identify drugs to potentially treat GI dysrhythmia. This information can be useful in applications including but not limited to drug discovery and development, food safety policies, and development of personalized drug therapy.

Embodiments can provide a highly standardized database, wherein standardized methodology can attract clients who would like to test their drugs and compare their drug profile with existing data in the database, or estimate the potential effects of novel drugs based on the predictive power of the database. Embodiments can provide a business model that includes providing drug testing services to help clients to test their drugs using a standardized methodology and automatic analytical pipelines and drug comparison services to compare their drugs with one or more selected private databases to generate revenue.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1O show certain effects of dopamine on pacemaker potentials along the gastrointestinal tract of Suncus murinus according to an embodiment of the subject invention. Slow-wave features including the (FIG. 1A) dominant frequency (DF), (FIG. 1B) average frequency (AF), and (FIG. 1C) dominant power (DP); (FIG. 1D) the average amplitude, (FIG. 1E) slope, and (FIG. 1F) period of the waveforms; (FIG. 1G) propagation velocity; (FIG. 1H) detrended fluctuation analysis (DFA) fluctuation function at smaller window scale (3-25) and (FIG. 1I) at larger window scale (26-46); and (FIG. 1J) sample entropy (SampEn) at smaller window scale (1-5) and (FIG. 1K) at larger window scale (6-50) are indicated as the percentage change after dopamine (0.1-100 μM) treatment, compared with baseline recordings. (FIGS. 1L-M) The effects of dopamine (100 μM) on pacemaker potentials determined by DFA in isolated Suncus murinus ilea, (FIG. 1L) DFA fluctuation functions of all 60 electrodes, where the scale is the same as the enlarged graph in (FIG. 1M), showing only channel 37. (FIGS. 1N-O) The effects of dopamine (100 μM) on pacemaker potentials determined by SampEn in isolated Suncus murinus ilea, (FIG. 1N) The SampEn of all 60 electrodes, where the scale is the same as the enlarged graph in (FIG. 1O), showing only channel 37.

FIGS. 2A-2E show certain effects of dopamine on pacemaker potentials determined by the frequency shifting behavior of the power spectrum according to an embodiment of the subject invention. Power spectra were generated using 5 min baseline data obtained from the (FIG. 2A) stomach and (FIG. 2B) colon using fast Fourier transform analysis with a bin size of 2048 and a Hanning window. (FIG. 2C) Stacked histograms showing the effects of dopamine (0.1-100 μM) on frequency partitioning along the gastrointestinal tract of Suncus murinus. (FIGS. 2D-E) Representative spectrograms showing the effects of 100 μM dopamine on the (FIG. 2D, left panel) stomach, and the effects of 10 μM dopamine on the (FIG. 2D, right panel) duodenum, (FIG. 2E, left panel) ileum, and (FIG. 2E, right panel) colon of Suncus murinus.

FIGS. 3A-3G show certain effects of dopamine on pacemaker potentials determined by the activation time pattern distribution according to an embodiment of the subject invention. (FIGS. 3A-C) Representative figures showing the three most dominant activation time patterns within the (FIG. 3A) baseline recordings and the (FIG. 3B) post-treatment recordings. (FIG. 3C) The counts of the appearance of each grouped activation time pattern determined at 20 s intervals. The pattern distribution of the dominant activation time patterns based on the (FIG. 3D) baseline and (FIG. 3E) post-treatment recordings. (FIG. 3F) The number of grouped activation time patterns within the baseline and post-treatment recordings. (FIG. 3G) The total percentage change of all activation time patterns induced by dopamine treatment is indicated as ActP.

FIG. 4 shows a radar diagram showing the profile of dopamine effects on pacemaker potentials along the gastrointestinal tract of Suncus murinus according to an embodiment of the subject invention. All data show the mean percentage change in slow-wave features, including the dominant frequency (DF); average frequency (AF); percentage of brady-rhythm, normal rhythm, and tachy-rhythm; dominant power (DP); average amplitude, slope, and period of the waveform; average propagation velocity; detrended fluctuation analysis (DFA) fluctuation function with small window scale 3-25 and large window scale 26-46; sample entropy (SampEn) with small window scale 1-5 and large window scale 6-50; and the total change in activation time pattern (ActP). The radar diagram provides an immediate indication of how dopamine affects GI pacemaker activity across selected parameters in one graph.

FIGS. 5A-5D. show an example application of a drug database according to an embodiment of the subject invention. A clustergram showing the 24 slow-wave features extracted from the duodenal data for a list of drugs (FIG. 5A) with known nausea-inducing or non-nausea-inducing properties and (FIG. 5B) known to be agonists or antagonists of the dopamine receptor. Features extracted with significant differences between (FIG. 5C) the nausea-inducing and non-nausea-inducing drugs and (FIG. 5D) the dopamine agonists and antagonists.

FIG. 6 shows the mRNA expression of dopamine receptors in Suncus murinus according to an embodiment of the subject invention.

FIGS. 7A-7I show representative spatio-temporal maps showing the effects of 100 μM dopamine on the stomach, and the effects of 10 μM dopamine on the duodenum, ileum, and colon of Suncus murinus compared to baseline according to an embodiment of the subject invention. (FIG. 7A) comparison matrix showing baseline and treatment for each of stomach, duodenum, ileum, and colon, and (FIGS. 7B-I) detailed views of: (FIG. 7B) stomach baseline; (FIG. 7C) stomach 100 μM dopamine; (FIG. 7D) duodenum baseline; (FIG. 7E) duodenum 10 μM dopamine; (FIG. 7F) ileum baseline; (FIG. 7G) ileum 10 μM dopamine; (FIG. 7H) colon baseline; (FIG. 7I) colon 10 μM dopamine.

FIG. 8 shows representative raw traces showing the effects of 100 μM dopamine on the stomach, and the effects of 10 μM dopamine on the duodenum, ileum, and colon of Suncus murinus according to an embodiment of the subject invention.

FIGS. 9A-9F illustrates representative data collected on ferrets according to an embodiment of the subject invention.

FIG. 10 illustrates representative raw data collected on rats according to an embodiment of the subject invention.

FIGS. 11A-11B illustrate representative progression of screen shots from a video clip showing the effects of 100 μM dopamine on wave propagation in the stomach over approximately two cycles according to an embodiment of the subject invention at (FIG. 11A) baseline and (FIG. 11B) after drug treatment. For each frame of FIG. 11A and FIG. 11B, respectively, the Distance in mm is shown from (0,0) in the top left corner to (1.4, 1.4) in the bottom right corner, and the Normalized Amplitude (%) color scale ranges from blue (−200) to green (0) to red (+200); as shown in FIG. 11A.

FIGS. 12A-12B illustrate representative progression of screen shots from a video clip showing the effects of 100 μM dopamine on wave propagation in the colon according to an embodiment of the subject invention at (FIG. 12A) baseline and (FIG. 12B) after drug treatment. For each frame of FIG. 12A and FIG. 12B, respectively, the Distance in mm is shown from (0,0) in the top left corner to (1.4, 1.4) in the bottom right corner, and the Normalized Amplitude (%) color scale ranges from blue (−200) to green (0) to red (+200); as shown in FIG. 11A.

FIGS. 13A-13B illustrate a flow chart showing the process from dataset preparation, to machine learning (ML) model training, to prediction result refinement for generating different types of ML models according to an embodiment of the subject invention.

FIGS. 14A-14C illustrate model comparisons according to an embodiment of the subject invention. (FIG. 14A) The prediction accuracy of models built using different dataset preparations for refined selected-AEs (n=10-13); (FIG. 14B) The prediction accuracy of different tissue models. ‘All’ column indicates average of all 4-tissue-type (n=2,016) and ‘Intestine’ column indicates average of 3 intestinal segments except stomach (n=1,554), stomach and ileum (n=462), duodenum and colon (n=546); (FIG. 14C) The prediction accuracy of model trained using different classification algorithms (n=336). Data represents the mean±S.D. Significant differences are indicated as * p<0.05, ** p<0.01, *** p<0.001 using paired t-tests.

FIGS. 15A-15L illustrate excitatory and inhibitory correlations to GI pacemaker activity in selected-AEs according to an embodiment of the subject invention. The percentage change of selected features in various AE-inducing drugs and non-AE-inducing drugs. Data represents the mean±S.D. Significant differences are indicated as * p<0.05, ** p<0.01, *** p<0.001 using unpaired t-tests (n=46-1,326).

FIGS. 16A-16D illustrate selected GI pacemaker features correlated with AEs according to an embodiment of the subject invention. (FIG. 16A) Network graph clustering selected drugs by refined EFs to an example AE: constipation. The length of black arrow represents the level that the model can distinguish between positive-correlated features and negative-correlated features in constipation (shaded in yellow). Blue arrow and Red arrow has a short distance to positive-correlated features of constipation, where these two drugs ondansetron (“ond”) and morphine (“mor”) are known in market to induce constipation. Drugs acting on similar receptors, such as prostaglandin E1 (“pge1”) and prostaglandin E2 (“pge2”) or substance P (“sp”) and neurokinin A (“nka”) are clustered close to each other based on EF drug profile (shaded in yellow). The color of the bubbles represents the clustered groups of drugs, which show the successful clustering on top of spatial information provided by the bubbles. (FIGS. 16B-D) The percentage change of selected features compared between AE-inducing drugs and non-AE-inducing drugs in (FIG. 16B) GI-related AEs including abdominal distension, upset stomach, and abdominal cramps, (FIG. 16C) psychology-related AEs including anxiety and depression, and (FIG. 16D) and Cardiovascular-related AEs including hypotension, hypertension. Data represents the mean±S.D. Significant differences are indicated as * p<0.05, ** p<0.01, *** p<0.001 using unpaired t-tests (n=228-1,300). NKA: neurokinin A; LPS: lipopolysaccharides.

### BRIEF DESCRIPTION OF THE SEQUENCES

SEQ ID NO: 1 Forward primer D1 Dopamine receptor (Suncus murinus)
SEQ ID NO: 2 Reverse primer D1 Dopamine receptor (Suncus murinus)
SEQ ID NO: 3 Amplified D1 Dopamine receptor DNA segment (Suncus murinus)
SEQ ID NO: 4 Forward primer D2 Dopamine receptor (Suncus murinus)
SEQ ID NO: 5 Reverse primer D2 Dopamine receptor (Suncus murinus)
SEQ ID NO: 6 Amplified D2 Dopamine receptor DNA segment (Suncus murinus)
SEQ ID NO: 7 Forward primer D3 Dopamine receptor (Suncus murinus)
SEQ ID NO: 8 Reverse primer D3 Dopamine receptor (Suncus murinus)
SEQ ID NO: 9 Amplified D3 Dopamine receptor DNA segment (Suncus murinus)
SEQ ID NO: 10 Forward primer D4 Dopamine receptor (Suncus murinus)
SEQ ID NO: 11 Reverse primer D4 Dopamine receptor (Suncus murinus)
SEQ ID NO: 12 Amplified D4 Dopamine receptor DNA segment (Suncus murinus)

### DETAILED DISCLOSURE OF THE INVENTION

The invention may be better understood by reference to certain non-limiting embodiments, including but not limited to the following.

Embodiment 1 can provide a method of testing effects of one or more substances on pacemaker activity on gastrointestinal tissues using a recording platform to determine whether the substances belong to one or more classes, the method comprising:
applying one or more substances for testing on at least one sub-segment of freshly isolated gastrointestinal tissue from a living organism;
maintaining the freshly isolated gastrointestinal tissue in oxygenated medium to maintain the viability of tissues;
recording electrical signals from a surface of the tissue using the recording platform to store a recorded digital signal;
storing the recorded digital signal in a data storage device;
analyzing the recorded digital signal using a set of machine-readable instructions that allow a computer to extract at least one feature from the recorded digital signal; generating a report of test results;
reporting the test results to a client;
storing the test results into one or more databases;
training one or more machine learning models based on the test results stored in the one or more databases, to create one or more trained models;
applying at least one of the one or more trained models for classifying, predicting, or comparing the one or more substances;
reporting a result of the classifying, predicting, or comparing results to a client.

Embodiment 2. The method according to Embodiment 1, wherein the one or more substances comprise one or more of drugs, pharmacological agents, chemical compounds, synthesized substances, food, remedies, herbs, extracts, and combinations of the above.

Embodiment 3. The method according to Embodiment 1, wherein the recording platform comprises a signal receiver, an amplifier, an internal filter, a grounding electrode and a microelectrode array chip; the microelectrode array chip comprising a multiplicity of microelectrodes embedded on a rigid substrate.

Embodiment 4. The method according to Embodiment 1, comprising predicting and classifying between agonist and antagonist actions of the one or more substances, or predicting and classifying between high-risk and low-risk in a set of selected side effects of the one or more substances.

Embodiment 5. The method according to Embodiment 4, the set of selected side effects comprising one or more of vomiting, emesis, nausea, diarrhea, constipation, abdominal discomfort, and dysrhythmia.

Embodiment 6. The method according to Embodiment 1, wherein the sub-segment of freshly isolated gastrointestinal tissue comprises tissue from one or more of an esophagus, stomach, duodenum, jejunum, ileum, rectum, caecum, or colon.

Embodiment 7. The method according to Embodiment 1, wherein the living organism is an organism having functional gastrointestinal organs.

Embodiment 8. The method according to Embodiment 1, wherein the living organism is human, mammalian, reptilian, or aquatic.

Embodiment 9. The method according to Embodiment 1, wherein the living organism is healthy; or diagnosed with a disease, genetic condition, or alteration; or pre-treated with at least one of the one or more substances prior to the applying one or more substances for testing.

Embodiment 10. The method according to embodiment 1, further comprising the step of removing contents from within the gastrointestinal tissue.

Embodiment 11. The method according to Embodiment 1, further comprising maintaining the temperature of the freshly isolated gastrointestinal tissue within a range of twenty to forty degrees Celsius.

Embodiment 12. The method according to Embodiment 1, further comprising recording a baseline signal for at least five minutes prior to the applying one or more substances for testing.

Embodiment 13. The method according to Embodiment 1, further comprising delivering one or more substances onto the sub-segment of freshly isolated gastrointestinal tissue.

Embodiment 14. The method according to Embodiment 13, wherein the delivering comprises either direct delivery using a handheld pipette or machine-controlled delivery using a machine-controlled perfusion system.

Embodiment 15. The method according to Embodiment 13, wherein the recording electrical signals occurs after the step of delivering one or more substances onto the sub-segment of freshly isolated gastrointestinal tissue.

Embodiment 16. The method according to Embodiment 12, further comprising comparing the baseline signals to the signals recorded after the step of delivering one or more substances.

Embodiment 17. The method according to Embodiment 1, wherein the recorded digital signal is created within less than one hour after the applying one or more substances for testing.

Embodiment 18. The method according to Embodiment 1, wherein the at least one feature from the recorded digital signal comprises one or more of:
the determination of a number of dominant propagation patterns using a factor of activation times found at each electrode within the baseline period and post substance delivery period into time interval between ten to sixty second;
the percentage of the dominant propagation patterns found based on the baseline period of signals and the post-substance delivery period of signals are derived; and
the change in the percentage of a first, second, or third propagation pattern based on baseline period and post-substance delivery period being compared between the baseline signals and signals recorded after the step of delivering one or more substances to determine one or more effects of the one or more substances on a pacemaker propagation pattern.

Embodiment 19. The method according to Embodiment 1, further comprising constructing one or more databases to store the at least one feature from the recorded digital signals for each of the one or more substances.

Embodiment 20. The method according to Embodiment 19, comprising building a trained machine learning model based on the one or more databases, and integrating the one or more databases with at least one other database or training model.

Embodiment 21. A method of testing effects of one or more substances on pacemaker activity on gastrointestinal tissues using a recording platform to determine whether the one or more substances belong to one or more classes, the method comprising:
applying a substance for testing on at least one sub-segment of freshly isolated gastrointestinal tissue from a living organism;
maintaining the tissue in oxygenated medium to maintain a viability of the tissue; recording electrical signals from a surface of the tissue using the recording platform to create a recorded digital signal;
storing the recorded digital signal in a data storage device;
generating a plurality of test results by analyzing the recorded digital signal using a set of machine-readable instructions that allow a computer to extract at least one feature from the recorded digital signal;
storing the plurality of test results into a database;
training one or more machine learning models based on the plurality of test results stored in the database, to create a trained model;
applying the trained model for classifying, predicting, or comparing the substance; reporting a result of the classifying, predicting, or comparing.

Embodiment 22. The method according to Embodiment 21, wherein the substance comprises one or more of drugs, pharmacological agents, chemical compounds, synthesized substances, food, remedies, herbs, extracts, and any combination thereof.

Embodiment 23. The method according to Embodiment 21, wherein the recording platform comprises a signal receiver, an amplifier, an internal filter, a grounding electrode and a microelectrode array chip; the microelectrode array chip comprising a multiplicity of microelectrodes embedded on a rigid substrate.

Embodiment 24. The method according to Embodiment 21, comprising predicting and classifying between agonist and antagonist actions of the one or more substances, or predicting and classifying between high-risk and low-risk in a set of selected side effects of the substance.

Embodiment 25. The method according to Embodiment 24, the set of selected side effects comprising one or more of vomiting, emesis, nausea, diarrhea, constipation, abdominal discomfort, and dysrhythmia.

Embodiment 26. The method according to Embodiment 21, wherein the sub-segment of freshly isolated gastrointestinal tissue comprises tissue from one of an esophagus, stomach, duodenum, jejunum, ileum, rectum, caecum, or colon.

Embodiment 27. The method according to Embodiment 21, wherein the living organism is an organism having functional gastrointestinal organs.

Embodiment 28. The method according to Embodiment 21, wherein the living organism is human, mammalian, reptilian, or aquatic.

Embodiment 29. The method according to Embodiment 21, wherein the living organism is healthy; or diagnosed with a disease, genetic condition, or alteration; or is pre-treated with the substance prior to the applying the substance for testing.

Embodiment 30. The method according to Embodiment 21, further comprising the step of removing contents from within the freshly isolated gastrointestinal tissue.

Embodiment 31. The method according to Embodiment 21, further comprising maintaining the temperature of the freshly isolated gastrointestinal tissue within a range of twenty to forty degrees Celsius.

Embodiment 32. The method according to Embodiment 21, further comprising recording a baseline signal for at least five minutes prior to the applying the substance for testing.

Embodiment 33. The method according to Embodiment 32, the applying the substance for testing comprising delivering a specified quantity of the substance onto the sub-segment of freshly isolated gastrointestinal tissue at a specified time after the recording of the baseline signal.

Embodiment 34. The method according to Embodiment 33, wherein the delivering comprises either direct delivery using a handheld pipette or machine-controlled delivery using a machine-controlled perfusion system.

Embodiment 35. The method according to Embodiment 33, wherein the recording electrical signals occurs after the delivering of the specified quantity of the substance onto the sub-segment of freshly isolated gastrointestinal tissue at the specified time, and wherein the recorded digital signal is a post-substance delivery signal.

Embodiment 36. The method according to Embodiment 35, further comprising comparing the baseline signal to the post-substance delivery signal.

Embodiment 37. The method according to Embodiment 21, wherein the recorded digital signal is created within less than one hour after the applying one or more substances for testing.

Embodiment 38. The method according to Embodiment 21, wherein the at least one feature from the recorded digital signal comprises one or more of:
the determination of a number of dominant propagation patterns using a factor of respective activation times found at each electrode within a baseline period and a post-substance delivery period, respectively, into a time interval between ten to sixty seconds;
the percentage of the dominant propagation patterns found in the baseline period and the post-substance delivery period, respectively; and
the change in the percentage of a first, second, or third propagation pattern based on a comparison between the baseline period and the post-substance delivery period.

Embodiment 39. The method according to Embodiment 21, the substance being a first substance and the database comprising (i) a first unique individual database section configured to store the at least one feature from the recorded digital signal for the first substance and (i) a second unique individual database section configured to store at least one feature from a recorded digital signal for a second substance.

Embodiment 40. The method according to Embodiment 39, comprising building a trained machine learning model based on the first unique individual database section and the second unique individual database section.

Embodiment 41. The method according to Embodiment 40, further comprising integrating the first unique individual database section and the second unique individual database section with at least one other database or training model.

Embodiment 42. A system for determining adverse effects (AEs) of one or more substances on a gastrointestinal (GI) tissue, the system comprising:
an electrical signal platform configured and adapted to create an electrical signal from the GI tissue;
a processor; and
a machine-readable medium in operable communication with the electrical signal platform and the processor and having instructions stored thereon that, when executed by the processor, perform the following steps:
   recording the electrical signal from the GI tissue to create a recorded digital signal;
   storing the recorded digital signal in the machine-readable medium;
   generating a plurality of test results by analyzing the recorded digital signal to extract at least one feature from the recorded digital signal;
   storing the plurality of test results into a database;
   training one or more machine learning models based on the plurality of test results stored in the database, to create a trained model;
   applying the trained model to determine the AEs by classifying, predicting, or comparing the substance;
   reporting a result of the AEs determined by classifying, predicting, or comparing the substance.

Embodiment 43. A system for determining adverse effects (AEs) or drug targets of one or more substances on a gastrointestinal (GI) tissue, the system comprising:
an electrical signal platform configured and adapted to create an electrical signal from the GI tissue;
a processor; and
a machine-readable medium in operable communication with the electrical signal platform and the processor and having instructions stored thereon that, when executed by the processor, perform the following steps:
   recording the electrical signal from the GI tissue to create a recorded digital signal;
   storing the recorded digital signal in the machine-readable medium;
   generating a plurality of test results by analyzing the recorded digital signal to extract at least one feature from the recorded digital signal;
   storing the plurality of test results into a database;
   training one or more machine learning models based on the plurality of test results stored in the database, to create a trained model;
   applying the trained model to determine the AEs or drug targets by classifying, predicting, or comparing the substance;
   reporting a result of the AEs or drug targets determined by classifying, predicting, or comparing the substance.

Embodiment 44. The method according to Embodiment 43, the instructions when executed by the processor performing the additional step of clustering two or more compounds together based on a common action to similar receptors or similar drug-induced AEs.

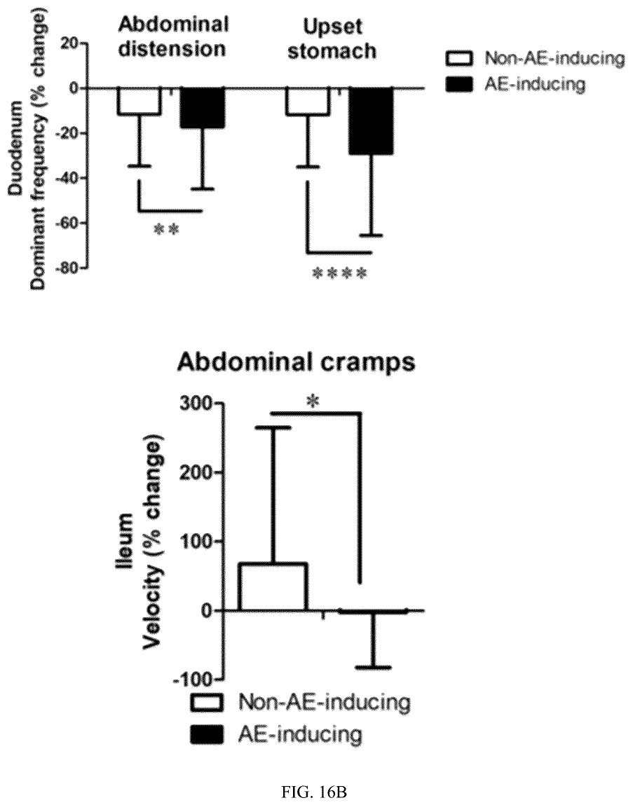

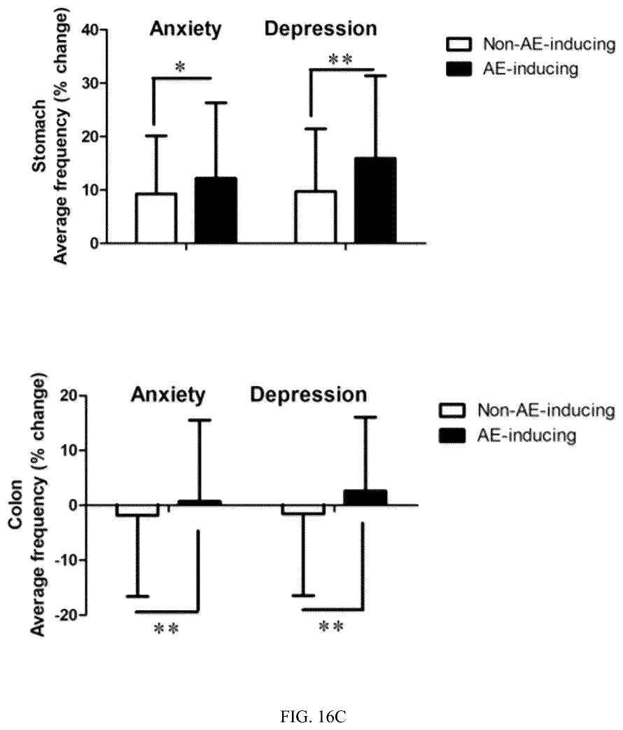

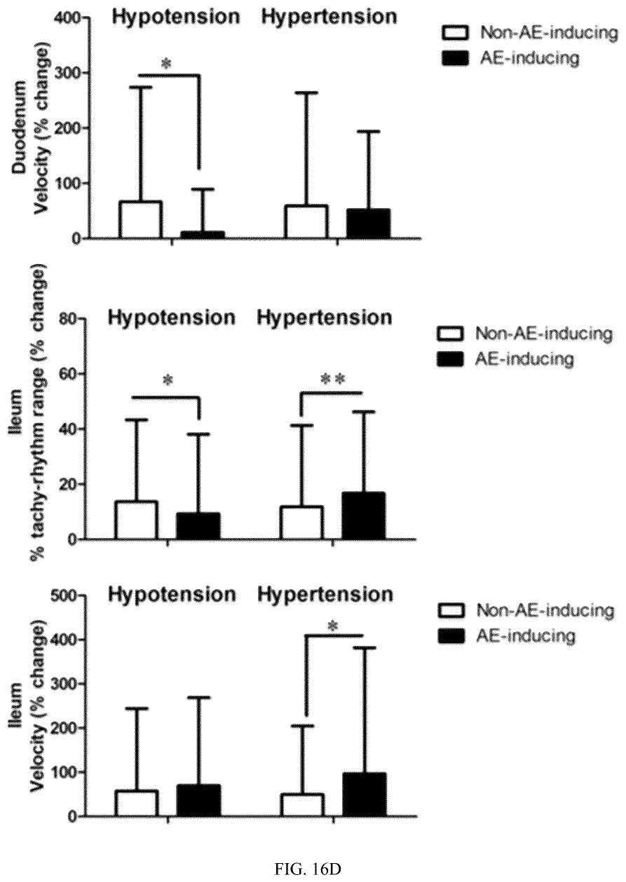

Embodiment 45. The method according to Embodiment 44, the step of clustering comprising application of a network graph cluster as shown in FIG. 16A.

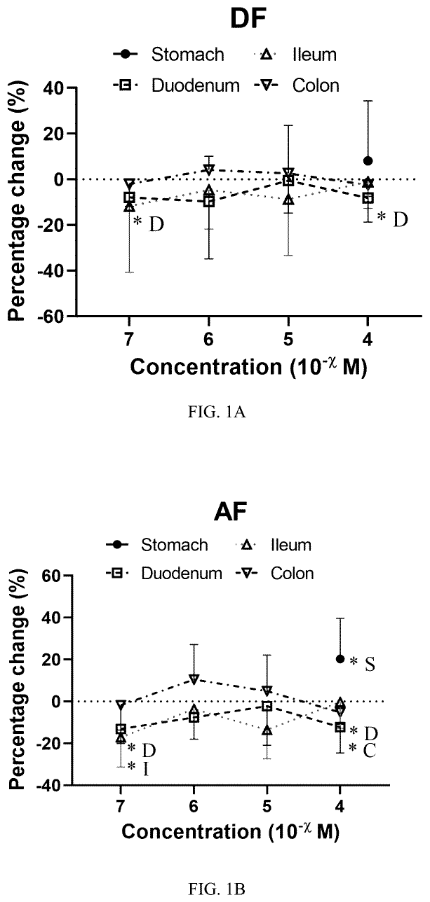

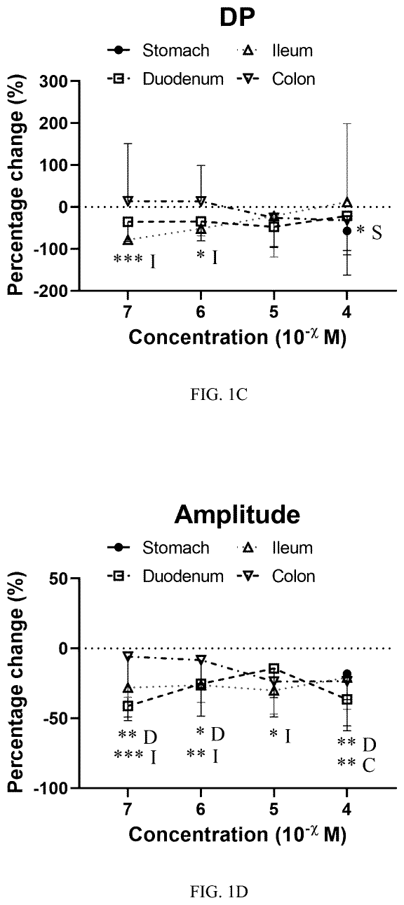

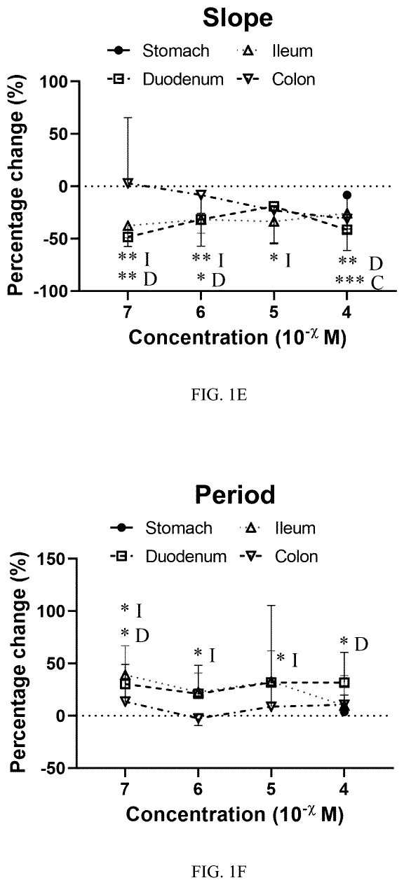

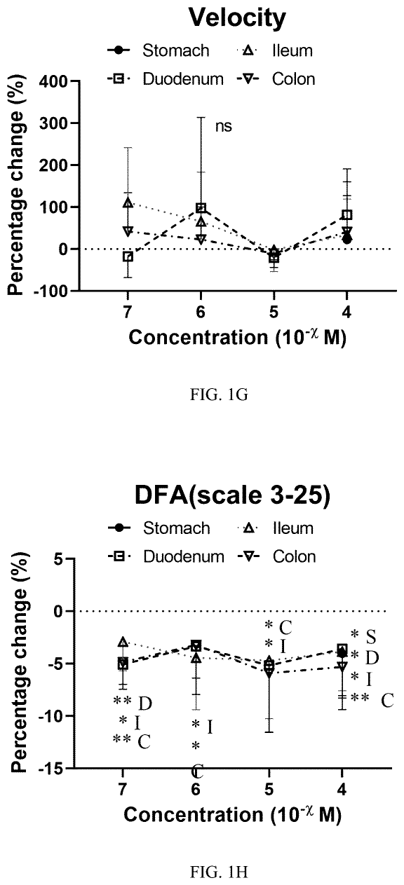

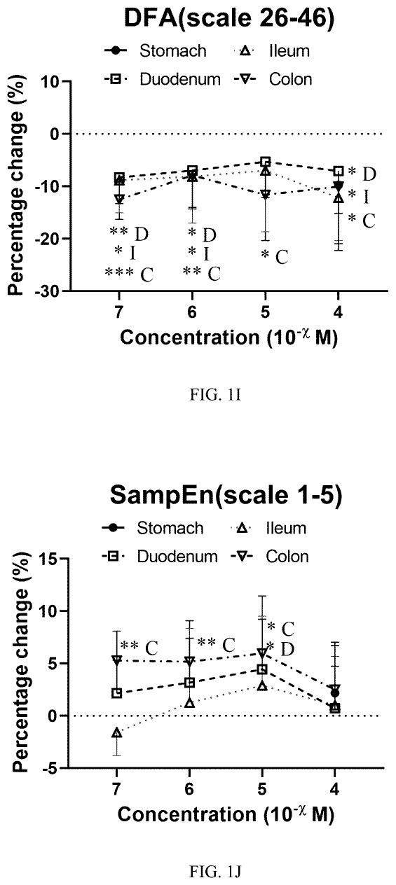

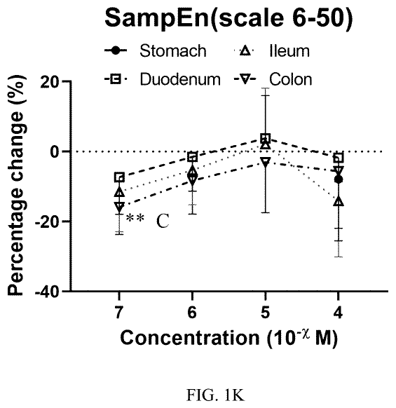

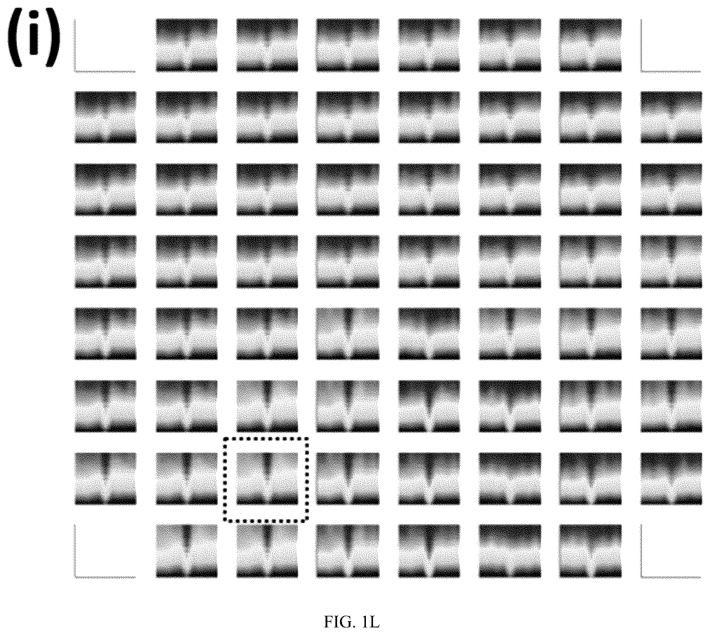

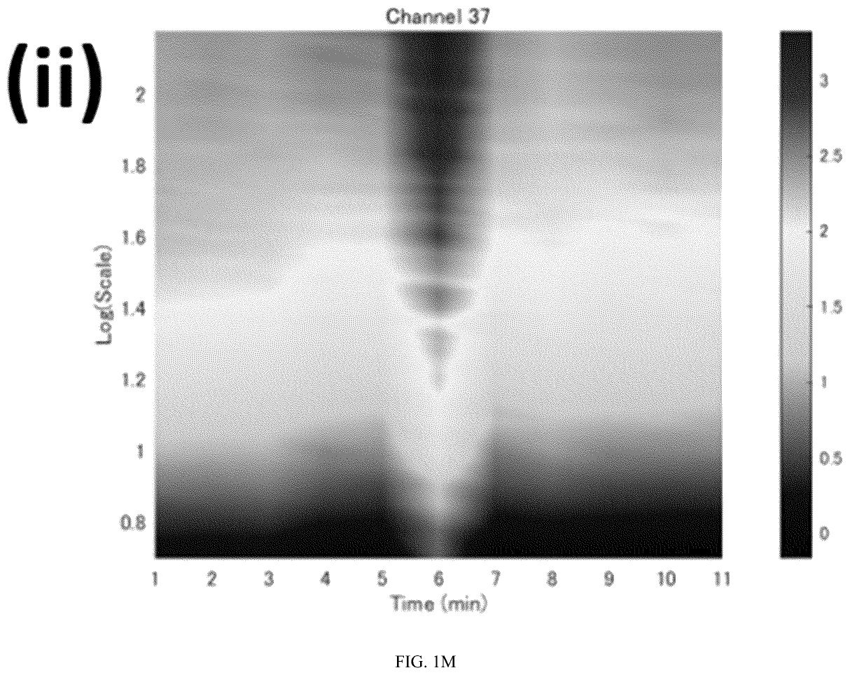

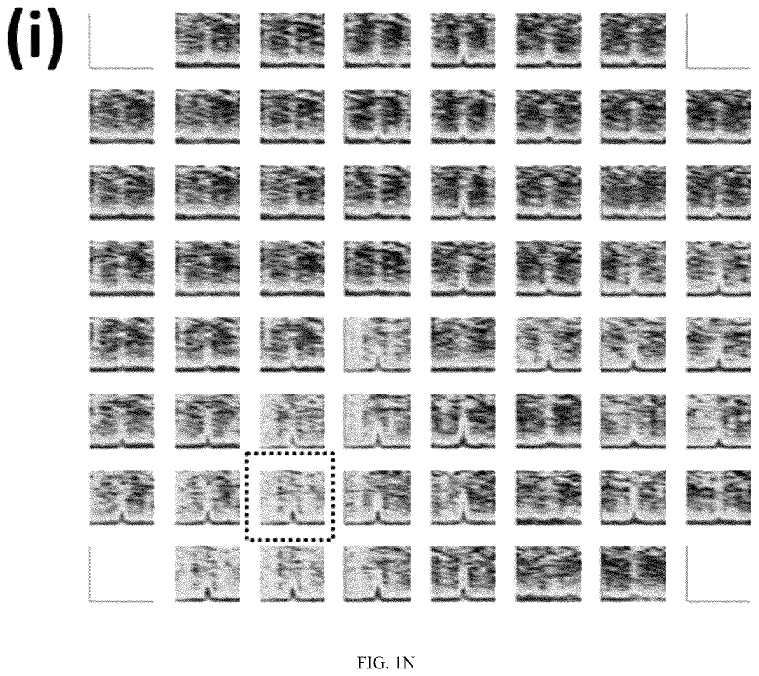

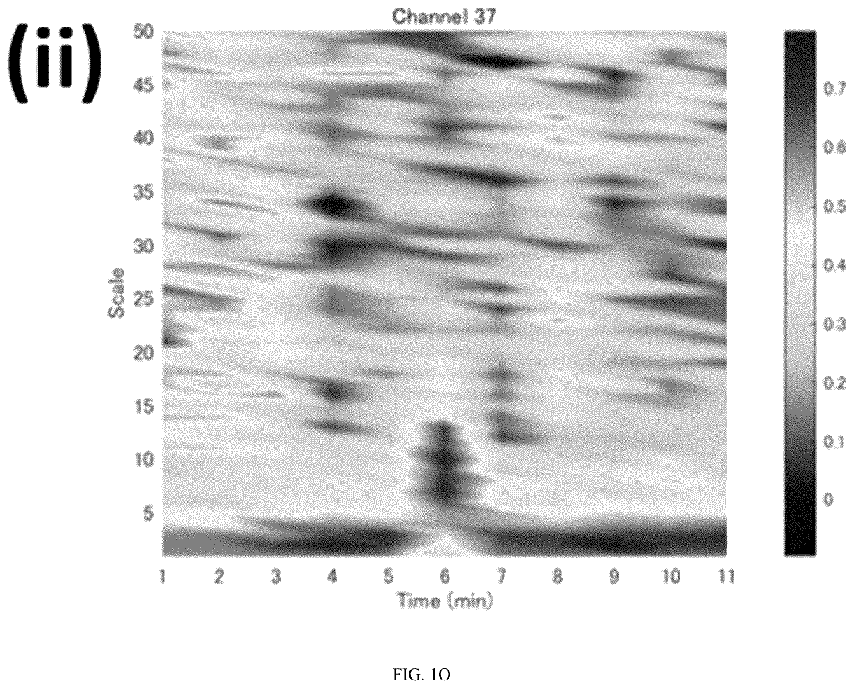

Turning now to the figures, FIGS. 1A-1O show certain effects of dopamine on pacemaker potentials along the gastrointestinal tract of Suncus murinus according to an embodiment of the subject invention. Slow-wave features including the (FIG. 1A) dominant frequency (DF), (FIG. 1B) average frequency (AF), and (FIG. 1C) dominant power (DP); the average (FIG. 1D) amplitude, (FIG. 1E) slope, and (FIG. 1F) period of the waveforms; (FIG. 1G) propagation velocity; detrended fluctuation analysis (DFA) fluctuation function (FIG. 1H) at smaller window scale (3-35) and (FIG. 1I) at larger window scale (26-46); and (FIG. 1J) sample entropy (SampEn) at smaller window scale (1-5) and (FIG. 1K) at larger window scale (5-60) are indicated as the percentage change after dopamine (0.1-100 μM) treatment, compared with baseline recordings. All data and error bars represent the means and the standard deviations, respectively. Significant differences in the true means of post-treatment data, relative to baseline data, are indicated as ‘*’ for p<0.05, ‘**’ for p<0.01, and ‘***’ for p<0.001 (paired Student's t-tests). No significant differences (ns) were identified for propagation velocity. (FIGS. 1L-M) The effects of dopamine (100 μM) on pacemaker potentials determined by DFA in isolated Suncus murinus ilea. For each scale and each time segment, the fluctuation functions of the corresponding sample were calculated. (FIG. 1L) DFA fluctuation functions of all 60 electrodes, where the scale is the same as the enlarged graph in (FIG. 1M), showing only channel 37. Drug administration artefacts occurring at approximately the 5-6 min time segment were not included in the statistical analyses. (FIGS. 1N-O) The effects of dopamine (100 μM) on pacemaker potentials determined by SampEn in isolated Suncus murinus ilea. Entropy analysis consisted of a scale from 0-50. (FIG. 1N) The SampEn of all 60 electrodes, where the scale is the same as the enlarged graph in (FIG. 1O), showing only channel 37. Drug administration artefacts occurring at approximately the 5-6 min time segment were not included in the statistical analyses.

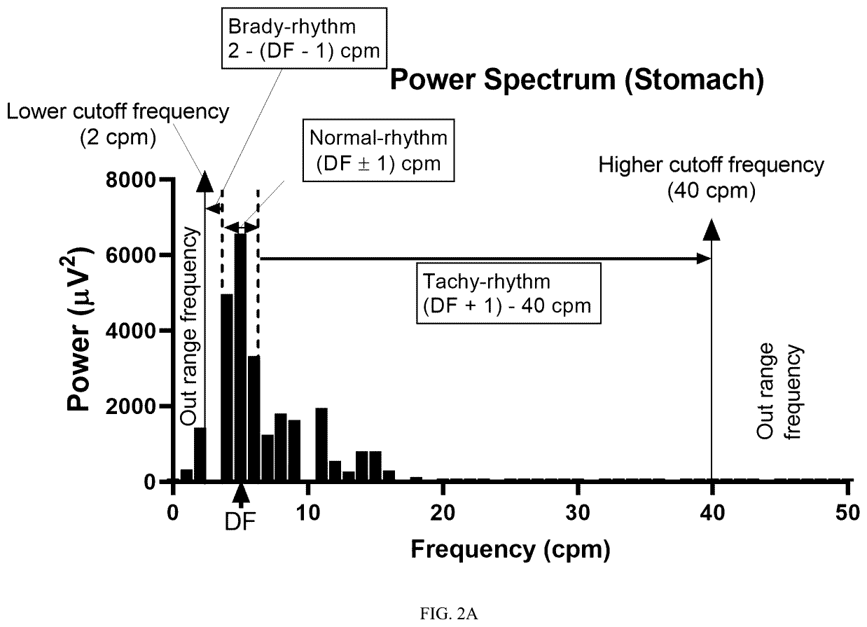

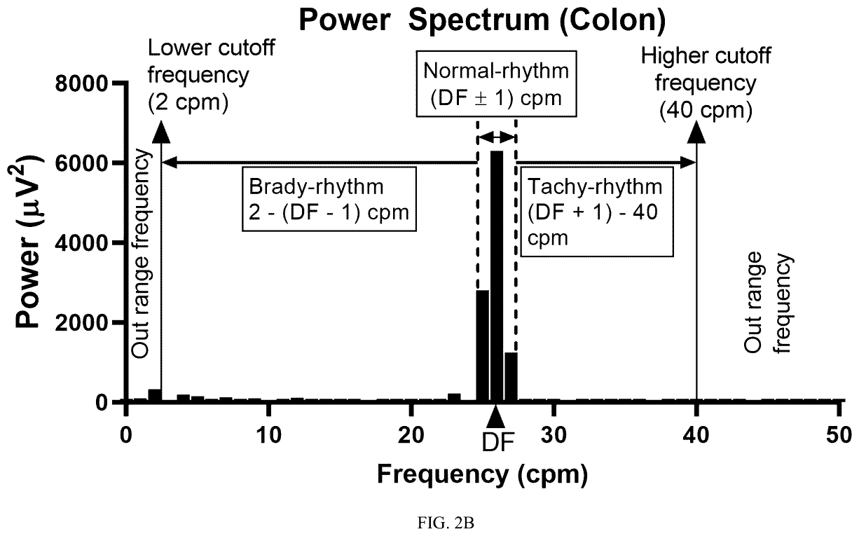

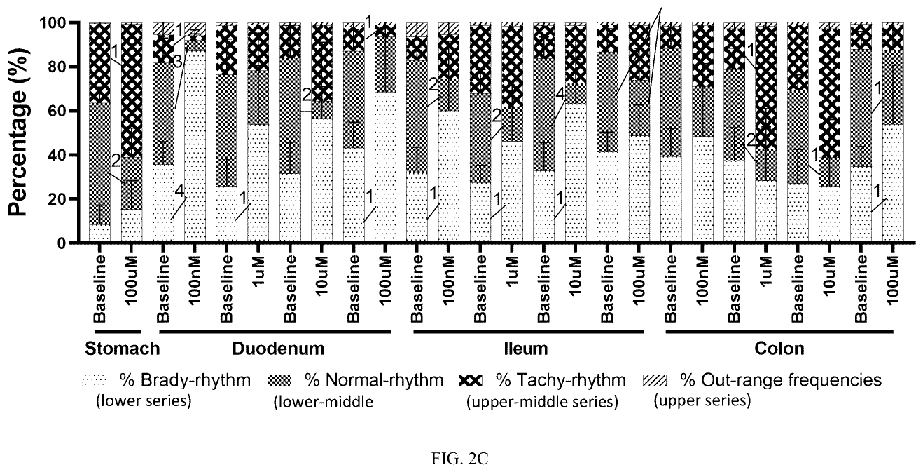

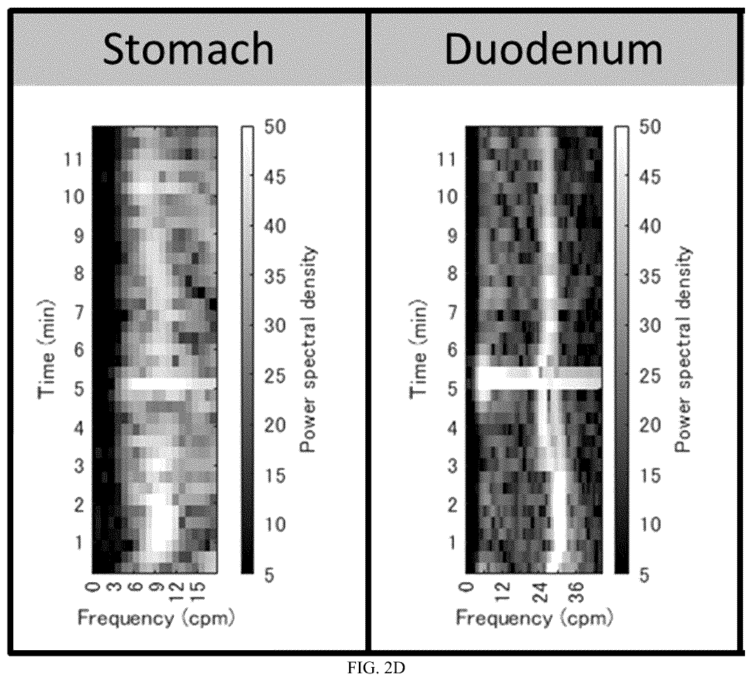

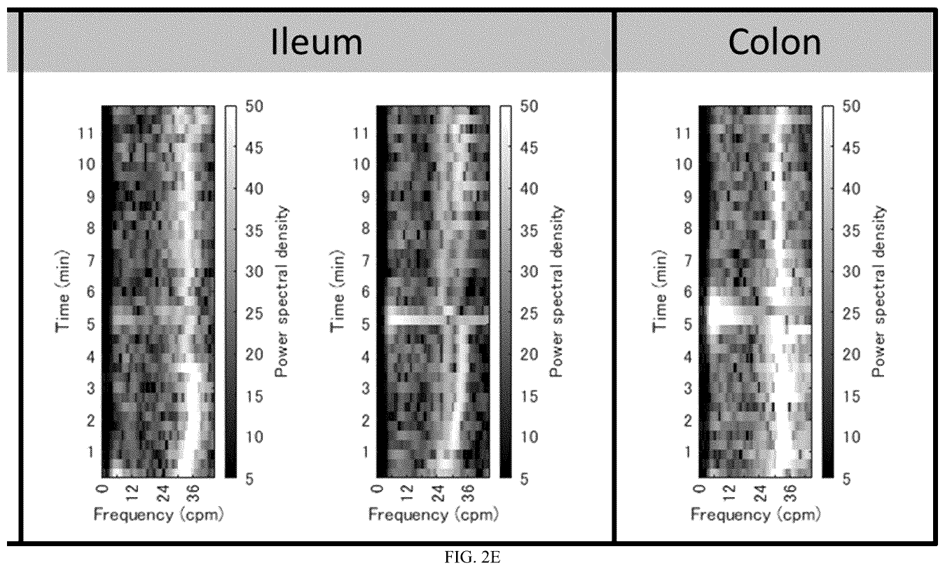

FIGS. 2A-2E show certain effects of dopamine on pacemaker potentials determined by the frequency shifting behavior of the power spectrum according to an embodiment of the subject invention. Power spectra were generated using 5 min baseline data obtained from the (FIG. 2A) stomach and (FIG. 2B) colon using fast Fourier transform analysis with a bin size of 2048 and a Hanning window. The dominant frequency (DF) was defined as the frequency bin with the highest power. The percentage of normal-rhythm range was defined as the percentage of power within DF+1 frequency bins over the total power (0-50 cpm). The percentage of brady-rhythm range was defined as the percentage of power within 2-(DF−1) cpm over the total power. The percentage of tachy-rhythm range was defined as the percentage of power within (DF+1)-40 cpm over the total power. Out-of-range frequencies were defined as the percentage of power<2 cpm and >40 cpm over the total power. (FIG. 2C) Stacked histograms showing the effects of dopamine (0.1-100 μM) on frequency partitioning along the gastrointestinal tract of Suncus murinus. The dominant frequency was first defined by the baseline, and changes in the percentage of frequency ranges were compared between the recordings at baseline and after drug treatment. All data and error bars represent the means and standard deviations, respectively. Significant differences relative to the baseline are indicated as ‘1’ for p<0.05, ‘2’ for p<0.01, and ‘3’ for p<0.001 (paired Student's t-tests). (FIGS. 2D-E) Representative spectrograms showing the effects of 100 M dopamine on the stomach, and the effects of 10 μM dopamine on the duodenum, ileum, and colon of Suncus murinus. Spectrograms were generated by subdividing the total length of raw data (11 min) into 0.55 min windows and plotting them against time, with a 50% overlap. Drug administration artefacts occurring at approximately the 5 min time segment were not included in the statistical analyses.

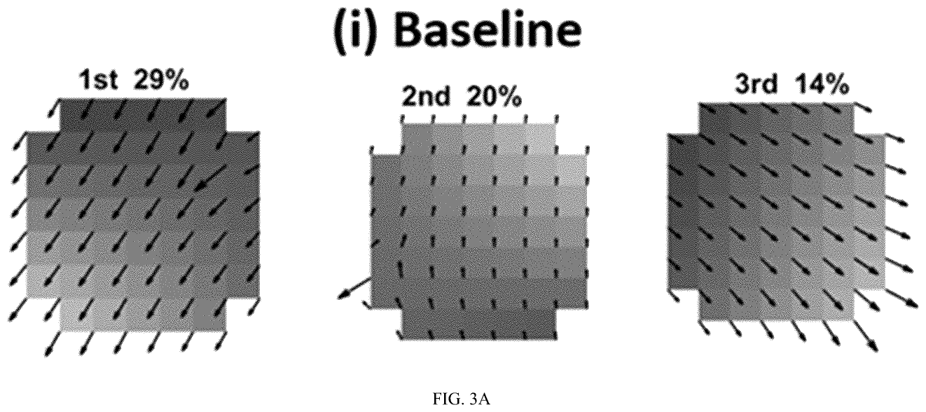

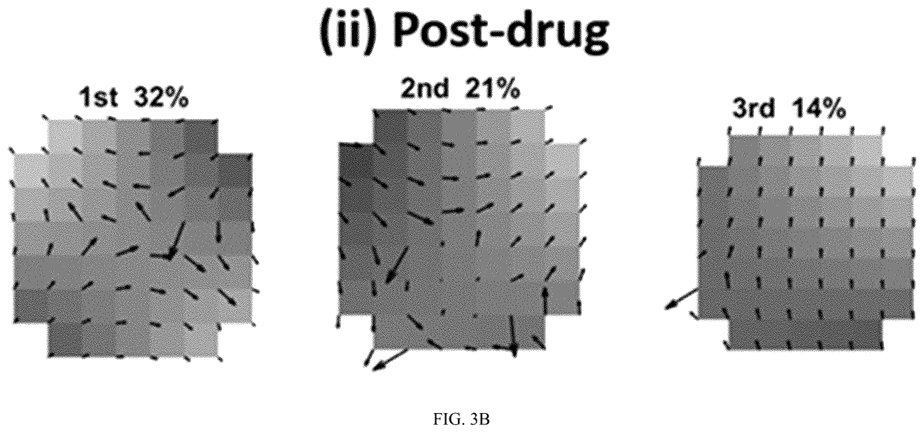

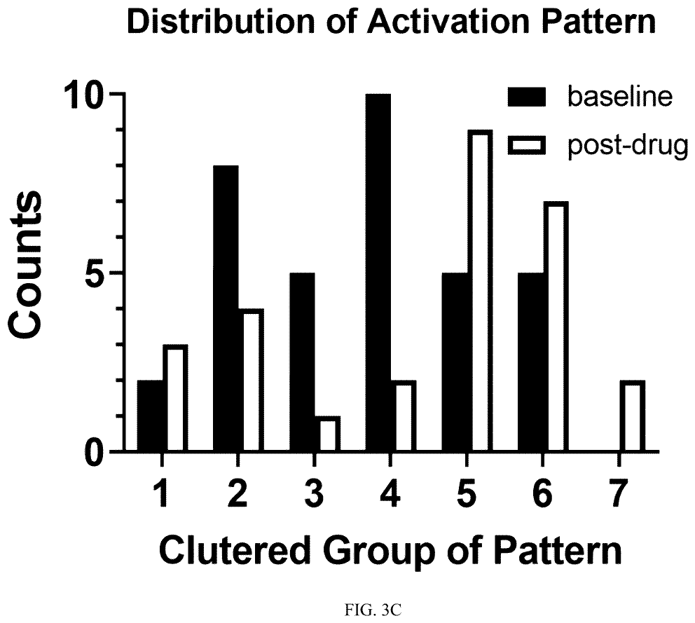

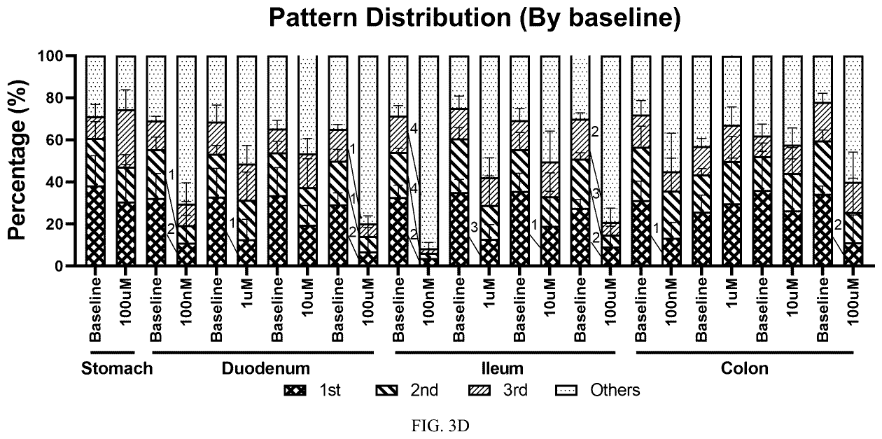

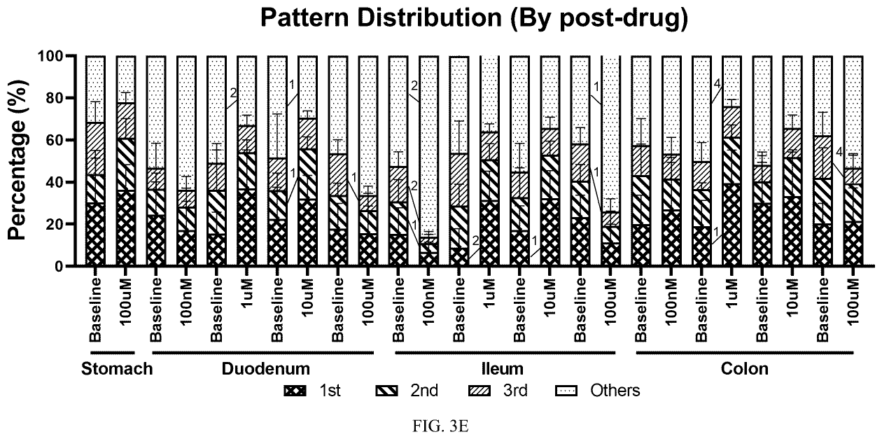

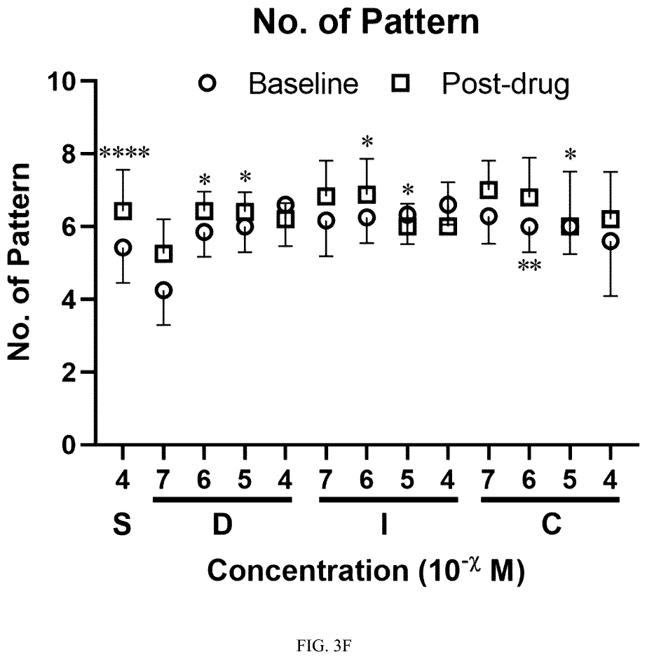

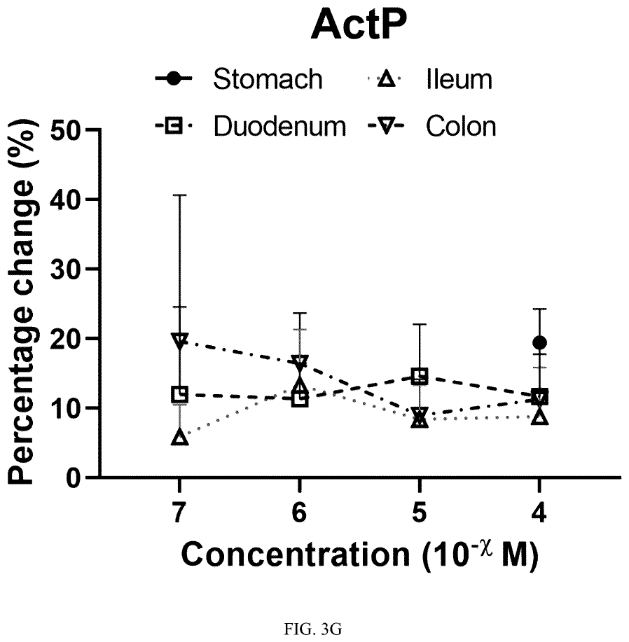

FIGS. 3A-3G show certain effects of dopamine on pacemaker potentials determined by the activation time pattern distribution according to an embodiment of the subject invention. (FIGS. 3A-C) Representative figures showing the three most dominant activation time patterns within the (FIG. 3A) baseline recordings and the (FIG. 3B) post-treatment recordings. (FIG. 3C) The counts of the appearance of each grouped activation time pattern determined at 20 s intervals. The pattern distribution of the dominant activation time patterns based on the (FIG. 3D) baseline and (FIG. 3E) post-treatment recordings. All data and error bars represent the means and standard deviations, respectively. Significant differences relative to the baseline are indicated as ‘1’ for p<0.05, ‘2’ for p<0.01, and ‘3’ for p<0.001 (paired Student's t-tests). (FIG. 3F) The number of grouped activation time patterns within the baseline and post-treatment recordings. All data and error bars represent the means and standard deviations, respectively. Significant differences relative to the baseline are indicated as ‘*’ for p<0.05, ‘**’ for p<0.01, and ‘****’ for p<0.0001 (paired Student's t-tests). (FIG. 3G) The total percentage change of all activation time patterns induced by dopamine treatment is indicated as ActP. No statistical analysis was performed.

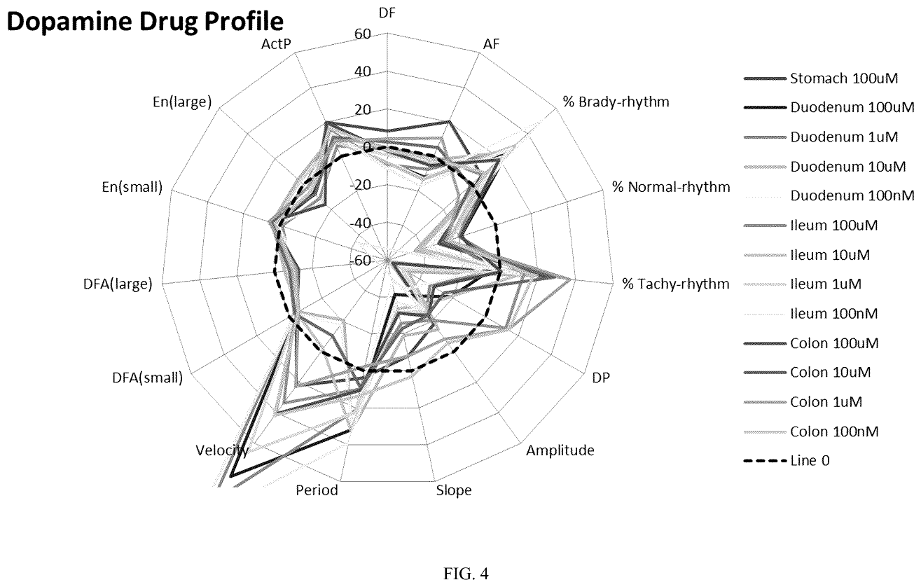

FIG. 4 shows a radar diagram showing the profile of dopamine effects on pacemaker potentials along the gastrointestinal tract of Suncus murinus according to an embodiment of the subject invention. All data show the mean percentage change in slow-wave features, including the dominant frequency (DF); average frequency (AF); percentage of brady-rhythm, normal rhythm, and tachy-rhythm; dominant power (DP); average amplitude, slope, and period of the waveform; average propagation velocity; detrended fluctuation analysis (DFA) fluctuation function with small window scale 3-25 and large window scale 26-46; sample entropy (SampEn) with small window scale 1-5 and large window scale 6-50; and the total change in activation time pattern (ActP). The radar diagram provides an immediate indication of how dopamine affects GI pacemaker activity across selected parameters in one graph. For example, at the AF, the red and purple lines (stomach and colon) lie above the line ‘0’, while the blue and green lines (duodenum and ileum) lie below line 0, indicating a potential tissue-segment-dependent effect of dopamine. To determine whether there are significant differences, p-values obtained using paired Student's t-tests can be used to compare pre- and post-treatment recordings (see Table 2).

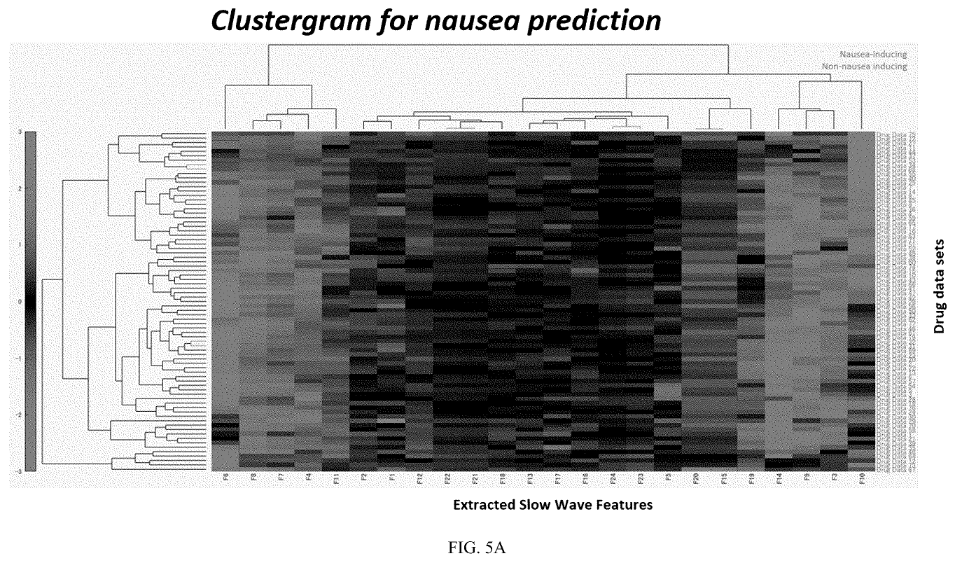

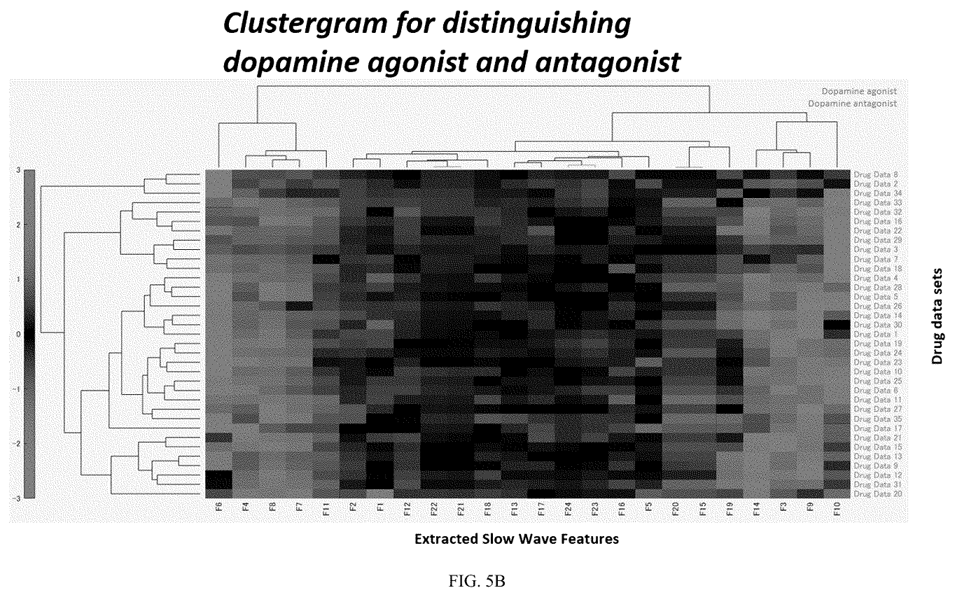

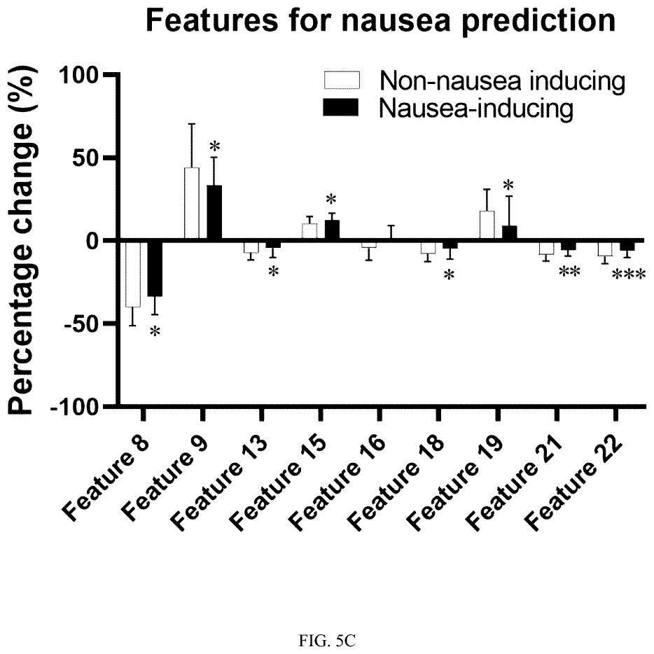

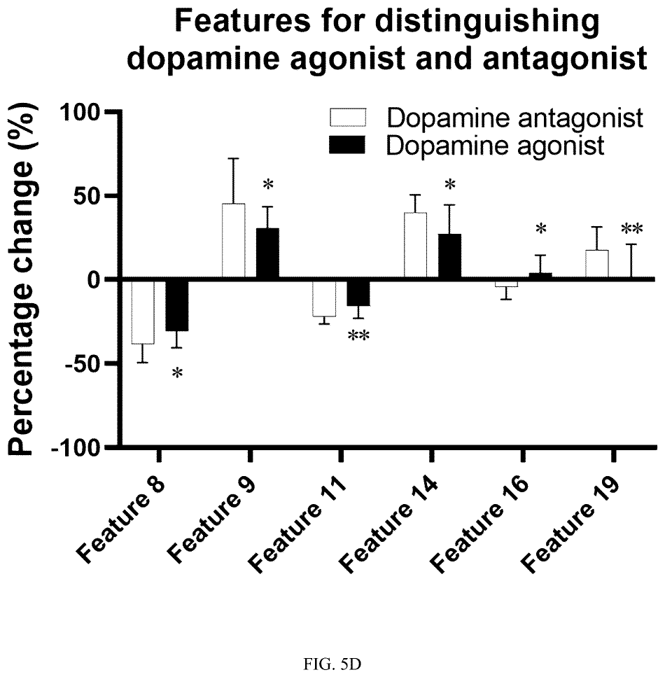

FIG. 5A-5D illustrate an example application of a drug database built under the standardized methodologies according to an embodiment of the subject invention. A clustergram showing the 24 slow-wave features extracted from the duodenal data for a list of drugs (FIG. 5A) with known nausea-inducing or non-nausea-inducing properties and (FIG. 5B) known to be agonists or antagonists of the dopamine receptor. Features extracted with significant differences between (FIG. 5C) the nausea-inducing and non-nausea-inducing drugs and (FIG. 5D) the dopamine agonists and antagonists. All data and error bars represent the means and standard deviations, respectively. Significant differences between the two groups are indicated as ‘*’ for p<0.05, ‘**’ for p<0.01, and ‘***’ for p<0.001 (unpaired Student's t-tests).

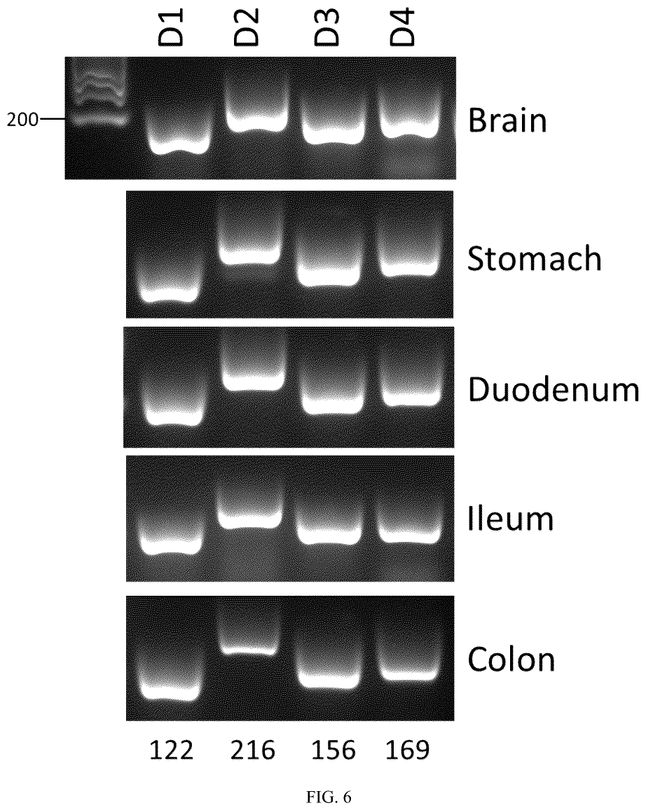

FIG. 6 illustrates the mRNA expression of dopamine receptors in Suncus murinus according to an embodiment of the subject invention.

GelRed® electrophoresis of PCR amplification products with expected sizes of 122, 216, 156, and 169 bp for dopamine receptor subtypes D1-D4 in the brain, stomach, duodenum, ileum, and colon of Suncus murinus.

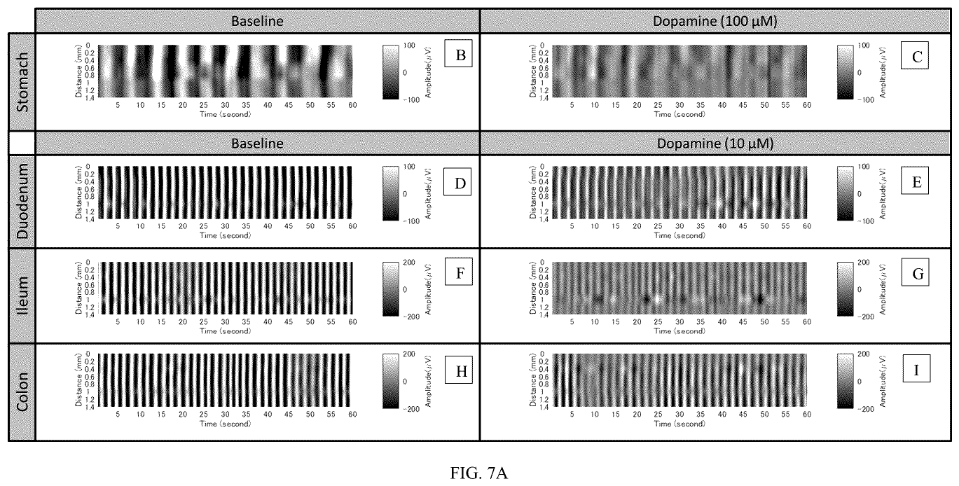

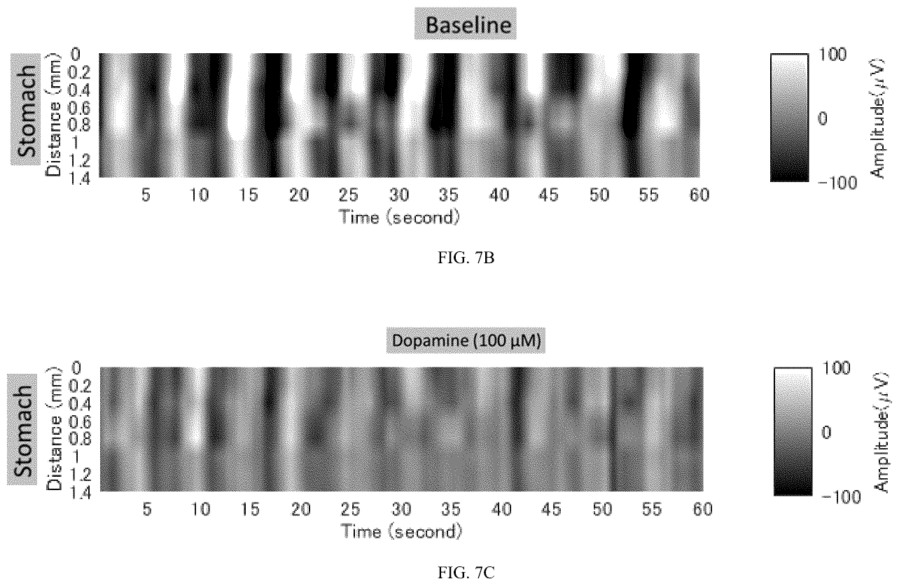

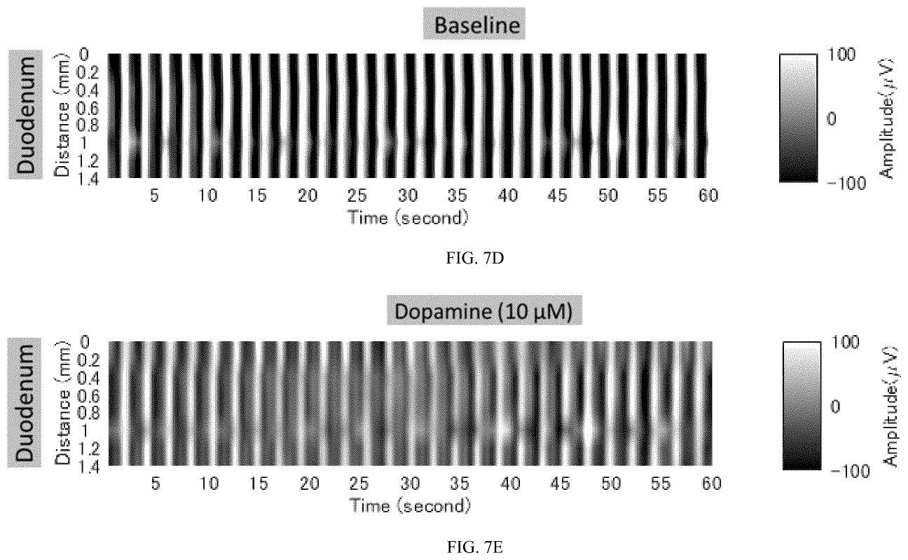

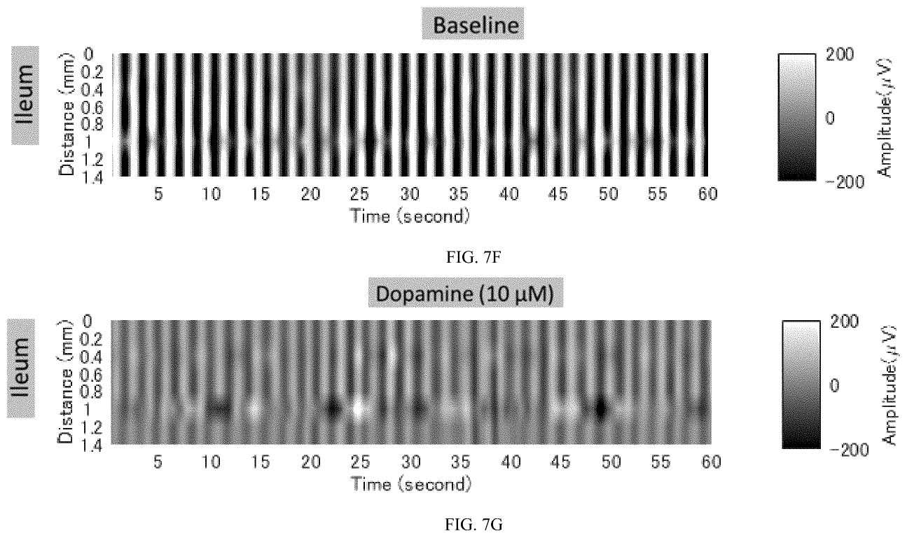

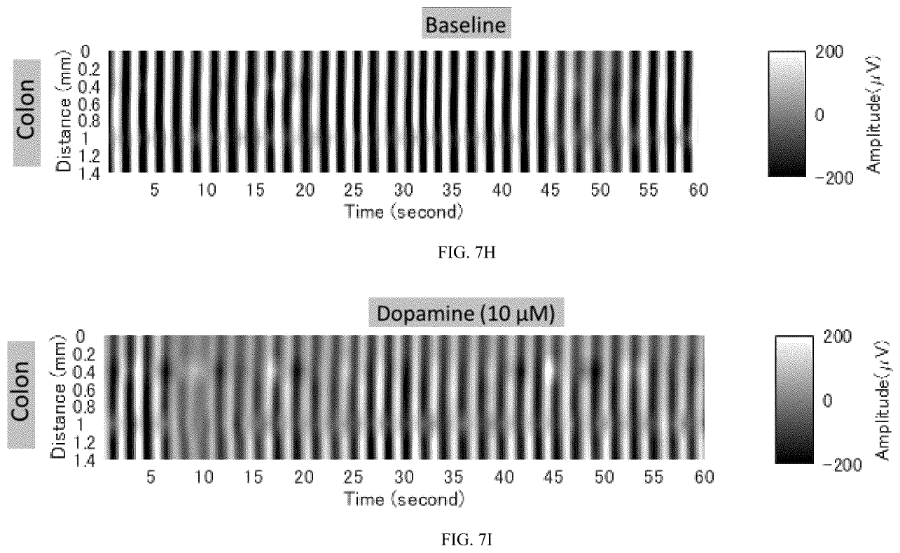

FIGS. 7A-7I illustrate representative spatio-temporal maps showing the effects of 100 M dopamine on the stomach, and the effects of 10 μM dopamine on the duodenum, ileum, and colon of Suncus murinus according to an embodiment of the subject invention. Spatio-temporal maps were generated by aligning raw traces extracted from a vertical or horizontal line of electrodes (total of eight electrodes), with a 0.2 mm gap between each electrode. The y-axis represents the distance in mm. One minute of representative data were extracted from the baseline and post-treatment recordings.

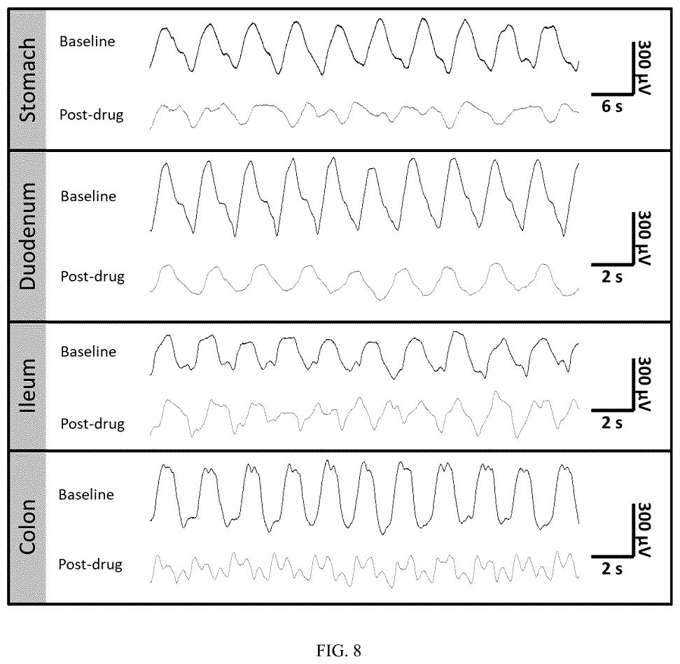

FIG. 8 illustrates representative raw traces showing the effects of 100 μM dopamine on the stomach, and the effects of 10 μM dopamine on the duodenum, ileum, and colon of Suncus murinus according to an embodiment of the subject invention. Sixty seconds of stomach and 20 s of intestinal raw traces were directly exported from unfiltered raw data recorded from the MEA. The troughs were manually aligned at the time segment extracted to allow better visualization.

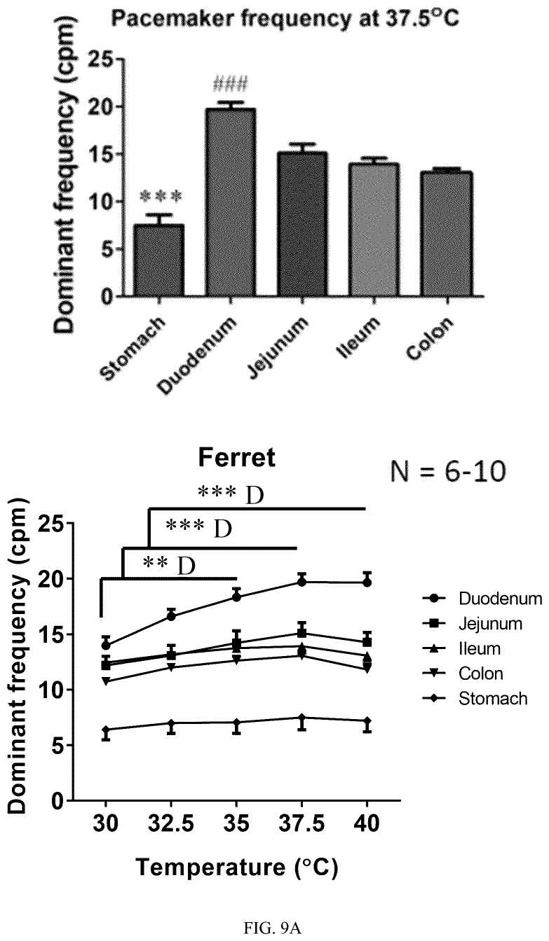

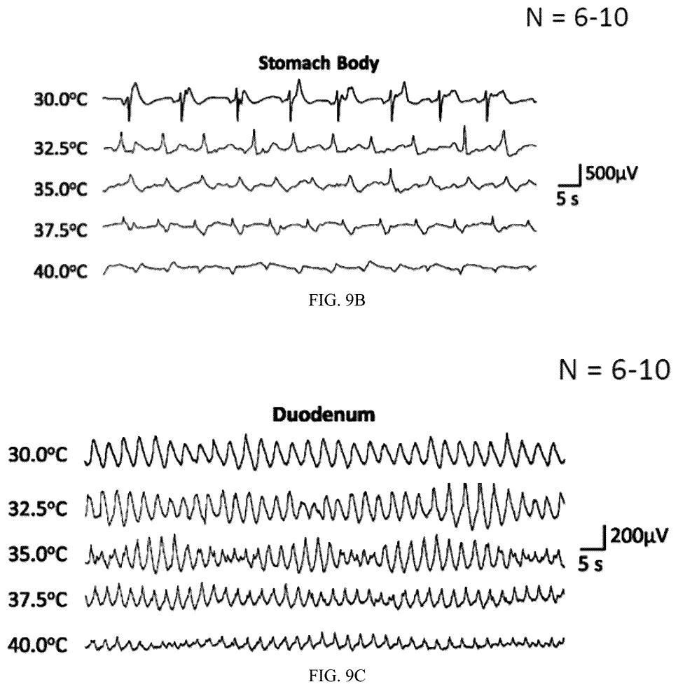

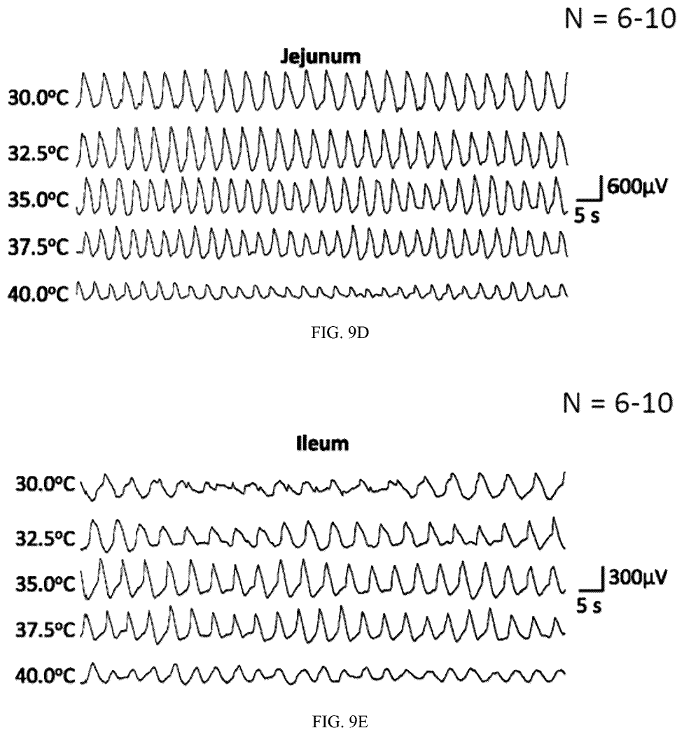

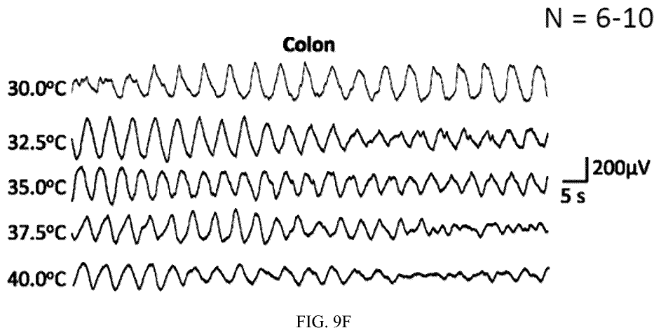

FIGS. 9A-9F illustrate representative data collected on ferrets according to an embodiment of the subject invention.

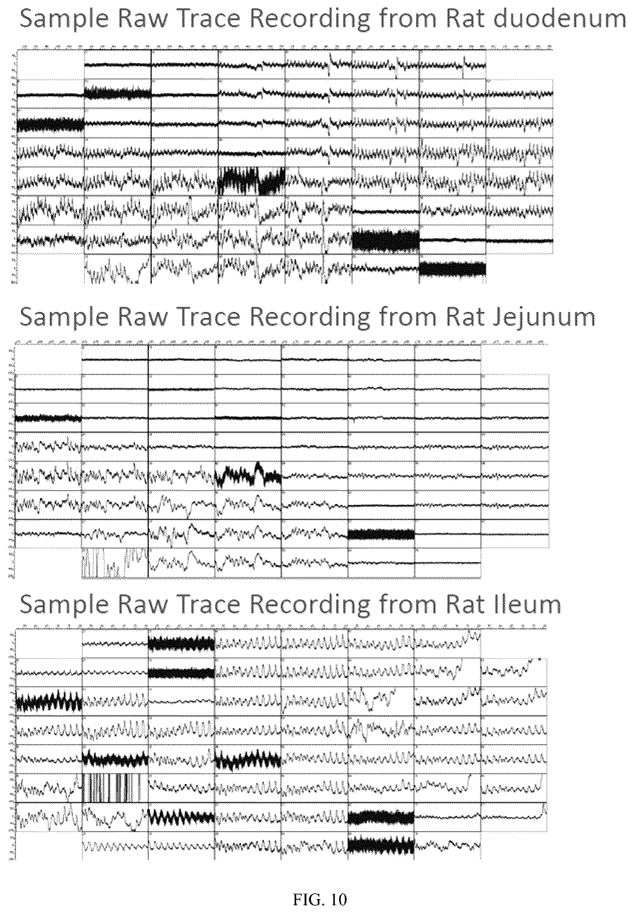

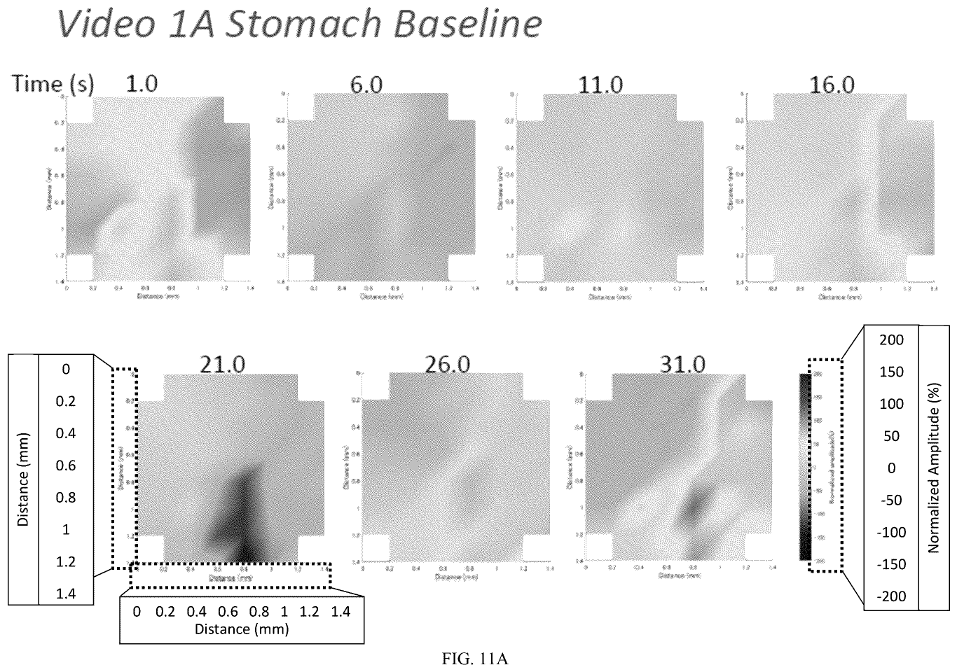

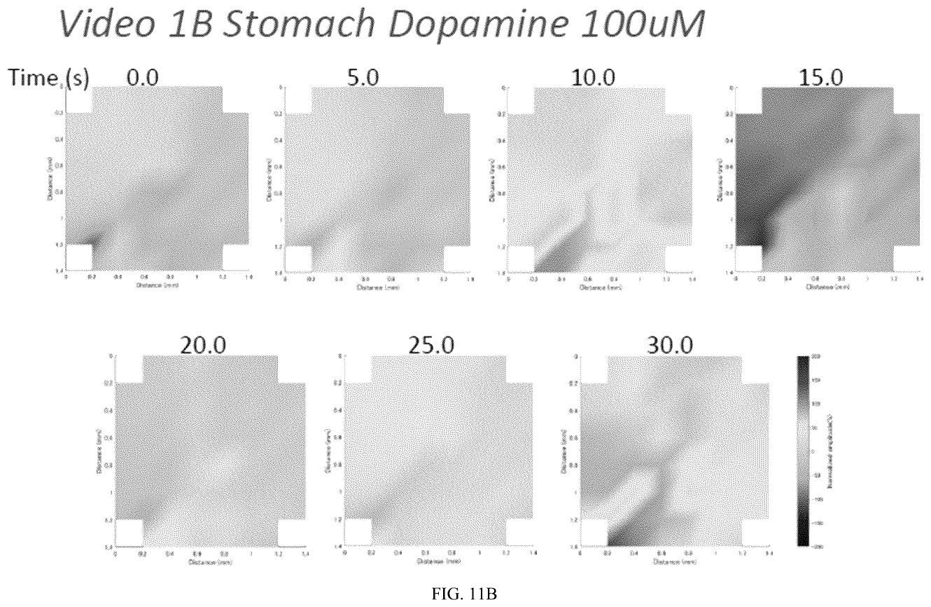

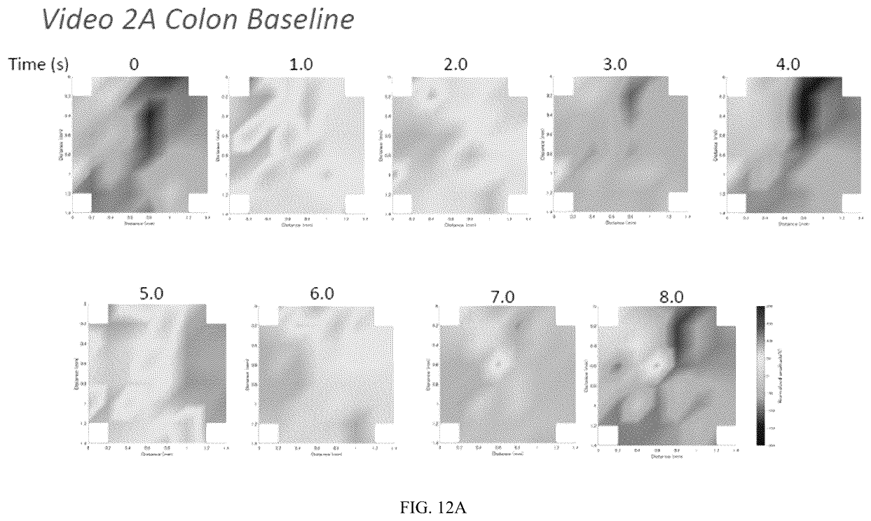

FIG. 10 illustrates representative raw data collected on rats according to an embodiment of the subject invention.

FIGS. 11A and 11B illustrate representative progression of screen shots from a video clip showing the effects of 100 μM dopamine on wave propagation in the stomach according to an embodiment of the subject invention at (FIG. 11A) baseline and (FIG. 11B) after drug treatment. The duration of the video is 30 s, with a frame rate of 10 and a ½ playback speed. The amplitude is normalized to the maximum amplitude in the baseline recording for each channel.

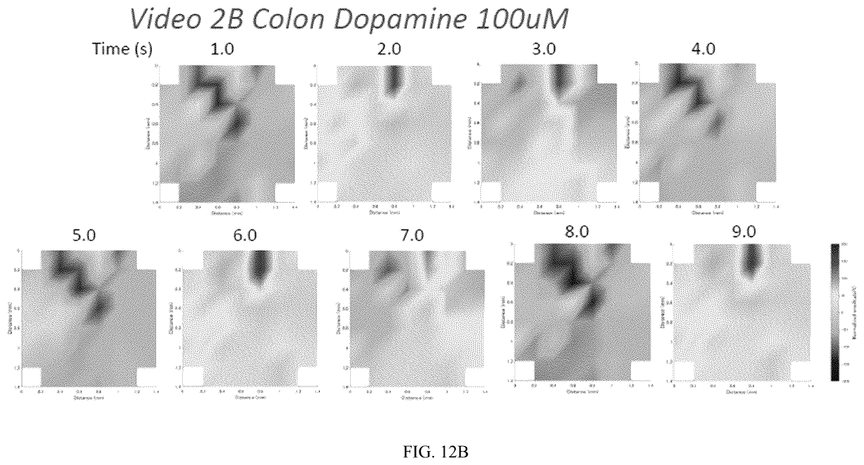

FIGS. 12A and 12B illustrate representative progression of screen shots from a video clip showing the effects of 100 μM dopamine on wave propagation in the colon according to an embodiment of the subject invention at (FIG. 12A) baseline and (FIG. 12B) after drug treatment. The duration of the video is 30 s, with a frame rate of 10 and a ½ playback speed. The amplitude is normalized to the maximum amplitude in the baseline recording for each channel.

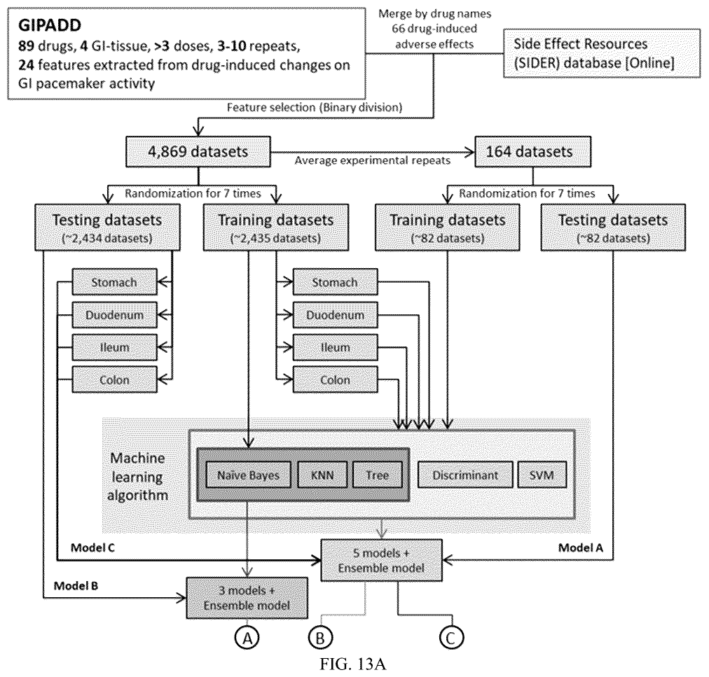

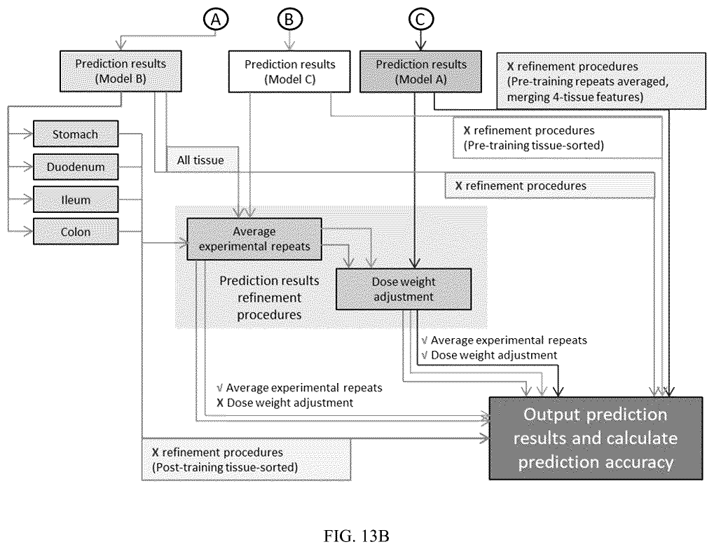

FIGS. 13A and 13B illustrate a flow chart showing the process from dataset preparation, to ML model training, to prediction result refinement for generating different types of ML models according to an embodiment of the subject invention.

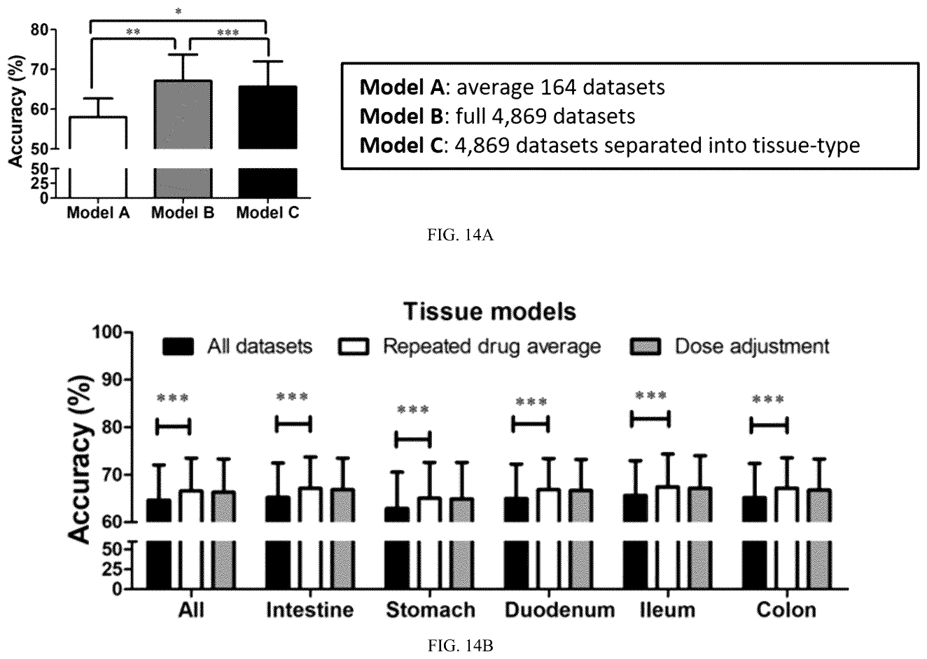

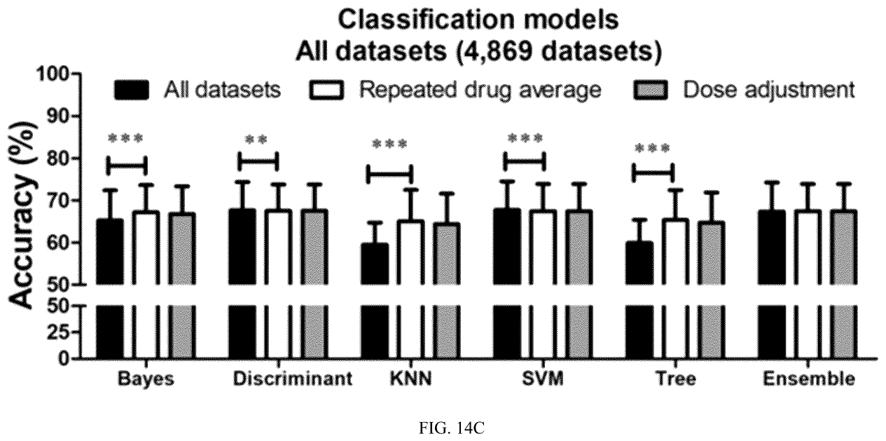

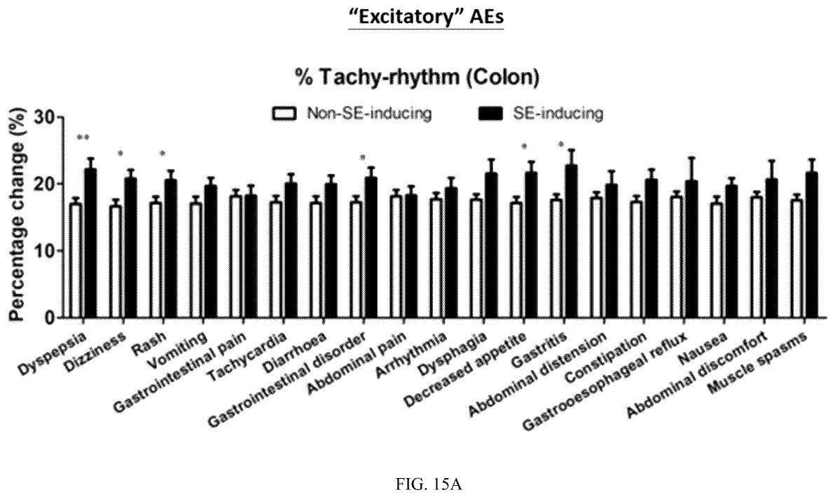

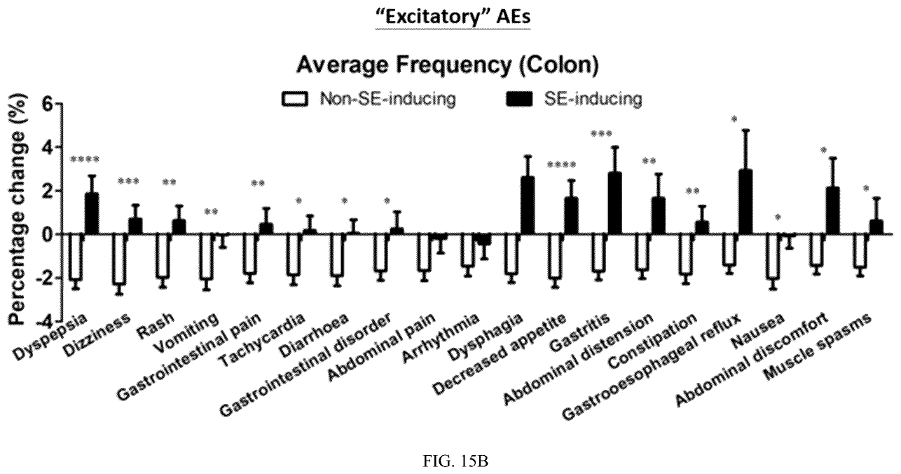

FIGS. 14A-14C illustrate model comparisons according to an embodiment of the subject invention. (FIG. 14A) The prediction accuracy of models built using different dataset preparations for refined selected-AEs (n=10-13); (FIG. 14B) The prediction accuracy of different tissue models. ‘All’ column indicates average of all 4-tissue-type (n=2,016) and ‘Intestine’ column indicates average of 3 intestinal segments except stomach (n=1,554), stomach and ileum (n=462), duodenum and colon (n=546); (FIG. 14C) The prediction accuracy of model trained using different classification algorithms (n=336). Data represents the mean±S.D. Significant differences are indicated as * p<0.05, ** p<0.01, *** p<0.001 using paired t-tests.

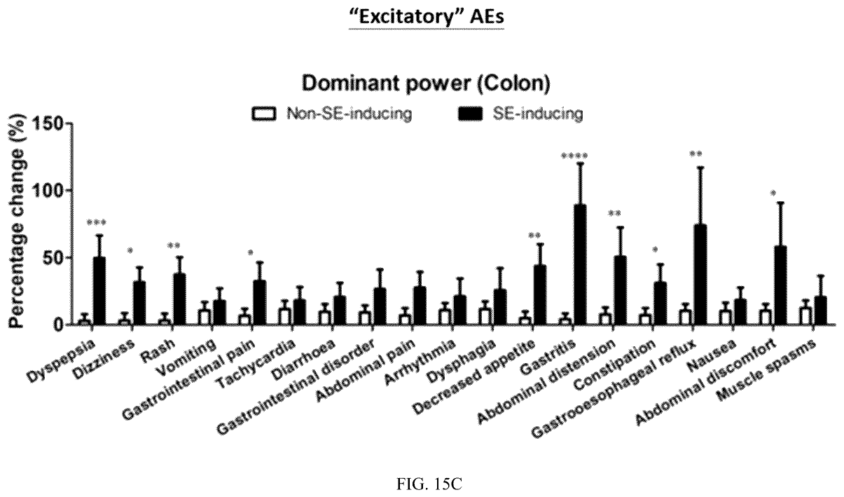

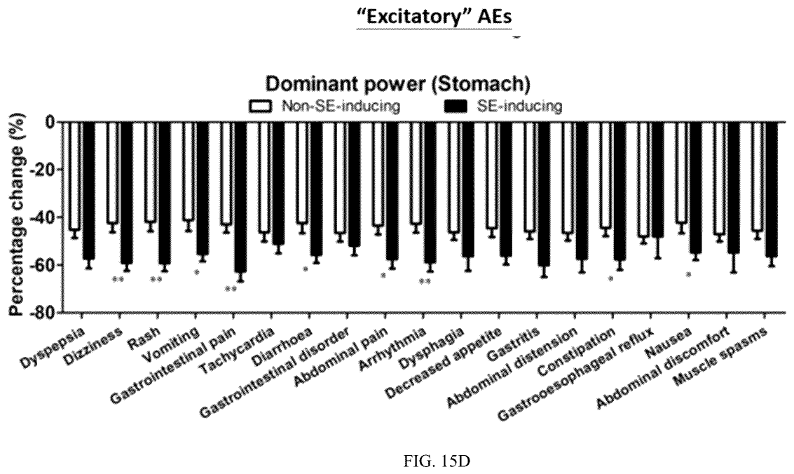

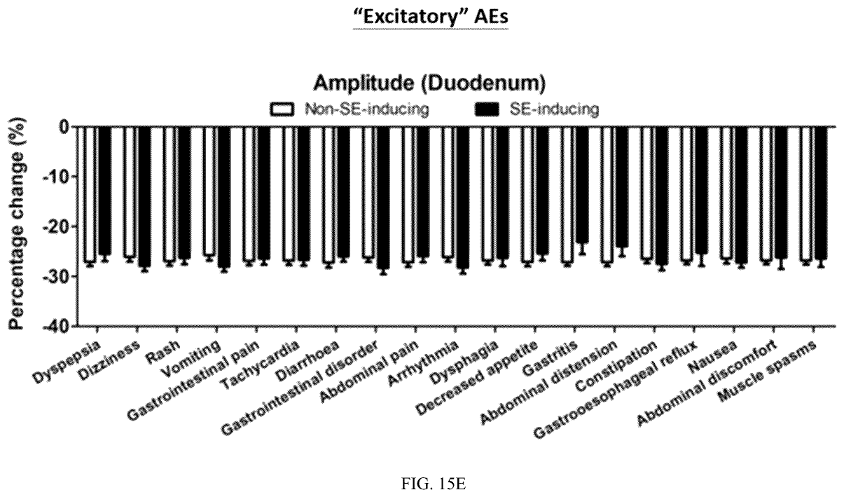

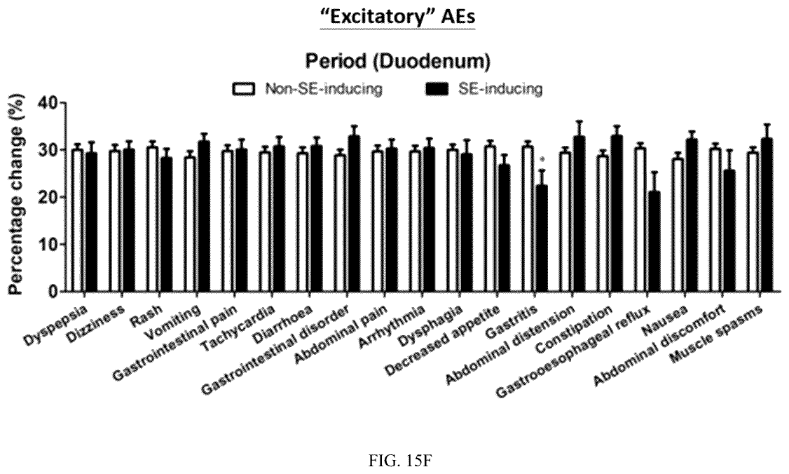

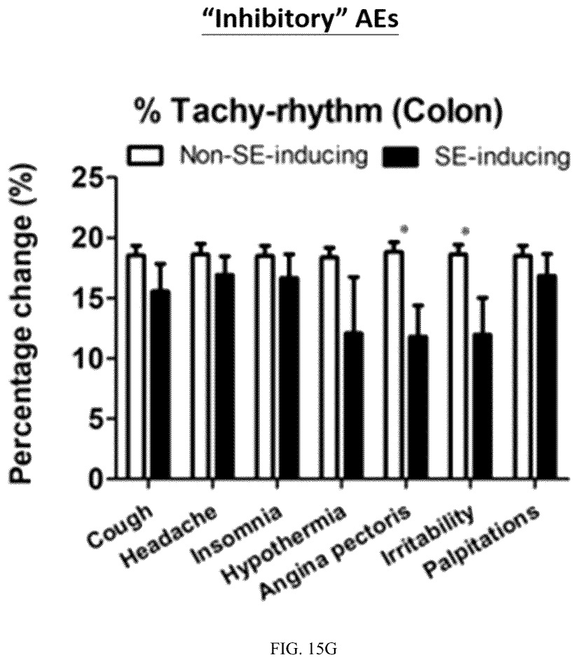

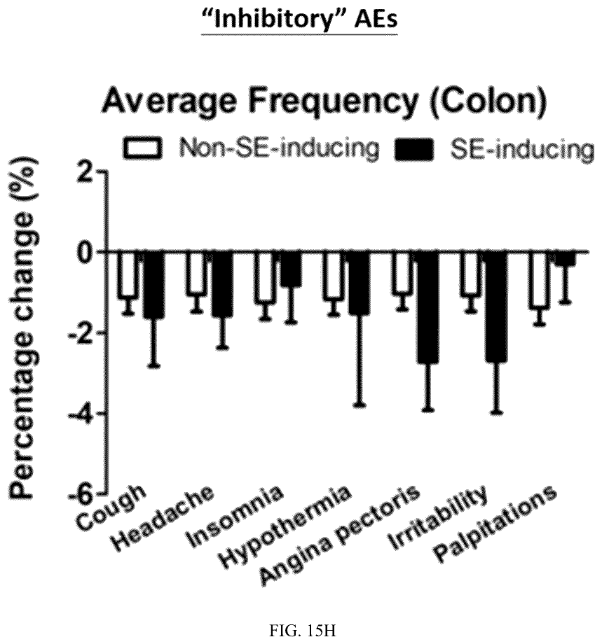

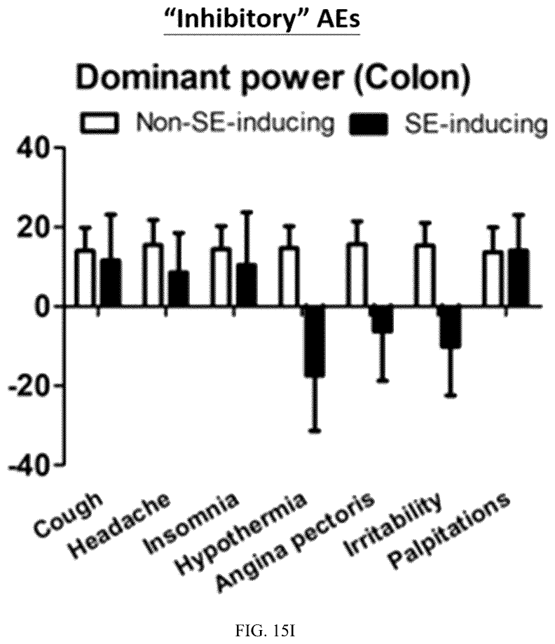

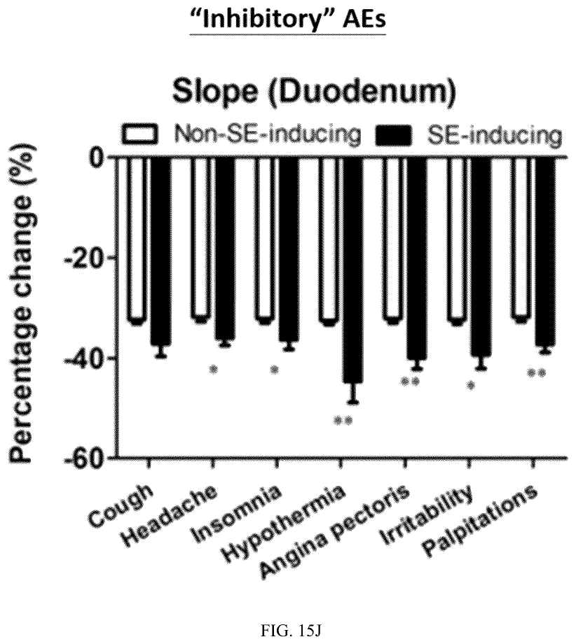

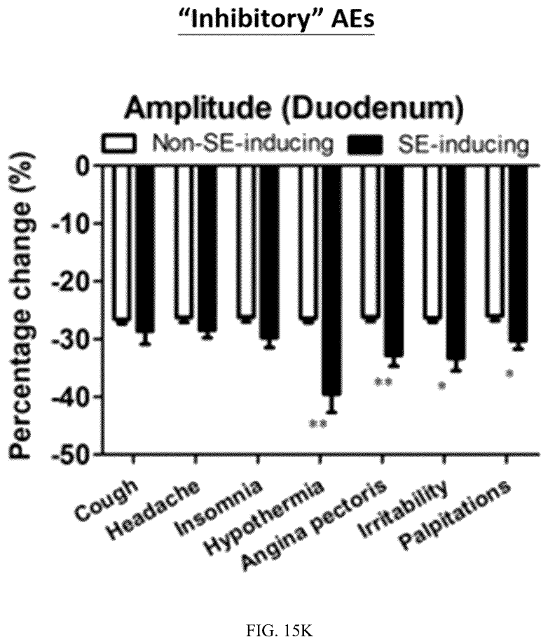

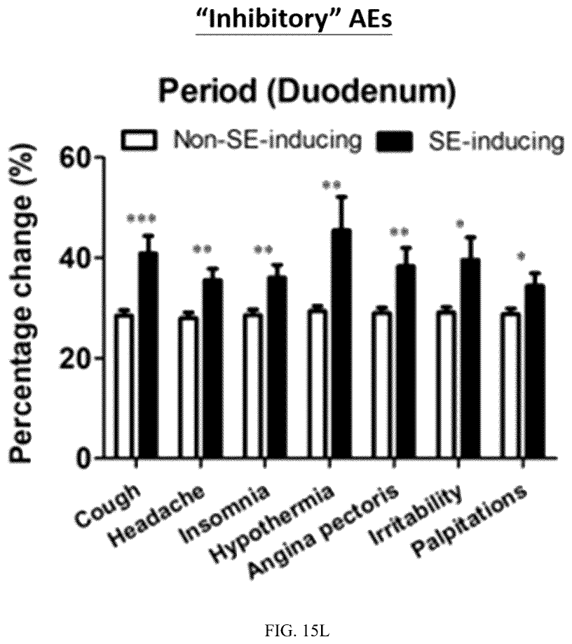

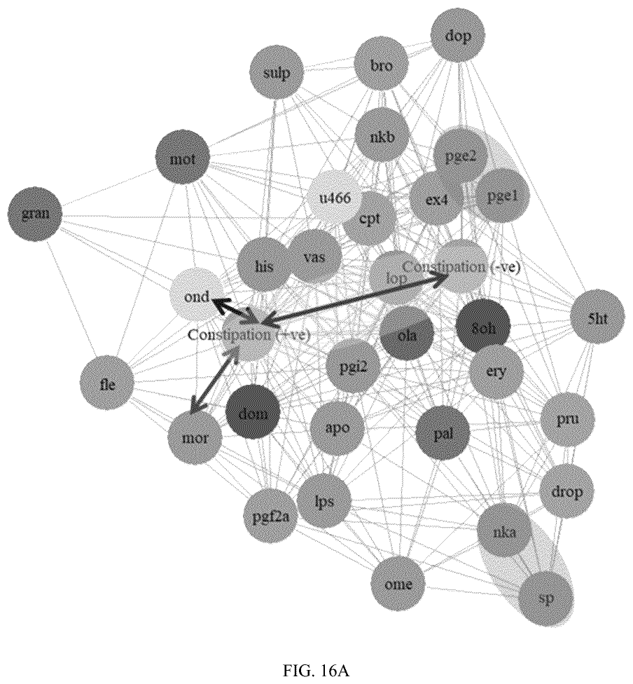

FIGS. 15A-15L illustrate excitatory and Inhibitory correlations to GI pacemaker activity in selected-AEs according to an embodiment of the subject invention. The percentage change of selected features in various AE-inducing drugs and non-AE-inducing drugs. Data represents the mean±S.D. Significant differences are indicated as * p<0.05, ** p<0.01, *** p<0.001 using unpaired t-tests (n=46-1,326).

FIGS. 16A-16D illustrate selected GI pacemaker features correlated with AEs according to an embodiment of the subject invention. (FIG. 16A) Network graph clustering selected drugs by refined EFs to an example AE: constipation. The length of black arrow represents how good the model can distinguish between positive-correlated features and negative-correlated features in constipation (shaded in yellow). Blue arrow and Red arrow has a short distance to positive-correlated features of constipation, where these two drugs ondansetron (“ond”) and morphine (“mor”) are known in market to induce constipation. Drugs acting on similar receptors, such as prostaglandin E1 (“pge1”) and prostaglandin E2 (“pge2”) or substance P (“sp”) and neurokinin A (“nka”) are clustered close to each other based on EF drug profile (shaded in yellow) (FIGS. 16B-D) The percentage change of selected features compared between AE-inducing drugs and non-AE-inducing drugs in (FIG. 16B) GI-related AEs including abdominal distension, upset stomach, and abdominal cramps, (FIG. 16C) psychology-related AEs, including anxiety and depression, and (FIG. 16D) Cardiovascular-related AEs including hypotension and hypertension. As demonstrated in this embodiment, the drug EF profile can be used to predict drug targets (e.g., PGE1 and PGE2 are acting on similar receptors, and they were clustered together in FIG. 16A.) Embodiments can determine both adverse effects and drug targets (i.e., potential therapeutic effects). Embodiments include predicting both bad and good effects of certain drugs, either together in the same analysis steps, or in separate analyses advantageously performed sequentially, serially, or asynchronously according to technical or commercial needs. Data represents the mean±S.D. Significant differences are indicated as * p<0.05, ** p<0.01, *** p<0.001 using unpaired t-tests (n=228-1,300). NKA: neurokinin A; LPS: lipopolysaccharides.

### Materials and Methods

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

Embodiments of the subject invention address the technical problem of predicting drug interactions and adverse effects being expensive, time consuming, and unpredictable. This problem is addressed by providing a standardized protocol to evaluate acute drug effects on gastrointestinal (GI) pacemaker activity using an index and guidelines on the potential effects of drugs to induce GI dysfunction using a standardized and efficient method for prediction using a learning model based on a novel database built using a standardized methodology, in which a machine learning method applying a combination of advanced techniques is utilized to predict drug adverse effects.

The transitional term “comprising,” “comprises,” or “comprise” is inclusive or open-ended and does not exclude additional, unrecited elements or method steps. By contrast, the transitional phrase “consisting of” excludes any element, step, or ingredient not specified in the claim. The phrases “consisting” or “consists essentially of” indicate that the claim encompasses embodiments containing the specified materials or steps and those that do not materially affect the basic and novel characteristic(s) of the claim. Use of the term “comprising” contemplates other embodiments that “consist” or “consisting essentially of” the recited component(s).

When ranges are used herein, such as for dose ranges, combinations and subcombinations of ranges (e.g., subranges within the disclosed range), specific embodiments therein are intended to be explicitly included. When the term “about” is used herein, in conjunction with a numerical value, it is understood that the value can be in a range of 95% of the value to 105% of the value, i.e., the value can be +/−5% of the stated value. For example, “about 1 kg” means from 0.95 kg to 1.05 kg.

The methods and processes described herein can be embodied as code and/or data. The software code and data described herein can be stored on one or more machine-readable media (e.g., computer-readable media), which may include any device or medium that can store code and/or data for use by a computer system. When a computer system and/or processor reads and executes the code and/or data stored on a computer-readable medium, the computer system and/or processor performs the methods and processes embodied as data structures and code stored within the computer-readable storage medium.

It should be appreciated by those skilled in the art that computer-readable media include removable and non-removable structures/devices that can be used for storage of information, such as computer-readable instructions, data structures, program modules, and other data used by a computing system/environment. A computer-readable medium includes, but is not limited to, volatile memory such as random access memories (RAM, DRAM, SRAM); and non-volatile memory such as flash memory, various read-only-memories (ROM, PROM, EPROM, EEPROM), magnetic and ferromagnetic/ferroelectric memories (MRAM, FeRAM), and magnetic and optical storage devices (hard drives, magnetic tape, CDs, DVDs); network devices; or other media now known or later developed that are capable of storing computer-readable information/data. Computer-readable media should not be construed or interpreted to include any propagating signals. A computer-readable medium of embodiments of the subject invention can be, for example, a compact disc (CD), digital video disc (DVD), flash memory device, volatile memory, or a hard disk drive (HDD), such as an external HDD or the HDD of a computing device, though embodiments are not limited thereto. A computing device can be, for example, a laptop computer, desktop computer, server, cell phone, or tablet, though embodiments are not limited thereto.

A greater understanding of the embodiments of the subject invention and of their many advantages may be had from the following examples, given by way of illustration. The following examples are illustrative of some of the methods, applications, embodiments, and variants of the present invention. They are, of course, not to be considered as limiting the invention. Numerous changes and modifications can be made with respect to embodiments of the invention.

### Example 1—Exemplary Measurement of Dopamine According to an Embodiment of the Subject Invention

Pacemaker potentials were recorded extracellularly on a 60-channel MEA system using full-thickness GI segments isolated from Suncus murinus. Basic slow-wave parameters, including frequency, amplitude, slope, period, and power partitions, were derived. Signal regularity was also evaluated using detrended fluctuation analysis and sample entropy analysis. Signal propagation, velocity, and activation time patterns were also constructed and compared before and after treatment with dopamine (0.1-100 μM).

Results showed that dopamine (1-10 μM) significantly reduced the dominant power, amplitude, and slope of the waveforms, but increased the period of the waveforms in tissue segments taken from the ileum and duodenum. In stomach segments, dopamine (100 μM) significantly increased the average frequency, signal irregularity, and tachy-rhythm percentage. Dopamine had biphasic effects on tissues taken from the colon, in which frequencies shifted towards a tachy-rhythm at 1-10 μM, but towards a brady-rhythm at 100 μM. Dopamine had a unique effect on the propagation of slow waves in tissues taken from the ileum and duodenum, being active at 100 nM and 100 μM, but not at intermediate doses from 1 to 10 μM. This indicates that dopamine can have a biphasic effect on slow waves in the upper gut.

Conclusions included dopamine differentially modulated slow waves in the GI tract and its tissue-specific profile can be related to its known side effect profile of nausea, emesis, and constipation. Slow-wave network analysis can generate new information on drug mechanisms affecting GI pacemaker activity. Data and methodology from this study can be beneficially applied, e.g., to compile a database to assist drug discovery and development.

Abbreviations referenced in these examples and throughout the specification can include the following, AF: average frequency; DF: dominant frequency; DFA: detrended fluctuation analysis; DP: dominant power; GI: gastrointestinal; ICCs: interstitial cells of Cajal; MEA: microelectrode array; PD: Parkinson's disease; SampEn: sample entropy; STM: spatio-temporal map; TAM: time activation map.

### Example 2—Detailed Study of Embodiments of the Subject Invention

New drugs are being developed for a wide variety of diseases. Following peripheral administration, drugs reach a variety of cell types, including those in the GI tract, by either absorption following oral or rectal administration or indirectly following distribution after parenteral administration. Interactions with the GI tract can result in dyspepsia and/or nausea and emesis via the disturbance of slow waves. It would be advantageous to be able to predict the GI-tract side effect profile of novel drugs, based on their known pharmacological profile, prior to experiments in whole animals or humans.

Most of the novel chemical entities are tested in vitro or ex vivo to determine whether they either contract or relax GI tissues. However, such experiments do not necessarily predict whether a drug has emetic liability or is likely to cause constipation or diarrhoea. For example, some drugs that inhibit contractions of the GI tract (e.g., anti-cholinergics, antihistamines) are anti-emetic, whilst others (e.g., opioids) are emetic. Gastric dysrhythmia can be associated with nausea and emesis, but it is technically difficult to study the effects of drugs on the electrical properties of the GI tract. Therefore, these experiments are not performed routinely and data from different laboratories have usually been generated under disparate experimental conditions, making associations with side effects unclear.

Dysrhythmia involves an alteration of rhythmic slow-wave pacemaker activity that is generated by interstitial cells of Cajal (ICCs). ICCs are located along the GI tract and they regulate peristalsis and motility. Hundreds of receptors are expressed on ICCs, and thousands of receptors are expressed on enteric neurons and smooth muscle cells, which interact with ICCs [1-3]. Drugs and/or mediators can interact with any of the receptors expressed within the GI to modulate or disrupt pacemaker activity. The inventors have established techniques to record slow waves in isolated GI tissues. To permit stable recordings over a microelectrode array (MEA), nifedipine (1 μM) is added to paralyse smooth muscle movement, while other receptors, ion channels, and cell-to-cell interactions remain intact to better model normal GI physiology. Slow-wave signals are then reliably recorded in the isolated GI tissue using a standard 60-channel MEA platform.

In this study, the inventors used Suncus murinus, the house musk shrew, as it is commonly used in anti-emetic research and it has tissues that produce more reliable pacemaker potential recordings than tissues from rodents. Moreover, rodents are incapable of emesis and therefore, have a lower translational value [4]. The data generated from the MEA platform were blinded and processed through a programmed slow-wave network analytical pipeline to derive slow-wave features, including frequency, amplitude, slope, period, power partition, activation times, propagation velocity, propagation pattern, waveform stability, and variability, which were used to construct a comprehensive pharmacological profile of the tested drug on various GI tissue sections. Dopamine was tested at 0.1-100 μM in the stomach, duodenum, ileum, and colon to collect data for the compilation of a database. The database was then subdivided into groups for classification machine learning to identify slow-wave features that differentiate between non-nausea-inducing and nausea-inducing drugs or between dopamine agonists and antagonists. It is contemplated within the scope of certain embodiments of the subject invention that the addition of further data sets to the database will continue to increase the power to predict mechanisms, therapeutic uses, and/or side effects.

Dopamine is well-known to modulate gut motility, but it has restricted use because of its side effect profile and relatively short half-life. However, dopamine receptor antagonists are established for treating gastric dysmotility, nausea, and emesis in humans [5,6]. Whilst the peripherally acting dopamine receptor antagonist, domperidone, is used to treat gastroparesis and gastroesophageal reflux, it is less effective at treating colon dysmotility [7], suggesting that dopamine is involved mainly in the upper GI tract in certain pathological conditions. However, constipation, which mainly involves the lower GI tract, is common in patients with Parkinson's disease (PD) [8,9]. Whilst dopamine agonists improve the PD symptoms associated with the central nervous system, some studies indicate that they tend to worsen constipation [8,10]. Therefore, the inventors hypothesised that dopamine can have differential actions on the GI tract due to region-specific effects on pacemaker activity and that this can, in turn, be a consequence of differences in the regional distribution of dopamine receptor subtypes.

Adult Suncus murinus (female, weighing 40-50 g) were obtained from the Laboratory Animal Services Centre, the Chinese University of Hong Kong. The animals were housed in plastic cages (1-5 per cage) in a temperature-(24±1° C.) and humidity (50±5%)-controlled room with artificial lighting provided from 06:00 to 18:00 h. Water and dry, pelleted cat chow (TriPro Feline Formula; American Pet Nutrition, Ogden, UT, USA) were given ad libitum. All experiments were conducted under a licence from the Government of the Hong Kong SAR and with permission from the Animal Experimentation Ethics Committee, The Chinese University of Hong Kong. The inventors used a randomisation protocol to test different concentrations of dopamine. Ten animals were used in this study.

Solutions and drugs included Krebs' medium (in mM: NaCl, 115; KCl, 4.7; KH₂PO₄, 1.2; MgSO₄·7H₂O, 1.2; CaCl₂·2H₂O, 2.5; glucose, 10; NaHCO₃, 25) used for all tissue manipulations. 3-Hydroxytyramine hydrochloride (dopamine) was purchased from Santa Cruz Biotechnology (Dallas, TX, USA), dissolved in distilled water to a concentration of 100 mM, and stored at −20° C. in aliquots. Dopamine was freshly diluted to the desired concentrations each day using freshly prepared Krebs' medium.

Electrical recordings were taken after the animals were euthanised by carbon dioxide asphyxiation. The entire GI tract was harvested and incubated at room temperature in Krebs' medium enriched with 95% 02 and 5% C02. The mesentery was removed, and the lumen contents were flushed with Krebs' medium. Segments (−1 cm) of duodenum, ileum, and colon were then isolated, but the stomach was kept intact. All tissue segments and the entire stomach were incubated in Krebs' medium containing nifedipine (1 μM; Sigma Aldrich, St Louis, MO, USA) for 15 min to inhibit smooth muscle activity. Pacemaker potentials were recorded using an MEA platform (MEA1060 1200×; Multichannel Systems, Reutlingen, Germany) as previously described [4]. Briefly, tissues were placed onto the electrode field of an MEA chip (60-channel, 8×8 configuration, 3D-tip-shaped electrodes with a height of 30 m and an inter-electrode distance of 200 μm; Ayanda Biosystems SA, Lausanne, Switzerland). Intestinal segments were aligned horizontally to the recording electrode field, while the stomach was oriented with the antrum facing the recording electrode field. The chamber temperature was maintained at 37° C. and the sampling frequency was 1 kHz. Five-minute baseline recordings were followed by 7-min recordings after the addition of dopamine. Specifically, duodenum, ileum, and colon sections were incubated with dopamine at 100 nM, 1 μM, 10 μM, and 100 μM. Due to the limited number of stomachs available and ethical issues, only one concentration of dopamine (100 μM) was tested on the stomach. All data were recorded and saved using MC_Rack (v 4.6.2, Multichannel Systems).

Data analysis included basic slow-wave feature extraction using a custom automated program. Raw data were converted into hierarchical data format version 5 (HDF5) file format, and imported into MATLAB (2020a/b, 2021a; Mathworks, Natick, MA, USA). The time point of drug administration was automatically identified based on the signal disturbance. The period of signal disturbance (20 s before and 40 s after the peak of the disturbance) was excluded from the analysis. Data were filtered using a passband between 0.1 Hz and 1 Hz and a stopband between 0.01 Hz and 2 Hz. The basic slow-wave features extracted were similar to those extracted in the inventors' previous studies [4,11,12]. However, instead of using several templates and scripts written in Microsoft Excel and Spike 2, a one-step automatic and blinded MATLAB programme was used for data analysis. There were four major changes to the data analysis process as compared to the inventors' previous studies [4,11,12]. First, auto-identification of the drug disturbance time point was used, rather than manual identification. Second, standardised signal filters were built in MATLAB to match the entire data analysis pipeline. Similar to the inventors' previous study, the filtered data were subjected to fast Fourier transformation (bin size, 2048) to identify the dominant frequency (DF) and dominant power. Frequency partitioning was performed by segmenting the power spectrum of 0-50 cpm into the following four segments: brady-rhythm (within the range 2-[DF−1] cpm), normal-rhythm ([DF±1] cpm), tachy-rhythm (within the range [DF+1]-40 cpm), and out-of-range frequencies (<2 cpm and >40 cpm). Third, waveforms were identified using the MATLAB ‘findpeaks’ function, with a minimum peak height of 30 V at baseline and 20 V after drug treatment, with a step time of 5.4 s for the intestine and 12 s for the stomach. The average peak-to-peak amplitude, slope, and period were derived for all detected waveforms. Fourth, propagation velocity was derived using phase-difference measurements, based on the auto-select seed electrode with the highest percentage of normal rhythms. When comparing the old and new analytical pipelines, the numerical data derived can be slightly different, but this does not affect the significant findings of the inventors' previous studies. More importantly, the time taken to perform the analyses and the possible number of human errors and amount of bias introduced into the data analysis were significantly reduced in the new approach.

Dominant activation pattern identification included a phase-based analysis performed as previously described (Liu et al., 2021). Briefly, high-frequency spikes with amplitudes greater than 200 V were masked. Continuous frequencies were identified using continuous wavelet transformation, and the transformed data were used to plot spectrograms. Channels were eliminated from the analysis if (1) the frequency was lower than the mean±two standard deviations, (2) the power was less than 10% of the mean, or (3) the number of outliers was greater than 3. High quality data were subjected to Hilbert transformation to identify the phase and frequency of the signals. Individual activation times were clustered using k-means clustering every 20 s and major activation patterns were grouped using a structural similarity index. The major patterns were first grouped based on baseline data, and patterns in the post-dopamine treatment data were matched to the baseline groups of patterns. If no matched pattern group was identified, new groups were created. The percentage changes in the first, second, and third dominant activation patterns based on the baseline or post-treatment data were calculated and compared, which allowed the inspection of the shifting behavior of activation patterns between pre- and post-dopamine treatment data.

Signal variability analysis was conducted. The inventors have previously applied detrended fluctuation analysis (DFA) and multiscale sample entropy (SampEn) analysis to evaluate signal fluctuation around the local trends and regularity of signals, respectively, in gastric myoelectric activity patterns recorded from conscious ferrets [13]. This approach was introduced in this study for the first time to analyse gastrointestinal slow-wave signals from isolated tissues measured using the MEA. This approach was useful in comparing normal and disturbed rhythms based on signal variability across time segments. DFA [14,15] and SampEn analysis [16] were performed based on previously described formulae. For DFA analysis, an averaged and extracted smaller window scale 3-25 and larger window scale 26-46 were used. For SampEn analysis, an averaged and extracted smaller window scale 1-5 and larger window scale 6-50 were used.

Statistical analyses were performed using MATLAB. Paired Student's t-tests were used to compare the baseline and post-treatment recordings. All numerical data were expressed as the means±standard deviations. A p-value<0.05 was considered statistically significant. The number of repeated experiments is indicated as ‘n’.

Graphical presentation included activation maps, spectrograms, DFA maps, and SampEn maps automatically generated for each dataset while running the data analysis programme. Representative graphs and maps were selected for publication. PRISM 8.0 (GraphPad, San Diego, CA, USA) was used to generate frequency distribution and pattern distribution maps. Selected slow-wave features were plotted on a radar diagram using Microsoft Excel.

Clustering and machine learning were conducted. Other drugs were tested and analysed using the same protocol used to test dopamine, which is described here as an example. The slow-wave features of the stomach, duodenum, ileum, and colon extracted for all tested drugs and concentrations were stored in a database. The database was used for clustering and machine learning in MATLAB to help test specific hypotheses. The following two example applications of the database are given in this publication: (1) the prediction of nausea-inducing drugs and (2) the classification of drugs as dopamine receptor agonists or antagonists.

Certain mRNA expression assays were conducted as follows. Primer pairs were designed for each of the target dopamine receptor genes, dopamine receptor subtypes 1, 2, 3, and 4 (D1-D4), based on a preliminary RNA sequencing study of Suncus murinus brain, stomach, duodenum, ileum, and colon (data not shown). Briefly, tissues were homogenised using a hand-held homogeniser. Forty milligrams of homogenised tissue was used for RNA extraction using TRIzol Reagent (Thermo Fisher Scientific, Waltham, MA, USA) and cDNA was prepared using TaqMan® reverse transcription reagents (Thermo Fisher Scientific) following the manufacturer's protocols. Standard polymerase chain reactions (PCRs) were performed with denaturation and activation steps at 95° C. for 2 min; followed by 50 cycles of amplification at 95° C. for 30 s, 55° C. for 30 s, and 72° C. for 45 s; and a final elongation step at 72° C. for 5 min using GoTaq® Green Master Mix (Promega, Madison, WI, USA). Gel electrophoresis was performed using a 2% agarose gel with GelRed® Nucleic Acid Gel Stain (Thermo Fisher Scientific).

Results included certain effects of dopamine on pacemaker potentials recorded from the gastrointestinal tract of Suncus murinus, such as dominant frequency (DF) and average frequency (AF). Dopamine significantly reduced the DF of slow waves in duodenal tissues from 27.1±2.2 cpm to 24.9±1.2 cpm at 100 nM (p<0.05, n=7), and from 28.6±4.1 cpm to 26.3±5.2 cpm at 100 μM (p<0.05, n=5). No significant differences were found in the stomach at 100 μM (p>0.05, n=8) or in ileal (p>0.05, n=6-7) or colonic tissues (p>0.05, n=6-9) at 0.1-100 μM. The AF significantly increased in the stomach from 9.8±1.2 cpm to 11.8±1.8 cpm at 100 μM (p<0.05, n=8). The AF was significantly reduced in duodenal tissues from 24.1±2.0 cpm to 20.9±0.7 cpm at 100 nM (p<0.05, n=7) and from 25.1±3.7 cpm to 22.2±5.4 cpm at 100 μM (p<0.05, n=5). The AF was significantly reduced in ileal tissues from 23.5±3.0 cpm to 19.4±3.6 cpm at 100 nM (p<0.05, n=7) and in colonic tissues from 23.9±2.4 cpm to 23.3±1.6 cpm at 100 nM (p<0.05, n=7, FIG. 1A-B).

Dopamine significantly reduced the dominant power (DP) of slow waves in the stomach from 888.0±691.5 μV² to 253.2±416.1 μV² at 100 μM (p<0.05, n=8). The DP was significantly reduced in ileal tissues from 780.4±276.9 μV² to 177.5±83.0 μV² at 100 nM (p<0.001, n=7) and from 1,070.5±745.1 μV² to 460.3±297.0 μV² at 1 μM (p<0.05, n=7). No significant differences were found in duodenal (p>0.05, n=5-9) or colonic tissues (p>0.05, n=6-9) at 0.1-100 μM (FIG. 1C).

Average amplitude, slope, and period of waveforms were observed. Dopamine significantly reduced the amplitude of slow waves in the duodenum from 193.0±54.9 μV to 114.1±37.7 μV at 100 nM (p<0.01, n=7), from 117.6±24.2 μV to 97.4±13.7 μV at 10 μM (p<0.05, n=9), and from 199.3±60.5 μV to 121.1±27.3 μV at 100 μM (p<0.01, n=5). The amplitude was significantly reduced in ileal tissues from 165.2±20.5 μV to 119.2±20.0 μV at 100 nM (p<0.001, n=7), from 187.1±76.9 μV to 138.0±66.2 μV at 1 μM (p<0.01, n=7), and from 229.6±115.9 μV to 149.1±56.7 μV at 10 μM (p<0.05, n=6). The amplitude was significantly reduced in colonic tissues from 267.3±80.9 μV to 199.7±48.8 μV at 100 μM (p<0.01, n=6). No significant differences were found in the stomach at 100 μM (p>0.05, n=8, FIG. 1D).

Dopamine significantly reduced the slope in duodenal tissues from 321.7±95.5 μV s⁻¹ to 166.9±57.7 μV s⁻¹ at 100 nM (p<0.01, n=7), from 202.4±50.6 μV s⁻¹ to 153.9±47.7 μV s⁻¹ at 10 μM (p<0.05, n=9), and from 322.2±108.6 μV s⁻¹ to 182.5±61.7 μV s⁻¹ at 100 μM (p<0.01, n=5). The slope was significantly reduced in ileal tissues from 257.5±59.8 μV s⁻¹ to 160.1±42.0 μV s⁻¹ at 100 nM (p<0.01, n=7), from 300.9±117.5 μV s⁻¹ to 204.4±91.9 μV s⁻¹ at 1 μM (p<0.01, n=7), and from 383.7±185.8 μV s⁻¹ to 238.2±95.7 μV s⁻¹ at 10 μM (p<0.05, n=6). The slope was significantly reduced in colonic tissues from 439.3±124.3 μV s⁻¹ to 299.0±76.5 μV s⁻¹ at 100 μM (p<0.001, n=6). No significant differences were found in the stomach at 100 μM (p>0.05, n=8, FIG. 1E).

Dopamine significantly reduced the waveform period in duodenal tissues from 2.52±0.21 s to 3.27±0.47 s at 100 nM (p<0.05, n=7) and from 2.44±0.72 s to 3.26±1.29 s at 100 μM (p<0.05, n=5). The waveform period was significantly increased in ileal tissues from 2.66±0.40 to 3.73±1.05 at 100 nM (p<0.05, n=7), from 2.53±0.50 s to 3.10±0.74 sat 1 M (p<0.05, n=7), and from 2.30±0.18 s to 3.05±0.68 s at 10 μM (p<0.05, n=6). No significant differences were found in the stomach at 100 μM (p>0.05, n=8) or in colonic tissues at 0.1-100 μM (p>0.05, n=6-9, FIG. 1F).

Dopamine did not affect propagation velocities in the stomach at 100 μM (p>0.05, n=8) or in duodenal (p>0.05, n=5-9), ileal (p>0.05, n=6-7), or colonic tissues (p>0.05, n=6-9) at 0.1-100 μM (FIG. 1G).

Dopamine significantly reduced the DFA fluctuation function (smaller scale 3-25) from 2.36±0.08 to 2.27±0.16 at 100 μM (p<0.05, n=7) in the stomach; reduced from 2.65±0.08 to 2.51±0.05 at 100 nM (p<0.01, n=6), from 2.26±0.16 to 2.18±0.16 at 100 M (p<0.05, n=9) in duodenal tissues; reduced from 2.35±0.14 to 2.28±0.11 at 100 nM (p<0.05, n=6), from 2.29±0.23 to 2.19±0.20 at 1 M (p<0.05, n=8), from 2.24±0.32 to 2.13±0.34 at 10 M (p<0.05, n=8), from 2.35±0.12 to 2.25±0.05 at 100 μM (p<0.05, n=7) in ileal tissues; reduced from 2.37±0.07 to 2.26±0.06 at 100 nM (p<0.01, n=6), from 2.30±0.33 to 2.23±0.34 at 1 μM (p<0.05, n=8), from 2.24±0.23 to 2.11±0.27 at 10 μM (p<0.05, n=6), from 2.31±0.12 to 2.19±0.13 at 100 μM in colonic tissues (FIG. 1H).

Dopamine significantly reduced the DFA fluctuation function (larger scale 26-46) from 2.17±0.07 to 1.98±0.11 at 100 nM (p<0.01, n=6), from 1.74±0.32 to 1.63±0.38 at 1 μM (p<0.05, n=7), from 1.46±0.29 to 1.36±0.31 at 100 μM (p<0.05, n=9) at duodenal tissues; reduced from 1.71±0.17 to 1.55±0.13 at 100 nM (p<0.05, n=6), from 1.78±0.24 to 1.64±0.28 at 1 μM (p<0.05, n=8), from 1.63±0.23 to 1.43±0.21 at 100 μM (p<0.05, n=7) at ileal tissues; reduced from 1.80±0.09 to 1.58±0.11 at 100 nM (p<0.001, n=6), from 1.74±0.26 to 1.61±0.29 at 1 M (p<0.01, n=8), from 1.73±0.16 to 1.53±0.19 at 10 M (p<0.05, n=6), from 1.63±0.19 to 1.46±0.21 at 100 μM (p<0.05, n=10) at colonic tissues. Dopamine did not change the DFA fluctuation function (larger scale 26-46) at the stomach at 100 μM (p>0.05, n=7) (FIG. 1I).

Dopamine significantly increased the average SampEn (smaller scale 1-5) from 0.58±0.05 to 0.60±0.04 at 10 μM (p<0.05, n=7) in duodenal tissues; increased from 0.59±0.03 to 0.62±0.03 at 100 nM (p<0.01, n=6), from 0.57±0.06 to 0.59±0.05 at 1 M (p<0.01, n=8), from 0.57±0.06 to 0.60±0.04 at 10 μM (p<0.05, n=6) in colonic tissues. Dopamine did not change the average SampEn (smaller scale 1-5) at the stomach at 100 uM (p>0.05, n=7) and in ileal tissues at 0.1-100 μM (p>0.05, n=6-8) (FIG. 1J).

Dopamine significantly reduced the average SampEn (larger scale 6-50) from 0.33±0.06 to 0.28±0.07 at 100 nM (p<0.01, n=6) in colonic tissues. Dopamine did not change the average SampEn (larger scale 6-50) at the stomach at 100 uM (p>0.05, n=7), in duodenal tissues at 0.1-100 μM (p>0.05, n=6-9), in ileal tissues at 0.1-100 μM (p>0.05, n=6-8) (FIG. 1K).

To allow visual and statistical analyses of the shifting behavior of frequencies after dopamine treatment, the power spectrum was segmented into percentages of brady-, normal-, and tachy-rhythm ranges, as shown in FIGS. 2A and 2B for the stomach and intestinal (colon) data. These data are presented graphically in stacked histograms (FIG. 2C).

In the stomach, dopamine at 100 μM significantly decreased the percentage of normal rhythms from 55.1±7.2% to 23.6±13.1% (p<0.01, n=8) and significantly increased the percentage of tachy-rhythms from 36.1±12.8% to 60.2±15.4% (p<0.05, n=8).

In duodenal tissues, dopamine increased the percentage of brady-rhythms from 35.7±10.3% to 87.2±7.7% (p<0.0001, n=7) at 100 nM, from 25.8±12.3% to 53.7±26.6% (p<0.05, n=6) at 1 μM, and from 43.3±11.6% to 68.6±29.0% (p<0.05, n=5) at 100 μM. The percentage of normal rhythms in duodenal tissues significantly decreased from 46.1±11.2% to 4.8±4.6% (p<0.001, n=7) at 100 nM and from 52.4±14.7% to 7.8±6.4% (p<0.05, n=9) at 10 μM, whereas the percentage of tachy-rhythms significantly decreased from 12.5±5.7% to 2.1±0.7% (p<0.05, n=7) at 100 nM and from 11.0±5.2% to 5.4±4.9% (p<0.05, n=5) at 100 M.

In ileal tissues, dopamine increased the percentage of brady-rhythms from 31.8±11.7% to 60.0±26.0% (p<0.05, n=7) at 100 nM, from 27.5±7.8% to 46.3±22.7% (p<0.05, n=7) at 1 μM, and from 32.8±12.9% to 63.3±28.5% (p<0.05, n=6) at 10 μM. Meanwhile, the percentage of normal rhythms significantly decreased from 46.1±5.6% to 4.8±17.0% (p<0.01, n=7) at 100 nM, from 50.6±10.8% to 24.5±11.8% (p<0.01, n=7) at 1 μM, and from 50.9±9.8% to 9.2±10.9% (p<0.0001, n=6) at 10 μM.

In colonic tissues, dopamine increased the percentage of brady-rhythms from 34.7±9.1% to 53.9±26.8% (p<0.05, n=6) at 100 μM, but significantly decreased the percentage of normal rhythms from 41.4±9.7% to 14.5±18.1% (p<0.01, n=6) at 1 μM, from 42.4±6.9% to 13.1±17.4% (p<0.05, n=9) at 10 μM, and from 53.1±8.1% to 33.4±23.3% (p<0.05, n=6) at 100 μM. The percentage of tachy-rhythms in colonic tissues significantly increased from 18.3±12.6% to 55.5±34.8% (p<0.05, n=6) at 1 μM dopamine.

Power spectra were also plotted against time to generate spectrograms. Representative spectrograms are shown in FIGS. 2D and 2E. Eleven minutes of data were plotted. Drug administration artefacts were seen in the spectrograms at the point of delivery at 5 min. In the stomach, dopamine (100 μM) reduced the DP of the DF, which translated to a reduced power spectral density from red to green and yellow. In duodenal tissues, the DF was slightly shifted towards a lower frequency by dopamine (10 μM). For ileal tissues, two representative spectrograms are shown. The spectrogram on the left shows that the DF was slightly shifted towards a higher frequency by dopamine (10 μM). The spectrogram on the right shows extra peaks appearing to the left of the baseline DF following dopamine (10 μM) administration, which was consistent with the overall decrease in the percentage of frequencies in the brady-rhythm range (above). In colonic tissues, extra peaks appeared to the right of the original DF after dopamine (10 μM) administration, which was consistent with the overall increase in the percentage of frequencies in the tachy-rhythm range (above). A limitation of this type of graphical analysis is that it only allows one representative channel from the recordings to be shown in each figure. In these examples, the inventors identified two effects of drug treatment on the final DF or AF. The first effect was an overall shift in the DF peak, and the second effect was the appearance of extra peaks at a different frequency than the frequency of the original DF.

Certain effects of dopamine on activation pattern distributions were observed. The three most dominant clustered groups of activation patterns were identified for each dataset based on their respective baseline (FIG. 3A “(i) Baseline”) and post-treatment data (FIG. 3B “(ii) Post-drug”). The post-treatment activation patterns were matched based on baseline-clustered groups (FIG. 3C “(iii) Distribution of Activation Pattern”). In this example, duodenal tissue was treated with 100 μM dopamine and the major patterns were shifted towards the minor patterns found in the baseline data. One new clustered activation pattern group was also formed, and the major patterns found in the baseline data were largely reduced. Using this analytical method to cluster and group activation patterns for all datasets, the percentages of the three most dominant patterns, based on baseline data (FIG. 3D), and the percentages of the three most dominant patterns, based on post-treatment data (FIG. 3E), were derived and plotted in a stacked histogram. Note that between each dataset, the dominant activation patterns were different. The major aim of this type of analysis was to derive the percentage change in the dominant activation patterns between pre- and post-treatment data, which represents the degree to which a drug disrupts the original (baseline) propagation pattern of pacemaker activities, and to create new propagation patterns of pacemaker activities based on post-treatment data.

In duodenal tissues, dopamine decreased the percentage of dominant baseline patterns from 32.3±11.8% to 11.1±13.0% (p<0.01, n=7) and from 23.3±5.7% to 8.4±11.2% (p<0.05, n=7) for the first and second patterns, respectively, at 100 nM; from 33.7±13.1% to 12.8±9.4% for the first pattern at 1 μM (p<0.05, n=6); and from 29.3±5.5% to 7.0±7.0% (p<0.01, n=4), 20.7±5.4% to 7.4±4.1% (p<0.05, n=4), and 15.2±2.1% to 5.9±3.6% (p<0.05, n=4) for the first, second, and third patterns, respectively, at 100 μM.

In ileal tissues, dopamine decreased the percentage of dominant patterns from 32.8±5.7% to 3.8±4.5% (p<0.01, n=7), 21.5±1.7% to 2.6±1.6% (p<0.0001, n=7), and 17.2±4.8% to 2.2±2.6% (p<0.0001, n=7) for the first, second, and third patterns, respectively, at 100 nM; from 35.2±6.0% to 13.0±6.7% (p<0.001, n=6) for the first pattern at 1 μM; from 35.7±8.4% to 19.2±16.4% (p<0.05, n=5) for the first pattern at 10 μM; and from 27.6±4.1% to 9.4±9.5% (p<0.01, n=4), 23.5±2.8% to 5.7±5.1% (p<0.001, n=4), and 19.1±2.8% to 5.9±6.6% (p<0.01, n=4) for the first, second, and third patterns, respectively, at 100 μM.

In colonic tissues, dopamine reduced the percentage of dominant patterns from 34.4±3.7% to 11.4±12.9% (p<0.01, n=4) for the first pattern at 100 μM. No significant differences were found in the percentage of dominant patterns compared with those at baseline in the stomach at 100 μM (p>0.05, n=4, FIG. 3D).

In duodenal tissues, the percentage of dominant patterns significantly increased from 10.2±9.1% to 18.5±2.3% (p<0.05, n=5) for the second pattern at 10 μM, and decreased from 20.3±10.8% to 7.5±5.8% (p<0.05, n=4) for the third pattern at 100 μM.

In ileal tissues, the percentage of dominant patterns significantly decreased from 15.6±10.3% to 4.2±4.2% (p<0.05, n=7), 16.8±6.6% to 3.0±2.6% (p<0.01, n=7) for the second and third patterns, respectively, at 100 nM; increased from 8.9±9.0% to 31.4±13.7% (p<0.01, n=6) for the first pattern at 1 μM; increased from 17.2±11.5% to 32.3±13.2% (p<0.05, n=5) for the first pattern at 10 μM; and decreased from 17.8±7.4% to 6.8±6.1% (p<0.05, n=4) for the third pattern at 100 μM.

In colonic tissues, the percentage of dominant patterns significantly increased from 19.1±12.2% to 39.6±15.6% (p<0.05, n=6) for the first pattern at 1 μM, and decreased from 20.3±10.8% to 7.5±5.8% (p<0.0001, n=5) for the third pattern at 100 μM. No significant differences were found in the percentage of dominant patterns in the stomach at 100 μM compared to those at baseline (p>0.05, n=4, FIG. 3E).

Dopamine increased the number of groups of patterns recorded from the stomach from 4.3±1.0 to 5.3±1.0 at 100 μM (p<0.05, n=8). In duodenal tissues, the number of groups of patterns significantly increased from 6.2±1.0 to 6.8±1.0 (p<0.05, n=6) at 1 μM and from 5.9±0.7 to 6.4±0.5 (p<0.05, n=9) at 10 μM. In ileal tissues, the number of groups of patterns significantly increased from 6.3±0.8 to 7±0.8 (p<0.05, n=7) at 1 μM and decreased from 9.3±0.7±to 6.9±1.0 (p<0.05, n=6) at 10 μM. In colonic tissues, the number of groups of patterns significantly increased from 5.4±1.0 to 6.4±1.1 (p<0.05, n=6) at 1 μM and from 6±0.7 to 6.8±1.1 (p<0.05, n=9) at 10 μM (FIG. 3F).

The total percentage change for all activation patterns is denoted as ActP in FIG. 3G. This value was obtained by calculating the difference between baseline and post-treatment data. No statistical analysis was performed for this value. However, the value can be compared with a vehicle treatment control.

Other conventional types of graphical presentations of wave propagation were constructed. The activation time patterns described herein represent one method to present wave propagation. Another common method is the use of spatio-temporal maps (STMs). In STMs, the x- and y-axes represent the time and the distance of a line of interest, respectively, while the z-axis (colour scale) represents the amplitude (FIGS. 7A-7I). The ‘line of interest’ represents a selected line of electrodes (either vertical or horizontal) across the MEA electrode field. For the 60-electrode MEA used in this study, the maximum distance of a horizontal or vertical line was 1.4 mm, with an inter-electrode distance of 0.2 mm, which represents the resolution of the y-axis of the STM. It is also possible to choose a slope line, but this requires the inter-electrode distance to be adjusted to 0.28 mm. In the STM, the centre of the white zone is where the peak of a waveform is located, while the centre of the black zone is where the trough of a waveform is located. Due to the very fine resolution of the inventors' MEA chip, the wave front of the peaks and troughs of the STM appeared at almost the same time. Slightly tilted wave fronts were observed in some cases, indicating leading or lagging propagation of the same wave front with time. The denser the appearance of the white and black zones, the higher the frequency of pacemaker potentials. In the representative STM for the stomach (FIGS. 7A-7I), approximately 9 and 10 wave fronts were counted before and after dopamine treatment (100 μM), respectively, indicating an increase in frequency. The number of wave fronts counted directly represented 9 cpm and 10 cpm within the specific time segment, because the x-axis showed an exact 1 min interval in these STMs. In duodenal tissues, 30 and 27 wave fronts were found before and after dopamine treatment (10 μM), indicating a decrease in frequency. Moreover, given the scale of amplitude indicated by the colour bar, the amplitudes of pacemaker potentials were reduced from the white and black zones towards the green and yellow zones. Similarly, 26 and 30 wave fronts were found in ileal tissues and 32 and 32 wave fronts were found in colonic tissues before and after dopamine (10 μM) treatment, respectively. A limitation of this method of presentation is that only one line of electrodes and one specific time frame can be used as representative data, and data from the other 50+ electrodes cannot be included.

To overcome this limitation, a video or a series of time-lapse images can be produced to represent all 60 channels of data (e.g., as shown in FIGS. 11A-12B). Amplitudes were normalised to the maximum amplitudes found within the baseline recordings. In these videos, the spread of the peak events (white zone) or the trough events (black zone) across the MEA recording area can be seen in slow motion (½ time). In the stomach, two slightly unsynchronised populations of pacemaker potentials were found in the baseline data (FIG. 11A). After dopamine treatment (100 M), wave propagation was inhibited, and amplitudes were reduced (FIG. 11B). The shift between white and black dominant time was faster in colonic tissues than in the stomach, indicating a higher pacemaker frequency (FIGS. 12A and 12B). The peaks (white zone) were propagated from left to right at baseline. The propagation direction and speed were relatively constant before and after dopamine treatment (10 μM). These videos are useful for visualising pacemaker activity propagation before and after drug treatment.

Conventional raw traces inspection was performed. Most electrophysiological studies using a single microelectrode or patch-clamp techniques permit the recording of basic raw traces, from which data can be extracted and examined (FIG. 8). However, it is important to emphasise that pacemaker potentials recorded using the MEA technique are extracellular potentials. They can present as numerous shapes, as they are affected by interference from waves generated from a network of ICCs. It is possible to inspect raw traces to visually derive frequency and amplitude changes. However, this is generally done for one channel at a time within a specific time segment and within one experiment.

The pharmacological profile of dopamine on GI pacemaker activities was observed. The following 15 slow-wave signal features were selected and plotted in a radar diagram representing the pharmacological profile of dopamine on GI pacemaker activities: (1) DF, (2) AF, and (3) DP; the power distribution as a percentage of (4) brady-rhythm, (5) normal rhythm, and (6) tachy-rhythm range frequencies; the average (7) amplitude, (8) slope, and (9) period of the waveforms; (10) propagation velocity; (11) average of the extracted scales of DFA fluctuation function; (scale 3-25); (12) average of the extracted scales of DFA fluctuation function (scale 26-46); (13) SampEn; (scale 1-5); (14) SampEn (scale 6-50); and (15) the total percentage change of all activation patterns (ActP, FIG. 4). All values shown in FIG. 4 indicate the percentage changes of each of the 15 signal features. This radar diagram provides an immediate indication of how dopamine affects GI pacemaker activity in one graph. For example, at the AF, the red and purple lines (stomach and colon) lie above the line ‘0’, while the blue and green lines (duodenum and ileum) lie below line 0, indicating there can be a tissue-segment-dependent effect of dopamine. To determine whether there are significant differences, p-values obtained using paired Student's t-tests can be used to compare pre- and post-treatment recordings (see Table 2).

Database design and construction was undertaken. All numerical data were stored in database files containing the values of all of the slow-wave features extracted above, including but not limited to the date of each experiment, the sequences of the experiment, the tissue type, the drug name, the drug dose, the number of active channels, the number of repeats for each experiment, the means, the standard deviations, and the p-values of statistical analyses. All numerical values for experiments using dopamine are presented in Table 2 as an example. Note that in some datasets, the propagation velocity and the activation pattern were not derived if the number of active channels after filtering was too low, while the other features were preserved for data analysis. All of the other drugs the inventors have previously tested, or will test in the future, can be stored in a similar manner in the database. The database can then be subjected to machine learning to test specific hypotheses. Two example applications are given in FIGS. 5A-5D.

Table 2. The numerical values of all slow-wave features showing the effects of dopamine on pacemaker potentials along the gastrointestinal tract of Suncus murinus. The Appended table contains eight sheets, including (Table 2.1) ‘True values (basic parameters)’, showing the true numerical values of 10 slow-wave features listed with the date and sequence of each experiment; (Table 2.2) ‘True values (DFA & SampEn)’, showing the calculated DFA and SampEn values listed with the date and sequence of each experiment; (Table 2.3) ‘True mean and SD’, showing the calculated means and standard deviations of all slow-wave features grouped by concentration and tissue type; (Table 2.4) ‘Percentage change mean and SD’, showing the calculated means and standard deviations of the percentage change of all slow-wave features grouped by concentration and tissue type; (Table 2.5) ‘p-value’, showing the calculated p-values of all slow-wave features for comparisons between the baseline and post-treatment recordings; (Table 2.6) ‘Pattern(B)’, showing the calculated percentage values of the dominant activation time pattern based on the baseline data and the respective p-value; (Table 2.7) ‘Pattern(P)’, showing the calculated percentage values of the dominant activation time pattern based on the post-treatment data and the respective p-values; and (Table 2.8) ‘Radar’, showing the percentage change values used for plotting the radar diagram in FIG. 4.

Prediction of the potential for a drug to induce nausea was made. The analysis used data extracted from the inventors' database that was generated from the effects of 25 drugs on duodenal tissues (see for example, FIGS. 5A-5D). A literature search was conducted to gather clinical data on the potential of each drug to induce nausea in humans. The 25 drugs were separated into two groups: nausea-inducing and non-nausea-inducing. All slow-wave features were subjected to clustering and machine learning. Principal component analysis was used to extract the major components that explain 95% of the differences between groups. All data were clustered to visualise the potential features used for classification (FIG. 5A). Features with significant differences between the two groups were identified (FIG. 5C). The prediction model can be applied for adverse effect prediction for novel drugs. The current model for nausea prediction has 68.8% accuracy. The performance of the model is expected to improve as more drugs are added to the database. Similar models can also be built for other GI-related adverse effects, such as vomiting, diarrhoea, and constipation, using different sub-sets of data extracted from the database and from current and future literature. The machine learning models are learned models computer-generated and stored in machine-readable codes when machines learn from the data stored in a database. These can be actively updated and improved over time. The drug profile of these other 25 drugs can be used with various embodiments of the subject invention as applied on dopamine to evaluate these and other drugs. Beneficial and effective embodiments are within the scope of the subject invention for most common animal models with a functional gastrointestinal tract or other functional organ or biological system that have pacemaker activities.

Classification of dopamine receptor agonists and antagonists was undertaken. The example analysis used five agonists and five antagonists of dopamine receptors, regardless of their affinity or selectivity for dopamine receptor subtypes. Depending on the hypothesis to be tested, the model can be further refined by testing a list of drugs that act on a specific dopamine receptor subtype(s) (e.g., D₂ receptor) or by separating drugs with known low or high affinity towards specific receptors (e.g., with pKi values<7 or >7). The current example identified specific features that were significantly different between dopamine agonists and antagonists (FIGS. 5B and 5D). This model can be used to predict the properties of newly synthesised drugs.

Expression of dopamine receptors in the gastrointestinal tract of Suncus murinus was observed. PCR was performed using specific primers for D₁, D₂, D₃, and D₄ dopamine receptors. All four types of receptors were found to be expressed in the stomach, duodenum, ileum, and colon of Suncus murinus, with brain tissue used as a reference for expression (Table 1). Based on the partial sequences obtained by RNA sequencing of a young adult male Suncus murinus (data not provided), homology with human D₁, D₂, and D₃ sequences was 89.4%, 93.6%, and 85.1%, respectively. The partial sequence aligned to D₄ was not sufficient to accurately determine the level of homology.

TABLE 1
Primer pairs specific for Suncus
murinus dopamine receptors

| | Forward primers | Reverse primers | size |
|---|---|---|---|
| D1 | 3′ -TCGCAGTCCAA | 3′ -TACCTGATCCC | 122 |
| | AATGACCGA-5′ | CCATTCCGT-5′ | bp |
| | (SEQ ID NO: 1) | (SEQ ID NO: 2) | |
| | TCGCAGTCCAAAATGACCGAGAGCGCTGGCGACAGT | | |
| | TTCTCCGAGGGCTTGGCCCCTCCCCCACCCACCTCC | | |
| | TCCTTCTTCTTCACATCTTCAGAGGAGCCCACGGAA | | |
| | TGGGGGATCAGGTA (SEQ ID NO: 3) | | |
| D2 | 3′ -CCATTGGGCAA | 3′ -GGAGCTGGAGA | 216 |
| | GGTCTGGAT-5′ | TGGAGATGC-5′ | bp |
| | (SEQ ID NO: 4) | (SEQ ID NO: 5) | |
| | CCATTGGGCAAGGTCTGGATCTCAAAGAACTTGGCA | | |
| | ATCCTGGAGTGGTCTCTGGCATGCCCATTCTTCTCT | | |
| | GGTTTGGCGGGGCTATCAGGGGTGCTGTGGAGGCCA | | |
| | TGTTGAGATGGGTCAGGGAGGGTCAGCTGGTGGTGG | | |
| | CTGGGTGGAATGGGACTGTAGCGGGTCCTCTCAGGC | | |
| | GGGCTGGTGCTGGACAGCATCTCCATCTCCAGCTCC | | |
| | (SEQ ID NO: 6) | | |
| D3 | 3′ -AAAAGGGCAG | 3′ -GGTTGCCTTCT | 156 |
| | GAAGGACTCG-5′ | TCTCCCGAA-5′ | bp |
| | (SEQ ID NO: 7) | (SEQ ID NO: 8) | |
| | AAAAGGGCAGGAAGGACTCGAAACTCTCTCAGCCCC | | |
| | AACTTGGCACCCAAGCTCAGCTTAGAAGTTCGAAAA | | |
| | CTCAGTAACGGCAGGCTGTCAACATCCCTAAAGTTG | | |
| | GGTCCACTGCAACCTAGATCAGTGCCACTTCGGGAG | | |
| | AAGAAGGCAACC (SEQ ID NO: 9) | | |
| D4 | 3′ -ACAGGCCTCTC | 3′ -TCTGGGCCTGG | 169 |
| | AGTGTCTCA-5′ | TTCTACTGT-5′ | bp |
| | (SEQ ID NO: 10) | (SEQ ID NO: 11) | |

ACAGGCCTCTCAGTGTCTCAGCACAGACAGACAGAC
AGACGTGCCTGCATCTGTCTTGTTCTCGCCCCACAC
CCGAGTCCCCCTGCCAGACGTCCCCTGCAACACTAC
CAGCCAAGGAGTTACTGCTGCTCGGGTGGCTGGAAC
CCACGACAGTAGAACCAGGCCCAGA
(SEQ ID NO: 12)

bp: base pairs. Partial sequences of the genes are shown.

Using dopamine as an example drug, the inventors demonstrated that pacemaker activity in the GI tract of Suncus murinus can be recorded using an MEA technique and that slow-wave features can be extracted using known analytical algorithms and a novel phase-based pattern distribution analysis. Moreover, the inventors showed that the collection of these slow-wave features can be used to build a drug database for drug screening and classification, and the development of predictive learning models, including but not limited to the prediction and interpretation of functional effects, drug adverse effects, and the classification of drug actions on specific receptors.

An exemplary business model was defined, wherein the standardised protocol from data collection to data analysis and the construction of a basic database in accordance with an embodiment of the subject invention, standardising the experimental process and constructing the automatic analytical pipeline to build databases for predictive and classification purposes to potentially generate revenue. Certain aims of the business model can include but are not limited to (1) helping clients test their target drugs using a standardised protocol; (2) to record, evaluate, analyse, interpret, and build similar databases to generate revenue; and (3) to regulate the use of the built databases under this standardised protocol for drug comparisons or model construction to generate revenue. The standardised protocol, including the experimental methodology and automatic analytical pipeline, is valuable in terms of its efficiency, accuracy, and reproducibility to decrease the number of human errors and reduce bias in data collection, analysis, and slow-wave feature extraction. Moreover, the database provides data consistency, reliability, and comprehensiveness for each drug tested. The predictive power of the database can be useful in aspects including, but not limited to, drug discovery and development, food safety, basic research, and the development of personalised therapy.

Flexibility of the standardised protocol includes but is not limited to animal models. The standardised protocol for pacemaker potential recording and analysis is not limited to applications using Suncus murinus. The protocol can also be applied to studies of mice [4, 11,12, 17-21], guinea pigs [22], rats (FIG. 10), and ferrets (FIGS. 9A-9F), and any species with a functional GI system, including humans.

Moreover, the current example study only shows the use of the technique in healthy animal subjects. However, the technique can also be used in disease, transgenic, or pre-treatment animal models, such as those developed to investigate inflammation, diabetes, chemotherapy, or neurodegeneration, to study different types of hypotheses.

Other tissue models are contemplated under various embodiments of the subject invention. The current example model-built drug database focused on the GI tract. However, similar models can be built using tissues other than those from the GI tract, including for example, cardiac, neuronal, and muscular tissue, to construct new databases for the prediction of other adverse effects, including for example, cardiac dysrhythmia and epilepsy in accordance with the teachings of the subject invention.

The current example used microelectrode array chips designed and produced by Ayanda Biosystems (S.A. Lausanne, Switzerland). The chips had 60 electrodes, with an inter-electrode distance of 200 μm, an electrode diameter of 30 μm, and an electrode height of 30 μm. The MEA system used in this study was designed and produced by Multichannel Systems (Reutlingen, Germany). The number of electrodes, the inter-electrode distance, and the diameter and height of the electrodes can be customised to record a larger area or a different resolution for similar drug testing purposes and database construction in accordance with the teachings of the subject invention.

The inventors extracted more than 24 slow-wave features that were then stored in the inventors' database, based on existing algorithms including fast Fourier transform, Hilbert transform, and continuous wavelet transform algorithms; DFA; and SampEn analysis. These features can be refined to suit different purposes and applications and to build different predictive models. Moreover, with further mathematical and technological advancements, other types of slow-wave features can be extracted to include in the database.

The application of the drug databases are not limited to the embodiments detailed herein. Two example applications, namely, the prediction of the potential of a drug to induce adverse effects and the classification of drug actions on a specific receptor, were given as examples. Other potential applications of the drug database include, but are not limited to, comparisons of synthesised drugs with different chemical formulations; comparisons of different drugs, food products, remedies, and therapies; comparisons of different animals, diseases, and transgenic and pre-treatment models; comparisons of mathematical models; and combinations of the aforementioned applications.

The animals were euthanised by carbon dioxide asphyxiation. Cervical dislocation was not performed due to ethical issues. An overdose of anaesthesia or other methods that would require the use of a chemical compound for euthanasia were avoided to minimise the effects of these compounds on pacemaker activity prior to the experiments. For example, sodium pentobarbital is known to alter GI motility [23].

Faecal content within the lumen of the GI was washed away using Krebs' medium. The cells were not digested or isolated and the mucosal layer was not removed. The full thickness of the GI tissues was used for recordings. The advantage of this method is that the ICCs remain intact to preserve the majority of their natural connections with other cells, including enteric neurons and smooth muscle cells, for the aim of drug screening. Moreover, this method had a greater success rate than previously published methods at producing high-quality pacemaker signals [17-21, 24, 25].

The use of the MEA over other techniques to record GI pacemaker activity has certain advantages in certain embodiments but is not necessarily limiting to all embodiments. The MEA technique is superior to conventional single microelectrode or patch-clamping techniques [26,27], because it allows the deduction of spatial and two-dimensional wave propagation information. The MEA technique allows recordings over a larger area, covering a network of ICCs, and is superior to calcium imaging techniques [28], in which only a few ICC cells can be examined at a time and that are limited by the power of the microscope and camera. For example, within the area (1.8 mm²) covered by 60 electrodes in the current protocol, there are >900 ICC cell bodies and numerous ICC fibres [4]. However, in contrast to single microelectrode, patch-clamping, and calcium imaging techniques, where currents within a single ICC can be recorded, the MEA records extracellularly over a network of ICCs. The exact currents of the slow waves produced by a single ICC cannot be measured by the MEA. For drug testing, the evaluation of networking behavior using an MEA is considered to be a more appropriate technique.

Certain methods were employed to minimise signal contamination. The signals recorded can include those from local electrically active cells other than the ICCs, including smooth muscle cells and neurons. The inventors aimed to preserve most or all of these potential targets to determine the final drug-induced effect on pacemaker activity. However, the inventors found that it was essential to keep the GI tissue static for stable and reliable recordings. The inventors added nifedipine (1 μM) to block smooth muscle activity and movement by blocking L-type calcium channels [22], to inhibit the tissue from moving away from the recording area due to smooth muscle contraction and relaxation. The inventors did not block high-frequency neuronal spiking activity by pharmacological means. Instead, the inventors performed filtering to remove high-frequency spikes during data analysis. This condition preserves most neuronal interactions that occur via neurotransmitters and receptors, and most ion channels. However, special attention is required when designing experiments on drugs that act on calcium or potassium ion channels, which are potentially blocked by nifedipine [4]. This is believed to be the optimal condition for the study of drug interactions with pacemaker potentials in isolated GI tissues under the current technical limitations.

Embodiments can provide advantageous sampling frequency and data filtering methods. According to the Nyquist sampling theorem, the sampling frequency should be at least two times the highest frequency of the signal of interest to enable the correct re-construction of the original signals. The pacemaker frequency the inventors recorded in different animal models, or after the administration of different drugs, never exceeded 50 cpm, i.e., 0.83 Hz. Therefore, theoretically, a sampling rate of 2 Hz is sufficient to determine the pacemaker frequency. However, within a waveform, minor fluctuations can be important features induced by drugs, because they can be controlled by a series of different ion channels [29,30]. These fluctuation features were analysed using DFA and SampEn analysis.

In certain embodiments, the lowest possible pacemaker frequency is 0 cpm and the highest possible pacemaker frequency is <50 cpm. The inventors set the stopband filter between 0.01 and 2 Hz, i.e., 0.6 to 120 cpm, which is sufficient to cover most or all possible pacemaker frequencies. Very low frequency contamination (<2 cpm), hypothetically coming from the environment, sometimes appears; therefore, 2 cpm was set as the lower cut-off frequency in the power spectrum analysis. The normal intestinal pacemaker frequency is approximately 20-30 cpm. The second periodic frequency peak in the power spectrum can have significant power in some intestinal recordings. To avoid including the peaks of the resonance frequencies, the inventors set the upper cut-off frequency at 40 cpm, which is sufficient to include most pacemaker frequencies and is very likely below the second periodic peak frequency. The cut-off filters can be changed accordingly to suit different target animal models or even human tissues.

Moreover, some channels can often appear to record artefacts, noise, or contaminating signals, due to various reasons, including air bubbles or dirt on the surface of the electrode; a partially broken electrode; poor contact between the tissue and the electrodes and between the electrodes and the system; and poor experimental technique, that can result in tissue damage. Some of these problems can be resolved by regularly replacing the MEA chips. However, channel filtering is essential to allow the removal of these artefacts. Bad channels affect the derivation of the dominant frequency or power spectrum analysis, that use data averaged from most or all 60 channels. The derivation of velocity or propagation pattern analyses is more severely affected by bad channels, which requires the relative derivation of networking behaviors across the 60 channels. Although the data from bad channels can be auto-reconstructed by taking data from the nearby channels, there is a limitation in doing so. Therefore, in some datasets, if the baseline data do not meet the requirements of bad channel filtering, either the entire dataset will be eliminated, or only the frequency or power spectrum data will be included in the database.

Embodiments can provide advantageous systems and methods for data interpretation. Dopamine was used as an example in this study. Previous studies have shown that both dopamine agonists and antagonists inhibit pacemaker activity in isolated ICCs from mouse ilea [31]. This is consistent with the inventors' data on the ileum of Suncus murinus, in which dopamine significantly reduced the DP, amplitude, and slope, while increasing the period of the waveforms. These inhibitory effects were also observed in duodenal segments. In the stomach, the AF, percentage of tachy-rhythms, and signal irregularity were significantly increased by dopamine. In humans, the precursor of dopamine, L-DOPA, can slow down stomach emptying [32], while dopamine antagonists, such as domperidone, facilitate peristalsis and gastric emptying [33]. Apomorphine, a dopamine receptor agonist and an anti-PD drug, is also well known to induce emesis. It has been suggested that dopamine agonists should be administered when the stomach is empty, or even via non-oral routes, for PD treatment [34,35]. These clinical suggestions can be related to the induction of irregular gastric slow waves by dopamine. However, it is important to note that the effects seen in the current study were on isolated gut tissues, which are independent of central reflexes.

Certain findings in colonic tissue were distinct from those in the stomach and proximal intestine. The inventors found that dopamine had a biphasic effect, in which the percentage of tachy-rhythms increased after dopamine administration at 1-10 μM, whereas the percentage of brady-rhythms increased after dopamine administration at 100 μM. These findings can help explain the effects of dopamine in the human colon, where it induces phasic contractions at lower concentrations, but acts as a relaxant at higher concentrations [36]. Due to these biphasic effects, the concentration of dopamine in the colon becomes important when interpreting colon-related adverse effects, such as constipation. It is noteworthy that transdermal dopamine therapy [37] appears to reduce the side effects of oral dopamine therapy at delaying gastric emptying or worsening constipation [10]. This is because the slow release of a low concentration of dopamine can relieve the side effects of high-dose dopamine therapy.

Dopamine also induced significant irregular signal shapes, as determined by the DFA fluctuation function in the stomach and along the gut. This provides further evidence for the role of dopamine in GI motility and the potential reasons for dopamine-induced GI dysrhythmia.

Using the inventors' novel phase-based pattern distribution analysis, the activation time pattern was found to be significantly altered in duodenal and ileal tissues treated with dopamine at 100 nM and 100 μM, but not at intermediate concentrations of 1 μM and 10 μM. This indicates a new type of biphasic effect of dopamine in the upper gut. There are less clinical data related to the duodenum and ileum, but alterations in slow-wave propagation patterns can induce dysrhythmic motility in the upper gut, which can be related to some unexplainable clinical effects, such as abdominal pain and discomfort. In colonic tissues, although the frequency distribution showed a significant biphasic effect, the pattern distribution was relatively stable.

In certain embodiments of the analytical pipeline developed in this study, standardised spectrograms and DFA and SampEn graphs were automatically plotted and saved as images for all datasets. These graphs can be useful in deep learning protocols for more advance feature extraction. Embodiments can provide certain weights for each feature to construct a reference index value, which will be named as the ‘GI Dysrhythmia Index’. This index value can be used to indicate the level of drug-induced GI dysrhythmia, based on pacemaker activity tested using this standardised methodology.

### Example 3 Predicting Drug Adverse Effects Using a New Gastro-Intestinal Pacemaker Activity Drug Database (GIPADD)

Background and aims of this example include demonstrating and better understanding embodiments of the subject invention where electrical data a source of big-data for training AI for drug discovery. A Gastro-Intestinal Pacemaker Activity Drug Database was built using a standardized methodology to test drug effects on EF of GI pacemaker activity. The current example used data obtained from 89 drugs with 4,867 datasets to evaluate the potential use of the GIPADD for predicting drug AEs using a ML approach and to explore correlations between AEs and GI pacemaker activity.

Methods included the following. Twenty-four EFs were extracted using an automated analytical pipeline from the electrical signals recorded before and after acute drug treatment at 3 concentrations (or more) on 4-types of GI tissues (stomach, duodenum, ileum and colon). Extracted features were normalized and merged with an online side-effect resource (SIDER) database. Sixty-six common AEs were selected for testing. Different algorithms of classification ML models, including Naïve Bayes, discriminant analysis, classification tree, k-nearest neighbors, support vector machine and an ensemble model were tested. Separated tissue models were also tested. Averaging experimental repeats and dose adjustment were performed to refine the prediction results. Random datasets were created for model validation.

Results included the following. After model validation, 9 AEs classification ML model were constructed with accuracy ranging from 67-80%. EFs can be further grouped into ‘excitatory’ and ‘inhibitory’ types of AEs. This provides a novel approach wherein drugs are clustered based on EFs. Drugs acting on similar receptors can share similar EF profile, indicating advantageous use of the database to predict drug targets.

Conclusions included the following. Embodiments of the subject invention advantageously apply GIPADD, a growing database where prediction accuracy is expected to improve, to develop ML models predicting drug AEs and other factors. Embodiments provide novel insights on how EFs can be used as a new source of big-data in health and disease.

Database construction proceeded as follows. Datasets accumulated in the database were produced using methodologies according to certain embodiments of the subject invention. (See, e.g., Liu J Y H, et al. Use of a microelectrode array to record extracellular pacemaker potentials from the gastrointestinal tracts of the ICR mouse and house musk shrew (Suncus murinus). Cell Calcium 2019; 80. Liu J Y H, et al. Acetylcholine exerts inhibitory and excitatory actions on mouse ileal pacemaker activity: Role of muscarinic versus nicotinic receptors. Am J Physiol—Gastrointest Liver Physiol 2020; 319:G97-107. Liu J Y H, et al. Involvement of TRPV1 and TRPA1 in the modulation of pacemaker potentials in the mouse ileum. Cell Calcium 2021; 97:102417. Liu J Y H, et al. A pipeline for phase-based analysis of in vitro micro-electrode array recordings of gastrointestinal slow waves. Annu Int Conf IEEE Eng Med Biol Soc IEEE Eng Med Biol Soc Annu Int Conf 2021; 2021:261-4. TuL, et al. Insights Into Acute and Delayed Cisplatin-Induced Emesis From a Microelectrode Array, Radiotelemetry and Whole-Body Plethysmography Study of Suncus murinus (House Musk Shrew). Front Pharmacol 2021; 12. Liu J Y H, et al. Regional differences of tachykinin effects on smooth muscle and pacemaker potentials of the stomach, duodenum, ileum and colon of an emetic model, the house musk shrews. Neuropeptides 2022; 97:102300. Each of which, respectively, is hereby incorporated by reference in its entirety, including all graphs and figures, to the extent such disclosure is not inconsistent with the explicit teachings of this application.)

A total of 24 features, including dominant frequency, average frequency, dominant power, amplitude, period, velocity; percentage of contribution in power spectrum divided into percentages of brady-rhythm, normal-rhythm, and tachy-rhythm; signal stability, and complexity features including multiscale sample entropy and detrended fluctuation analysis divided into various time window scale; and wave propagation features including change in dominant propagation patterns were automatically extracted using customized and automated analytical programs according to an embodiment of the subject invention. Standardized and optimized filters and thresholds settings were included in certain analytical programs to remove datasets that did not meet baseline signal quality requirements.

Learning datasets construction proceeded as follows. All 24 features were normalized into percentage change values (e.g., by [(X_{post-drug}−X_{Baseline})/X_{Baseline}×100%] for data with specific units, and (% X_{post-drug}−% X_{Baseline}) for features which were originally presented in percentage of contribution). Normalized features and the drug name, tested dose and tissue-type were further merged with the online side effect resource (SIDER) database (version downloaded in October 2021)[46] based on matched drug names. Sixty-six common AEs were selected for this study. Each respective drug was first marked as ‘1’ indicating a positive correlation with each respective one of the 66 AEs. If the drug was clinically used with an indication for treatment of the listed AEs, a negative correlation ‘0’ was marked regardless of the positive correlation to the AEs. This step was to minimize the potential false reports of AEs due to existing conditions in patients. Otherwise, a negative correlation ‘0’ was marked for all drugs without the listed side effect. A limitation of this step was the potential of missing positive correlations (e.g., a false negative) of drugs that are seldom used, or have limited entries by virtue of being newly-launched into the market. Imbalanced datasets were identified using a ratio calculated using the following formula:

$$Balance Ratio = \frac{Number of positive correlated drugs}{Total number of drugs}$$

The acceptable range of dataset balance ratios for this study was set to 0.25-0.75. Balance ratios of <0.25 or >0.75 were considered as imbalanced datasets in the current study, and such datasets were not included in any statistical analysis in model comparisons. It is contemplated within the scope of the subject invention that certain embodiments will reach closer to a ratio of 0.5, or closer to another ratio or range of ratios (e.g., 0.26<balance ratio<0.74; 0.3<balance ratio<0.7; 0.4<balance ratio<0.6; 0.45<balance ratio<0.55; 0.48<balance ratio<0.53; 0.49<balance ratio<0.51; including ranges, divisions, and combinations thereof) with more datasets added into the database. Certain embodiments can advantageously provide a range of 0.4<balance ratio>0.6, and this target ratio range can be applied when more drugs are added into the database. This initial study was designed for a proof of concept for the application of the database, and therefore advantageously applied the more inclusive balance ratio range of 0.25-0.75. Features refinement proceeded as follows. Useful features were selected based on binary division of positive and negative datasets with significant differences on the mean of the two groups with p-value (p<0.05) using student's t-test. Selected features were then used for training ML models. Two types of learning datasets were built: (i) 164 average datasets through averaging data obtained from the same drug and same dose, aligning 24×4=96 features obtained from 4-types of tissue tested; (ii) 4,869 single datasets containing 3-10 experimental repeating datasets testing the 89 drugs, >3 doses and 4 types of GI tissue in different experiment preparations.

Machine learning models development proceeded as follows. Several ML models were built and compared: (a) models learnt by the 164 average datasets in (i), (b) models learnt by full 4,869 datasets in (ii), (c) models learnt by datasets in (ii) separated into tissue-type: stomach, duodenum, ileum and colon before training. Three types of classification algorithms were used for (b): naïve Bayes, classification tree and k-nearest neighbor (KNN) and 5 types of classification algorithms were used for (a) and (c): naïve Bayes, classification tree and KNN, discriminant analysis and support vector machine (SVM). Discriminant analysis and SVM were not applied in (b) because the dataset contains empty features which are not readily handled by these algorithms. An additional ensemble model was built through averaging prediction results obtained by either the 3 models or 5 models, respectively. Datasets were randomly separated into half for training and another half for testing. Randomization and training were repeated for seven iterations and results were evaluated to identify the best model. A random dataset of the same sample size was generated based on normal distribution using mean and sample standard deviation of each feature. Models generated using the random dataset were then compared with models generated using the actual dataset. This step validated potential biased prediction accuracy using imbalanced datasets with overfitting problems. Models which did not pass the validation test were discarded.

Prediction results refinement proceeded as follows. Repeated experimental datasets of the same drug and same dose in the prediction results of (b) and (c) were averaged and value>0.5 was listed as a positive prediction result ‘1’, otherwise as a negative prediction ‘0’. Dose weight adjustment was also performed based on the assumption that higher dose can induce more severe AEs. Prediction results were adjusted by an approximately 2-fold weight: 1, 0.5, 0.3, 0.1 and 0.05 in descending order of dose tested (in most cases these were separated by 10-fold). Dose weight adjustment in this study is a preliminary proof of concept to test whether the tested doses of drugs can contribute to final prediction result to improve prediction accuracy, and this study describes a simple method on how it can be incorporated into the ML models to adjust final prediction results. Values of weights can be adjusted using methods including but not limited to feedback Neural Network when more data is available for training in the future. Prediction accuracy was compared between prediction results with or without the above averaging and adjustment to show if these procedures can improve predictions. The flow of steps for selecting high performing ML models for selected AEs is summarized in FIGS. 13A and 13B.

Example applications of selected ML models proceeded as follows. The selected models were tested for the application to generate an adverse effect prediction report for several selected drugs. Selected models included 5 properties: the selected AEs for prediction, selected dataset type (a), (b) or (c) for training, algorithm-used, tissue-type, and presence or absence of prediction refinement procedures. Seven randomized predictions were performed to create correlated probability presented in percentage of chance that a certain drug can induce the tested AEs. The AE prediction results were compared with the SIDER database. Another 3 drugs which were not included in training ML model due to lack of matching drugs in SIDER were also applied for testing. Models with imbalanced datasets, or those that failed to pass the validation test using random datasets, were not included in the final sets of AEs prediction output. Re-construction and re-training of failed models in this study is contemplated within the scope of the subject invention as the database grow larger or as additional data becomes available.

Data analysis proceeded as follows. Statistical analysis comparing different ML models was performed using PRISM 8.0 software (GraphPad Software, San Diego, CA). Machine learning was performed using MATLAB 2020b The MathWorks, Inc., Natick, MA). All numerical data are expressed as mean±standard deviation and p<0.05 was considered statistically significant. Network cluster graph was plotted using a custom program written in R version 4.2.2.

Model comparison with different pre-training and post-training refinement procedures proceeded as follows. A total of 89 drugs were matched with the SIDER database, and the ratio of positive-correlated datasets over total number of datasets for 66 selected common AEs were calculated and listed in Table 3, which shows a list of selected adverse effects (AEs) analyzed according to an embodiment of the subject invention. AEs with data ratio [number of positive-correlated datasets/total number of datasets] of between 0.25-0.75 were considered acceptable for this example and were selected for further model comparison studies which refined 14 selected AEs. Within the 14 AEs, the average prediction accuracy was compared between different models: (A) 164 average datasets averaging experimental repeats before training (B) 4,869 single datasets, (C) 4,869 single datasets separated into tissue-type before training (FIG. 14A). Model B showed the best accuracy 67.1±6.6% (n=14) regardless of training algorithm and tissue-type. Model A only showed a 58.0±4.7% accuracy and model C showed 65.7±6.4% accuracy (n=14). This result shows that pre-averaging experimental repeated datasets or pre-separating tissue-type before ML had generally reduced the final prediction accuracy. Between Model B and C, the difference is whether or not to isolate single tissue-type in training, and including all tissues in B improved accuracy only approximately 2%. It is contemplated within the scope of the subject invention that these pre-training procedures can be effective and advantageous for predicting certain tissue-specific AEs.

TABLE 3
Adverse Events

| | Colon | | | Duodenum | | | Ileum | | | Stomach | | | All | | |
|---|---|---|---|---|---|---|---|---|---|---|---|---|---|---|---|
| Adverse effects | −ve | +ve | ratio | −ve | +ve | ratio | −ve | +ve | ratio | −ve | +ve | ratio | −ve | +ve | ratio |
| Abdominal cramps | 1357 | 184 | 0.12 | 1284 | 171 | 0.12 | 1293 | 182 | 0.12 | 343 | 53 | 0.13 0 | 4277 | 590 | 0.12 |
| Abdominal discomfort | 1430 | 111 | 0.07 | 1339 | 116 | 0.08 | 1362 | 113 | 0.08 | 350 | 46 | 0.12 | 4481 | 386 | 0.08 |
| Abdominal distension | 1326 | 215 | 0.14 | 1254 | 201 | 0.14 | 1261 | 214 | 0.15 | 343 | 53 | 0.13 | 4184 | 683 | 0.14 |
| Abdominal pain | 1029 | 512 | 0.33 | 963 | 492 | 0.34 | 969 | 506 | 0.34 | 268 | 128 | 0.32 | 3229 | 1638 | 0.34 |
| Agitation | 1352 | 189 | 0.12 | 1274 | 181 | 0.12 | 1288 | 187 | 0.13 | 352 | 44 | 0.11 | 4266 | 601 | 0.12 |
| Amnesia | 1436 | 105 | 0.07 | 1359 | 96 | 0.07 | 1382 | 93 | 0.06 | 352 | 44 | 0.11 | 4529 | 338 | 0.07 |
| Angina pectoris | 1403 | 138 | 0.09 | 1319 | 136 | 0.09 | 1341 | 134 | 0.09 | 355 | 41 | 0.10 | 4418 | 449 | 0.09 |
| Anxiety | 1148 | 393 | 0.26 | 1095 | 360 | 0.25 | 1109 | 366 | 0.25 | 264 | 132 | 0.33 | 3616 | 1251 | 0.26 |
| Arrhythmia | 1107 | 434 | 0.28 | 1028 | 427 | 0.29 | 1056 | 419 | 0.28 | 266 | 130 | 0.33 | 3457 | 1410 | 0.29 |
| Back pain | 1337 | 204 | 0.13 | 1267 | 188 | 0.13 | 1282 | 193 | 0.13 | 333 | 63 | 0.16 | 4219 | 648 | 0.13 |
| Blood pressure | 1541 | 0 | 0.00 | 1455 | 0 | 0.00 | 1475 | 0 | 0.00 | 396 | 0 | 0.00 | 4867 | 0 | 0.00 |
| abnormal | | | | | | | | | | | | | | | |
| Blood pressure | 1541 | 0 | 0.00 | 1455 | 0 | 0.00 | 1475 | 0 | 0.00 | 396 | 0 | 0.00 | 4867 | 0 | 0.00 |
| fluctuation | | | | | | | | | | | | | | | |
| Bradycardia | 1217 | 324 | 0.21 | 1146 | 309 | 0.21 | 1163 | 312 | 0.21 | 298 | 98 | 0.25 | 3824 | 1043 | 0.21 |
| Cardiac failure | 1449 | 92 | 0.06 | 1365 | 90 | 0.06 | 1381 | 94 | 0.06 | 379 | 17 | 0.04 | 4574 | 293 | 0.06 |
| Constipation | 1120 | 421 | 0.27 | 1049 | 406 | 0.28 | 1056 | 419 | 0.28 | 288 | 108 | 0.27 | 3513 | 1354 | 0.28 |
| Cough | 1366 | 175 | 0.11 | 1292 | 163 | 0.11 | 1306 | 169 | 0.11 | 356 | 40 | 0.10 | 4320 | 547 | 0.11 |
| Decreased appetite | 1191 | 350 | 0.23 | 1138 | 317 | 0.22 | 1146 | 329 | 0.22 | 278 | 118 | 0.30 | 3753 | 1114 | 0.23 |
| Depression | 1406 | 135 | 0.09 | 1331 | 124 | 0.09 | 1340 | 135 | 0.09 | 364 | 32 | 0.08 | 4441 | 426 | 0.09 |
| Diarrhoea | 968 | 573 | 0.37 | 903 | 552 | 0.38 | 922 | 553 | 0.37 | 231 | 165 | 0.42 | 3024 | 1843 | 0.38 |
| Dizziness | 969 | 572 | 0.37 | 911 | 544 | 0.37 | 917 | 558 | 0.38 | 263 | 133 | 0.34 | 3060 | 1807 | 0.37 |
| Dry eye | 1472 | 69 | 0.04 | 1388 | 67 | 0.05 | 1405 | 70 | 0.05 | 376 | 20 | 0.05 | 4641 | 226 | 0.05 |
| Dry mouth | 1221 | 320 | 0.21 | 1167 | 288 | 0.20 | 1171 | 304 | 0.21 | 317 | 79 | 0.20 | 3876 | 991 | 0.20 |
| Dysgeusia | 1358 | 183 | 0.12 | 1279 | 176 | 0.12 | 1294 | 181 | 0.12 | 345 | 51 | 0.13 | 4276 | 591 | 0.12 |
| Dyspepsia | 1188 | 353 | 0.23 | 1145 | 310 | 0.21 | 1133 | 342 | 0.23 | 304 | 92 | 0.23 | 3770 | 1097 | 0.23 |
| Dysphagia | 1321 | 220 | 0.14 | 1234 | 221 | 0.15 | 1247 | 228 | 0.15 | 330 | 66 | 0.17 | 4132 | 735 | 0.15 |
| Flushing | 1312 | 229 | 0.15 | 1245 | 210 | 0.14 | 1260 | 215 | 0.15 | 333 | 63 | 0.16 | 4150 | 717 | 0.15 |
| Gastritis | 1365 | 176 | 0.11 | 1309 | 146 | 0.10 | 1309 | 166 | 0.11 | 338 | 58 | 0.15 | 4321 | 546 | 0.11 |
| Gastrointestinal | 1142 | 399 | 0.26 | 1088 | 367 | 0.25 | 1097 | 378 | 0.26 | 290 | 106 | 0.27 | 3617 | 1250 | 0.26 |
| disorder | | | | | | | | | | | | | | | |
| Gastrointestinal pain | 1122 | 419 | 0.27 | 1043 | 412 | 0.28 | 1048 | 427 | 0.29 | 295 | 101 | 0.26 | 3508 | 1359 | 0.28 |
| Gastrooesophageal | 1459 | 82 | 0.05 | 1380 | 75 | 0.05 | 1400 | 75 | 0.05 | 372 | 24 | 0.06 | 4611 | 256 | 0.05 |
| reflux | | | | | | | | | | | | | | | |
| Headache | 1155 | 386 | 0.25 | 1092 | 363 | 0.25 | 1104 | 371 | 0.25 | 305 | 91 | 0.23 | 3656 | 1211 | 0.25 |
| Heart rate abnormal | 1541 | 0 | 0.00 | 1455 | 0 | 0.00 | 1475 | 0 | 0.00 | 396 | 0 | 0.00 | 4867 | 0 | 0.00 |
| Heart rate irregular | 1541 | 0 | 0.00 | 1455 | 0 | 0.00 | 1475 | 0 | 0.00 | 396 | 0 | 0.00 | 4867 | 0 | 0.00 |
| Hypersensitivity | 1093 | 448 | 0.29 | 1044 | 411 | 0.28 | 1049 | 426 | 0.29 | 270 | 126 | 0.32 | 3456 | 1411 | 0.29 |
| Hypertension | 1171 | 370 | 0.24 | 1114 | 341 | 0.23 | 1123 | 352 | 0.24 | 304 | 92 | 0.23 | 3712 | 1155 | 0.24 |
| Hypotension | 1300 | 24 | 0.16 | 1221 | 234 | 0.16 | 1247 | 228 | 0.15 | 347 | 49 | 0.12 | 4115 | 752 | 0.15 |
| Hypothermia | 1496 | 45 | 0.03 | 1410 | 45 | 0.03 | 1430 | 45 | 0.03 | 390 | 6 | 0.02 | 4726 | 141 | 0.03 |
| Increased urination | 1541 | 0 | 0.00 | 1455 | 0 | 0.00 | 1475 | 0 | 0.00 | 396 | 0 | 0.00 | 4867 | 0 | 0.00 |
| Insomnia | 1286 | 255 | 0.17 | 1208 | 247 | 0.17 | 1227 | 248 | 0.17 | 338 | 58 | 0.15 | 4059 | 808 | 0.17 |
| Irritability | 1442 | 99 | 0.06 | 1352 | 103 | 0.07 | 1373 | 102 | 0.07 | 376 | 20 | 0.05 | 4543 | 324 | 0.07 |
| Menstrual disorder | 1454 | 87 | 0.06 | 1365 | 90 | 0.06 | 1386 | 89 | 0.06 | 373 | 23 | 0.06 | 4578 | 289 | 0.06 |
| Menstruation delayed | 1541 | 0 | 0.00 | 1455 | 0 | 0.00 | 1475 | 0 | 0.00 | 396 | 0 | 0.00 | 4867 | 0 | 0.00 |
| Menstruation irregular | 1449 | 92 | 0.06 | 1355 | 100 | 0.07 | 1381 | 94 | 0.06 | 371 | 25 | 0.06 | 4556 | 311 | 0.06 |
| Motor restlessness | 1541 | 0 | 0.00 | 1455 | 0 | 0.00 | 1475 | 0 | 0.00 | 396 | 0 | 0.00 | 4867 | 0 | 0.00 |
| Muscle spasms | 1301 | 240 | 0.16 | 1237 | 218 | 0.15 | 1253 | 222 | 0.15 | 307 | 89 | 0.22 | 4098 | 769 | 0.16 |
| Nasopharyngitis | 1450 | 91 | 0.06 | 1362 | 93 | 0.06 | 1382 | 93 | 0.06 | 380 | 16 | 0.04 | 4574 | 293 | 0.06 |
| Nausea | 876 | 665 | 0.43 | 822 | 633 | 0.44 | 833 | 642 | 0.44 | 213 | 183 | 0.46 | 2744 | 2123 | 0.44 |
| Nervousness | 1399 | 142 | 0.09 | 1314 | 141 | 0.10 | 1334 | 141 | 0.10 | 361 | 35 | 0.09 | 4408 | 459 | 0.09 |
| Oedema | 1263 | 278 | 0.18 | 1200 | 255 | 0.18 | 1221 | 254 | 0.17 | 301 | 95 | 0.24 | 3985 | 882 | 0.18 |
| Palpitations | 1255 | 286 | 0.19 | 1175 | 280 | 0.19 | 1193 | 282 | 0.19 | 329 | 67 | 0.17 | 3952 | 915 | 0.19 |
| Pregnancy | 1424 | 117 | 0.08 | 1328 | 127 | 0.09 | 1358 | 117 | 0.08 | 374 | 22 | 0.06 | 4484 | 383 | 0.08 |
| Rash | 1067 | 474 | 0.31 | 1016 | 439 | 0.30 | 1021 | 454 | 0.31 | 257 | 139 | 0.35 | 3361 | 1506 | 0.31 |
| Renal impairment | 1541 | 0 | 0.00 | 1455 | 0 | 0.00 | 1475 | 0 | 0.00 | 396 | 0 | 0.00 | 4867 | 0 | 0.00 |
| Respiration abnormal | 1527 | 14 | 0.01 | 1441 | 14 | 0.01 | 1463 | 12 | 0.01 | 392 | 4 | 0.01 | 4823 | 44 | 0.01 |
| Retching | 1478 | 63 | 0.04 | 1394 | 61 | 0.04 | 1412 | 63 | 0.04 | 372 | 24 | 0.06 | 4656 | 211 | 0.04 |
| Sexual dysfunction | 1507 | 34 | 0.02 | 1427 | 28 | 0.02 | 1441 | 34 | 0.02 | 385 | 11 | 0.03 | 4760 | 107 | 0.02 |
| Somnolence | 1284 | 257 | 0.17 | 1205 | 250 | 0.17 | 1217 | 258 | 0.17 | 350 | 46 | 0.12 | 4056 | 811 | 0.17 |
| Sweating | 1260 | 281 | 0.18 | 1186 | 269 | 0.18 | 1200 | 275 | 0.19 | 311 | 85 | 0.21 | 3957 | 910 | 0.19 |
| Swelling | 1397 | 144 | 0.09 | 1318 | 137 | 0.09 | 1340 | 135 | 0.09 | 357 | 39 | 0.10 | 4412 | 455 | 0.09 |
| Tachycardia | 1028 | 513 | 0.33 | 976 | 479 | 0.33 | 996 | 479 | 0.32 | 256 | 140 | 0.35 | 3256 | 1611 | 0.33 |
| Temperature | 1517 | 24 | 0.02 | 1433 | 22 | 0.02 | 1449 | 26 | 0.02 | 387 | 9 | 0.02 | 4786 | 81 | 0.02 |
| intolerance | | | | | | | | | | | | | | | |
| Upset stomach | 1497 | 44 | 0.03 | 1410 | 45 | 0.03 | 1430 | 45 | 0.03 | 379 | 17 | 0.04 | 4716 | 151 | 0.03 |
| Urinary incontinence | 1541 | 0 | 0.00 | 1455 | 0 | 0.00 | 1475 | 0 | 0.00 | 396 | 0 | 0.00 | 4867 | 0 | 0.00 |
| Urination impaired | 1496 | 45 | 0.03 | 1410 | 45 | 0.03 | 1430 | 45 | 0.03 | 390 | 6 | 0.02 | 4726 | 141 | 0.03 |
| Vision blurred | 1541 | 0 | 0.00 | 1455 | 0 | 0.00 | 1475 | 0 | 0.00 | 396 | 0 | 0.00 | 4867 | 0 | 0.00 |
| Vomiting | 878 | 663 | 0.43 | 817 | 638 | 0.44 | 827 | 648 | 0.44 | 206 | 190 | 0.48 | 2728 | 2139 | 0.44 |

Based on Model B, the average prediction accuracy and best prediction accuracy was compared between models built using actual datasets and random datasets for the 14 AEs. In 3 out of 14 AEs' this test failed to identify significant features for model building using random datasets. In 6 out of 11 AEs this test produced >0.5% better accuracy using actual datasets compared with random datasets in either average or best prediction accuracy (Table-4). Pre-averaging training models that contain only 164 datasets suffered from limited model size between training and testing during randomization. Only models with >80% total drugs after randomization were considered in model accuracy comparison. The prediction accuracy of the final 9 selected models was 67.4-79.8%, true positive rate 62-100% and true negative rate 65-83% (Table-5). These 9 AEs ML models predict anxiety (accuracy: 79.8%), gastrointestinal disorder (79.0%), constipation (76.4%), gastrointestinal pain (75.9%), arrhythmia (74.2%), vomiting (74.1%), dizziness (70.4%), rash (70.1%) and diarrhea (69.7%). In which, anxiety is related to psychology, arrhythmia is cardiology and rash is immunology, while the rest are GI-related AEs. Arrhythmia is easy to relate, because cardiac pacemaking activity shares very similar pacemaking mechanisms with the GI [47]. For psychology-related AEs, while not being bound by theory, the inventors hypothesize this could be due to the shared receptor expression between the brain and the gut, as well as other parts of our body. For example, serotonin controls emotions and serotonin receptors are also important drug target receptors for anti-emetic therapies [12,13]. Rash is an immunity-related AEs, where GI contributed to 70% of our body immunity expressing many types of pre-active immune cells ready to fight against toxic substances and pathogens invasion from ingested materials [50].

Table-4 Comparison between models built using actual datasets and random datasets. Table shows the average accuracy (left columns) and the best accuracy (right column) of all models created with different algorithm-type, tissue-type and with seven randomized training.

(Note at #1 No significant features were identified from the randomized datasets for model building, therefore, no prediction accuracy data were available.)

TABLE 4

| | Average | Average | | Best | Best | |
|---|---|---|---|---|---|---|
| | accuracy | accuracy | | accuracy | accuracy | |
| | (Actual | (Random | | (Actual | (Random | |
| Adverse effects | datasets) | datasets) | Difference | datasets) | datasets) | Difference |
| Abdominal pain | 65.5 | 65.3 | 0.2 | 68.5 | 68.5 | 0.0 |
| Anxiety | 74.1 | 72.9 | 1.3 | 77.5 | 75.3 | 2.2 |
| Arrhythmia | 70.5 | 68.6 | 1.9 | 74.2 | 71.9 | 2.2 |
| Constipation | 72.1 | #1 | | 76.4 | | |
| Diarrhoea | 62.1 | 62.0 | 0.1 | 69.7 | 66.3 | 3.4 |
| Dizziness | 59.7 | | | 67.4 | | |
| Gastrointestinal disorder | 72.7 | | | 78.1 | | |
| Gastrointestinal pain | 71.4 | 71.0 | 0.3 | 74.6 | 73.0 | 1.5 |
| Headache | 75.9 | 76.0 | −0.1 | 78.7 | 79.0 | −0.4 |
| Hypersensitivity | 68.4 | 68.3 | 0.1 | 71.9 | 71.9 | 0.0 |
| Nausea | 56.6 | 56.8 | −0.2 | 61.4 | 62.9 | −1.6 |
| Rash | 65.4 | 64.7 | 0.7 | 69.1 | 69.2 | −0.1 |
| Tachycardia | 67.1 | 66.9 | 0.2 | 70.5 | 70.8 | −0.3 |
| Vomiting | 56.4 | 57.0 | −0.6 | 68.2 | 66.3 | 1.9 |

Table-5 Table showing the final selected useful ML model for predicting 9 AEs, and the properties of the selected models. TPR: true positive rate; TNR: true negative rate; FPR: false positive rate; FNR: false negative rate; TP: true positive count; TN: true negative count; FP: false positive count; FN: false negative count.
#1 A: 164 average datasets through averaging data obtained from the same drug and same dose, aligning 24×4=96 features obtained from 4-type of tissue tested; B: 4,869 single datasets with experimental repeated datasets testing the same drug, same dose and same tissue in different preparation for 3-10 times. C: 4,869 single datasets with repeating datasets and trained separately for different tissue-type.
#2 Prediction adjustment 1: Combine and average the prediction results of repeated datasets of the same treatment. Y: Yes; N: No.
#3 Prediction adjustment 2: Dose weight adjustment based on simple hypothesis that higher dose had higher chance in side effects induction by 1, 0.5, 0.3, 0.1, 0.05, currently no drugs are tested for more than 5 doses. Y: Yes; N: No.
#4 Representative tissue for classification: s: stomach; d: duodenum; i: ileum; c: colon; a: all.

TABLE 5

| | | | Model | Average | Dose | Ratio of | | | |
|---|---|---|---|---|---|---|---|---|---|
| Adverse effects | Accuracy | Algorithm | Type^{#1} | Repeats ^{#2} | Adjust ^{#3} | testing data | | | |
| Anxiety | 79.8 | KNN | B | Y | N | 0.75 | | | |
| Arrhythmia | 74.2 | Bayes | B | Y | Y | 0.29 | | | |
| Constipation | 76.4 | Tree | B | Y | Y | 0.27 | | | |
| Diarrhoea | 69.7 | Tree | B | Y | Y | 0.36 | | | |
| Dizziness | 70.4 | Bayes | A | N | N | 0.63 | | | |
| | 67.4 | Ensemble | B | Y | Y | 0.39 | | | |
| Gastrointestinal | 79.0 | Bayes | C | Y | N | 0.26 | | | |
| disorder | | | | | | | | | |
| Gastrointestinal | 75.9 | KNN | C | Y | N | 0.29 | | | |
| pain | | | | | | | | | |
| Rash | 70.1 | Tree | C | Y | Y | 0.34 | | | |
| Vomiting | 74.1 | Bayes | A | Y | Y | 0.69 | | | |
| | 71.0 | Tree | C | Y | N | 0.44 | | | |
| Adverse effects | Tissue ^{#4} | TPR | TNR | FPR | FNR | TP | TN | FP | FN |
| Anxiety | c | 100 | 79 | 21 | 0 | 4 | 67 | 18 | 0 |
| Arrhythmia | a | 71 | 74 | 26 | 29 | 5 | 61 | 21 | 2 |
| Constipation | c | 76 | 80 | 24 | 20 | 20 | 4 | 64 | 20 |
| Diarrhoea | a | 62 | 72 | 28 | 38 | 13 | 49 | 19 | 8 |
| Dizziness | a | 71 | 67 | 33 | 29 | 45 | 12 | 6 | 18 |
| | a | 100 | 65 | 35 | 0 | 6 | 54 | 29 | 0 |
| Gastrointestinal | s | 100 | 78 | 22 | 0 | 3 | 46 | 13 | 0 |
| disorder | | | | | | | | | |
| Gastrointestinal | d | 100 | 75 | 25 | 0 | 4 | 62 | 21 | 0 |
| pain | | | | | | | | | |
| Rash | d | 75 | 70 | 30 | 25 | 6 | 55 | 24 | 2 |
| Vomiting | a | 73 | 83 | 17 | 27 | 55 | 5 | 1 | 20 |
| | S | 74 | 70 | 30 | 26 | 14 | 30 | 13 | 5 |

Based on these 9 AEs and Model B, ML models were further compared across tissue-type (FIG. 14B) and algorithm-type (FIG. 14C). Comparisons included the refinement procedures of prediction results through averaging experimental repeated datasets and dose-weight adjustment. The result shows that the procedure of dose-weight adjustment generally did not improve the accuracy of predictions in all tested models, but rather slightly reduced the accuracy with range between 0.01-0.43%. Without being bound by theory, the inventors hypothesize that the effect of dose weight adjustment can be improved with further investigation to optimize the weight values, which can be tested and proven to show advantageous effects (e.g., with larger database.) Across different tissue models, averaging repeated datasets significantly improved the average prediction accuracy for all types of tissue models by 1.8-2.2% (p<0.001, n=294-1,428). This improvement is expected, as more experimental repeats should help improving data accuracy, provided that there is a correlation between EFs and tested AEs. Across different classification algorithms, averaging repeated datasets significantly improved the accuracy only in KNN (+5.55%, p<0.001, n=238), classification tree (+5.45%, p<0.001, n=238), Naïve Bayes (+1.98%, p<0.001, n=238). However, this refinement procedure did not improve the prediction accuracy in an ensemble model (+0.11%, insignificant, n=238), discriminant analysis (−0.08%, p<0.01, n=238) and SVM model (−0.29%, p<0.001, n=238). Among all tissue models, the ileum had the best accuracy at 67.4±6.9%, compared to the stomach at 65.1±7.6%. Across different classification algorithms, the best algorithm is discriminant analysis (67.6±6.7%) and SVM (67.8±6.8%) before merging experimental repeats, while KNN model showed the lowest accuracy (65.1±7.4%) after merging experimental repeats. Note that the above model comparison only represents the general trend. Specific refinement procedures were sometimes found useful in improving accuracy for predicting certain AEs.

Feature selection and comparison proceeded as follows. Binary division between negative and positive AEs correlated datasets was used to select and refine features for training the ML model. Although ML models have not yet been successfully built for all 66 selected AEs (while note being bound by theory, the inventors hypothesize this is due to an observed significant imbalance in available datasets, and it is therefore within the scope of the subject invention with increased availability of data and development or recognition of new datasets, embodiments can provide new, better, and more reliable ML models for additional AEs), the feature selection process can identify correlated patterns of change in EFs of GI pacemaker activity for different groups of AEs. The identified significant features could be important factors to correlate GI pacemaker activities to health and disease. The correlated patterns can be divided into two major groups of AEs, ‘excitatory’ and ‘inhibitory’ AEs (Table-6, FIGS. 15A-15L). AE-inducing drugs in ‘excitatory’ AEs group (19 selected AEs) had more excitatory actions on the colon, which increased average frequency, further increased tachy-rhythm power and dominant power of the colon tissues, and also further reduced dominant power of stomach tissues compare to non-AE-inducing drugs. On the other hand, AE-inducing drugs in ‘inhibitory’ AEs group (7 selected AEs) had opposite effects which did not increase average frequency, tachy-rhythm power and dominant power on the colon tissues. However, the AE-inducing drugs in the 7 ‘inhibitory’ AEs shared common inhibitory effects on the duodenal tissues to further reduce the slope and amplitude, while increasing the period of waveform compared to non-AE-inducing drugs, where these changes were not identified in the ‘excitatory’ AEs. This phenomenon indicated that common patterns of change in GI pacemaker activity can be found in correlated sets of AEs. These patterns of change can be correlated to common receptor activation or inhibition to generally excite and inhibit GI pacemaker activity at different GI segments.

TABLE 6
List of “excitatory” AEs and “inhibitory” AEs sharing
similar change of pattern based on EF drug profile.

| “Excitatory” AEs | “Inhibitory” AEs |
|---|---|
| Common actions: | Common actions: |
| excitatory actions on the colon, | inhibitory effects on |
| reduced dominant power of the stomach | the duodenum |
| Dyspepsia | Cough |
| Dizziness | Headache |
| Rash | Insomnia |
| Vomiting | Hypothermia |
| Gastrointestinal pain | Angina pectoris |
| Tachycardia | Irritability |
| Diarrhoea | Palpitations |
| Gastrointestinal disorder | |
| Abdominal pain | |
| Arrhythmia | |
| Dysphagia | |
| Decreased appetite | |
| Gastritis | |
| Abdominal distension | |
| Constipation | |
| Gastroesophageal reflux | |
| Nausea | |
| Abdominal discomfort | |
| Muscle spasms | |

This study also provides a novel graphical representation on how drugs can be correlated with AE-related EFs. Drugs can be clustered based on refined EFs and plotted into a network graph. An example for constipation network model is shown in FIG. 16A. Two circles (shaded in yellow) show positive-correlation and negative-correlation with constipation, respectively based on the averaged and refined supervised EFs. The larger the distance between these two circles (black arrow), the better the model can distinguish between the constipation-inducing properties of drugs. Ondansetron (blue arrow, labelled with “ond”) and morphine (red arrow, “mor”) are two drugs-in-market that are known to induce constipation as AE. Based on this graph, we can also observe that drugs known to act on similar receptors are clustered together based on EFs (shaded in green), such as prostaglandin E1 (“pge1”) and prostaglandin E2 (“pge2”), or substance P (“sp”) and neurokinin A (“nka”), providing evidences that embodiments including GIPADD can also predict drug targeting receptors.

Other than the ‘excitatory’ and ‘inhibitory’ EF drug profiles, other interesting significant feature differences were identified. In GI-related AEs, drugs inducing abdominal distension and upset stomach had reduced the duodenal dominant pacemaker frequency at a higher level compared to non-AEs inducing drugs; and drugs inducing abdominal cramps did not alter ileum propagating velocity, but non-AE-inducing drugs generally induced it (FIG. 16B). In psychological AEs, drugs that induced anxiety and depression had increased the colon average pacemaker frequency, and further induced stomach average pacemaker frequency compared to non-AEs inducing drugs (FIG. 16C). In blood pressure-related AEs, hypotension-inducing drugs reduced duodenal propagating velocity, but not hypertension-inducing drugs. On the other hand, hypotension-inducing drugs reduced percentage of tachy-rhythm at the ileum, while hypertension-inducing drugs induced it. Hypotension-inducing drugs did not change the ileal propagation velocity, but hypertension-inducing drugs induced it significantly (FIG. 16D). Although these AE-to-EF correlations can be weak or in certain cases, very weak, these can improve brainstorming of novel connections and hypothesis beyond unaided human efforts. These correlations are advantageously generated based on computer calculations without the inherent bias in human-driven hypothesis.

Drug report generation proceeded as follows. The 9 selected ML models were applied for making predictions. Four drugs, apomorphine, atorvastatin, oxytocin, and amlodipine, were used for the trained models in AE prediction (Table-7). Models accurately predicted positive correlations in gastrointestinal disorder and gastrointestinal pain for amlodipine and negative correlations for apomorphine and atorvastatin. Models also predicted positive correlations in vomiting for apomorphine, constipation for amlodipine, rash for oxytocin. However, positive correlations in anxiety, arrhythmia, diarrhea and dizziness were not correctly identified for these selected drugs. In addition, another three drugs, neurokinin A (NKA), peptide YY and lipopolysaccharide (LPS), which were not used in training models due to missing side effect profile in SIDER were also tested, in which potential vomiting-inducing properties were predicted for NKA and peptide YY with 43% randomized prediction results showing positive correlations, while LPS showed very minor positive correlations with rash (14%) and constipation (14%).

Table-7. Example drug AE prediction report. A table showing the prediction results of 9 selected AEs for 4 selected drugs used in model training compared with AEs occurrence listed in SIDER, where ‘0’ indicates negative correlations and ‘1’ indicates positive correlations (left column), and the prediction results of another 3 drugs which was not included in SIDER and model training (right column).

TABLE 7

| | | | | | | | | | Drugs not included | | |
|---|---|---|---|---|---|---|---|---|---|---|---|
| | | | | | | | | | in training models | | |
| Adverse | Drugs used in training models | | | | | | | | | Peptide | |
| effects | Apomorphine | | Atorvastatin | | Oxytocin | | Amlodipine | | NKA | YY | LPS |
| Anxiety | 0% | 1 | 0% | 0 | 0% | 0 | 0% | 1 | 0% | 0% | 0% |
| Arrhythmia | 0% | 0 | 0% | 0 | 0% | 1 | 0% | 1 | 0% | 0% | 0% |
| Constipation | 29% | 1 | 0% | 0 | 0% | 0 | 57% | 1 | 0% | 0% | 14% |
| Diarrhea | 0% | 1 | 0% | 0 | 0% | 0 | 0% | 1 | 0% | 0% | 0% |
| Dizziness | 0% | 1 | 14% | 0 | 0% | 0 | 0% | 1 | 0% | 0% | 0% |
| Gastrointestinal | 0% | 0 | 0% | 0 | 0% | 1 | 100% | 1 | 0% | 0% | 0% |
| disorder | | | | | | | | | | | |
| Gastrointestinal | 0% | 0 | 0% | 0 | 0% | 0 | 100% | 1 | 0% | 0% | 0% |
| pain | | | | | | | | | | | |
| Rash | 0% | 0 | 0% | 0 | 71% | 1 | 0% | 1 | 0% | 0% | 14% |
| Vomiting | 71% | 1 | 0% | 0 | 0% | 1 | 0% | 1 | 43% | 43% | 0% |

Conclusions are summarized as follows. This example describes a selection process and prediction refinement procedures for creating the best classification ML model to predict selected AEs for drugs using the GIPADD database integrating with the SIDER database. This example also emphasizes the advantages of using standardized drug screening methodology to create EF drug databases, allowing highly-consistent and massive amount of comparisons to be performed between numerous of drugs simultaneously, which could quickly advance the unexplored knowledge on drug-induced effects on EF of GI pacemaker activity, or even discover novel correlations which we had never considered before. Using our established standardized drug testing methodology with the MEA technology [1-6] and automated data analytical pipeline [4], any novel EF drug profile and AE prediction result can be created in two to three days. This is expected to bring game-changing impact towards decision making in drug discovery.

Addition of one new drug profile into the database allows thousands of new comparative calculations. Within the cutoff-database used in this example, there were >6 billion comparisons and calculations made for each selected-AEs. Predictive accuracy is expected to improve with the growing GIPADD database. Its application can further extend to drug reposition, prediction of drug targets and therapeutic effects. This example only listed some of the interesting examples for the AE-to-EF correlation found using GIPADD. Within the scope of the subject invention, the development of additional advantageous and beneficial AE-to-EF correlations, ML models, and resulting AE predictions are contemplated.

Within the scope of the subject invention are contemplated additional improvements with respect to the AI algorithms, refinement procedures, and training data preparation processes in this example. As one illustrative but non-limiting example, with more data, the prediction results can shift from a classification model to a regression model to predict actual probability or frequency of occurrence for certain AEs. Multi-label classification models were also tested to potentially predict all listed AEs at once, although this example separated each respective AE for AI model creation to rule out overfitting and data imbalance due to limited data size.

Another limitation of the current method is that some AEs of interest can have weak correlation to GI pacemaker activity. GIPADD focuses only on the GI pacemaker activity, while there are many other types of electrical signals produced from other tissues and organs that embodiments of the subject invention can translate and decode. Within the scope of the subject invention, the inventors contemplate building similar electrophysiological drug databases for pacemaker activity found in other organs, such as the heart and uterus. In certain embodiments, EF big-data can advantageously improve drug discovery and scientific development with respect to a variety of AEs and EFs.

Embodiments can provide information targeting for personalised drug therapy. One goal of drug profiling based on GI pacemaker activity can be to enable the development of personalised drug therapy. In the digital era, numerous drug databases are being and can be constructed, including (1) a network medicine approach to search the existing literature to reposition drugs [38]; (2) deep-learning approaches to study drug docking with potential target receptors [39,40]; (3) drug adverse effects databases, such as the Side Effects Resource (SIDER) and the Food and Drug Administration's Adverse Event Reporting System; and (4) the International Union of Basic and Clinical Pharmacology (IUPHAR) guide to pharmacological listings. Research in the field of predicting drug adverse effect is still at an early stage [41]. Each drug database can have its strengths and limitations. For example, affinity values do not always correlate to the degree of physiological response of a drug. The EC50 or IC50 values are not universal in different cell, tissue, or animal models. Literature search approach can have over-focus on the explored area of research. The inventors believed creating a standardized test on drug physiological response could allow more reliable drug comparison within a certain system. Starting from the GI tract, the physiological effects of drugs on pacemaker activity could play a part in providing comprehensive information on potential drug effects on GI motility. Other standardized physiological databases can be developed for other tissue model, such as the brain and the heart. With or without the aid of artificial intelligence, clinicians and basic scientists can use these reference databases to identify the best drug therapy for their patients in the future.

Embodiments can provide a business model, system, or method that includes a unique drug-testing service on pacemaker potentials in gastrointestinal tissue, and consultancy based on expertise and a proprietary database. Pacemaker potentials can be recorded simultaneously from multiple microelectrodes embedded on a chip. Embodiments can provide systems and methods to enable an automatic, efficient, reliable, minimal error, and bias-free analysis technique to extract numerous of useful features from pacemaker signals for database construction. As more drugs are analyzed, more data are added into the database. The growing database becomes a more powerful resource and its predictive power to identify functional-effects increases. Thus, the profile of a novel chemical entity can be predicted with a high degree of accuracy. Embodiments can provide bespoke testing of drugs, chemical compounds, remedies, extracts or combination of the above, using a standardized protocol, as well as multiple potential applications of the database to create different machine learning models.

Embodiments can apply the MEA to record pacemaker activity. Embodiments can employ the use of the MEA technology for large-scale drug screening. Embodiments can advantageously employ data extracted from multiple novel features derived from pacemaker activity of gastrointestinal tissues recorded using the MEA technology for the construction of databases for predictive and classification purposes using a standardized protocol.

One example application is that the inventors' methods could provide insights into the potential of a drug to induce GI-related side effects. The predictive power is unique and has the potential to identify problem compounds early, to save millions of dollars for pharmaceutical companies engaged in drug discovery with a drug testing report generated quickly (e.g., generated in less than 3 days, for certain embodiments; alternatively in less than 1 week; alternatively in less than 24 hours; alternatively in less than 1 hour.) Using the predictive power of the inventors' current database, the inventors have already refined an analytical model that predicts whether a drug could induce nausea or diarrhea with almost 70% accuracy. Embodiments can provide more powerful models, predicting a wider variety of effects, from a broader selection of candidates, with greater accuracy and confidence.

Embodiments can provide insights on the potentials on whether a drug could ameliorate GI dysrhythmia or treat GI-related side effects. Medicines or remedies can be tested in combination with known drugs that caused dysrhythmia. Other disease, transgenic or pre-treatment animal models can also be advantageously tested or predicted. The standardized method and analytical pipelines can also be applied in these experiment protocols for evaluation of GI dysrhythmia.

Embodiments of the database and related systems and methods can provide further improvements including but not limited to:
(1) The service speed can be significantly improved by having multiple MEA platforms and a greater network of technicians, clinicians, or researchers driving data collection. (e.g., Having multiple MEA headstages connected to one MEA machine can improve speed and throughput. For example, one current MEA system produced by Multichannel system has 4 headstages connected to one system. It is possible for one researcher to operate at least 4 or more headstages at the same time. Certain embodiments including development, testing, and examples disclosed herein were completed on a system having only 1 headstage connected. Connecting additional headstages, such as two, three, four, or more than four headstages is contemplated within the scope of the subject invention.)
(2) The predictive power can improve with more drugs added into the database. (e.g., (1) Drugs that are known to cause certain side effects listed in the SIDER Side Effect Resource database can be added to improve the database by reference to known or expected results. (2) Drugs that are known to have affinity towards certain receptor-of-interest listed in the IUPHAR/BPS Guide to Pharmacology can be added to improve the database by reference to known or expected results. Additional known results, or additional drugs with or without known results can be added to improve performance.)
(3) Additional applications of the database can be added for testing other types of hypothesis. (e.g., (1) Evaluating drugs' potential to induce GI dysrhythmia. (2) Predicting and classifying drugs' agonistic or antagonistic actions. (3) Predicting drugs' adverse effects.)
(4) Application can be expanded to include pharmaceuticals, food industry, research units, traditional remedies, or other industries and companies that can benefit from testing products (e.g., food, chemicals, drugs, traditional Chinese medicine, remedies, and related or unrelated products.)

Embodiments have already been applied to test >100 exemplar drugs and the data of these drugs has already been analyzed automatically using a program for extracting at least 24 slow wave features stored in the inventors' proprietary database to provide basic predictive power as referenced herein.

Embodiments can advantageously employ advanced or improved hardware (e.g., upgrading from a 60 channel platform to a 256 channel platform.) It is within the scope of certain embodiments of the subject invention that algorithms and parameters for slow wave features extractions can be improved for refinement to fit particular hypothesis and situation. Specific classification or predictive learning models can be improved and refined by using a selected sub-set of highly-focused data. The inventors have built a specific embodiment with 4 sub-databases focusing on the drug effects on the stomach, duodenum, ileum, and colon. The databases can be further extended (e.g., to use the heart, muscles, or central nervous system.) Embodiments can be further optimized for evaluating adverse effects such as cardiac dysrhythmia and epilepsy.

In certain embodiments, business models in accordance with the subject invention can provide one or more protocols that can be altered based upon a clients' instruction and requirement. These altered protocols can provide advantageously provide highly valuable experiments and data analysis, and special orders can be received as a specific source of revenue. However, the standardized protocol for adding drugs into the database can be maintained in certain embodiments, to maintain high consistency of the drug profile stored in the database and advantageously enhance the clinical or commercial value thereof.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### APPENDED TABLE 2

The following pages contain the data of Table 2. The numerical values of all slow-wave features showing the effects of dopamine on pacemaker potentials along the gastrointestinal tract of Suncus murinus, subdivided into sub-tables 2.1 through 2.8.
Table 2.1 ‘True values (basic parameters)’, showing the true numerical values of 10 slow-wave features listed with the date and sequence of each experiment;
Table 2.2 ‘True values (DFA & SampEn)’, showing the calculated DFA and SampEn values listed with the date and sequence of each experiment;
Table 2.3 ‘True mean and SD’, showing the calculated means and standard deviations of all slow-wave features grouped by concentration and tissue type;
Table 2.4 ‘Percentage change mean and SD’, showing the calculated means and standard deviations of the percentage change of all slow-wave features grouped by concentration and tissue type;
Table 2.5 ‘p-value’, showing the calculated p-values of all slow-wave features for comparisons between the baseline and post-treatment recordings;
Table 2.6 ‘Pattern(B)’, showing the calculated percentage values of the dominant activation time pattern based on the baseline data and the respective p-value;
Table 2.7 ‘Pattern(P)’, showing the calculated percentage values of the dominant activation time pattern based on the post-treatment data and the respective p-values; and
Table 2.8 ‘Radar’, showing the percentage change values used for plotting the radar diagram in FIG. 4.

TABLE 2.1

| | A | B | C | D | E | F | G | H | I |
|---|---|---|---|---|---|---|---|---|---|
| 1 | True values of ten features and their percentage change b | | | | | | | | |
| 2 | | | | | | | | Dominant | frequency |
| 3 | Date | File no. | Drug | Tissue | Dose | Dose | Active Ch. | Pre | Post |
| 4 | May 14, 2019 | 23 | dop | stomach | 100 uM | 4 | 22 | 6.445313 | 7.723722 |
| 5 | May 15, 2019 | 13 | dop | stomach | 100 uM | 4 | 35 | 7.366071 | 10.94866 |
| 6 | May 20, 2019 | 13 | dop | stomach | 100 uM | 4 | 54 | 8.810764 | 6.456163 |
| 7 | May 21, 2019 | 13 | dop | stomach | 100 uM | 4 | 5 | 6.914063 | 7.03125 |
| 8 | May 1, 2019 | 26 | dop | stomach | 100 uM | 4 | 37 | 9.739231 | 10.23015 |
| 9 | May 15, 2019 | 27 | dop | stomach | 100 uM | 4 | 5 | 6.445313 | 8.203125 |
| 10 | May 20, 2019 | 23 | dop | stomach | 100 uM | 4 | 27 | 7.942708 | 6.445313 |
| 11 | Nov. 26, 2019 | 10 | dop | duodenum | 100 nM | 7 | 1 | 26.36719 | 24.60938 |
| 12 | Nov. 27, 2019 | 1 | dop | duodenum | 100 nM | 7 | 5 | 25.19531 | 24.02344 |
| 13 | Nov. 28, 2019 | 1 | dop | duodenum | 100 nM | 7 | 8 | 30.46875 | 25.92773 |
| 14 | Nov. 26, 2019 | 4 | dop | duodenum | 100 nM | 7 | 1 | 25.19531 | 23.4375 |
| 15 | Nov. 25, 2019 | 1 | dop | duodenum | 100 nM | 7 | 2 | 28.125 | 26.36719 |
| 16 | Apr. 23, 2019 | 1 | dop | duodenum | 100 uM | 4 | 2 | 26.95313 | 26.95313 |
| 17 | Apr. 24, 2019 | 4 | dop | duodenum | 100 uM | 4 | 3 | 31.05469 | 26.5625 |
| 18 | Apr. 24, 2019 | 14 | dop | duodenum | 100 uM | 4 | 29 | 27.53906 | 25.82166 |
| 19 | May 2, 2019 | 14 | dop | duodenum | 100 uM | 4 | 21 | 31.05469 | 29.82701 |
| 20 | May 3, 2019 | 4 | dop | duodenum | 100 uM | 4 | 1 | 30.46875 | 30.46875 |
| 21 | Apr. 23, 2019 | 21 | dop | duodenun | 100 uM | 4 | 1 | 25.78125 | 26.36719 |
| 22 | May 2, 2019 | 1 | dop | duodenum | 100 uM | | 3 | 28.71094 | 20.11719 |
| 23 | Apr. 15, 2019 | 7 | dop | duodenum | 100 uM | 4 | 8 | 35.15625 | 33.98438 |
| 24 | Apr. 23, 2019 | 11 | dop | duodenum | 100 uM | 4 | 10 | 20.56641 | 16.93359 |
| 25 | Aug. 5, 2019 | 7 | dop | duodenun | 10 uM | 5 | 45 | 28.90625 | 30.85938 |
| 26 | Aug. 5, 2019 | 19 | dop | duodenum | 10 uM | 5 | 38 | 25.51912 | 20.60033 |
| 27 | Aug. 6, 2019 | 4 | dop | duodenum | 10 uM | 5 | 2 | 30.46875 | 36.32813 |
| 28 | Aug. 6, 2019 | 17 | dop | duodenum | 10 uM | 5 | 57 | 31.05469 | 26.97368 |
| 29 | Aug. 26, 2019 | 14 | dop | duodenun | 10 uM | 5 | 36 | 29.81771 | 28.71094 |
| 30 | Aug. 15, 2019 | 13 | dop | duodenum | 10 uM | 5 | 34 | 29.88281 | 31.91636 |
| 31 | Aug. 12, 2019 | 7 | dop | duodenum | 1 um | 6 | 5 | 32.22656 | 33.98438 |
| 32 | Aug. 12, 2019 | 18 | dop | duodenum | 1 um | 6 | 48 | 28.71094 | 27.53906 |
| 33 | Aug. 13, 2019 | 17 | dop | duodenum | 1 um | 6 | 57 | 32.22656 | 31.05469 |
| 34 | Aug. 14, 2019 | 1 | dop | duodenum | 1 um | 6 | 28 | 27.28795 | 26.01144 |
| 35 | Aug. 26, 2019 | 1 | dop | duodenum | 1 um | 6 | 4 | 25.78125 | 26.66016 |
| 36 | Aug. 14, 2019 | 14 | dop | duodenum | 1 um | 6 | 10 | 27.53906 | 9.375 |
| 37 | Aug. 13, 2019 | 4 | dop | duodenum | 1 um | 6 | 56 | 32.8125 | 33.39844 |
| 38 | Nov. 25, 2019 | 2 | dop | ileum | 100 nM | 7 | 10 | 25.19531 | 26.95313 |
| 39 | Nov. 26, 2019 | 5 | dop | ileum | 100 nM | 7 | 50 | 32.80078 | 24.63281 |
| 40 | Nov. 27, 2019 | 2 | dop | ileum | 100 nM | 7 | 49 | 25.1714 | 25.18335 |
| 41 | Nov. 28, 2019 | 2 | dop | ileum | 100 nM | 7 | 42 | 24.59542 | 28.04129 |
| 42 | Nov. 28, 2019 | 8 | dop | ileum | 100 nM | 7 | 55 | 25.19531 | 24.62003 |
| 43 | Nov. 26, 2019 | 11 | dop | ileum | 100 nM | 7 | 9 | 24.60938 | 8.723958 |
| 44 | Apr. 23, 2019 | 2 | dop | ileum | 100 uM | 4 | 3 | 27.53906 | 32.8125 |
| 45 | Apr. 24, 2019 | 5 | dop | ileum | 100 uM | 4 | 12 | 28.125 | 26.95313 |
| 46 | Apr. 24, 2019 | 15 | dop | ileum | 100 uM | 4 | 6 | 26.36719 | 26.36719 |
| 47 | May 2, 2019 | 2 | dop | ileum | 100 uM | 4 | 7 | 27.20424 | 26.95313 |
| 48 | Apr. 15, 2019 | 8 | dop | ileum | 100 uM | | 1 | 30.46875 | 29.29688 |
| 49 | Apr. 23, 2019 | 12 | dop | ileum | 100 uM | 4 | 3 | 27.92969 | 23.24219 |
| 50 | Aug. 5, 2019 | 8 | dop | ileum | 10 uM | 5 | 22 | 30.84162 | 29.50994 |
| 51 | Aug. 5, 2019 | 20 | dop | ileum | 10 uM | 5 | 56 | 27.58092 | 26.95313 |
| 52 | Aug. 6, 2019 | 5 | dop | ileum | 10 uM | 5 | 36 | 30.46875 | 29.41081 |
| 53 | Oct. 14, 2019 | 5 | dop | ileum | 10 uM | 5 | 20 | 27.53906 | 25.78125 |
| 54 | Oct. 14, 2019 | 13 | dop | ileum | 10 uM | 5 | 58 | 26.37729 | 29.88281 |
| 55 | Aug. 15, 2019 | 2 | dop | ileum | 10 uM | 5 | 3 | 24.60938 | 25.78125 |
| 56 | Aug. 15, 2019 | 14 | dop | ileum | 10 uM | 5 | 25 | 33.98438 | 12.75 |
| 57 | Aug. 13, 2019 | 5 | dop | ileum | 1 um | 6 | 38 | 25.93544 | 27.53906 |
| 58 | Aug. 13, 2019 | 18 | dop | ileum | 1 um | 6 | 57 | 29.29688 | 32.22656 |
| 59 | Aug. 26, 2019 | 2 | dop | ileum | 1 um | 6 | 4 | 25.19531 | 24.16992 |
| 60 | Aug. 26, 2019 | 15 | dop | ileum | 1 um | 6 | 38 | 29.29688 | 29.37397 |
| 61 | Aug. 12, 2019 | 8 | dop | ileum | 1 um | 6 | 6 | 30.46875 | 32.42188 |
| 62 | Aug. 12, 2019 | 19 | dop | ileum | 1 um | 6 | 56 | 29.88281 | 26.42997 |
| 63 | Aug. 14, 2019 | 2 | dop | ileum | 1 um | 6 | 47 | 11.13281 | 6.694648 |
| 64 | Nov. 25, 2019 | 3 | dop | colon | 100 nM | 7 | 15 | 26.36719 | 28.04688 |
| 65 | Nov. 26, 2019 | 6 | dop | colon | 100 nM | 7 | 1 | 29.88281 | 28.71094 |
| 66 | Nov. 26, 2019 | 12 | dop | colon | 100 nM | 7 | 50 | 26.36719 | 25.58203 |
| 67 | Nov. 28, 2019 | 9 | dop | colon | 100 nM | 7 | 56 | 23.4375 | 24.60938 |
| 68 | Nov. 27, 2019 | 3 | dop | colon | 100 nM | 7 | 3 | 26.95313 | 26.36719 |
| 69 | Nov. 28, 2019 | 3 | dop | colon | 100 nM | 7 | 51 | 31.05469 | 26.52803 |
| 70 | Apr. 23, 2019 | 3 | dop | colon | 100 uM | 4 | 7 | 28.71094 | 25.27902 |
| 71 | Apr. 23, 2019 | 13 | dop | colon | 100 uM | 4 | 34 | 25.78125 | 25.78125 |
| 72 | Apr. 24, 2019 | 6 | dop | colon | 100 uM | 4 | 40 | 28.125 | 28.22754 |
| 73 | Apr. 24, 2019 | 16 | dop | colon | 100 uM | 4 | 20 | 27.59766 | 26.95313 |
| 74 | May 2, 2019 | 3 | dop | colon | 100 uM | 4 | 16 | 27.53906 | 27.53906 |
| 75 | May 2, 2019 | 16 | dop | colon | 100 uM | 4 | 28 | 29.29688 | 28.08315 |
| 76 | May 3, 2019 | 3 | dop | colon | 100 uM | 4 | 8 | 26.95313 | 25.78125 |
| 77 | May 3, 2019 | 18 | dop | colon | 100 uM | 4 | 16 | 28.125 | 29.88281 |
| 78 | Apr. 15, 2019 | 9 | dop | colon | 100 uM | 4 | 2 | 31.05469 | 29.29688 |
| 79 | Aug. 5, 2019 | 9 | dop | colon | 10 uM | 5 | 11 | 29.88281 | 32.9723 |
| 80 | Aug. 6, 2019 | 6 | dop | colon | 10 uM | 5 | 35 | 28.71094 | 33.95089 |
| 81 | Aug. 6, 2019 | 19 | dop | colon | 10 uM | 5 | 34 | 32.22656 | 32.8125 |
| 82 | Aug. 15, 2019 | 3 | dop | colon | 10 uM | 5 | 31 | 25.78125 | 27.53906 |
| 83 | Aug. 15, 2019 | 15 | dop | colon | 10 uM | 5 | 12 | 30.22461 | 18.60352 |
| 84 | Aug. 5, 2019 | 21 | dop | colon | 10 uM | 5 | 35 | 30.60268 | 35.74219 |
| 85 | Aug. 12, 2019 | 9 | dop | colon | 1 um | 6 | 3 | 38.08594 | 35.35156 |
| 86 | Aug. 13, 2019 | 6 | dop | colon | 1 um | 6 | 15 | 33.98438 | 35.97656 |
| 87 | Aug. 14, 2019 | 3 | dop | colon | 1 um | 6 | 10 | 29.88281 | 31.05469 |
| 88 | Aug. 26, 2019 | 3 | dop | colon | 1 um | 6 | 47 | 25.19531 | 26.95313 |
| 89 | Aug. 13, 2019 | 19 | dop | colon | 1 um | 6 | 5 | 29.29688 | 32.8125 |
| 90 | Aug. 26, 2019 | 16 | dop | colon | 1 um | 6 | 5 | 29.88281 | 29.29688 |
| 91 | Aug. 12, 2019 | 20 | dop | colon | 1 um | 6 | 35 | 27.60603 | 29.07924 |
| 92 | Aug. 14, 2019 | 16 | dop | colon | 1 um | 6 | 12 | 29.88281 | 32.22656 |
| | J | K | L | M | N | O | P | Q | R |
| 1 | between pre and post drug treatment | | | | | | | | |
| 2 | Average frequency(cₗ | | Brady-rhythm(%) | | Normal-rhythm(%) | | Tachy-rhythm(%) | | Dominant |
| 3 | Pre | Post | Pre | Post | Pre | Post | Pre | Post | Pre |
| 4 | 9.561708 | 11.26781 | 0.702724 | 0.95783 | 55.53314 | 29.48913 | 43.39607 | 68.72364 | 1227.218 |
| 5 | 8.647831 | 12.9371 | 7.528376 | 9.013905 | 67.02229 | 8.325051 | 25.0048 | 81.76457 | 458.7314 |
| 6 | 10.38604 | 10.59835 | 24.47333 | 38.09719 | 52.93075 | 17.96585 | 21.79778 | 43.56968 | 1744.789 |
| 7 | 9.794111 | 9.306986 | 4.916558 | 9.888803 | 48.83686 | 45.17027 | 45.73647 | 44.67631 | 100.7546 |
| 8 | 10.89319 | 12.21992 | 16.01149 | 16.82949 | 62.56991 | 34.37458 | 21.05162 | 48.12517 | 497.7641 |
| 9 | 8.874609 | 12.20006 | 0.247932 | 6.766241 | 49.89498 | 16.574 | 49.75311 | 74.93873 | 1793.166 |
| 10 | 10.77698 | 13.79893 | 5.038936 | 26.35707 | 48.96856 | 13.31605 | 45.72098 | 59.56522 | 393.4822 |
| 11 | 23.01509 | 20.91156 | 37.89212 | 86.85953 | 53.31098 | 2.712224 | 4.356228 | 2.599518 | 417.118 |
| 12 | 22.48877 | 20.85175 | 35.00877 | 83.44448 | 42.59103 | 10.97897 | 8.732479 | 1.010794 | 1147.234 |
| 13 | 27.4351 | 21.27277 | 38.16942 | 93.00501 | 44.82778 | 0.781853 | 16.25106 | 1.988016 | 1443.707 |
| 14 | 24.1382 | 19.63767 | 19.47013 | 76.57593 | 59.58656 | 8.435004 | 16.68372 | 2.721179 | 873.2814 |
| 15 | 23.51808 | 21.59824 | 47.95507 | 95.99937 | 30.27727 | 1.174188 | 16.72746 | 2.186344 | 198.3337 |
| 16 | 24.18206 | 18.80943 | 23.08264 | 52.84548 | 57.93899 | 44.06731 | 18.64436 | 2.455235 | 574.6286 |
| 17 | 28.37057 | 20.19272 | 37.89886 | 94.66879 | 41.96888 | 1.074101 | 19.84343 | 3.28245 | 774.1595 |
| 18 | 24.55507 | 19.77358 | 45.85188 | 81.74657 | 46.43024 | 12.18443 | 6.396083 | 5.091291 | 3815.678 |
| 19 | 25.00091 | 22.67415 | 35.11847 | 93.19102 | 57.09641 | 4.110334 | 7.238548 | 1.932962 | 1074.115 |
| 20 | 27.77748 | 29.154 | 47.58485 | 20.55675 | 43.72677 | 76.50087 | 7.835652 | 2.590627 | 623.7462 |
| 21 | 24.63347 | 23.13298 | 45.95207 | 23.94544 | 40.27593 | 57.82702 | 12.09501 | 15.78294 | 600.9052 |
| 22 | 24.03309 | 18.78526 | 42.13317 | 79.86209 | 44.59376 | 17.88097 | 12.35006 | 1.837673 | 1192.399 |
| 23 | 30.21622 | 32.15102 | 66.35003 | 89.38949 | 27.31384 | 5.869405 | 5.376236 | 4.059066 | 275.646 |
| 24 | 17.21501 | 14.89922 | 45.39172 | 81.28258 | 35.24876 | 5.420653 | 9.507025 | 11.16407 | 616.2309 |
| 25 | 26.40578 | 26.14276 | 37.13837 | 38.58378 | 46.03734 | 17.16079 | 16.29396 | 43.53401 | 309.4348 |
| 26 | 22.96496 | 21.75055 | 25.26747 | 74.13106 | 58.74137 | 1.641425 | 10.70122 | 22.87859 | 318.9673 |
| 27 | 29.74202 | 31.8498 | 16.92845 | 14.59392 | 40.86769 | 6.786203 | 41.26258 | 74.64229 | 63.98298 |
| 28 | 28.94213 | 25.02344 | 37.36323 | 98.78689 | 59.16838 | 0.317464 | 3.077602 | 0.587098 | 1151.063 |
| 29 | 28.12954 | 20.35077 | 18.17385 | 86.48223 | 74.82886 | 8.481198 | 6.740015 | 4.151272 | 496.2128 |
| 30 | 21.33653 | 27.1472 | 54.26617 | 27.14185 | 34.62257 | 12.57223 | 10.23487 | 59.71816 | 142.74561 |
| 31 | 31.45344 | 34.02021 | 17.71402 | 9.453614 | 41.59315 | 16.07745 | 39.75197 | 73.07456 | 150.7896 |
| 32 | 27.12635 | 26.61375 | 15.91368 | 81.18864 | 77.91959 | 17.32737 | 5.867268 | 1.238002 | 1635.2931 |
| 33 | 29.87376 | 27.41205 | 23.63806 | 80.36939 | 61.20529 | 18.63229 | 14.50213 | 0.71264 | 394.8158 |
| 34 | 26.81874 | 25.42445 | 23.44339 | 42.00246 | 46.3375 | 37.12503 | 29.49039 | 20.14541 | 1348.785 |
| 35 | 23.66523 | 18.12443 | 21.30036 | 52.50475 | 56.14845 | 10.34308 | 21.60151 | 35.58082 | 606.5954 |
| 36 | 20.31598 | 16.81574 | 52.64321 | 73.71424 | 43.09806 | 13.93247 | 3.473164 | 9.75916 | 2274.674 |
| 37 | 32.73585 | 30.8792 | 25.80182 | 36.9451 | 27.753 | 57.91925 | 41.40798 | 4.408742 | 517.2236 |
| 38 | 21.5008 | 15.6716 | 28.04274 | 69.03422 | 50.43 | 3.398043 | 15.05664 | 26.84341 | 823.8119 |
| 39 | 29.49786 | 21.58062 | 39.96421 | 91.91163 | 54.47505 | 0.158058 | 5.241688 | 0.65703 | 1065.657 |
| 40 | 22.44392 | 19.95531 | 23.57812 | 47.56762 | 58.985 | 33.17797 | 8.69058 | 7.062949 | 1103.877 |
| 41 | 23.26789 | 24.15997 | 14.91305 | 24.15507 | 51.34031 | 10.31048 | 26.49862 | 63.20838 | 635.8233 |
| 42 | 22.22284 | 20.36602 | 37.18505 | 43.60992 | 49.7192 | 38.57004 | 3.401657 | 11.75232 | 678.0197 |
| 43 | 21.90659 | 14.66174 | 46.83729 | 83.88278 | 42.21614 | 2.16001 | 5.234741 | 10.49075 | 375.429 |
| 44 | 22.01549 | 24.25948 | 34.91184 | 39.14774 | 55.89276 | 2.46986 | 8.688286 | 55.38769 | 539.3218 |
| 45 | 25.44041 | 23.81375 | 34.11563 | 65.8486 | 46.74299 | 28.5543 | 18.82791 | 4.715564 | 2646.308 |
| 46 | 23.97462 | 18.94477 | 34.66778 | 60.37298 | 55.3223 | 26.06684 | 9.139108 | 12.06272 | 434.2366 |
| 47 | 24.3039 | 23.56202 | 44.43525 | 48.60932 | 44.36138 | 46.40256 | 10.39721 | 4.48638 | 1034.608 |
| 48 | 26.70159 | 28.15263 | 43.94828 | 50.77686 | 41.88166 | 18.18352 | 13.53993 | 30.43492 | 702.3499 |
| 49 | 23.48155 | 26.51379 | 56.861 | 28.04064 | 27.34696 | 28.61078 | 13.53112 | 42.58756 | 73.88376 |
| 50 | 29.3046 | 25.17941 | 27.80785 | 80.36584 | 46.63693 | 15.36637 | 24.89147 | 2.528434 | 320.4661 |
| 51 | 27.18617 | 23.46757 | 12.9167 | 46.28354 | 70.84217 | 31.42071 | 15.19972 | 18.5881 | 559.6343 |
| 52 | 27.49004 | 26.9353 | 46.30426 | 49.20339 | 45.866 | 5.694503 | 5.716511 | 43.80599 | 242.7488 |
| 53 | 25.72788 | 22.37078 | 33.74669 | 95.20372 | 46.98657 | 1.251167 | 18.96669 | 2.878589 | 2471.307 |
| 54 | 25.50479 | 27.00689 | 20.87148 | 15.35338 | 56.91899 | 0.908526 | 21.77765 | 83.30626 | 3550.393 |
| 55 | 19.61153 | 15.71014 | 43.05477 | 65.7096 | 42.9074 | 3.6381 | 9.487383 | 29.80715 | 2550.74 |
| 56 | 29.56015 | 18.35914 | 44.97835 | 90.91006 | 46.3969 | 6.39318 | 8.304006 | 1.955372 | 430.1626 |
| 57 | 24.39005 | 25.7971 | 26.3842 | 27.64547 | 47.63962 | 4.23857 | 24.73712 | 66.97909 | 521.4505 |
| 58 | 28.48681 | 29.13575 | 18.22729 | 18.76671 | 47.42673 | 4.290776 | 34.04526 | 76.29107 | 1348.042 |
| 59 | 22.68226 | 20.78601 | 37.14951 | 69.06186 | 50.88879 | 22.72164 | 7.664293 | 1.914616 | 2170.976 |
| 60 | 27.38278 | 25.0637 | 16.96646 | 42.90912 | 49.9792 | 37.92754 | 32.75037 | 18.3245 | 595.0529 |
| 61 | 28.40995 | 26.98669 | 31.60834 | 52.26899 | 26.21879 | 11.70518 | 41.50777 | 34.91441 | 24.22939 |
| 62 | 28.70599 | 26.30138 | 35.28087 | 81.12237 | 31.22298 | 13.13944 | 33.21641 | 5.235753 | 1116.183 |
| 63 | 14.89803 | 14.44632 | 26.62858 | 32.05949 | 30.03805 | 14.35364 | 42.14034 | 52.93241 | 1717.478 |
| 64 | 21.91618 | 23.63703 | 45.05987 | 26.32658 | 51.48506 | 14.05074 | 2.698067 | 59.08785 | 794.3071 |
| 65 | 26.47561 | 22.79427 | 40.48592 | 85.69764 | 44.8252 | 7.160775 | 13.89316 | 5.937764 | 313.0396 |
| 66 | 24.07018 | 20.46492 | 23.94774 | 75.66113 | 69.0487 | 18.87442 | 6.330044 | 2.393511 | 4266.695 |
| 67 | 22.00919 | 23.57862 | 24.45042 | 7.339112 | 60.88265 | 8.653445 | 9.402291 | 82.51471 | 4703.476 |
| 68 | 21.7334 | 24.15375 | 54.24205 | 24.29163 | 31.54276 | 67.69084 | 11.40679 | 6.889918 | 151.8095 |
| 69 | 27.20392 | 25.02445 | 48.29437 | 71.3197 | 34.60031 | 19.27502 | 16.50752 | 9.045152 | 1236.506 |
| 70 | 25.72217 | 22.79209 | 49.1448 | 95.09784 | 48.36238 | 1.326688 | 2.246251 | 1.425888 | 2580.04 |
| 71 | 22.02441 | 22.95608 | 36.30052 | 25.14785 | 58.93185 | 66.43853 | 3.409913 | 7.89333 | 2533.357 |
| 72 | 26.3914 | 24.44574 | 27.2814 | 27.18348 | 66.17106 | 50.69786 | 6.36905 | 21.53319 | 7210.319 |
| 73 | 25.94239 | 24.52726 | 17.60202 | 27.80757 | 48.43337 | 68.0555 | 33.52019 | 3.690399 | 695.4764 |
| 74 | 24.36212 | 21.47674 | 38.37525 | 54.68222 | 55.0869 | 33.37123 | 5.499504 | 11.11587 | 2999.823 |
| 75 | 24.89009 | 23.77947 | 32.46554 | 79.54411 | 62.12981 | 15.07362 | 5.0554 | 4.656427 | 3502.515 |
| 76 | 24.61988 | 22.29292 | 34.63721 | 74.21758 | 51.85196 | 19.1005 | 12.54569 | 6.209923 | 1017.461 |
| 77 | 24.73906 | 25.37517 | 43.65331 | 31.49113 | 45.3639 | 25.1467 | 10.27239 | 42.64712 | 250.1025 |
| 78 | 29.55943 | 28.55308 | 33.06713 | 70.19816 | 41.92666 | 22.00668 | 23.68203 | 6.010998 | 1092.505 |
| 79 | 30.67517 | 31.71247 | 12.13458 | 14.0577 | 41.95848 | 14.31628 | 44.38948 | 69.5837 | 782.9606 |
| 80 | 29.84783 | 31.77773 | 7.745447 | 11.74236 | 43.99969 | 2.4495 | 47.37744 | 81.78756 | 1397.745 |
| 81 | 32.12547 | 32.04054 | 22.58195 | 21.8576 | 41.4262 | 47.47349 | 34.18464 | 29.74224 | 2608.058 |
| 82 | 20.53619 | 25.80517 | 42.45769 | 13.46912 | 51.5152 | 7.132113 | 5.410597 | 78.86244 | 9696.359 |
| 83 | 24.99209 | 19.05651 | 45.45818 | 86.75482 | 45.11614 | 4.435274 | 8.865949 | 7.770988 | 713.4226 |
| 84 | 29.35024 | 34.74601 | 31.53189 | 7.018991 | 30.47643 | 2.751965 | 36.86332 | 82.27797 | 341.7797 |
| 85 | 35.6375 | 34.24565 | 27.97466 | 80.68776 | 40.50235 | 9.670105 | 17.57384 | 3.777305 | 213.6953 |
| 86 | 32.62014 | 34.05263 | 30.13049 | 18.69604 | 43.16979 | 19.73727 | 23.14059 | 57.30871 | 726.26181 |
| 87 | 25.92098 | 25.8232 | 54.74521 | 33.43821 | 41.17399 | 5.7991 | 3.355128 | 59.45696 | 2978.365 |
| 88 | 22.65473 | 24.55266 | 23.97898 | 15.74663 | 61.0391 | 1.642471 | 13.00342 | 82.36136 | 6045.057 |
| 89 | 28.29088 | 32.63799 | 19.02978 | 2.283395 | 42.45101 | 0.974505 | 38.26148 | 96.37144 | 1761.5621 |
| 90 | 27.64394 | 28.06296 | 51.52971 | 42.14129 | 42.15195 | 55.13875 | 5.876477 | 2.448153 | 721.282 |
| 91 | 26.52632 | 28.90267 | 34.97629 | 13.96258 | 30.30604 | 20.28998 | 33.9857 | 65.11567 | 475.6059 |
| 92 | 18.95195 | 28.27993 | 57.34058 | 19.62094 | 30.14803 | 2.642974 | 11.57971 | 77.01969 | 1178.321 |
| | S | T | U | V | W | X | Y | Z | AA |
| 1 | | | | | | | | | |
| 2 | Power(uV2 | PPAmp(uV) | | Slope(V/s) | | Period(s) | | Velocity (mm/s) | |
| 3 | Post | Pre | Post | Pre | Post | Pre | Post | Pre | Post |
| 4 | 114.5817 | 165.9297 | 107.3751 | 130.9438 | 99.65345 | 7.075897 | 7.818339 | 10.54026 | 5.247322 |
| 5 | 95.3892 | 126.4181 | 129.1386 | 83.04502 | 115.438 | 8.343485 | 6.864122 | 13.08662 | 10.45441 |
| 6 | 1189.991 | 263.6145 | 202.1354 | 191.8213 | 161.2498 | 6.501189 | 6.719058 | 2.255657 | 1.850111 |
| 7 | 163.7252 | 125.7761 | 203.0095 | 95.12597 | 132.3394 | 8.259471 | 6.711759 | 2.378948 | 7.82299 |
| 8 | 142.7906 | 132.8589 | 108.8174 | 103.7243 | 98.85302 | 7.443923 | 9.28293 | 1.482276 | 1.973113 |
| 9 | 26.2538 | 195.0974 | 75.14287 | 137.624 | 61.88166 | 7.182138 | 7.864453 | | |
| 10 | 39.61768 | 129.1616 | 62.59825 | 92.4259 | 59.0803 | 7.359625 | 8.408058 | 33.9049 | 19.83138 |
| 11 | 323.1121 | 153.7171 | 106.1835 | 265.3663 | 150.4249 | 2.602563 | 3.777865 | 19.52631 | 10.71464 |
| 12 | 778.3611 | 241.9834 | 155.551 | 353.9343 | 228.7753 | 2.704899 | 2.77253 | 9.120996 | 13.39762 |
| 13 | 402.9964 | 262.1883 | 150.7297 | 467.6659 | 225.7625 | 2.15127 | 2.948424 | 17.17456 | 5.636935 |
| 14 | 136.8399 | 142.6976 | 72.02247 | 218.7107 | 102.2567 | 2.56477 | 3.765535 | 4.376614 | 4.155531 |
| 15 | 269.8224 | 164.4351 | 86.22445 | 302.8666 | 127.0351 | 2.567276 | 3.08918 | | |
| 16 | 136.5489 | 160.6492 | 96.3345 | 267.3938 | 159.1954 | 2.555149 | 3.324514 | 0.26342 | 0.769102 |
| 17 | 120.9378 | 177.2793 | 106.8458 | 304.6884 | 154.2015 | 2.1633 | 3.852423 | 5.751987 | 4.911966 |
| 18 | 535.7254 | 341.5446 | 164.8908 | 542.7577 | 214.1793 | 2.382746 | 3.229378 | 13.57859 | 13.0901 |
| 19 | 344.6335 | 167.8949 | 132.8325 | 282.525 | 207.787 | 2.070571 | 2.83071 | 1.215978 | 1.526288 |
| 20 | 514.2087 | 216.4564 | 126.6293 | 412.9497 | 250.4974 | 2.113444 | 2.024607 | 0.197216 | 0.62709 |
| 21 | 389.8946 | 236.9042 | 111.2261 | 387.0567 | 167.1171 | 2.367375 | 2.449807 | | |
| 22 | 96.86454 | 156.8366 | 96.59675 | 255.4911 | 129.7168 | 2.226301 | 3.671468 | | |
| 23 | 1238.814 | 150.2463 | 160.9008 | 271.0603 | 279.602 | 1.828155 | 1.792791 | 5.697241 | 15.935 |
| 24 | 119.382 | 186.231 | 93.20696 | 176.177 | 80.39183 | 4.267108 | 6.129933 | 56.11221 | 40.67752 |
| 25 | 116.5098 | 122.5289 | 86.93794 | 225.6775 | 144.5061 | 2.321544 | 2.931393 | 31.05167 | 24.30357 |
| 26 | 36.12908 | 115.2835 | 89.01767 | 203.8758 | 97.17546 | 2.638557 | 5.966667 | 14.52993 | 16.47798 |
| 27 | 56.51848 | 82.67876 | 96.39202 | 146.9318 | 185.2119 | 3.060334 | 2.597685 | 1.689907 | 0.900179 |
| 28 | 365.4525 | 152.1231 | 119.8154 | 277.6937 | 218.047 | 2.837064 | 2.389311 | 23.53547 | 12.61442 |
| 29 | 60.73251 | 132.0147 | 84.86065 | 215.3113 | 103.3986 | 2.495328 | 5.438827 | 11.03163 | 10.66112 |
| 30 | 191.6138 | 100.8303 | 107.2834 | 144.6977 | 174.984 | 4.709191 | 2.46135 | 24.80975 | 20.37699 |
| 31 | 198.5221 | 103.202 | 125.0108 | 187.8575 | 224.9658 | 2.009352 | 1.854321 | 8.624594 | 9.610183 |
| 32 | 884.6228 | 240.0076 | 136.3531 | 454.1182 | 210.3007 | 2.461381 | 2.22742 | 4.473829 | 27.69106 |
| 33 | 486.3765 | 128.489 | 113.7238 | 219.5026 | 182.9395 | 2.472732 | 3.233809 | 5.510669 | 12.9647 |
| 34 | 459.7058 | 237.836 | 136.0856 | 386.1336 | 206.6121 | 2.158745 | 2.596941 | 9.003392 | 6.260467 |
| 35 | 132.4678 | 167.8975 | 110.0723 | 297.8451 | 161.8776 | 2.404422 | 3.998268 | 0.910399 | 0.946663 |
| 36 | 433.2889 | 248.2676 | 174.8678 | 404.8222 | 266.4118 | 2.456137 | 3.434789 | | |
| 37 | 396.2034 | 185.9384 | 117.1964 | 340.9864 | 192.1192 | 1.886906 | 2.022532 | 46.23518 | 21.72633 |
| 38 | 173.6875 | 165.0947 | 125.0003 | 225.6506 | 129.4994 | 2.738254 | 5.263444 | 7.345775 | 8.223719 |
| 39 | 282.6735 | 184.8864 | 137.6931 | 323.0908 | 207.8763 | 2.190773 | 2.803142 | 9.279636 | 10.59234 |
| 40 | 253.4256 | 184.7064 | 124.7412 | 291.0957 | 188.7427 | 2.585893 | 3.16586 | 14.11728 | 33.36407 |
| 41 | 181.3495 | 161.7087 | 133.9181 | 221.8828 | 184.8274 | 2.439162 | 2.76411 | 6.729791 | 29.62461 |
| 42 | 111.9631 | 165.9219 | 110.7474 | 311.2537 | 153.3792 | 2.651738 | 3.649255 | 12.41127 | 13.21107 |
| 43 | 62.05303 | 128.8378 | 83.14867 | 172.2187 | 96.04136 | 3.370983 | 4.746814 | 12.97174 | 33.41779 |
| 44 | 122.6701 | 140.0502 | 156.839 | 216.2564 | 198.6001 | 2.377403 | 2.187568 | 0.300642 | 0.34974 |
| 45 | 1044.424 | 305.448 | 169.4265 | 478.1394 | 236.7072 | 2.177965 | 2.788364 | 9.421553 | 10.54687 |
| 46 | 78.22819 | 134.7922 | 111.6934 | 187.9285 | 131.0513 | 2.845029 | 4.334366 | 19.80925 | 16.86316 |
| 47 | 273.5222 | 211.3909 | 110.9285 | 303.418 | 166.0152 | 2.328703 | 2.720836 | 0.29928 | 0.846151 |
| 48 | 522.826 | 181.668 | 171.0447 | 313.385 | 289.8117 | 2.029871 | 1.989574 | | |
| 49 | 362.9651 | 129.5328 | 101.0068 | 184.0508 | 160.0681 | 3.152846 | 2.251582 | 3.793807 | 2.714422 |
| 50 | 240.5102 | 131.4992 | 132.2815 | 228.2261 | 239.4007 | 2.320671 | 2.783344 | 37.48715 | 31.93905 |
| 51 | 71.37566 | 142.4964 | 95.01166 | 244.2492 | 151.2174 | 2.469433 | 4.186888 | 12.06478 | 13.65904 |
| 52 | 94.91146 | 135.3635 | 109.0977 | 240.9907 | 193.1793 | 2.086123 | 2.972784 | 14.27186 | 5.671977 |
| 53 | 931.0778 | 323.6532 | 157.088 | 534.2644 | 230.7204 | 2.40527 | 2.774037 | 12.50303 | 23.86599 |
| 54 | 1624.65 | 391.4931 | 222.4924 | 679.7269 | 407.8716 | 2.161768 | 2.095084 | 19.05636 | 12.71085 |
| 55 | 1084.71 | 339.1799 | 229.2742 | 507.1531 | 311.8588 | 2.544906 | 2.86057 | | |
| 56 | 1274.025 | 143.2558 | 98.27781 | 251.2834 | 133.1093 | 2.125833 | 3.663375 | 10.97005 | 10.09436 |
| 57 | 343.1104 | 133.2285 | 109.5375 | 211.2772 | 145.7456 | 2.534374 | 2.881355 | 9.843357 | 8.861728 |
| 58 | 812.751 | 197.4446 | 165.8517 | 341.9993 | 290.7222 | 2.10914 | 2.250775 | 30.93573 | 108.4671 |
| 59 | 624.8647 | 252.0604 | 149.0236 | 368.4705 | 204.7116 | 2.455393 | 3.026183 | 0.482641 | 1.237183 |
| 60 | 146.7519 | 147.66 | 106.9751 | 232.7025 | 164.4119 | 2.330963 | 3.128523 | 12.0509 | 8.267479 |
| 61 | 16.57772 | 67.00093 | 47.93216 | 112.7038 | 77.16624 | 2.817529 | 4.418039 | | |
| 62 | 588.1552 | 218.4045 | 124.9097 | 407.0866 | 195.4186 | 1.977173 | 2.375484 | 10.94663 | 18.63569 |
| 63 | 689.6141 | 293.5812 | 261.9851 | 431.9896 | 352.5149 | 3.487382 | 3.584747 | 36.4162 | 20.88166 |
| 64 | 650.0229 | 185.7794 | 144.9923 | 252.9082 | 249.5184 | 2.349213 | 2.596775 | 6.876612 | 8.191511 |
| 65 | 149.7697 | 176.2134 | 123.1894 | 281.488 | 182.2804 | 2.328648 | 3.50807 | 14.35902 | 4.353836 |
| 66 | 403.722 | 290.9637 | 176.6312 | 500.671 | 236.8424 | 2.412022 | 2.889371 | 14.08453 | 35.11098 |
| 67 | 6346.002 | 349.3109 | 464.9393 | 463.7021 | 691.9626 | 2.544697 | 2.340555 | 8.152552 | 19.75282 |
| 68 | 576.1343 | 117.6686 | 198.0989 | 182.729 | 374.1664 | 2.360742 | 2.277285 | 19.85148 | 31.21643 |
| 69 | 369.6639 | 265.4292 | 145.8128 | 461.1794 | 242.4655 | 1.977726 | 2.218092 | 18.11598 | 9.447945 |
| 70 | 1612.128 | 329.1611 | 226.7319 | 462.8919 | 317.1157 | 2.214458 | 2.341692 | 0.584388 | 0.730249 |
| 71 | 1871.52 | 276.7989 | 266.4024 | 471.253 | 397.3228 | 2.280536 | 2.281396 | 10.31011 | 11.69463 |
| 72 | 1787.827 | 422.7414 | 248.1864 | 638.8179 | 376.1577 | 2.109036 | 2.374731 | 8.602977 | 8.064947 |
| 73 | 1719.941 | 217.1532 | 175.786 | 352.1141 | 287.2303 | 2.242094 | 2.310789 | 18.45824 | 15.69399 |
| 74 | 475.4371 | 308.24 | 184.8928 | 579.7324 | 328.3644 | 2.275548 | 2.364138 | 1.272656 | 1.249231 |
| 75 | 688.5715 | 245.7697 | 194.8439 | 437.6195 | 303.2619 | 2.232123 | 2.355961 | 6.325067 | 9.626219 |
| 76 | 353.6664 | 231.4232 | 172.9365 | 371.5585 | 226.1931 | 2.802044 | 2.845814 | 0.346498 | 1.47046 |
| 77 | 109.4568 | 134.6899 | 102.8972 | 215.6064 | 143.0274 | 2.190859 | 3.461695 | 36.36143 | 12.73328 |
| 78 | 1002.6 | 239.6791 | 225.0186 | 423.9153 | 312.0998 | 1.889763 | 1.989571 | | |
| 79 | 149.2984 | 189.4024 | 143.6341 | 372.8339 | 280.9362 | 2.115422 | 2.08285 | 14.49993 | 12.26728 |
| 80 | 141.6908 | 214.5119 | 113.6276 | 394.5327 | 206.5147 | 1.874584 | 2.211204 | 7.943391 | 7.17274 |
| 81 | 2555.805 | 421.1839 | 254.5048 | 718.2305 | 404.8793 | 1.818608 | 1.907673 | 11.0747 | 6.903926 |
| 82 | 15634.01 | 458.3796 | 535.8893 | 725.3523 | 934.4866 | 2.519453 | 2.132585 | 12.63855 | 11.93817 |
| 83 | 100.9446 | 185.3204 | 104.1683 | 309.3891 | 150.7654 | 2.438218 | 3.52994 | 14.84196 | 14.83116 |
| 84 | 490.2913 | 199.9551 | 190.3956 | 359.6217 | 361.3398 | 1.82889 | 1.864687 | 21.13738 | 22.24272 |
| 85 | 84.54273 | 108.777 | 92.22566 | 217.0438 | 170.0818 | 1.957476 | 2.068392 | 5.342699 | 10.54298 |
| 86 | 311.0559 | 190.9859 | 176.9006 | 365.2557 | 336.6746 | 1.791417 | 1.867827 | 13.21651 | 15.7851 |
| 87 | 4571.453 | 373.3604 | 394.4544 | 656.0935 | 665.7796 | 2.026475 | 2.105662 | 14.48009 | 12.00594 |
| 88 | 5798.707 | 433.8095 | 367.6474 | 690.9838 | 596.7559 | 2.242203 | 2.131975 | 5.650442 | 13.79302 |
| 89 | 2526.957 | 271.0322 | 284.6706 | 468.0134 | 512.0608 | 1.886076 | 1.772943 | 0.348905 | 0.619782 |
| 90 | 530.1671 | 234.731 | 136.6338 | 376.7453 | 220.1576 | 2.060946 | 2.06026 | 4.282803 | 4.145504 |
| 91 | 305.8722 | 145.415 | 132.3897 | 227.43 | 217.4613 | 2.275413 | 2.048201 | 11.47573 | 3.32435 |
| 92 | 3498.434 | 257.4237 | 285.7518 | 431.6641 | 485.2503 | 2.180801 | 1.91454 | 109.8668 | 36.49852 |
| | AB | AC | AD | AE | AF | AG | AH | Al | AJ |
| 1 | | | | | | | | | |
| 2 | Percentage change (%) | | | | | | | | |
| 3 | DF | AF | B | N | T | DP | PPAmp | Slope | Period |
| 4 | 19.83471 | 17.84305 | 0.255106 | −26.044 | 25.32757 | −90.6633 | −35.2888 | −23.896 | 10.49255 |
| 5 | 48.63636 | 49.59933 | 1.485529 | −58.6972 | 56.75977 | −79.2059 | 2.15198 | 39.00654 | −17.7308 |
| 6 | −26.7241 | 2.044148 | 13.62386 | −34.9649 | 21.7719 | −31.7974 | −23.3216 | −15.9375 | 3.351217 |
| 7 | 1.694915 | −4.97364 | 4.972245 | −3.6666 | −1.06016 | 62.499 | 61.40546 | 39.12015 | −18.7386 |
| 8 | 5.04065 | 12.17945 | 0.817998 | −28.1953 | 27.07355 | −71.3136 | −18.0955 | −4.69639 | 24.70481 |
| 9 | 27.27272 | 37.47152 | 6.518309 | −33.321 | 25.18562 | −98.5359 | −61.4844 | −55.0357 | 9.500166 |
| 10 | −18.8524 | 28.04079 | 21.31813 | −35.6525 | 13.84424 | −89.9315 | −51.5349 | −36.0782 | 14.24574 |
| 11 | −6.66666 | −9.13979 | 48.96741 | −50.5988 | −1.75671 | −22.537 | −30.9228 | −43.3142 | 45.15941 |
| 12 | −4.65114 | −7.27928 | 48.43571 | −31.6121 | −7.72169 | −32.1532 | −35.7183 | −35.3622 | 2.500315 |
| 13 | −14.9039 | −22.4615 | 54.83559 | −44.0459 | −14.263 | −72.086 | −42.5109 | −51.7257 | 1 37.05504 |
| 14 | −6.97673 | −18.6448 | 57.1058 | −51.1516 | −13.9625 | −84.3304 | 49.5279 | −53.2457 | 46.81765 |
| 15 | −6.24999 | −8.16325 | 48.0443 | −29.1031 | −14.5411 | 36.04466 | −47.5632 | −58.0558 | 20.3291 |
| 16 | 0 | −22.2174 | 29.76285 | −13.8717 | −16.1891 | −76.237 | −40.0343 | −40.464 | 30.11039 |
| 17 | −14.4654 | −28.8251 | 56.76993 | −40.8948 | −16.561 | −84.3782 | −39.7302 | −49.3904 | 78.08083 |
| 18 | −6.23624 | −19.4725 | 35.89469 | −34.2458 | −1.30479 | −85.9599 | −51.722 | −60.5387 | 35.53175 |
| 19 | −3.95328 | −9.30669 | 58.07255 | −52.9861 | −5.30559 | −67.9147 | −20.8835 | −26.4536 | 36.71155 |
| 20 | 0 | 4.955501 | −27.0281 | 32.7741 | −5.24503 | −17.5612 | −41.4989 | −39.3395 | −4.2034 |
| 21 | 2.272737 | −6.09127 | −22.0066 | 17.55109 | 3.68793 | −35.1155 | −53.0502 | −56.8236 | 3.482 |
| 22 | −29.932 | −21.8359 | 37.72892 | −26.7128 | −10.5124 | −91.8765 | −38.4093 | −49.2284 | 64.91337 |
| 23 | −3.33332 | 6.403183 | 23.03946 | −21.4444 | −1.31717 | 349.4221 | 7.091356 | 3.151218 | −1.93441 |
| 24 | −17.6639 | −13.4522 | 35.89086 | −29.8281 | 1.657045 | −80.6271 | −49.9509 | −54.3687 | 43.65545 |
| 25 | 6.756774 | −0.99607 | 1.44541 | −28.8766 | 27.24005 | −62.3475 | −29.047 | −35.9679 | 26.26911 |
| 26 | −19.2749 | −5.2881 | 48.86359 | −57.0999 | 12.17737 | −88.6731 | −22.7837 | −52.336 | 126.1337 |
| 27 | 19.23079 | 7.086876 | −2.33453 | −34.0815 | 33.37971 | −11.6664 | 16.58619 | 26.05297 | −15.1176 |
| 28 | −13.1414 | −13.5397 | 61.42366 | −58.8509 | −2.4905 | −68.2509 | −21.2379 | −21.4793 | −15.7823 |
| 29 | −3.71179 | −27.6534 | 68.30838 | −66.3477 | −2.58874 | −87.7608 | −35.7188 | −51.9772 | 117.9604 |
| 30 | 6.805083 | 27.23343 | −27.1243 | −22.0503 | 49.48329 | 34.23447 | 6.399961 | 20.93074 | −47.7331 |
| 31 | 5.454569 | 8.160538 | −8.26041 | −25.5157 | 33.32259 | 31.65503 | 21.13215 | 19.75343 | −7.71547 |
| 32 | −4.08165 | −1.88968 | 65.27496 | −60.5922 | −4.62927 | −45.9043 | −43.188 | −53.6903 | −9.50527 |
| 33 | −3.63635 | −8.24038 | 56.73133 | −42.573 | −13.7895 | 23.19074 | −11.4914 | −16.6573 | 30.77879 |
| 34 | −4.67793 | −5.19894 | 18.55907 | −9.21247 | 9.34498 | −65.917 | −42.7817 | −46.4921 | 20.29865 |
| 35 | 3.409105 | −23.4133 | 31.20439 | −45.8054 | 13.97931 | −78.1621 | −34.4408 | −45.6504 | 66.28811 |
| 36 | −65.9574 | −17.229 | 21.07103 | −29.1656 | 6.285996 | −80.9516 | −29.5648 | −34.1904 | 39.84517 |
| 37 | 1.785722 | −5.67161 | 11.14328 | 30.16625 | −36.9992 | −23.398 | −36.9703 | −43.6578 | 7.187745 |
| 38 | 6.976775 | −27.1115 | 40.99148 | −47.032 | 11.78677 | −78.9166 | −24.2857 | −42.6107 | 92.21898 |
| 39 | −24.9018 | −26.84 | 51.94742 | −54.317 | −4.58466 | −73.4743 | −25.5256 | −35.6601 | 27.95219 |
| 40 | 0.047475 | −11.0881 | 23.9895 | −25.807 | −1.62763 | −77.0422 | −32.4651 | −35.1613 | 22.42811 |
| 41 | 14.01021 | 3.833953 | 9.24202 | −41.0298 | 36.70976 | −71.478 | −17.1856 | −16.7004 | 13.32212 |
| 42 | −2.28328 | −8.35546 | 6.42487 | −11.1492 | 8.350663 | −83.4867 | −33.2533 | −50.7221 | 37.61748 |
| 43 | −64.5503 | −33.0716 | 37.04549 | −40.0561 | 5.256009 | −83.4714 | −35.4625 | −44.2329 | 40.81394 |
| 44 | 19.14894 | 10.19274 | 4.235902 | −53.4229 | 46.69941 | −77.2548 | 11.98769 | −8.16451 | −7.98494 |
| 45 | −4.16667 | −6.39401 | 31.73298 | −18.1887 | −14.1123 | −60.5328 | −44.5318 | −50.4941 | 28.02614 |
| 46 | 0 | −20.9799 | 25.7052 | −29.2555 | 2.923611 | −81.9849 | −17.1366 | −30.2653 | 52.34873 |
| 47 | −0.92308 | −3.05252 | 4.174069 | 2.041184 | −5.91083 | −73.5627 | −47.5245 | −45.285 | 16.83914 |
| 48 | −3.84614 | 5.434283 | 6.82858 | −23.6981 | 16.89499 | −25.5605 | −5.84765 | −7.52215 | −1.9852 |
| 49 | −16.7832 | 12.91329 | −28.8204 | 1.26382 | 29.05644 | 391.2651 | −22.0222 | −13.0305 | −28.5857 |
| 50 | 4.3178 | −14.0769 | 52.55799 | −31.2706 | −22.363 | −24.9499 | 0.594909 | 4.896285 | 19.93704 |
| 51 | −2.27617 | −13.6783 | 33.36684 | −39.4215 | 3.38838 | −87.246 | −33.3235 | −38.0889 | 69.54856 |
| 52 | −3.47221 | −2.01797 | 2.89913 | 40.1715 | 38.08948 | −60.9014 | −19.4039 | −19.8395 | 42.50282 |
| 53 | −6.38297 | −13.0485 | 61.45703 | −45.7354 | −16.0881 | −62.3245 | −51.4641 | −56.8153 | 15.33163 |
| 54 | 13.28992 | 5.889482 | −5.5181 | −56.0105 | 61.52861 | −54.2403 | −43.1682 | −39.9948 | −3.0847 |
| 55 | 4.761883 | −19.8933 | 22.65483 | −39.2693 | 20.31977 | −57.4747 | −32.4034 | −38.508 | 12.40376 |
| 56 | −62.4828 | −37.8923 | 45.93171 | −40.0037 | −6.34863 | 196.1729 | −31.397 | −47.0282 | 72.32657 |
| 57 | 6.183122 | 5.768951 | 1.26127 | −43.4011 | 42.24197 | −34.2008 | −17.7822 | −31.0169 | 13.69099 |
| 58 | 9.999973 | 2.278037 | 0.53942 | −43.136 | 42.24581 | −39.7088 | −16.0009 | −14.9933 | 6.715296 |
| 59 | −4.06977 | −8.36006 | 31.91235 | −28.1672 | −5.74968 | −71.2173 | −40.8778 | −44.4429 | 23.24638 |
| 60 | 0.263134 | −8.46912 | 25.94266 | −12.0517 | −14.4259 | −75.338 | −27.5531 | −29.3467 | 34.2159 |
| 61 | 6.410273 | −5.00972 | 20.66065 | −14.5136 | −6.59336 | −31.5801 | −28.4605 | −31.5318 | 56.80545 |
| 62 | −11.5546 | −8.37668 | 45.8415 | −18.0835 | −27.9807 | −47.3066 | −42.8081 | −51.9958 | 20.14548 |
| 63 | −39.8656 | −3.03201 | 5.43091 | −15.6844 | 10.79207 | −59.8473 | −10.7623 | −18.3974 | 2.791922 |
| 64 | 6.370379 | 7.851961 | −18.7333 | −37.4343 | 56.38978 | −18.1648 | −21.9546 | −1.34033 | 10.53808 |
| 65 | −3.92155 | −13.9046 | 45.21172 | −37.6644 | −7.9554 | −52.1563 | −30.0908 | −35.244 | 50.64836 |
| 66 | −2.97779 | −14.9781 | 51.71339 | −50.1743 | −3.93653 | −90.5378 | −39.2944 | −52.695 | 19.79041 |
| 67 | 5.000021 | 7.130794 | −17.1113 | −52.2292 | 73.11242 | 34.92153 | 33.10186 | 49.22568 | −8.02225 |
| 68 | −2.17392 | 11.13655 | −29.9504 | 36.14808 | −4.51687 | 279.5114 | 68.35324 | 104.7657 | −3.5352 |
| 69 | −14.5764 | −8.0116 | 23.02533 | −15.3253 | −7.46237 | −70.1042 | −45.0653 | −47.4249 | 12.15366 |
| 70 | −11.9534 | −11.3913 | 45.95304 | −47.0357 | −0.82036 | −37.5154 | −31.1182 | −31.4925 | 5.745585 |
| 71 | 0 | 4.230143 | −11.1527 | 7.506679 | 4.483417 | −26.1249 | −3.756 | −15.688 | 0.037718 |
| 72 | 0.364583 | −7.37229 | −0.09792 | −15.4732 | 15.16414 | −75.2046 | −41.2912 | −41.1166 | 12.59793 |
| 73 | −2.33546 | −5.4549 | 10.20556 | 19.62212 | −29.8298 | 147.3041 | −19.0498 | −18.4269 | 3.063875 |
| 74 | 0 | −11.8437 | 16.30698 | −21.7157 | 5.61637 | −84.1512 | −40.0166 | −43.3593 | 3.893126 |
| 75 | −4.14286 | −4.46212 | 47.07857 | −47.0562 | −0.39897 | −80.3407 | −20.7209 | −30.7019 | 5.548018 |
| 76 | −4.34783 | −9.45153 | 39.58037 | −32.7515 | −6.33577 | −65.2403 | −25.2726 | −39.1232 | 1.562057 |
| 77 | 6.25 | 2.571282 | −12.1622 | 20.2172 | 32.37473 | −56.2352 | −23.6044 | −33.6627 | 58.0063 |
| 78 | −5.66037 | −3.4045 | 37.13103 | −19.92 | −17.671 | −8.22925 | −6.11672 | −26.3768 | 5.281509 |
| 79 | 10.33869 | 3.381562 | 1.92312 | −27.6422 | 25.19422 | −80.9316 | −24.1646 | −24.6484 | −1.53974 |
| 80 | 18.25071 | 6.465797 | 3.996913 | −41.5502 | 34.41012 | −89.8629 | −47.0297 | −47.6559 | 17.95705 |
| 81 | 1.81819 | −0.26437 | −0.72435 | 6.04729 | −4.4424 | −2.00352 | −39.5739 | −43.6282 | 4.897427 |
| 82 | 6.818172 | 25.65705 | −28.9886 | −44.3831 | 73.45184 | 61.23588 | 16.9095 | 28.8321 | −15.3552 |
| 83 | −38.4491 | −23.7498 | 41.29664 | −40.6809 | −1.09496 | −85.8507 | −43.7902 | −51.27 | 44.77541 |
| 84 | 16.79431 | 18.38407 | −24.5129 | −27.7245 | 45.41465 | 43.45243 | −4.78082 | 0.477752 | 1.957307 |
| 85 | −7.1795 | −3.90558 | 52.7131 | −30.8322 | −13.7965 | −60.4377 | −15.2158 | −21.6371 | 5.666276 |
| 86 | 5.862046 | 4.391428 | −11.4345 | −23.4325 | 34.16812 | −57.1703 | −7.37505 | −7.82496 | 4.265339 |
| 87 | 3.921586 | −0.37722 | −21.307 | −35.3749 | 56.10183 | 53.48868 | 5.649769 | 1.476329 | 3.907623 |
| 88 | 6.976775 | 8.377632 | −8.23235 | −59.3966 | 69.35794 | −4.07523 | −15.2514 | −13.6368 | −4.91606 |
| 89 | 11.99998 | 15.36576 | −16.7464 | −41.4765 | 58.10996 | 43.44979 | 5.032022 | 9.411568 | −5.99833 |
| 90 | −1.96076 | 1.515775 | −9.38842 | 12.9868 | −3.42832 | −26.4966 | −41.7913 | −41.5633 | −0.03329 |
| 91 | 5.336551 | 8.958461 | −21.0137 | −10.0161 | 31.12997 | −35.6879 | −8.95733 | −4.38319 | −9.98553 |
| 92 | 7.843138 | 49.2191 | −37.7196 | −27.5051 | 65.43998 | 196.8999 | 11.00446 | 12.41387 | −12.2093 |

| | AK |
|---|---|
| 1 | |
| 2 | |
| 3 | Velocity |
| 4 | −50.2164 |
| 5 | −20.1137 |
| 6 | −17.9791 |
| 7 | 228.8425 |
| 8 | 33.11374 |
| 9 | −41.5088 |
| 10 | −45.1272 |
| 11 | |
| 12 | 46.88769 |
| 13 | −67.1786 |
| 14 | −5.05146 |
| 15 | 191.9683 |
| 16 | −14.604 |
| 17 | |
| 18 | −3.59747 |
| 19 | 25.51931 |
| 20 | 217.9712 |
| 21 | 179.6968 |
| 22 | −27.5068 |
| 23 | |
| 24 | |
| 25 | −21.7318 |
| 26 | 13.40715 |
| 27 | −46.732 |
| 28 | −46.4025 |
| 29 | −3.35862 |
| 30 | −17.867 |
| 31 | 11.42766 |
| 32 | 518.9566 |
| 33 | 135.2654 |
| 34 | −30.4655 |
| 35 | 3.983308 |
| 36 | −53.0091 |
| 37 | |
| 38 | 11.95169 |
| 39 | 14.14607 |
| 40 | 136.335 |
| 41 | 340.201 |
| 42 | 6.444143 |
| 43 | 157.6199 |
| 44 | 16.33104 |
| 45 | 11.94411 |
| 46 | −14.8723 |
| 47 | 182.7292 |
| 48 | |
| 49 | −28.4512 |
| 50 | −14.8 |
| 51 | 13.21417 |
| 52 | −60.2576 |
| 53 | 90.88165 |
| 54 | −33.2986 |
| 55 | |
| 56 | −7.98255 |
| 57 | −9.9725 |
| 58 | 250.6208 |
| 59 | 156.3361 |
| 60 | −31.3953 |
| 61 | |
| 62 | 70.24134 |
| 63 | −42.6583 |
| 64 | 19.12132 |
| 65 | −69.6787 |
| 66 | 149.2876 |
| 67 | 142.29 |
| 68 | 57.24989 |
| 69 | −47.8475 |
| 70 | 24.95963 |
| 71 | 13.42876 |
| 72 | −6.254 |
| 73 | −14.9757 |
| 74 | −1.8406 |
| 75 | 52.19158 |
| 76 | 324.377 |
| 77 | −64.9814 |
| 78 | |
| 79 | −15.3977 |
| 80 | −9.70179 |
| 81 | −37.6604 |
| 82 | −5.54162 |
| 83 | −0.07277 |
| 84 | 5.229314 |
| 85 | 97.33434 |
| 86 | 19.43471 |
| 87 | −17.0866 |
| 88 | 144.1052 |
| 89 | 77.63632 |
| 90 | −3.20582 |
| 91 | −71.0315 |
| 92 | −66.7793 |

TABLE 2.2

| | A | B | C | D | E | F | G | H |
|---|---|---|---|---|---|---|---|---|
| 1 | True values of DFA and SampEn and their percentage | | | | | | | |
| 2 | | | | | | | DFA(small) | |
| 3 | Date | File no. | Drug | Tissue | Dose | Dose | Pre | Post |
| 4 | May 14, 2019 | 23 | dop | stomach | 100 uM | 4 | 2.375477 | 2.270766 |
| 5 | May 15, 2019 | 13 | dop | stomach | 100 uM | 4 | 2.480475 | 2.433182 |
| 6 | May 15, 2019 | 27 | dop | stomach | 100 uM | 4 | 2.265233 | 2.146812 |
| 7 | May 20, 2019 | 13 | dop | stomach | 100 uM | 4 | 2.389863 | 2.376932 |
| 8 | May 20, 2019 | 23 | dop | stomach | 100 uM | 4 | 2.409517 | 2.281367 |
| 9 | May 21, 2019 | 13 | dop | stomach | 100 uM | 4 | 2.239034 | 1.982043 |
| 10 | May 21, 2019 | 26 | dop | stomach | 100 uM | 4 | 2.390247 | 2.403438 |
| 11 | Nov. 25, 2019 | 1 | dop | duodenum | 100 nM | 7 | 2.593867 | 2.485594 |
| 12 | Nov. 26, 2019 | 4 | dop | duodenum | 100 nM | 7 | 2.70446 | 2.546411 |
| 13 | Nov. 26, 2019 | 10 | dop | duodenun | 100 nM | 7 | 2.596299 | 2.569456 |
| 14 | Nov. 27, 2019 | 1 | dop | duodenum | 100 nM | 7 | 2.675295 | 2.486405 |
| 15 | Nov. 28, 2019 | 1 | dop | duodenun | 100 nM | 7 | 2.752703 | 2.542899 |
| 16 | Nov. 28, 2019 | 7 | dop | duodenum | 100 nM | 7 | 2.557251 | 2.437394 |
| 17 | Apr. 15, 2019 | 7 | dop | duodenun | 100 uM | 4 | 2.516845 | 2.48424 |
| 18 | Apr. 23, 2019 | 1 | dop | duodenun | 100 uM | 4 | 2.333873 | 2.222587 |
| 19 | Apr. 23, 2019 | 11 | dop | duodenum | 100 uM | 4 | 1.942346 | 1.973908 |
| 20 | Apr. 23, 2019 | 21 | dop | duodenum | 100 uM | 4 | 2.303862 | 2.079284 |
| 21 | Apr. 24, 2019 | 4 | dop | duodenum | 100 uM | 4 | 2.244339 | 2.109334 |
| 22 | Apr. 24, 2019 | 14 | dop | duodenum | 100 uM | 4 | 2.206425 | 2.309379 |
| 23 | May 2, 2019 | 1 | dop | duodenum | 100 uM | 4 | 2.163072 | 1.993088 |
| 24 | May 2, 2019 | 14 | dop | duodenun | 100 uM | 4 | 2.289508 | 2.242578 |
| 25 | May 3, 2019 | 4 | dop | duodenun | 100 uM | 4 | 2.341756 | 2.18128 |
| 26 | Aug. 5, 2019 | 7 | dop | duodenun | 10 uM | 5 | 2.251281 | 2.18103 |
| 27 | Aug. 5, 2019 | 19 | dop | duodenum | 10 uM | 5 | 1.953193 | 1.900518 |
| 28 | Aug. 6, 2019 | 4 | dop | duodenum | 10 uM | 5 | 2.216923 | 2.115073 |
| 29 | Aug. 6, 2019 | 17 | dop | duodenun | 10 uM | 5 | 2.232422 | 2.017462 |
| 30 | Aug. 15, 2019 | 1 | dop | duodenun | 10 uM | 5 | 2.339852 | 2.40225 |
| 31 | Aug. 15, 2019 | 13 | dop | duodenun | 10 uM | 5 | 2.174971 | 1.803828 |
| 32 | Aug. 26, 2019 | 14 | dop | duodenun | 10 uM | 5 | 2.235364 | 2.194831 |
| 33 | Aug. 12, 2019 | 7 | dop | duodenum | 1 um | 6 | 2.236064 | 2.125898 |
| 34 | Aug. 12, 2019 | 18 | dop | duodenum | 1 um | 6 | 2.284731 | 2.285666 |
| 35 | Aug. 13, 2019 | 4 | dop | duodenum | 1 um | 6 | 2.277113 | 2.298803 |
| 36 | Aug. 13, 2019 | 17 | dop | duodenum | 1 um | 6 | 2.734587 | 2.618149 |
| 37 | Aug. 14, 2019 | 1 | dop | duodenum | 1 um | 6 | 1.870655 | 1.795451 |
| 38 | Aug. 14, 2019 | 14 | dop | duodenum | 1 um | 6 | 2.512637 | 2.533317 |
| 39 | Aug. 26, 2019 | 1 | dop | duodenum | 1 um | 6 | 2.48273 | 2.183902 |
| 40 | Nov. 25, 2019 | 2 | dop | ileum | 100 nM | 7 | 2.398484 | 2.312075 |
| 41 | Nov. 26, 2019 | 5 | dop | ileum | 100 nM | 7 | 2.287849 | 2.157114 |
| 42 | Nov. 26, 2019 | 11 | dop | ileum | 100 nM | 7 | 2.312753 | 2.23203 |
| 43 | Nov. 27, 2019 | 2 | dop | ileum | 100 nM | 7 | 2.55829 | 2.454889 |
| 44 | Nov. 28, 2019 | 2 | dop | ileum | 100 nM | 7 | 2.394969 | 2.3231261 |
| 45 | Nov. 28, 2019 | 8 | dop | ileum | 100 nM | 7 | 2.122513 | 2.173117 |
| 46 | Apr. 15, 2019 | 8 | dop | ileum | 100 uM | 4 | 2.372357 | 2.331469 |
| 47 | Apr. 23, 2019 | 2 | dop | ileum | 100 uM | 4 | 2.137868 | 2.192187 |
| 48 | Apr. 23, 2019 | 12 | dop | ileum | 100 uM | 4 | 2.256948 | 2.19182 |
| 49 | Apr. 24, 2019 | 5 | dop | ileum | 100 uM | 4 | 2.379063 | 2.213733 |
| 50 | Apr. 24, 2019 | 15 | dop | ileum | 100 uM | 4 | 2.504558 | 2.284726 |
| 51 | May 2, 2019 | 2 | dop | ileum | 100 uM | 4 | 2.392191 | 2.256827 |
| 52 | May 2, 2019 | 15 | dop | ileum | 100 uM | 4 | 2.375075 | 2.291227 |
| 53 | Aug. 5, 2019 | 8 | dop | ileum | 10 uM | 5 | 2.06631 | 1.828172 |
| 54 | Aug. 5, 2019 | 20 | dop | ileum | 10 uM | 5 | 1.989387 | 1.746187 |
| 55 | Aug. 6, 2019 | 5 | dop | ileum | 10 uM | 5 | 1.798989 | 1.83384 |
| 56 | Aug. 6, 2019 | 18 | dop | ileum | 10 uM | 5 | 2.214779 | 2.045148 |
| 57 | Aug. 15, 2019 | 2 | dop | ileum | 10 uM | 5 | 2.690534 | 2.622836 |
| 58 | Aug. 15, 2019 | 14 | dop | ileum | 10 uM | 5 | 2.077315 | 2.118478 |
| 59 | Oct. 14, 2019 | 5 | dop | ileum | 10 uM | 5 | 2.627464 | 2.581398 |
| 60 | Oct. 14, 2019 | 13 | dop | ileum | 10 uM | 5 | 2.432251 | 2.289129 |
| 61 | Aug. 12, 2019 | 8 | dop | ileum | 1 um | 6 | 1.973491 | 2.092242 |
| 62 | Aug. 12, 2019 | 19 | dop | ileum | 1 um | 6 | 2.278646 | 2.05274 |
| 63 | Aug. 13, 2019 | 5 | dop | ileum | 1 um | 6 | 2.29267 | 2.113318 |
| 64 | Aug. 13, 2019 | 18 | dop | ileum | 1 um | 6 | 2.607369 | 2.469243 |
| 65 | Aug. 14, 2019 | 2 | dop | ileum | 1 um | 6 | 2.273746 | 2.102621 |
| 66 | Aug. 14, 2019 | 15 | dop | ileum | 1 um | 6 | 2.596701 | 2.469932 |
| 67 | Aug. 26, 2019 | 2 | dop | ileum | 1 um | 6 | 2.303953 | 2.277369 |
| 68 | Aug. 26, 2019 | 15 | dop | ileum | 1 um | 6 | 2.01874 | 1.917585 |
| 69 | Nov. 25, 2019 | 3 | dop | colon | 100 nM | 7 | 2.496056 | 2.358825 |
| 70 | Nov. 26, 2019 | 6 | dop | colon | 100 nM | 7 | 2.33981 | 2.289689 |
| 71 | Nov. 26, 2019 | 12 | dop | colon | 100 nM | 7 | 2.310353 | 2.209165 |
| 72 | Nov. 27, 2019 | 3 | dop | colon | 100 nM | 7 | 2.419197 | 2.262553 |
| 73 | Nov. 28, 2019 | 3 | dop | colon | 100 nM | 7 | 2.323912 | 2.25464 |
| 74 | Nov. 28, 2019 | 9 | dop | colon | 100 nM | 7 | 2.356429 | 2.174525 |
| 75 | Apr. 15, 2019 | 9 | dop | colon | 100 uM | 4 | 2.395288 | 2.395899 |
| 76 | Apr. 23, 2019 | 3 | dop | colon | 100 uM | 4 | 2.245757 | 2.18083 |
| 77 | Apr. 23, 2019 | 13 | dop | colon | 100 uM | 4 | 2.107773 | 2.102317 |
| 78 | Apr. 24, 2019 | 6 | dop | colon | 100 uM | 4 | 2.412763 | 2.148345 |
| 79 | Apr. 24, 2019 | 16 | dop | colon | 100 uM | 4 | 2.20718 | 2.13021 |
| 80 | May 2, 2019 | 3 | dop | colon | 100 uM | 4 | 2.281605 | 2.04834 |
| 81 | May 2, 2019 | 16 | dop | colon | 100 uM | 4 | 2.252331 | 2.117861 |
| 82 | May 3, 2019 | 3 | dop | colon | 100 uM | 4 | 2.49695 | 2.427454 |
| 83 | May 3, 2019 | 18 | dop | colon | 100 uM | 4 | 2.41524 | 2.253491 |
| 84 | May 14, 2019 | 12 | dop | colon | 100 uM | 4 | 2.295039 | 2.068013 |
| 85 | Aug. 5, 2019 | 9 | dop | colon | 10 uM | 5 | 2.058255 | 2.117343 |
| 86 | Aug. 5, 2019 | 21 | dop | colon | 10 uM | 5 | 2.165906 | 1.925279 |
| 87 | Aug. 6, 2019 | 6 | dop | colon | 10 uM | 5 | 2.100329 | 1.950882 |
| 88 | Aug. 6, 2019 | 19 | dop | colon | 10 uM | 5 | 2.234107 | 1.967164] |
| 89 | Aug. 15, 2019 | 3 | dop | colon | 10 uM | 5 | 2.687172 | 2.639234 |
| 90 | Aug. 15, 2019 | 15 | dop | colon | 10 uM | 5 | 2.171826 | 2.034206 |
| 91 | Aug. 12, 2019 | 9 | dop | colon | 1 um | 6 | 1.653962 | 1.536201 |
| 92 | Aug. 12, 2019 | 20 | dop | colon | 1 um | 6 | 2.275473 | 2.163102 |
| 93 | Aug. 13, 2019 | 6 | dop | colon | 1 um | 6 | 2.100363 | 2.13258 |
| 94 | Aug. 13, 2019 | 19 | dop | colon | 1 um | 6 | 2.319622 | 2.246482 |
| 95 | Aug. 14, 2019 | 3 | dop | colon | 1 um | 6 | 2.70853 | 2.696801 |
| 96 | Aug. 14, 2019 | 16 | dop | colon | 1 um | 6 | 2.491164 | 2.485831 |
| 97 | Aug. 26, 2019 | 3 | dop | colon | 1 um | 6 | 2.580063 | 2.437676 |
| 98 | Aug. 26, 2019 | 16 | dop | colon | 1 um | 6 | 2.290536 | 2.155816 |

| | I | J | K | L | M | N | O | P | Q |
|---|---|---|---|---|---|---|---|---|---|
| 1 | e change between pre and post drug treatment | | | | | | | | |
| 2 | IDFAPC(sm DFA(large) | | | DFAPC(large En(small) | | | EnPC(small En(large) | | |
| 3 | | Pre | Post | | Pre | Post | | Pre | Post |
| 4 | −4.40797 | 1.555201 | 1.447997 | −6.89327 | 0.630284 | 0.618207 | −1.91617 | 0.226994 | 0.225094 |
| 5 | −1.90659 | 1.950144 | 1.735804 | −10.991 | 0.558758 | 0.611933 | 9.516653 | 0.347875 | 0.248886 |
| 6 | −5.22776 | 1.684564 | 1.517283 | −9.93023 | 0.583134 | 0.613732 | 5.247173 | 0.30989 | 0.290998 |
| 7 | −0.54107 | 1.780118 | 1.79313 | 0.730959 | 0.608376 | 0.578756 | −4.86874 | 0.266003 | 0.315741 |
| 8 | −5.3185 | 1.850171 | 1.651321 | −10.7476 | 0.561407 | 0.582799 | 3.81039 | 0.322611 | 0.291461 |
| 9 | −11.4778 | 1.644917 | 1.116494 | −32.1246 | 0.583576 | 0.60595 | 3.833906 | 0.301904 | 0.206229 |
| 10 | 0.551838 | 1.785735 | 1.815448 | 1.663927 | 0.581777 | 0.579189 | −0.44495 | 0.293435 | 0.302187 |
| 11 | −4.17418 | 2.060131 | 1.869669 | −9.24515 | 0.602843 | 0.610246 | 1.228069 | 0.368136 | 0.301817 |
| 12 | −5.84403 | 2.26328 | 2.081942 | −8.01216 | 0.506873 | 0.516922 | 1.982574 | 0.393273 | 0.388466 |
| 13 | −1.03391 | 2.098459 | 2.076489 | −1.04697 | 0.535006 | 0.517152 | −3.33718 | 0.392925 | 0.403028 |
| 14 | −7.06052 | 2.186056 | 1.840034 | −15.8286 | 0.591902 | 0.618988 | 4.576112 | 0.408201 | 0.313264 |
| 15 | −7.62174 | 2.208992 | 1.978747 | −10.423 | 0.53718 | 0.567051 | 5.56069 | 0.3919 | 0.379347 |
| 16 | −4.68693 | 2.175512 | 2.061139 | −5.25727 | 0.539885 | 0.555934 | 2.972725 | 0.421196 | 0.41794 |
| 17 | −1.29549 | 1.865813 | 1.841644 | −1.29539 | 0.529242 | 0.544784 | 2.936554 | 0.305199 | 0.311977 |
| 18 | −4.76828 | 1.308061 | 1.235784 | −5.52551 | 0.570909 | 0.603086 | 5.636049 | 0.169272 | 0.169761 |
| 19 | 1.624949 | 0.958044 | 0.947264 | −1.12524 | 0.595237 | 0.580169 | −2.53148 | 0.164396 | 0.154986 |
| 20 | −9.74791 | 1.261692 | 1.103245 | −12.5583 | 0.565337 | 0.593448 | 4.972465 | 0.161681 | 0.179651 |
| 21 | −6.01536 | 1.575035 | 1.408496 | −10.5737 | 0.574788 | 0.591112 | 2.840065 | 0.297403 | 0.268122 |
| 22 | 4.666074 | 1.512726 | 1.590326 | 5.129844 | 0.600778 | 0.580983 | −3.29485 | 0.262541 | 0.254313 |
| 23 | −7.85848 | 1.223508 | 0.965885 | −21.0561 | 0.592822 | 0.567108 | −4.3375 | 0.18722 | 0.173111 |
| 24 | −2.04982 | 1.663851 | 1.619547 | −2.66276 | 0.608743 | 0.588601 | −3.30869 | 0.3075 | 0.321418 |
| 25 | −6.85281 | 1.779177 | 1.531247 | −13.9351 | 0.528122 | 0.547219 | 3.616079 | 0.351819 | 0.324295 |
| 26 | −3.12047 | 1.861237 | 1.735724 | −6.74351 | 0.565842 | 0.601795 | 6.353912 | 0.403726 | 0.412489 |
| 27 | −2.69686 | 1.46643 | 1.36351 | −7.01836 | 0.622282 | 0.625375 | 0.497066 | 0.36904 | 0.34477 |
| 28 | −4.59418 | 1.750662 | 1.699174 | −2.94108 | 0.532511 | 0.53643 | 0.735859 | 0.382722 | 0.41926 |
| 29 | −9.62898 | 1.73069 | 1.487251 | −14.066 | 0.505985 | 0.564637 | 11.59157 | 0.310047 | 0.344945 |
| 30 | 2.666766 | 1.526976 | 1.62681 | 6.538005 | 0.635518 | 0.630208 | −0.83539 | 0.218012 | 0.196852 |
| 31 | −17.0643 | 1.222035 | 1.079384 | −11.6732 | 0.601794 | 0.657933 | 9.328561 | 0.200661 | 0.250489 |
| 32 | −1.81326 | 1.220568 | 1.205609 | −1.22553 | 0.576923 | 0.596694 | 3.42713 | 0.169065 | 0.160167 |
| 33 | −4.92681 | 1.82399 | 1.57049 | −13.8981 | 0.543609 | 0.599545 | 10.28978 | 0.415484 | 0.377352 |
| 34 | 0.040959 | 1.838204 | 1.868653 | 1.656454 | 0.503711 | 0.5073 | 0.712626 | 0.394878 | 0.390711 |
| 35 | 0.952527 | 1.76103 | 1.774633 | 0.772431 | 0.493999 | 0.504415 | 2.108343 | 0.333435 | 0.359044 |
| 36 | −4.25798 | 2.325804 | 2.251181 | −3.20847 | 0.45267 | 0.466522 | 3.060045 | 0.379278 | 0.387365 |
| 37 | −4.0202 | 1.32638 | 1.142241 | −13.8828 | 0.58778 | 0.624582 | 6.261265 | 0.349187 | 0.281755 |
| 38 | 0.823045 | 1.688774 | 1.609451 | −4.69708 | 0.634683 | 0.614681 | −3.15138 | 0.185402 | 0.200723 |
| 39 | −12.0362 | 1.444747 | 1.219606 | −15.5835 | 0.546498 | 0.562412 | 2.912008 | 0.176071 | 0.177596 |
| 40 | −3.60264 | 1.719582 | 1.542286 | −10.3104 | 0.659386 | 0.624055 | −5.35816 | 0.255668 | 0.2025 |
| 41 | −5.71432 | 1.650368 | 1.395267 | −15.4572 | 0.637126 | 0.636413 | −0.11198 | 0.281224 | 0.232822 |
| 42 | −3.49034 | 1.670554 | 1.484733 | −11.1233 | 0.639077 | 0.625624 | −2.10511 | 0.310463 | 0.247562 |
| 43 | −4.04182 | 1.917036 | 1.733831 | −9.5567 | 0.630653 | 0.618602 | −1.91089 | 0.280719 | 0.237322 |
| 44 | −2.99973 | 1.839688 | 1.660837 | −9.72178 | 0.603583 | 0.611386 | 1.292669 | 0.317882 | 0.3093 |
| 45 | 2.384135 | 1.434383 | 1.477117 | 2.979247 | 0.648635 | 0.640727 | −1.21914 | 0.249257 | 0.268195 |
| 46 | −1.7235 | 1.762623 | 1.786532 | 1.356479 | 0.570951 | 0.549758 | −3.71192 | 0.276536 | 0.325291 |
| 47 | 2.54078 | 1.289071 | 1.200777 | −6.84941 | 0.604211 | 0.584066 | −3.33412 | 0.227995 | 0.166506 |
| 48 | −2.88565 | 1.346722 | 1.203287 | −10.6507 | 0.607413 | 0.584904 | −3.70573 | 0.194241 | 0.171845 |
| 49 | −6.9494 | 1.710763 | 1.365628 | −20.1744 | 0.590876 | 0.625057 | 5.784865 | 0.247217 | 0.19228 |
| 50 | −8.77726 | 1.933114 | 1.508481 | −21.9662 | 0.594455 | 0.612781 | 3.082775 | 0.344237 | 0.239261 |
| 51 | −5.65858 | 1.624671 | 1.35131 | −16.8256 | 0.599994 | 0.610287 | 1.715557 | 0.230423 | 0.195068 |
| 52 | −3.53029 | 1.76734 | 1.587917 | −10.1522 | 0.586827 | 0.629541 | 7.278741 | 0.286229 | 0.257945 |
| 53 | −11.5248 | 1.621287 | 1.341665 | −17.2469 | 0.611083 | 0.599587 | −1.88132 | 0.406119 | 0.391948 |
| 54 | −12.2249 | 1.391843 | 1.025695 | −26.3067 | 0.63019 | 0.625709 | −0.71097 | 0.321018 | 0.278161 |
| 55 | 1.937234 | 1.20904 | 1.276923 | 5.614572 | 0.60912 | 0.616475 | 1.207551 | 0.341577 | 0.329867 |
| 56 | −7.65907 | 1.820164 | 1.603908 | −11.8811 | 0.550756 | 0.628849 | 14.17931 | 0.366251 | 0.393218 |
| 57 | −2.51618 | 1.775015 | 1.740497 | −1.94462 | 0.590411 | 0.610251 | 3.360405 | 0.203225 | 0.203119 |
| 58 | 1.981524 | 1.385402 | 1.51281 | 9.196502 | 0.621733 | 0.582588 | −6.29607 | 0.230152 | 0.319246 |
| 59 | −1.75326 | 2.159032 | 2.059595 | −4.60566 | 0.564217 | 0.581071 | 2.987213 | 0.389184 | 0.388773 |
| 60 | −5.88434 | 1.880831 | 1.715058 | −8.8138 | 0.522672 | 0.576734 | 10.34344 | 0.378325 | 0.345646 |
| 61 | 6.017328 | 1.59561 | 1.769628 | 10.90606 | 0.548515 | 0.497484 | −9.30351 | 0.396404 | 0.417711 |
| 62 | −9.91404 | 1.796498 | 1.571162 | −12.5431 | 0.514729 | 0.553815 | 7.593633 | 0.321801 | 0.343043 |
| 63 | −7.82288 | 1.961493 | 1.739858 | −11.2993 | 0.506672 | 0.488954 | −3.49705 | 0.415776 | 0.403526 |
| 64 | −5.29753 | 2.23684 | 2.170579 | −2.96228 | 0.504566 | 0.499899 | −0.92494 | 0.392384 | 0.398007 |
| 65 | −7.52611 | 1.758216 | 1.493034 | −15.0825 | 0.546975 | 0.568131 | 3.867757 | 0.35071 | 0.334672 |
| 66 | −4.88193 | 1.833695 | 1.695645 | −7.52848 | 0.637435 | 0.653644 | 2.542868 | 0.240702 | 0.210149 |
| 67 | −1.15386 | 1.573427 | 1.420024 | −9.74961 | 0.670255 | 0.646627 | −3.52513 | 0.228128 | 0.184566 |
| 68 | −5.01081 | 1.490674 | 1.237718 | −16.9692 | 0.561628 | 0.636081 | 13.25657 | 0.325305 | 0.270703 |
| 69 | −5.49794 | 1.78072 | 1.469065 | −17.5016 | 0.593634 | 0.62418 | 5.145529 | 0.241829 | 0.175392 |
| 70 | −2.14212 | 1.896698 | 1.712262 | −9.72406 | 0.564054 | 0.620259 | 9.964431 | 0.393342 | 0.34524 |
| 71 | −4.37977 | 1.641866 | 1.448601 | −11.7711 | 0.59593 | 0.631129 | 5.906548 | 0.288437 | 0.230264 |
| 72 | −6.47505 | 1.859477 | 1.6307 | −12.3033 | 0.618532 | 0.637981 | 3.144458 | 0.346156 | 0.298192 |
| 73 | −2.98085 | 1.810078 | 1.67453 | −7.48856 | 0.532792 | 0.563304 | 5.726664 | 0.379374 | 0.36286 |
| 74 | −7.71946 | 1.817416 | 1.521694 | −16.2715 | 0.615956 | 0.626884 | 1.774039 | 0.360215 | 0.297912 |
| 75 | 0.025501 | 1.717829 | 1.728338 | 0.611763 | 0.582048 | 0.57638 | −0.97384 | 0.268617 | 0.26956 |
| 76 | −2.8911 | 1.416343 | 1.351365 | −4.58773 | 0.565317 | 0.578455 | 2.323932 | 0.21053 | 0.196323 |
| 77 | −0.25889 | 1.296761 | 1.427783 | 10.10375 | 0.601986 | 0.583226 | −3.11633 | 0.22283 | 0.273527 |
| 78 | −10.9592 | 1.786351 | 1.414733 | −20.8032 | 0.533796 | 0.594569 | 11.38512 | 0.324098 | 0.266194 |
| 79 | −3.48723 | 1.433383 | 1.267976 | −11.5397 | 0.604947 | 0.619089 | 2.337827 | 0.225865 | 0.187214 |
| 80 | −10.2237 | 1.58787 | 1.290369 | −18.7359 | 0.55998 | 0.581709 | 3.880315 | 0.268642 | 0.260756 |
| 81 | −5.97027 | 1.654739 | 1.332826 | −19.454 | 0.596355 | 0.604515 | 1.368303 | 0.284937 | 0.220284 |
| 82 | −2.78322 | 1.765648 | 1.797617 | 1.810576 | 0.583191 | 0.573137 | −1.72391 | 0.266234 | 0.315469 |
| 83 | −6.69701 | 1.8921 | 1.723152 | −8.92916 | 0.554599 | 0.572114 | 3.158014 | 0.352413 | 0.333891 |
| 84 | −9.89203 | 1.747928 | 1.242084 | −28.9397 | 0.581094 | 0.618247 | 6.393548 | 0.318057 | 0.237805 |
| 85 | 2.870801 | 1.539844 | 1.581635 | 2.713916 | 0.608604 | 0.630316 | 3.567506 | 0.351275 | 0.357745 |
| 86 | −11.1097 | 1.733949 | 1.391893 | −19.727 | 0.600708 | 0.606792 | 1.012699 | 0.40859 | 0.371477 |
| 87 | −7.11539 | 1.687337 | 1.502383 | −10.9613 | 0.522508 | 0.565576 | 8.242471 | 0.404453 | 0.399858 |
| 88 | −11.9485 | 1.876434 | 1.572435 | −16.2009 | 0.4681 | 0.542972 | 15.99498 | 0.326245 | 0.389935 |
| 89 | −1.78396 | 1.953314 | 1.833414 | −6.1383 | 0.614047 | 0.627676 | 2.219586 | 0.240473 | 0.228318 |
| 90 | −6.3366 | 1.600699 | 1.291865 | −19.2937 | 0.598197 | 0.626111 | 4.666407 | 0.299868 | 0.226297 |
| 91 | −7.11992 | 1.154421 | 0.943692 | −18.2541 | 0.594627 | 0.625168 | 5.136202 | 0.363526 | 0.302723 |
| 92 | −4.93836 | 1.890245 | 1.679924 | −11.1267 | 0.487953 | 0.532206 | 9.069094 | 0.361793 | 0.381468 |
| 93 | 1.533853 | 1.613414 | 1.5551 | −3.61427 | 0.540718 | 0.585713 | 8.321407 | 0.375177 | 0.325876 |
| 94 | −3.15311 | 1.910936 | 1.842728 | −3.56937 | 0.490721 | 0.516616 | 5.276998 | 0.416077 | 0.41972 |
| 95 | −0.43304 | 1.914105 | 1.851753 | −3.25751 | 0.622295 | 0.633254 | 1.761111 | 0.21702 | 0.221351 |
| 96 | −0.2141 | 1.751625 | 1.745163 | −0.36892 | 0.635142 | 0.618864 | −2.56287 | 0.258533 | 0.230997 |
| 97 | −5.51875 | 1.903718 | 1.705245 | −10.4255 | 0.59253 | 0.628752 | 6.113032 | 0.274934 | 0.231854 |
| 98 | −5.88159 | 1.770428 | 1.547118 | −12.6133 | 0.560885 | 0.607588 | 8.326661 | 0.360268 | 0.293246 |

| | R | S |
|---|---|---|
| 1 | | |
| 2 | EnPC(large) | |
| 3 | | |
| 4 | −0.83687 | |
| 5 | −28.4554 | |
| 6 | −6.0963 | |
| 7 | 18.69839 | |
| 8 | −9.65576 | |
| 9 | −31.6906 | |
| 10 | 2.982644 | |
| 11 | −18.0149 | |
| 12 | −1.22246 | |
| 13 | 2.571176 | |
| 14 | −23.2574 | |
| 15 | −3.20313 | |
| 16 | −0.77299 | |
| 17 | 2.220542 | |
| 18 | 0.289238 | |
| 19 | −5.7242 | |
| 20 | 11.11438 | |
| 21 | −9.84555 | |
| 22 | −3.13372 | |
| 23 | −7.536 | |
| 24 | 4.526144 | |
| 25 | −7.82347 | |
| 26 | 2.170534 | |
| 27 | −6.57652 | |
| 28 | 9.546939 | |
| 29 | 11.25596 | |
| 30 | −9.70572 | |
| 31 | 24.83231 | |
| 32 | −5.26284 | |
| 33 | −9.1778 | |
| 34 | −1.0554 | |
| 35 | 7.68032 | |
| 36 | 2.132157 | |
| 37 | −19.3112 | |
| 38 | 8.26356 | |
| 39 | 0.865946 | |
| 40 | −20.7958 | |
| 41 | −17.2112 | |
| 42 | −20.2605 | |
| 43 | −15.4593 | |
| 44 | −2.69973 | |
| 45 | 7.597714 | |
| 46 | 17.63059 | |
| 47 | −26.9692 | |
| 48 | −11.5301 | |
| 49 | −22.2221 | |
| 50 | −30.4952 | |
| 51 | −15.3436 | |
| 52 | −9.88172 | |
| 53 | −3.48932 | |
| 54 | −13.3504 | |
| 55 | −3.42825 | |
| 56 | 7.362848 | |
| 57 | −0.05189 | |
| 58 | 38.71093 | |
| 59 | −0.10575 | |
| 60 | −8.63784 | |
| 61 | 5.375095 | |
| 62 | 6.601074 | |
| 63 | −2.94632 | |
| 64 | 1.433143 | |
| 65 | −4.57297 | |
| 66 | −12.6932 | |
| 67 | −19.0952 | |
| 68 | −16.7846 | |
| 69 | −27.4725 | |
| 70 | −12.2292 | |
| 71 | −20.1683 | |
| 72 | −13.8563 | |
| 73 | −4.35298 | |
| 74 | −17.2961 | |
| 75 | 0.351069 | |
| 76 | −6.74797 | |
| 77 | 22.75172 | |
| 78 | −17.8662 | |
| 79 | −17.1123 | |
| 80 | −2.93538 | |
| 81 | −22.69 | |
| 82 | 18.49332 | |
| 83 | −5.25566 | |
| 84 | −25.232 | |
| 85 | 1.841865 | |
| 86 | −9.08336 | |
| 87 | −1.1362 | |
| 88 | 19.52213 | |
| 89 | −5.05459 | |
| 90 | −24.5344 | |
| 91 | −16.7258 | |
| 92 | 5.438235 | |
| 93 | −13.1406 | |
| 94 | 3 0.875661 | |
| 95 | 1.995919 | |
| 96 | −10.651 | |
| 97 | −15.6691 | |
| 98 | −18.6033 | |

TABLE 2.3
True mean and standard derivations

| | A | B | C | D | E | F | G | H |
|---|---|---|---|---|---|---|---|---|
| 1 | | | | | | | | |
| 2 | | | | | | Mean | | |
| 3 | | | | | | DF | | AF |
| 4 | Drug | Tissue | Dose | Dose | Repeat No | Pre | Post | Pre |
| 5 | dop | stomach | 100 uM | 4 | 8 | 7.666209 | 8.148341 | 9.847781 |
| 6 | dop | duodenum | 100 nM | 7 | 7 | 27.07031 | 24.87305 | 24.11905 |
| 7 | dop | duodenum | 1 uM | 6 | 6 | 29.51212 | 26.86045 | 27.42705 |
| 8 | dop | duodenum | 10 uM | 5 | 9 | 29.27489 | 29.23147 | 26.25349 |
| 9 | dop | duodenum | 100 uM | 4 | 5 | 28.58724 | 26.33727 | 25.10932 |
| 10 | dop | ileum | 100 nM | 7 | 7 | 26.26127 | 23.02576 | 23.47332 |
| 11 | dop | ileum | 1 uM | 6 | 7 | 25.88698 | 25.55086 | 24.9937 |
| 12 | dop | ileum | 10 uM | 5 | 6 | 28.77163 | 25.72417 | 26.34074 |
| 13 | dop | ileum | 100 uM | 4 | 6 | 27.93899 | 27.60417 | 24.31959 |
| 14 | dop | colon | 100 nM | 7 | 7 | 27.34375 | 26.64074 | 23.90141 |
| 15 | dop | colon | 1 uM | 6 | 6 | 30.47712 | 31.59389 | 27.28081 |
| 16 | dop | colon | 10 uM | 5 | 9 | 29.57148 | 30.27008 | 27.92117 |
| 17 | dop | colon | 100 uM | 4 | 6 | 28.13151 | 27.4249 | 25.36122 |

| | I | J | K | L | M | N | O | P |
|---|---|---|---|---|---|---|---|---|
| 1 | | | | | | | | |
| 2 | | | | | | | | |
| 3 | | B | | N | | T | | DP |
| 4 | Post | Pre | Post | Pre | Post | Pre | Post | Pre |
| 5 | 11.76131 | 8.417049 | 15.41579 | 55.10807 | 23.60213 | 36.06583 | 60.19476 | 887.9864 |
| 6 | 20.8544 | 35.6991 | 87.17686 | 46.11872 | 4.816448 | 12.55019 | 2.10117 | 815.9348 |
| 7 | 25.61283 | 25.77922 | 53.73974 | 50.57929 | 24.47956 | 22.2992 | 20.70276 | 989.7395 |
| 8 | 25.37742 | 31.52292 | 56.61996 | 52.3777 | 7.826552 | 14.71837 | 34.2519 | 413.7344 |
| 9 | 22.17471 | 43.26263 | 68.6098 | 43.84373 | 24.99279 | 11.03182 | 5.355146 | 1060.834 |
| 10 | 19.39921 | 31.75341 | 60.02687 | 51.19428 | 14.6291 | 10.68732 | 20.00247 | 780.4363 |
| 11 | 24.07385 | 27.46361 | 46.262 | 40.48774 | 15.4824 | 30.86594 | 36.65598 | 1070.487 |
| 12 | 22.71846 | 32.81144 | 63.28993 | 50.93642 | 9.238937 | 14.9062 | 26.12427 | 1446.493 |
| 13 | 24.20774 | 41.48996 | 48.79936 | 45.25801 | 25.04798 | 12.35393 | 24.94581 | 905.118 |
| 14 | 23.27551 | 39.4134 | 48.4393 | 48.73078 | 22.61754 | 10.03965 | 27.64482 | 1910.972 |
| 15 | 29.56971 | 37.46321 | 28.32211 | 41.36778 | 14.48689 | 18.34704 | 55.48241 | 1762.519 |
| 16 | 29.18974 | 26.98496 | 25.81677 | 42.41536 | 13.0931 | 29.51525 | 58.33748 | 2590.054 |
| 17 | 24.02206 | 34.72524 | 53.92999 | 53.13977 | 33.46859 | 11.40005 | 11.68702 | 2431.289 |
| | Q | R | S | T | U | V | W | X |
| 1 | | | | | | | | |
| 2 | | | | | | | | |
| 3 | | PPAmp | | Slope | | Period | | Velocity |
| 4 | Post | Pre | Post | Pre | Post | Pre | Post | Pre |
| 5 | 253.1927 | 162.6938 | 126.8881 | 119.2443 | 104.0708 | 7.452247 | 7.66696 | 10.60811 |
| 6 | 382.2264 | 193.0043 | 114.1422 | 321.7088 | 166.8509 | 2.518156 | 3.270707 | 12.54962 |
| 7 | 427.3125 | 187.3769 | 130.4728 | 327.3237 | 206.461 | 2.264239 | 2.766869 | 12.45968 |
| 8 | 137.826 | 117.5765 | 97.38451 | 202.3646 | 153.8872 | 3.010336 | 3.630872 | 17.77473 |
| 9 | 388.5566 | 199.338 | 121.0515 | 322.2333 | 182.5209 | 2.441572 | 3.256181 | 11.83095 |
| 10 | 177.5254 | 165.1927 | 119.2081 | 257.5321 | 160.0611 | 2.662801 | 3.732104 | 10.47592 |
| 11 | 460.2607 | 187.0543 | 138.0307 | 300.8899 | 204.3844 | 2.530279 | 3.095015 | 16.77924 |
| 12 | 760.18 | 229.563 | 149.0748 | 383.6991 | 238.1939 | 2.302001 | 3.048012 | 17.72554 |
| 13 | 400.7727 | 183.8137 | 136.8231 | 280.5297 | 197.0423 | 2.485303 | 2.712049 | 6.724907 |
| 14 | 1415.886 | 230.8942 | 208.944 | 357.113 | 329.5393 | 2.328841 | 2.638358 | 13.57336 |
| 15 | 2203.399 | 251.9418 | 233.8342 | 429.1537 | 400.5277 | 2.052601 | 1.996225 | 20.583 |
| 16 | 3178.673 | 278.1256 | 223.7033 | 479.9934 | 389.8203 | 2.099196 | 2.288157 | 13.68932 |
| 17 | 1069.016 | 267.2952 | 199.744 | 439.2788 | 298.9748 | 2.248496 | 2.480643 | 10.28267 |
| | Y | Z | AA | AB | AC | AD | AE | AF |
| 1 | | | | | | | | |
| 2 | | | | | | | | |
| 3 | | DFA(small) | | DFA(large) | | En(small) | | En(large) |
| 4 | Post | Pre | Post | Pre | Post | Pre | Post | Pre |
| 5 | 7.863222 | 2.364264 | 2.270649 | 1.750121 | 1.582497 | 0.586759 | 0.598652 | 0.29553 |
| 6 | 8.476182 | 2.646646 | 2.51136 | 2.165405 | 1.98467 | 0.552282 | 0.564382 | 0.395939 |
| 7 | 13.1999 | 2.342645 | 2.263027 | 1.744133 | 1.633751 | 0.537564 | 0.554208 | 0.3191051 |
| 8 | 14.22238 | 2.200572 | 2.087856 | 1.5398 | 1.45678 | 0.577265 | 0.601867 | 0.293325 |
| 9 | 11.07672 | 2.260225 | 2.177297 | 1.460879 | 1.360382 | 0.573998 | 0.57739 | 0.245226 |
| 10 | 21.4056 | 2.34581 | 2.275392 | 1.705269 | 1.549012 | 0.63641 | 0.626134 | 0.282536 |
| 11 | 27.72514 | 2.293164 | 2.186881 | 1.780806 | 1.637206 | 0.561347 | 0.568079 | 0.333901 |
| 12 | 16.32354 | 2.237129 | 2.133148 | 1.655327 | 1.534519 | 0.587523 | 0.602658 | 0.329481 |
| 13 | 6.264069 | 2.345437 | 2.251713 | 1.633472 | 1.429133 | 0.593532 | 0.599485 | 0.258126 |
| 14 | 18.01225 | 2.374293 | 2.258233 | 1.801043 | 1.576142 | 0.586817 | 0.61729 | 0.334892 |
| 15 | 12.0894 | 2.302464 | 2.231811 | 1.738612 | 1.60884 | 0.565609 | 0.59352 | 0.328416 |
| 16 | 12.55933 | 2.236266 | 2.105685 | 1.73193 | 1.528937 | 0.568694 | 0.599907 | 0.338484 |
| 17 | 7.657876 | 2.310993 | 2.187276 | 1.629895 | 1.457624 | 0.576331 | 0.590144 | 0.274222 |
| | AG | AH | AI | AJ | AK | AL | AM | AN |
| 1 | | | | | | | | |
| 2 | | | | SD | | | | |
| 3 | No. of Pattern | | | DF | | AF | | B |
| 4 | Post | Pre | Post | Pre | Post | Pre | Post | Pre |
| 5 | 0.268657 | 4.25 | 5.25 | 1.246386 | 1.796788 | 0.886723 | 1.503159 | 8.814569 |
| 6 | 0.36731 | 6.6 | 6.2 | 2.246519 | 1.244685 | 1.951554 | 0.743646 | 10.30748 |
| 7 | 0.310649 | 6.166667 | 6.833333 | 2.858614 | 8.347153 | 4.388634 | 6.27373 | 12.34336 |
| 8 | 0.304139 | 5.857143 | 6.428571 | 1.974787 | 5.293136 | 3.403889 | 4.102591 | 14.23534 |
| 9 | 0.239737 | 6 | 6.4 | 4.105791 | 5.190629 | 3.686442 | 5.418081 | 11.58547 |
| 10 | 0.249617 | 6.6 | 6 | 3.2165 | 7.140039 | 3.010187 | 3.605719 | 11.7358 |
| 11 | 0.320297 | 6.285714 | 7 | 6.811515 | 8.837247 | 5.016491 | 4.941626 | 7.847156 |
| 12 | 0.331247 | 6.25 | 6.875 | 3.171016 | 5.98698 | 3.35513 | 4.301762 | 12.90536 |
| 13 | 0.221171 | 6.333333 | 6 | 1.385982 | 3.20614 | 1.616205 | 3.135401 | 8.890236 |
| 14 | 0.284977 | 5.6 | 6.2 | 2.739949 | 1.523009 | 2.440634 | 1.561005 | 12.6085 |
| 15 | 0.300904 | 5.428571 | 6.428571 | 3.94223 | 3.131414 | 5.246546 | 3.68319 | 14.94392 |
| 16 | 0.328938 | 6 | 6.8 | 2.179115 | 6.337592 | 4.338948 | 5.765392 | 15.56547 |
| 17 | 0.256102 | 6 | 6 | 1.491106 | 1.615037 | 2.01387 | 2.090824 | 9.082381 |
| | AO | AP | AQ | AR | AS | AT | AU | AV |
| 1 | | | | | | | | |
| 2 | | | | | | | | |
| 3 | | N | | T | | DP | | PPAmp |
| 4 | Post | Pre | Post | Pre | Post | Pre | Post | Pre |
| 5 | 12.85519 | 7.150139 | 13.1418 | 12.77476 | 15.37677 | 691.5369 | 416.1357 | 51.54097 |
| 6 | 7.717109 | 11.15991 | 4.610922 | 5.699737 | 0.678554 | 511.7694 | 241.7115 | 54.94503 |
| 7 | 26.59627 | 16.16153 | 17.05453 | 15.3143 | 26.23043 | 777.2335 | 242.8829 | 57.6751 |
| 8 | 34.45836 | 14.68734 | 6.415646 | 13.72937 | 30.11574 | 391.4272 | 124.9463 | 24.20744 |
| 9 | 28.99872 | 9.649683 | 27.53768 | 5.23054 | 4.856233 | 1068.995 | 363.143 | 60.50537 |
| 10 | 25.99388 | 5.565017 | 16.89323 | 8.781134 | 22.86379 | 276.8679 | 83.04047 | 20.50846 |
| 11 | 22.68142 | 10.7719 | 11.75371 | 11.79988 | 29.68901 | 745.0875 | 296.9765 | 76.94858 |
| 12 | 28.47327 | 9.805598 | 10.91488 | 7.31322 | 29.81622 | 1368.234 | 623.5598 | 115.9093 |
| 13 | 13.80326 | 10.4843 | 14.41483 | 3.802777 | 21.2692 | 909.5176 | 354.4524 | 67.56446 |
| 14 | 32.90603 | 14.68621 | 22.64546 | 5.03226 | 34.30716 | 2034.571 | 2421.565 | 85.63749 |
| 15 | 24.40735 | 9.569504 | 18.12787 | 12.64545 | 34.75676 | 1941.012 | 2229.239 | 109.5894 |
| 16 | 30.23675 | 6.875124 | 17.40244 | 18.01958 | 31.76622 | 3571.094 | 6174.521 | 126.1738 |
| 17 | 26.82664 | 8.068495 | 23.34169 | 10.55194 | 12.99555 | 2118.499 | 690.4111 | 80.90805 |
| | AW | AX | AY | AZ | BA | BB | BC | BD |
| 1 | | | | | | | | |
| 2 | | | | | | | | |
| 3 | | Slope | | Period | | Velocity | | DFA(small) |
| 4 | Post | Pre | Post | Pre | Post | Pre | Post | Pre |
| 5 | 56.24314 | 37.84681 | 36.62551 | 0.654939 | 0.972795 | 12.40686 | 6.747541 | 0.084161 |
| 6 | 37.65019 | 95.49879 | 57.73372 | 0.212831 | 0.47092 | 7.038413 | 4.318973 | 0.075793 |
| 7 | 22.1352 | 98.04194 | 33.41138 | 0.244024 | 0.804897 | 16.81004 | 9.939141 | 0.272006 |
| 8 | 13.69744 | 50.56903 | 47.74121 | 0.87138 | 1.624232 | 10.71144 | 8.21397 | 0.11994 |
| 9 | 27.30013 | 108.6049 | 61.73344 | 0.715875 | 1.288476 | 20.08436 | 14.42521 | 0.155852 |
| 10 | 19.97053 | 59.79322 | 41.95114 | 0.396504 | 1.049011 | 3.114373 | 11.93892 | 0.144674 |
| 11 | 66.23917 | 117.4847 | 91.92218 | 0.504512 | 0.73849 | 13.82808 | 40.21133 | 0.229615 |
| 12 | 56.67104 | 185.7691 | 95.6629 | 0.180441 | 0.679422 | 10.09134 | 9.727716 | 0.317259 |
| 13 | 32.30968 | 111.9528 | 58.08007 | 0.427331 | 0.854037 | 8.210103 | 7.204446 | 0.1163 |
| 14 | 128.1597 | 134.0056 | 188.4703 | 0.1888 | 0.493319 | 5.199237 | 12.85374 | 0.070679 |
| 15 | 114.6535 | 174.8819 | 189.6278 | 0.172286 | 0.128412 | 36.40332 | 11.25197 | 0.325164 |
| 16 | 162.6866 | 189.3884 | 282.9499 | 0.314344 | 0.622639 | 4.43656 | 5.663909 | 0.229168 |
| 17 | 48.82833 | 124.2854 | 76.45891 | 0.240079 | 0.427954 | 12.18542 | 5.831211 | 0.117284 |
| | BE | BF | BG | BH | BI | BJ | BK | BL |
| 1 | | | | | | | | |
| 2 | | | | | | | | |
| 3 | | DFA(large) | | En(small) | | En(large) | | No. of Pat [symbol] |
| 4 | Post | Pre | Post | Pre | Post | Pre | Post | Pre |
| 5 | 0.160255 | 0.132631 | 0.247108 | 0.025287 | 0.017633 | 0.039357 | 0.041925 | 0.957427 |
| 6 | 0.049711 | 0.074259 | 0.107629 | 0.037063 | 0.043925 | 0.017854 | 0.048253 | 1.140175 |
| 7 | 0.272229 | 0.321731 | 0.381649 | 0.060993 | 0.06205 | 0.098401 | 0.09102 | 0.983192 |
| 8 | 0.20018 | 0.255818 | 0.251865 | 0.046968 | 0.041219 | 0.096516 | 0.102815 | 0.690066 |
| 9 | 0.161056 | 0.294623 | 0.314464 | 0.029417 | 0.020408 | 0.074587 | 0.070876 | 0.707107 |
| 10 | 0.111511 | 0.16776 | 0.126211 | 0.018951 | 0.010931 | 0.027798 | 0.036235 | 0.547723 |
| 11 | 0.200006 | 0.240728 | 0.279765 | 0.061422 | 0.06991 | 0.070109 | 0.089598 | 0.755929 |
| 12 | 0.339189 | 0.313661 | 0.320371 | 0.038025 | 0.020758 | 0.074834 | 0.065925 | 0.707107 |
| 13 | 0.054178 | 0.234973 | 0.213106 | 0.012324 | 0.028207 | 0.049023 | 0.056947 | 0.816497 |
| 14 | 0.064116 | 0.087937 | 0.111249 | 0.032928 | 0.027139 | 0.058279 | 0.070769 | 1.516575 |
| 15 | 0.343834 | 0.258811 | 0.291734 | 0.055928 | 0.04535 | 0.06906 | 0.072974 | 0.9759 |
| 16 | 0.270414 | 0.158747 | 0.186046 | 0.059661 | 0.037014 | 0.064308 | 0.080061 | 0.707107 |
| 17 | 0.131644 | 0.191748 | 0.210655 | 0.022732 | 0.017991 | 0.046905 | 0.047282 | 0.755929 |
| | BM | | | | | | | |
| 1 | | | | | | | | |
| 2 | | | | | | | | |
| 3 | ern | | | | | | | |
| 4 | Post | | | | | | | |
| 5 | 0.957427 | | | | | | | |
| 6 | 0.447214 | | | | | | | |
| 7 | 0.983192 | | | | | | | |
| 8 | 0.534522 | | | | | | | |
| 9 | 0.547723 | | | | | | | |
| 10 | 1.224745 | | | | | | | |
| 11 | 0.816497 | | | | | | | |
| 12 | 0.991031 | | | | | | | |
| 13 | 0.632456 | | | | | | | |
| 14 | 1.30384 | | | | | | | |
| 15 | 1.133893 | | | | | | | |
| 16 | 1.095445 | | | | | | | |
| 17 | 1.511858 | | | | | | | |

[symbol]

indicates data missing or illegible when filed

TABLE 2.4
Mean and SD of percentage change

| | A | B | C | D | E | F | G | H | I |
|---|---|---|---|---|---|---|---|---|---|
| 1 | | | | | | | | | |
| 2 | | | | | | Mean percentage change (%) | | | |
| 3 | Drug | Tissue | Dose | Dose | Repeat No | DF | AF | B | N |
| 4 | dop | stomach | 100 uM | 4 | 7 | 8.128967 | 20.31495 | 6.99874 | −31.5059 |
| 5 | dop | duodenum | 100 nM | 7 | 5 | −7.88968 | −13.1377 | 51.47776 | −41.3023 |
| 6 | dop | duodenum | 1 uM | 6 | 7 | −9.672 | −7.64033 | 27.96052 | −26.0997 |
| 7 | dop | duodenum | 10 uM | 5 | 6 | −0.5559 | −2.19283 | 25.09703 | −44.5512 |
| 8 | dop | duodenum | 100 uM | 4 | 9 | −8.1457 | −12.2047 | 25.34717 | −18.8509 |
| 9 | dop | ileum | 100 nM | 7 | 6 | −11.7835 | −17.1055 | 28.27346 | −36.5652 |
| 10 | dop | ileum | 1 uM | 6 | 7 | −4.66192 | −3.60009 | 18.79839 | −25.0053 |
| 11 | dop | ileum | 10 uM | 5 | 7 | −8.69716 | −13.5311 | 30.47849 | −41.6975 |
| 12 | dop | ileum | 100 uM | 4 | 6 | −1.09503 | −0.31435 | 7.309394 | −20.21 |
| 13 | dop | colon | 100 nM | 7 | 6 | −2.04655 | −1.79584 | 9.025904 | −26.1132 |
| 14 | dop | colon | 1 uM | 6 | 8 | 4.099977 | 10.44317 | −9.14111 | −26.8809 |
| 15 | dop | colon | 10 uM | 5 | 6 | 2.595162 | 4.979046 | −1.16819 | −29.3223 |
| 16 | dop | colon | 100 uM | 4 | 9 | −2.42503 | −5.17544 | 19.20475 | −19.6712 |

| | J | K | L | M | N | O | P | Q | R |
|---|---|---|---|---|---|---|---|---|---|
| 1 | | | | | | | | | |
| 2 | | | | | | | | | |
| 3 | T | DP | PPAmp | Slope | Period | Velocity | DFA(small) | DFA(large) | En(small) |
| 4 | 24.12893 | −56.9927 | −18.024 | −8.21673 | 3.689298 | 22.02304 | −4.04683 | −9.75598 | 2.168323 |
| 5 | −10.449 | −35.0124 | −41.2486 | −48.3407 | 30.3723 | −17.6174 | −5.07022 | −8.3022 | 2.163832 |
| 6 | −1.59644 | −34.2125 | −25.3293 | −31.5121 | 21.02539 | 97.69308 | −3.34639 | −6.9773 | 3.170384 |
| 7 | 19.53353 | −47.4107 | −14.3002 | −19.1294 | 31.95505 | −20.4475 | −5.17875 | −5.30424 | 4.442673 |
| 8 | −5.67668 | −21.1387 | −36.4653 | −41.4951 | 31.81639 | 81.34962 | −3.58857 | −7.06691 | 0.72541 |
| 9 | 9.315152 | −77.9782 | −28.0296 | −37.5146 | 39.0588 | 111.1163 | −2.91079 | −8.86503 | −1.56877 |
| 10 | 5.790041 | −51.3141 | −26.3207 | −31.675 | 22.51592 | 65.52867 | −4.44873 | −8.15354 | 1.251276 |
| 11 | 11.21807 | −21.5663 | −30.0807 | −33.6255 | 32.70938 | −2.0405 | −4.70547 | −6.99847 | 2.898694 |
| 12 | 12.59188 | 12.06158 | −20.8458 | −25.7936 | 9.776357 | 33.53616 | −3.85484 | −12.1803 | 1.015738 |
| 13 | 17.60517 | 13.91164 | −5.825 | 2.881199 | 13.59551 | 41.7371 | −4.86586 | −12.51 | 5.276945 |
| 14 | 37.13537 | 13.74634 | −8.36309 | −8.21794 | −2.41291 | 22.55092 | −3.21563 | −7.90371 | 5.180204 |
| 15 | 28.82225 | −25.6601 | −23.7383 | −22.9821 | 8.782035 | −10.5241 | −5.9039 | −11.6012 | 5.950608 |
| 16 | 0.286971 | −31.7486 | −23.4385 | −31.1053 | 10.63735 | 40.86316 | −5.31371 | −10.0463 | 2.5032981 |
| | S | T | U | V | W | X | Y | Z | AA |
| 1 | | | | | | | | | |
| 2 | | | SD percentage change | | | | | | |
| 3 | En(large) | ActP | DF | AF | B | N | T | DP | PPAmp |
| 4 | −7.86484 | 19.45 | 26.26159 | 19.39606 | 7.824624 | 16.26674 | 17.42516 | 57.09071 | 40.935 |
| 5 | −7.31661 | 11.96 | 4.021961 | 6.933814 | 4.192089 | 10.41251 | 5.626532 | 47.48587 | 7.861639 |
| 6 | −1.51463 | 11.35 | 25.13922 | 10.27668 | 25.69461 | 29.74949 | 22.34331 | 46.56287 | 23.13059 |
| 7 | 3.751525 | 14.61429 | 14.26834 | 18.65488 | 39.47316 | 18.43169 | 20.86462 | 48.85024 | 20.87482 |
| 8 | −1.76807 | 11.68 | 10.55228 | 12.29901 | 30.50501 | 27.59579 | 7.350898 | 141.1962 | 18.98083 |
| 9 | −11.4715 | 5.88 | 28.99624 | 14.14942 | 18.20473 | 15.61097 | 14.74401 | 4.996288 | 6.929485 |
| 10 | −5.33538 | 13.32857 | 17.15903 | 5.683626 | 17.21165 | 13.47704 | 27.44219 | 17.69718 | 12.32416 |
| 11 | 2.126289 | 8.375 | 24.66621 | 13.80947 | 25.2055 | 7.595621 | 30.49392 | 97.72505 | 16.83827 |
| 12 | −14.1159 | 8.8 | 11.6118 | 12.56933 | 21.25927 | 20.77215 | 22.81168 | 186.8873 | 22.76359 |
| 13 | −15.8959 | 19.6 | 7.508601 | 11.82407 | 35.49789 | 33.21273 | 36.9336 | 137.3658 | 45.88399 |
| 14 | −8.31001 | 16.45714 | 6.01572 | 16.79101 | 26.71444 | 21.52806 | 31.44938 | 85.28964 | 16.7506 |
| 15 | −3.07409 | 8.96 | 21.02257 | 17.11654 | 25.13421 | 18.77098 | 29.3703 | 68.82866 | 25.32645 |
| 16 | −5.62434 | 11.2875 | 5.033787 | 5.683459 | 23.9697 | 22.2896 | 17.93054 | 71.93298 | 13.08632 |

| | AB | AC | AD | AE | AF | AG | AH | AI |
|---|---|---|---|---|---|---|---|---|
| 1 | | | | | | | | |
| 2 | | | | | | | | |
| 3 | Slope | Period | Velocity | DFA(small | DFA(large) | En(small) | En(large) | ActP |
| 4 | 35.95585 | 16.30484 | 105.3829 | 4.0079 | 11.19137 | 4.864503 | 17.6571 | 4.810059 |
| 5 | 8.994522 | 18.78484 | 50.10624 | 2.379576 | 4.980522 | 3.136293 | 10.61327 | 12.60488 |
| 6 | 25.55933 | 27.22803 | 216.441 | 4.594116 | 7.34454 | 4.231854 | 9.787561 | 5.11576 |
| 7 | 34.97704 | 73.68006 | 23.71082 | 6.386011 | 6.889621 | 4.775442 | 12.28703 | 7.464455 |
| 8 | 19.7191 | 28.84447 | 109.5171 | 4.704409 | 8.096829 | 4.008516 | 6.877634 | 6.108764 |
| 9 | 11.72818 | 27.90511 | 130.7452 | 2.756726 | 6.199121 | 2.244841 | 11.4334 | 4.578428 |
| 10 | 13.14282 | 18.41824 | 117.7509 | 4.961903 | 8.84829 | 7.115043 | 9.88398 | 7.994939 |
| 11 | 20.31301 | 29.38597 | 51.82981 | 5.548586 | 11.69619 | 6.613618 | 16.0061 | 4.947943 |
| 12 | 19.06595 | 28.70816 | 85.44971 | 3.731649 | 8.158478 | 4.660051 | 15.98521 | 7.038466 |
| 13 | 62.64031 | 20.8974 | 92.59481 | 2.113782 | 3.811308 | 2.807358 | 7.811232 | 21.06264 |
| 14 | 17.59326 | 6.844384 | 77.81314 | 3.160843 | 6.113264 | 3.917319 | 9.55699 | 7.226769 |
| 15 | 31.85314 | 20.64228 | 15.11973 | 5.643804 | 8.735398 | 5.509515 | 14.4159 | 5.206054 |
| 16 | 9.632743 | 18.10985 | 119.4503 | 4.068197 | 12.15713 | 4.206257 | 16.29155 | 7.871547 |

TABLE 2.5
p-values using paired t-test using mean of each slow wav

[symbol]

| | A | B | C | D | E | F | G | H |
|---|---|---|---|---|---|---|---|---|
| 1 | | | | | | | | |
| 2 | Drug | Tissue | Dose | Dose | n | DF | AF | B |
| 3 | dop | stomach | 100 uM | 4 | 7 | 0.546939 | 0.02628 | 0.055787 |
| 4 | dop | duodenum | 100 nM | 7 | 5 | 0.021174 | 0.021182 | 1.05E−05 |
| 5 | dop | duodenum | 1 uM | 6 | 7 | 0.351322 | 0.105884 | 0.028092 |
| 6 | dop | duodenum | 10 uM | 5 | 6 | 0.980343 | 0.667897 | 0.180112 |
| 7 | dop | duodenum | 100 uM | 4 | 9 | 0.049079 | 0.028582 | 0.037361 |
| 8 | dop | ileum | 100 nM | 7 | 6 | 0.331316 | 0.034552 | 0.012574 |
| 9 | dop | ileum | 1 uM | 6 | 7 | 0.761093 | 0.155367 | 0.027706 |
| 10 | dop | ileum | 10 uM | 5 | 7 | 0.364818 | 0.051336 | 0.018618 |
| 11 | dop | ileum | 100 uM | 4 | 6 | 0.809235 | 0.93059 | 0.438099 |
| 12 | dop | colon | 100 nM | 7 | 6 | 0.467934 | 0.61206 | 0.560704 |
| 13 | dop | colon | 1 uM | 6 | 8 | 0.147196 | 0.092618 | 0.365372 |
| 14 | dop | colon | 10 uM | 5 | 6 | 0.79703 | 0.490439 | 0.913789 |
| 15 | dop | colon | 100 uM | 4 | 9 | 0.180925 | 0.020294 | 0.042933 |
| 16 | | | | | | | | |
| 17 | Drug | Tissue | Dose | Dose | n | DF | AF | B |
| 18 | dop | stomach | 100 uM | 4 | 7 | ns | * | ns |
| 19 | dop | duodenum | 100 nM | 7 | 5 | * | * | **** |
| 20 | dop | duodenum | 1 uM | 6 | 7 | ns | ns | * |
| 21 | dop | duodenum | 10 uM | 5 | 6 | ns | ns | ns |
| 22 | dop | duodenum | 100 uM | 4 | 9 | * | * | * |
| 23 | dop | ileum | 100 nM | 7 | 6 | ns | * | * |
| 24 | dop | ileum | 1 uM | 6 | 7 | ns | ns | * |
| 25 | dop | ileum | 10 uM | 5 | 7 | ns | ns | * |
| 26 | dop | ileum | 100 uM | 4 | 6 | ns | ns | ns |
| 27 | dop | colon | 100 nM | 7 | 6 | ns | ns | ns |
| 28 | dop | colon | 1 uM | 6 | 8 | ns | ns | ns |
| 29 | dop | colon | 10 uM | 5 | 6 | ns | ns | ns |
| 30 | dop | colon | 100 uM | 4 | 9 | ns | * | * |
| | I | J | K | L | M | N | O | P |
| 1 | [symbol] e features between baseline and post-drug data | | | | | | | |
| 2 | N | T | DP | Amp | S | P | V | DFA(small) |
| 3 | 0.002169 | 0.010534 | 0.033191 | 0.181696 | 0.354502 | 0.672369 | 0.355113 | 0.03331 |
| 4 | 0.000892 | 0.01423 | 0.101034 | 0.00163 | 0.003187 | 0.024016 | 0.34939 | 0.003989 |
| 5 | 0.059354 | 0.856294 | 0.071478 | 0.01666 | 0.013854 | 0.089787 | 0.911403 | 0.112629 |
| 6 | 0.001959 | 0.070366 | 0.078602 | 0.099783 | 0.135798 | 0.512522 | 0.1271 | 0.0782 |
| 7 | 0.074586 | 0.04917 | 0.112981 | 0.00224 | 0.002237 | 0.010584 | 0.79997 | 0.04573 |
| 8 | 0.002253 | 0.182402 | 0.000743 | 0.000179 | 0.001939 | 0.022362 | 0.058228 | 0.040529 |
| 9 | 0.002687 | 0.596887 | 0.021401 | 0.009316 | 0.007898 | 0.027559 | 0.459724 | 0.027601 |
| 10 | 6.68E−06 | 0.367986 | 0.117095 | 0.020662 | 0.016144 | 0.02511 | 0.657371 | 0.034431 |
| 11 | 0.062915 | 0.234282 | 0.110588 | 0.106071 | 0.071034 | 0.523728 | 0.556643 | 0.032799 |
| 12 | 0.112079 | 0.295613 | 0.541276 | 0.60967 | 0.755909 | 0.184179 | 0.419499 | 0.00264 |
| 13 | 0.009576 | 0.012423 | 0.260585 | 0.280938 | 0.289005 | 0.306746 | 0.397612 | 0.020595 |
| 14 | 0.012293 | 0.061333 | 0.611936 | 0.171809 | 0.274537 | 0.39723 | 0.193907 | 0.046492 |
| 15 | 0.029366 | 0.962882 | 0.067402 | 0.006155 | 0.00043 | 0.116932 | 0.419768 | 0.002688 |
| 16 | | | | | | | | |
| 17 | N | T | DP | Amp | S | P | V | DFA(small) |
| 18 | ** | * | * | ns | ns | ns | ns | * |
| 19 | *** | * | ns | ** | ** | * | ns | ** |
| 20 | ns | ns | ns | * | * | ns | ns | ns |
| 21 | ** | ns | ns | ns | ns | ns | ns | ns |
| 22 | ns | * | ns | ** | ** | * | ns | * |
| 23 | ** | ns | *** | *** | ** | * | ns | * |
| 24 | ** | ns | * | ** | ** | * | ns | * |
| 25 | **** | ns | ns | * | * | * | ns | * |
| 26 | ns | ns | ns | ns | ns | ns | ns | * |
| 27 | ns | ns | ns | ns | ns | ns | ns | ** |
| 28 | ** | * | ns | ns | ns | ns | ns | * |
| 29 | * | ns | ns | ns | ns | ns | ns | * |
| 30 | * | ns | ns | ** | *** | ns | ns | ** |

| | Q | R | S | T | U | V | W |
|---|---|---|---|---|---|---|---|
| 1 | | | | | | | |
| 2 | DFA(large) | En(small) | En(large) | NoP | | | |
| 3 | 0.05486 | 0.306732 | 0.239327 | 0 | | | |
| 4 | 0.009738 | 0.145475 | 0.155234 | 0.476621 | | | |
| 5 | 0.041452 | 0.11854 | 0.521299 | 0.025031 | | | |
| 6 | 0.089212 | 0.046987 | 0.377447 | 0.03002 | | | |
| 7 | 0.029339 | 0.671587 | 0.356144 | 0.177808 | | | |
| 8 | 0.013212 | 0.145173 | 0.050319 | 0.426317 | | | |
| 9 | 0.026237 | 0.644379 | 0.218658 | 0.046528 | | | |
| 10 | 0.083975 | 0.285725 | 0.906923 | 0.049174 | | | |
| 11 | 0.012798 | 0.588098 | 0.082111 | 0.363217 | | | |
| 12 | 0.000467 | 0.004705 | 0.001048 | 0.070484 | | | |
| 13 | 0.004377 | 0.007923 | 0.050199 | 0.00376 | | | |
| 14 | 0.01957 | 0.027587 | 0.631766 | 0.01613 | | | |
| 15 | 0.024597 | 0.093112 | 0.229564 | 1 | | | |
| 16 | | | | | | | |
| 17 | DFA(large) | En(small) | En(large) | NoP | | | |
| 18 | ns | ns | ns | **** | | | |
| 19 | ** | ns | ns | ns | | | |
| 20 | * | ns | ns | * | | | |
| 21 | * | * | ns | * | | | |
| 22 | * | ns | ns | ns | | | |
| 23 | * | ns | ns | ns | | | |
| 24 | * | ns | ns | * | | | |
| 25 | ns | ns | ns | * | | | |
| 26 | * | ns | ns | ns | | p > 0.05 | ns |
| 27 | *** | ** | ** | ns | | p < 0.05 | * |
| 28 | ** | ** | ns | ** | | p < 0.01 | ** |
| 29 | * | * | ns | * | | p < 0.001 | *** |
| 30 | * | ns | ns | ns | | p < 0.0001 | **** |

[symbol]

indicates data missing or illegible when filed

TABLE 2.6
Percentage of activation pattern distrib

[symbol]

| | A | B | C | D | E | F | G | H |
|---|---|---|---|---|---|---|---|---|
| 1 | | | | | | | | |
| 2 | | | | | 1st | | | 2nd |
| 3 | Drug | Tissue | Dose | Repeat No | mean | sd | n | mean |
| 4 | dop | stomach | Baseline | 4 | 38.35 | 14.10969 | 4 | 22.6 |
| 5 | dop | stomach | 100 uM | 4 | 30.6 | 17.80468 | 4 | 16.7 |
| 6 | dop | duodenum | Baseline | 7 | 32.26 | 11.80458 | 5 | 23.3 |
| 7 | dop | duodenum | 100 nM | 7 | 11.1 | 13.03438 | 5 | 8.4 |
| 8 | dop | duodenum | Baseline | 6 | 33.05 | 13.41682 | 6 | 20.41667 |
| 9 | dop | duodenum | 1 uM | 6 | 12.76667 | 9.446834 | 6 | 18.78333 |
| 10 | dop | duodenum | Baseline | 5 | 33.72857 | 13.07781 | 7 | 20.31429 |
| 11 | dop | duodenum | 10 uM | 5 | 19.62857 | 9.165827 | 7 | 17.94286 |
| 12 | dop | duodenum | Baseline | 4 | 29.32 | 5.496544 | 5 | 20.74 |
| 13 | dop | duodenum | 100 uM | 4 | 6.96 | 7.049326 | 5 | 7.44 |
| 14 | dop | ileum | Baseline | 7 | 32.82 | 5.679084 | 5 | 21.46 |
| 15 | dop | ileum | 100 nM | 7 | 3.8 | 4.500556 | 5 | 2.56 |
| 16 | dop | ileum | Baseline | 6 | 35.2 | 6.015258 | 7 | 25.54286 |
| 17 | dop | ileum | 1 uM | 6 | 13.01429 | 6.707317 | 7 | 16.02857 |
| 18 | dop | ileum | Baseline | 5 | 35.7 | 8.411047 | 8 | 19.95 |
| 19 | dop | ileum | 10 uM | 5 | 19.1625 | 16.36127 | 8 | 14.0375 |
| 20 | dop | ileum | Baseline | 4 | 27.61667 | 4.064193 | 6 | 23.46667 |
| 21 | dop | ileum | 100 uM | 4 | 9.4 | 9.525965 | 6 | 5.683333 |
| 22 | dop | colon | Baseline | 7 | 31.28 | 9.104779 | 5 | 25.52 |
| 23 | dop | colon | 100 nM | 7 | 13.44 | 7.345951 | 5 | 22.44 |
| 24 | dop | colon | Baseline | 6 | 25.81429 | 7.864356 | 7 | 17.7 |
| 25 | dop | colon | 1 uM | 6 | 29.78571 | 21.62741 | 7 | 20.18571 |
| 26 | dop | colon | Baseline | 5 | 36.24 | 18.27192 | 5 | 15.98 |
| 27 | dop | colon | 10 uM | 5 | 26.5 | 24.44831 | 5 | 17.7 |
| 28 | dop | colon | Baseline | 4 | 34.4 | 3.668398 | 8 | 25.3375 |
| 29 | dop | colon | 100 uM | 4 | 11.3875 | 12.94366 | 8 | 14.3375 |
| 30 | | | | | | | | |
| 31 | | | | | | | | |
| 32 | | | | | | | | |
| | I | J | K | L | M | N | O | P |
| 1 | [symbol] ution based on baseline data | | | | | | | |
| 2 | | | 3rd | | | Other | | |
| 3 | sd | n | mean | sd | n | mean | sd | n |
| 4 | 7.779889 | 4 | 10.225 | 5.62161 | 4 | 28.825 | 6.601704 | 4 |
| 5 | 5.665686 | 4 | 27.225 | 9.265483 | 4 | 25.475 | 17.66586 | 4 |
| 6 | 5.724945 | 5 | 13.7 | 1.936492 | 5 | 30.74 | 15.2487 | 5 |
| 7 | 11.17989 | 5 | 10.2 | 9.957911 | 5 | 70.3 | 24.7876 | 5 |
| 8 | 3.825398 | 6 | 15.28333 | 7.776224 | 6 | 31.25 | 14.01824 | 6 |
| 9 | 13.15955 | 6 | 17.15 | 8.551667 | 6 | 51.3 | 15.2364 | 6 |
| 10 | 5.329925 | 7 | 11.35714 | 3.968567 | 7 | 34.6 | 14.28822 | 7 |
| 11 | 13.49974 | 7 | 16.01429 | 6.998435 | 7 | 46.41429 | 12.10722 | 7 |
| 12 | 5.430285 | 5 | 15.16 | 2.111398 | 5 | 34.78 | 11.78121 | 5 |
| 13 | 4.099756 | 5 | 5.88 | 3.585666 | 5 | 79.72 | 12.96831 | 5 |
| 14 | 1.740115 | 5 | 17.22 | 4.75731 | 5 | 28.5 | 2.774887 | 5 |
| 15 | 1.596246 | 5 | 2.2 | 2.614383 | 5 | 91.44 | 8.324842 | 5 |
| 16 | 5.060915 | 7 | 14.47143 | 5.609431 | 7 | 24.78571 | 6.981267 | 7 |
| 17 | 13.86311 | 7 | 13.27143 | 9.170917 | 7 | 57.68571 | 18.82449 | 7 |
| 18 | 7.931132 | 8 | 13.7625 | 5.58747 | 8 | 30.5875 | 9.468359 | 8 |
| 19 | 11.1291 | 8 | 16.5375 | 14.39255 | 8 | 50.2625 | 23.31039 | 8 |
| 20 | 2.770319 | 6 | 19.05 | 2.771823 | 6 | 29.86667 | 7.115242 | 6 |
| 21 | 5.087403 | 6 | 5.866667 | 6.586856 | 6 | 79.05 | 20.62947 | 6 |
| 22 | 9.723014 | 5 | 15.36 | 6.510991 | 5 | 27.84 | 16.64476 | 5 |
| 23 | 27.348 | 5 | 9.18 | 6.226315 | 5 | 54.94 | 30.68832 | 5 |
| 24 | 2.863564 | 7 | 13.48571 | 3.668073 | 7 | 43 | 12.35867 | 7 |
| 25 | 11.66554 | 7 | 17.32857 | 8.311581 | 7 | 32.7 | 21.53617 | 7 |
| 26 | 6.276305 | 5 | 9.92 | 5.31432 | 5 | 37.86 | 11.25291 | 5 |
| 27 | 12.1918 | 5 | 13.52 | 7.968814 | 5 | 42.28 | 22.53746 | 5 |
| 28 | 4.987681 | 8 | 18.275 | 4.076325 | 8 | 21.9875 | 9.821614 | 8 |
| 29 | 16.07873 | 8 | 14.35 | 14.10906 | 8 | 59.925 | 33.53164 | 8 |
| 30 | | | | | | | | |
| 31 | | | | | | | | |
| 32 | | | | | | | | |
| | Q | R | S | T | U | V | W | X |
| 1 | | | | | | | | |
| 2 | | | | | | | P-values | |
| 3 | | Drug | Tissue | Dose | Dose | Repeat No | 1st | 2nd |
| 4 | | dop | stomach | 100 uM | 4 | 4 | 0.249079 | 0.172625 |
| 5 | | dop | duodenum | 100 nM | 7 | 5 | 0.002134 | 0.023244 |
| 6 | | dop | duodenum | 1 uM | 6 | 6 | 0.054219 | 0.810914 |
| 7 | | dop | duodenum | 10 uM | 5 | 7 | 0.046071 | 0.684683 |
| 8 | | dop | duodenum | 100 uM | 4 | 5 | 0.008876 | 0.030922 |
| 9 | | dop | ileum | 100 nM | 7 | 5 | 0.001043 | 3.31E−05 |
| 10 | | dop | ileum | 1 uM | 6 | 7 | 0.00016 | 0.103121 |
| 11 | | dop | ileum | 10 uM | 5 | 8 | 0.029534 | 0.300338 |
| 12 | | dop | ileum | 100 uM | 4 | 6 | 0.003408 | 0.000549 |
| 13 | | dop | colon | 100 nM | 7 | 5 | 0.019009 | 0.739025 |
| 14 | | dop | colon | 1 uM | 6 | 7 | 0.518363 | 0.608652 |
| 15 | | dop | colon | 10 uM | 5 | 5 | 0.082449 | 0.798552 |
| 16 | | dop | colon | 100 uM | 4 | 8 | 0.001843 | 0.081556 |
| 17 | | | | | | | | |
| 18 | | | | | | | P-values | |
| 19 | | Drug | Tissue | Dose | Dose | Repeat No | 1st | 2nd |
| 20 | | dop | stomach | 100 uM | 4 | 4 | ns | ns |
| 21 | | dop | duodenum | 100 nM | 7 | 5 | ** | * |
| 22 | | dop | duodenum | 1 uM | 6 | 6 | ns | ns |
| 23 | | dop | duodenum | 10 uM | 5 | 7 | * | ns |
| 24 | | dop | duodenum | 100 uM | 4 | 5 | ** | * |
| 25 | | dop | ileum | 100 nM | 7 | 5 | ** | **** |
| 26 | | dop | ileum | 1 uM | 6 | 7 | *** | ns |
| 27 | | dop | ileum | 10 uM | 5 | 8 | * | ns |
| 28 | | dop | ileum | 100 uM | 4 | 6 | ** | *** |
| 29 | | dop | colon | 100 nM | 7 | 5 | * | ns |
| 30 | | dop | colon | 1 uM | 6 | 7 | ns | ns |
| 31 | | dop | colon | 10 uM | 5 | 5 | ns | ns |
| 32 | | dop | colon | 100 uM | 4 | 8 | ** | ns |

| | Y | Z | AA | AB | AC |
|---|---|---|---|---|---|
| 1 | | | | | |
| 2 | | | | | |
| 3 | 3rd | Other | | | |
| 4 | 0.062244 | 0.7695 | | | |
| 5 | 0.512983 | 0.015095 | | | |
| 6 | 0.505499 | 0.108136 | | | |
| 7 | 0.175317 | 0.25529 | | | |
| 8 | 0.016345 | 0.012734 | | | |
| 9 | 0.000756 | 1.63E−05 | | | |
| 10 | 0.753257 | 0.002915 | | | |
| 11 | 0.635381 | 0.077567 | | | |
| 12 | 0.001127 | 0.001019 | | | |
| 13 | 0.081291 | 0.081031 | | | |
| 14 | 0.23183 | 0.144967 | | | |
| 15 | 0.385834 | 0.669597 | | | |
| 16 | 0.360213 | 0.010722 | | | |
| 17 | | | | | |
| 18 | | | | | |
| 19 | 3rd | Other | | | |
| 20 | ns | ns | | | |
| 21 | ns | * | | | |
| 22 | ns | ns | | | |
| 23 | ns | ns | | | |
| 24 | * | * | | | |
| 25 | *** | **** | | | |
| 26 | ns | ** | | | |
| 27 | ns | ns | | | |
| 28 | ** | ** | | p > 0.05 | ns |
| 29 | ns | ns | | p < 0.05 | * |
| 30 | ns | ns | | p < 0.01 | ** |
| 31 | ns | ns | | p < 0.001 | *** |
| 32 | ns | * | | p < 0.0001 | **** |

[symbol]

indicates data missing or illegible when filed

TABLE 2.7
Percentage of activation pattern distributio

[symbol]

| | A | B | C | D | E | F | G | H | I |
|---|---|---|---|---|---|---|---|---|---|
| 1 | | | | | | | | | |
| 2 | | | | | 1st | | | 2nd | |
| 3 | | | | | mean | sd | n | mean | sd |
| 4 | dop | stomach | Baseline | 4 | 30.275 | 21.08671 | 4 | 13.65 | 11.13508 |
| 5 | dop | stomach | 100 uM | 4 | 36.325 | 11.92766 | 4 | 24.8 | 8.968463 |
| 6 | dop | duodenum | Baseline | 7 | 24.5 | 15.66652 | 5 | 12.3 | 7.475627 |
| 7 | dop | duodenum | 100 nM | 7 | 17.12 | 13.74962 | 5 | 11.22 | 8.790734 |
| 8 | dop | duodenum | Baseline | 6 | 15.45 | 10.29811 | 6 | 20.83333 | 18.99028 |
| 9 | dop | duodenum | 1 uM | 6 | 36.95 | 18.30418 | 6 | 17.25 | 5.807151 |
| 10 | dop | duodenum | Baseline | 5 | 22.54286 | 15.00587 | 7 | 13.67143 | 8.086968 |
| 11 | dop | duodenum | 10 uM | 5 | 32.22857 | 10.9608 | 7 | 23.92857 | 5.287947 |
| 12 | dop | duodenum | Baseline | 4 | 17.82 | 12.05268 | 5 | 15.96 | 5.77434 |
| 13 | dop | duodenum | 100 uM | 4 | 15.74 | 12.87296 | 5 | 10.94 | 7.91189 |
| 14 | dop | ileum | Baseline | 7 | 15.3 | 12.48779 | 5 | 15.56 | 10.33625 |
| 15 | dop | ileum | 100 nM | 7 | 6.76 | 4.657038 | 5 | 4.22 | 4.185929 |
| 16 | dop | ileum | Baseline | 6 | 8.871429 | 8.981966 | 7 | 20.04286 | 10.12881 |
| 17 | dop | ileum | 1 uM | 6 | 31.44286 | 13.73643 | 7 | 19.47143 | 7.311569 |
| 18 | dop | ileum | Baseline | 5 | 17.2 | 11.49621 | 8 | 15.6375 | 13.98131 |
| 19 | dop | ileum | 10 uM | 5 | 32.325 | 13.16551 | 8 | 20.7375 | 6.40601 |
| 20 | dop | ileum | Baseline | 4 | 23.3 | 10.50505 | 6 | 17.43333 | 7.639284 |
| 21 | dop | ileum | 100 uM | 4 | 11.31667 | 8.608929 | 6 | 7.85 | 7.007068 |
| 22 | dop | colon | Baseline | 7 | 20.06 | 13.73619 | 5 | 23.4 | 14.61865 |
| 23 | dop | colon | 100 nM | 7 | 26.96 | 24.73041 | 5 | 14.74 | 7.633348 |
| 24 | dop | colon | Baseline | 6 | 19.07143 | 12.19354 | 7 | 17.55714 | 3.070753 |
| 25 | dop | colon | 1 uM | 6 | 39.57143 | 15.61695 | 7 | 22.08571 | 5.456931 |
| 26 | dop | colon | Baseline | 5 | 30.2 | 22.36716 | 5 | 10.18 | 9.054943 |
| 27 | dop | colon | 10 uM | 5 | 33.4 | 21.85921 | 5 | 18.54 | 2.341581 |
| 28 | dop | colon | Baseline | 4 | 20.35 | 9.562576 | 8 | 21.6625 | 14.31053 |
| 29 | dop | colon | 100 uM | 4 | 21.4625 | 15.75427 | 8 | 18.0125 | 14.06154 |
| 30 | | | | | | | | | |
| 31 | | | | | | | | | |
| 32 | | | | | | | | | |
| | J | K | L | M | N | O | P | Q | R |
| 1 | [symbol] n based on post-drug data | | | | | | | | |
| 2 | | 3rd | | | Other | | | | |
| 3 | n | mean | sd | n | mean | sd | n | | Drug |
| 4 | 4 | 24.675 | 9.564997 | 4 | 31.4 | 9.25887 | 4 | | dop |
| 5 | 4 | 16.85 | 4.597463 | 4 | 22.025 | 13.45297 | 4 | | dop |
| 6 | 5 | 10.04 | 11.63972 | 5 | 53.16 | 11.36257 | 5 | | dop |
| 7 | 5 | 7.98 | 6.422383 | 5 | 63.68 | 26.83816 | 5 | | dop |
| 8 | 6 | 13 | 9.027292 | 6 | 50.71667 | 9.774951 | 6 | | dop |
| 9 | 6 | 12.83333 | 4.782747 | 6 | 32.96667 | 12.20486 | 6 | | dop |
| 10 | 7 | 15.48571 | 20.64836 | 7 | 48.3 | 12.93342 | 7 | | dop |
| 11 | 7 | 14.41429 | 3.257519 | 7 | 29.42857 | 13.27877 | 7 | | dop |
| 12 | 5 | 20.04 | 6.312923 | 5 | 46.18 | 16.46123 | 5 | | dop |
| 13 | 5 | 7.22 | 4.178157 | 5 | 66.1 | 24.70739 | 5 | | dop |
| 14 | 5 | 16.84 | 6.636867 | 5 | 52.3 | 16.30583 | 5 | | dop |
| 15 | 5 | 2.98 | 2.559687 | 5 | 86.04 | 10.93929 | 5 | | dop |
| 16 | 7 | 24.98571 | 15.07552 | 7 | 46.1 | 12.88966 | 7 | | dop |
| 17 | 7 | 13.17143 | 3.554675 | 7 | 35.91429 | 20.22279 | 7 | | |
| 18 | 8 | 12.2 | 13.30725 | 8 | 54.9625 | 17.48542 | 8 | | Drug |
| 19 | 8 | 12.7625 | 5.002553 | 8 | 34.175 | 17.87047 | 8 | | |
| 20 | 6 | 17.75 | 7.385594 | 6 | 41.51667 | 7.664311 | 6 | | dop |
| 21 | 6 | 6.766667 | 6.116753 | 6 | 74.06667 | 21.01111 | 6 | | dop |
| 22 | 5 | 14.2 | 12.506 | 5 | 42.34 | 20.29773 | 5 | | dop |
| 23 | 5 | 11.92 | 7.566836 | 5 | 46.38 | 32.74572 | 5 | | dop |
| 24 | 7 | 13.45714 | 8.69269 | 7 | 49.91429 | 14.16032 | 7 | | dop |
| 25 | 7 | 14.4 | 3.171225 | 7 | 23.94286 | 12.78943 | | | dop |
| 26 | 5 | 7.9 | 6.0469 | 5 | 51.72 | 24.63518 | 5 | | dop |
| 27 | 5 | 13.86 | 6.098606 | 5 | 34.2 | 20.25734 | 5 | | dop |
| 28 | 8 | 20.3 | 10.78663 | 8 | 37.6875 | 19.61315 | 8 | | dop |
| 29 | 8 | 7.475 | 5.830401 | 8 | 53.05 | 32.58606 | 8 | | dop |
| 30 | | | | | | | | | dop |
| 31 | | | | | | | | | dop |
| 32 | | | | | | | | | dop |
| | S | T | U | V | W | X | Y | Z | AA |
| 1 | | | | | | | | | |
| 2 | | | | | P-values | | | | |
| 3 | Tissue | Dose | Dose | Repeat No | 1st | 2nd | 3rd | Other | |
| 4 | stomach | 100 uM | 4 | 4 | 0.343533 | 0.265717 | 0.220861 | 0.362481 | |
| 5 | duodenum | 100 nM | 7 | 5 | 0.111337 | 0.86037 | 0.806166 | 0.529939 | |
| 6 | duodenum | 1 uM | 6 | 6 | 0.102748 | 0.717753 | 0.956698 | 0.004598 | |
| 7 | duodenum | 10 uM | 5 | 7 | 0.322739 | 0.037619 | 0.894758 | 0.036909 | |
| 8 | duodenum | 100 uM | 4 | 5 | 0.844608 | 0.201568 | 0.043767 | 0.287598 | |
| 9 | ileum | 100 nM | 7 | 5 | 0.280785 | 0.034291 | 0.005958 | 0.008145 | |
| 10 | ileum | 1 uM | 6 | 7 | 0.003247 | 0.899983 | 0.058657 | 0.084268 | |
| 11 | ileum | 10 uM | 5 | 8 | 0.015832 | 0.343704 | 0.878453 | 0.030297 | |
| 12 | ileum | 100 uM | 4 | 6 | 0.065058 | 0.148239 | 0.025431 | 0.013724 | |
| 13 | colon | 100 nM | 7 | 5 | 0.320799 | 0.256631 | 0.765864 | 0.773748 | |
| 14 | colon | 1 uM | 6 | 7 | 8.6E−05 | 0.116466 | 0.766714 | 7.82E−05 | |
| 15 | colon | 10 uM | 5 | 5 | 0.665868 | 0.157214 | 0.231409 | 0.279054 | |
| 16 | colon | 100 uM | 4 | 8 | 0.818439 | 0.625241 | 0.030278 | 0.191407 | |
| 17 | | | | | | | | | |
| 18 | | | | | P-values | | | | |
| 19 | Tissue | Dose | Dose | Repeat No | 1st | 2nd | 3rd | Other | |
| 20 | stomach | 100 uM | 4 | 4 | ns | ns | ns | ns | |
| 21 | duodenum | 100 nM | 7 | 5 | ns | ns | ns | ns | |
| 22 | duodenum | 1 uM | 6 | 6 | ns | ns | ns | ** | |
| 23 | duodenum | 10 uM | 5 | 7 | ns | * | ns | * | |
| 24 | duodenum | 100 uM | 4 | 5 | ns | ns | * | ns | |
| 25 | ileum | 100 nM | 7 | 5 | ns | * | ** | ** | |
| 26 | ileum | 1 uM | 6 | 7 | ** | ns | ns | ns | |
| 27 | ileum | 10 uM | 5 | 8 | * | ns | ns | * | |
| 28 | ileum | 100 uM | 4 | 6 | ns | ns | * | * | |
| 29 | colon | 100 nM | 7 | 5 | ns | ns | ns | ns | |
| 30 | colon | 1 uM | 6 | 7 | **** | ns | ns | **** | |
| 31 | colon | 10 uM | 5 | 5 | ns | ns | ns | ns | |
| 32 | colon | 100 uM | 4 | 8 | ns | ns | * | ns | |

| | AB | AC |
|---|---|---|
| 1 | | |
| 2 | | |
| 3 | | |
| 4 | | |
| 5 | | |
| 6 | | |
| 7 | | |
| 8 | | |
| 9 | | |
| 10 | | |
| 11 | | |
| 12 | | |
| 13 | | |
| 14 | | |
| 15 | | |
| 16 | | |
| 17 | | |
| 18 | | |
| 19 | | |
| 20 | | |
| 21 | | |
| 22 | | |
| 23 | | |
| 24 | | |
| 25 | | |
| 26 | | |
| 27 | | |
| 28 | p > 0.05 | ns |
| 29 | p < 0.05 | * |
| 30 | p < 0.01 | ** |
| 31 | p < 0.001 | *** |
| 32 | p < 0.0001 | **** |

[symbol]

indicates data missing or illegible when filed

### REFERENCES

[1] M. Y. Lee, S. E. Ha, C. Park, P. J. Park, R. Fuchs, L. Wei, B. G. Jorgensen, D. Redelman, S. M. Ward, K. M. Sanders, S. Ro, Transcriptome of interstitial cells of Cajal reveals unique and selective gene signatures, PLoS One. 12 (2017) 1-25. https://doi.org/10.1371/journal.pone.0176031.
[2] M. Y. Lee, C. Park, R. M. Berent, P. J. Park, R. Fuchs, H. Syn, A. Chin, J. Townsend, C. C. Benson, D. Redelman, T. Shen, J. K. Park, J. M. Miano, K. M. Sanders, S. Ro, Smooth muscle cell genome browser: Enabling the identification of novel serum response factor target genes, PLoS One. 10 (2015). https://doi.org/10.1371/journal.pone.0133751.
[3] E. Drokhlyansky, C. S. Smillie, N. VanWittenberghe, M. Ericsson, G. K. Griffin, G. Eraslan, D. Dionne, M. S. Cuoco, M. N. Goder-Reiser, T. Sharova, O. Kuksenko, A. J. Aguirre, G. M. Boland, D. Graham, O. Rozenblatt-Rosen, R. J. Xavier, A. Regev, The Human and Mouse Enteric Nervous System at Single-Cell Resolution, Cell. 182 (2020) 1606-1622.e23. https://doi.org/10.1016/j.cell.2020.08.003.
[4] J. Y. H. Liu, P. Du, W. Y. Chan, J. A. Rudd, Use of a microelectrode array to record extracellular pacemaker potentials from the gastrointestinal tracts of the ICR mouse and house musk shrew (Suncus murinus), Cell Calcium. 80 (2019). https://doi.org/10.1016/j.ceca.2019.05.002.
[5] C. Owyang, W. L. Hasler, Physiology and pathophysiology of the interstitial cells of cajal: From bench to bedside VI. Pathogenesis and therapeutic approaches to human gastric dysrhythmias, Am. J. Physiol.—Gastrointest. Liver Physiol. 283 (2002). https://doi.org/10.1152/ajpgi.00095.2002.
[6] G. J. Sanger, P. L. R. Andrews, A history of drug discovery for treatment of nausea and vomiting and the implications for future research, Front. Pharmacol. 9 (2018). https://doi.org/10.3389/fphar.2018.00913.
[7] W. E. Longo, A. M. Vernava, Prokinetic agents for lower gastrointestinal motility disorders, Dis. Colon Rectum. 36 (1993) 696-708. https.//doi.org/10.1007/13F02238599.
[8] E. Schaeffer, D. Berg, Dopaminergic Therapies for Non-motor Symptoms in Parkinson's Disease, CNS Drugs. 31 (2017) 551-570. https://doi.org/10.1007/s40263-017-0450-z.
[9] M. Verny, F. Blanc, Maladie à corps de Lewy avec troubles neurocognitifs majeurs: le traitement selon la mèdecine basée sur les preuves et en pratique, Geriatr. Psychol. Neuropsychiatr. Vieil. 17 (2019) 189-197. https://doi.org/10.1684/pnv.2019.0803.
[10] J. L. Barboza, M. S. Okun, B. Moshiree, The treatment of gastroparesis, constipation and small intestinal bacterial overgrowth syndrome in patients with Parkinson's disease, Expert Opin. Pharmacother. 16 (2015) 2449-2464. https://doi.org/10.1517/14656566.2015.1086747.
[11] J. Y. H. Liu, P. Du, Z. Lu, J. S. C. Kung, I. B. Huang, J. C. M. Hui, H. S. H. Ng, M. P. Ngan, D. Cui, B. Jiang, S. W. Chan, J. A. Rudd, Involvement of TRPV1 and TRPA1 in the modulation of pacemaker potentials in the mouse ileum, Cell Calcium. 97 (2021) 102417. https://doi.org/10.1016/j.ceca.2021.102417.
[12] J. Y. H. Liu, P. Du, J. A. Rudd, Acetylcholine exerts inhibitory and excitatory actions on mouse ileal pacemaker activity: Role of muscarinic versus nicotinic receptors, Am. J. Physiol. Gastrointest. Liver Physiol. 319 (2020) G97-G107. https://doi.org/10.1152/ajpgi.00003.2020.
[13] L. Z, Z. Y, T. L, C. S W, N. M P, C. D, L. Y H J, H. I B, K. J S C, H. C M J, R. J A, Sulprostone-Induced Gastric Dysrhythmia in the Ferret: Conventional and Advanced Analytical Approaches, Front. Physiol. 11 (2021). https://doi.org/10.3389/FPHYS.2020.583082.
[14] P. C K, B. S V, H. S, S. M, S. H E, G. A L, Mosaic organization of DNA nucleotides, Phys. Rev. E. Stat. Phys. Plasmas. Fluids. Relat. Interdiscip. Topics. 49 (1994) 1685-1689. https://doi.org/10.1103/PHYSREVE.49.1685.
[15] J. W. Kantelhardt, E. Koscielny-Bunde, H. I. A. Rego, S. Havlin, A. Bunde, Detecting long-range correlations with detrended fluctuation analysis, Phys. A Stat. Mech. Its Appl. 295 (2001) 441-454. https://doi.org/10.1016/S0378-4371(01)00144-3.
[16] C. M, G. A L, P. C K, Multiscale entropy analysis of complex physiologic time series, Phys. Rev. Lett. 89 (20029. https://doi.org/10.1103/PHYSREVLETT.89.068102.
[17] H. N. Liu, S. Ohya, Y. Nishizawa, K. Sawamnura, S. Iino, M. M. Syed, K. Goto, Y. Imaizumi, S. Nakayama, Serotonin augments gut pacemaker activity via 5-HT 3 receptors, PLoS One. 6 (2011). https://doi.org/10.1371/journal.pone.0024928.
[18] N. S, O. R, S. K, W. K, H. K, Microelectrode array evaluation of gut pacemaker activity in wild-type and W/W(v) mice, Biosens. Bioelectron. 25 (2009) 61-67. https://doi.org/10.1016/J. BIOS.2009.06.006.
[19] H. Morishita, N. Iwata, C. Takai, N. Mochizuki, N. Kaji, M. Hori, S. Kajioka, S. Nakayama, Micro-Coordination of Pacemaker Potentials in the Intestine of the Mouse, Gastroenterology. 152 (2017) 1831-18331e4. https://doi.org/10.1053/J.GASTRO.2017.04.016.
[20] T. M, K. S, S. H B, S. K, N. S, Spatial analysis of slowly oscillating electric activity in the gut of mice using low impedance arrayed microelectrodes, PLoS One. 8 (2013). https://doi.org/10.1371/JOURNAL.PONE.0075235.
[21] I. N, F. T, T. C, O. K, K S, N. S, Dialysis membrane-enforced microelectrode array measurement of diverse gut electrical activity, Biosens. Bioelectron. 94 (2017) 312-320. https://doi.org/10.1016/J. BIOS.2017.03.002.
[22] S. Nakayama, K. Shimono, H. N. Liu, H. Jiko, N. Katayama, T. Tomita, K. Goto, Pacemaker phase shift in the absence of neural activity in guinea-pig stomach: A microelectrode array study, J. Physiol. (2006). https://doi.org/10.1113/jphysiol.2006.118893.
[23] C.-S. C D, S. J J, Oral absorption of sodium pentobarbital and effects on gastrointestinal function, Pharmacol. Toxicol. 64 (1989) 23-27. https://doi.org/10.1111/JT 1600-0773.1989.TB00594.X.
[24] S. T, K. T, H. K, M. T, Y. H, O. S, S. Y, N. S, I. Y, Conversion of Ca 2+ oscillation into propagative electrical signals by Ca 2+-activated ion channels and connexin as a reconstituted Ca 2+ clock model for the pacemaker activity, Biochem. Biophys. Res. Commun. 510 (2019) 242-247. https://doi.org/10.1016/J.BBRC.2019.01.080.
[25] S. H B, S. H, I. S, N. S, Acceleration of ileal pacemaker activity in mice lacking interleukin 10, Inflamm. Bowel Dis. 19 (2013) 1577-1585. https://doi.org/10.1097/MIB.0B013E31828EEDF5.
[26] L. P, W. S M, C. A., N. M A, S. K M, Spontaneous electrical activity of interstitial cells of Cajal isolated from canine proximal colon, Proc. Natl. Acad. Sci. U.S.A. 86 (1989) 7280-7284. https://doi.org/10.1073/PNAS.86.18.7280.
[27] S. K M, S. T K, Motoneurones of the submucous plexus regulate electrical activity of the circular muscle of canine proximal colon, J. Physiol. 380 (1986) 293-310. https://doi.org/10.1113/JPHYSIOL.1986.SP016286.
[28] P. K J, H. G W, L. H T, S. N J, W. S M, S. T K, S. K M, Spatial and temporal mapping of pacemaker activity in interstitial cells of Cajal in mouse ileum in situ, Am J. Physiol. Cell Physiol. 290 (2006). https://doi.org/10.1152/AJPCELL.00447.2005.
[29] S Nakayama, S. Kajioka, K. Goto, M. Takaki, H.-N. Liu, Calcium-associated mechanisms in gut pacemaker activity, J. Cell. Mol. Med. 11 (2007) 958. https://doi.org/10.1111/J.1582-4934.2007.00107.X.
[30] K. M. Sanders, S. D. Koh, S. M. Ward, Interstitial cells of Cajal as pacemakers in the gastrointestinal tract, Annu. Rev. Physiol. 68 (2006) 307-343. https://doi.org/10.1146/annurev.physiol.68.040504.091718.

H. N. Liu, H. Hirata, Y. Okuno, M. Okabe, K. Furukawa, Dopamine and Serotonin Receptors Cooperatively Modulate Pacemaker Activity of Intestinal Cells of Cajal, Chin. J. Physiol. 61 (2018) 302-312. https://doi.org/10.4077/CJP.2018.BAH-607.
[32] R. Hardoff, M. Sula, A. Tamir, A. Soil, A. Front, S. Badarna, S. Honigman, N. Giladi, Gastric emptying time and gastric motility in patients with Parkinson's disease, Mov. Disord. (2001). https://doi.org/10.1002/mds.1203.
[33] H. Phan, A. DeReese, A. J. Day, M. Carvalho, The dual role of domperidone in gastroparesis and lactation, it. J. Pharm. Compd. 18 (2014) 203-7. http://wvw.ncbi.nlm.nih.gov/pubmed/25306766 (accessed Jul. 9, 2020).
[34] H. V. Gupta, K. E. Lyons, R. Pahxwa, Old Drugs, New Delivery Systems in Parkinson's Disease, Drugs and Aging. (2019). https://doi.org/10.1007/s40266-019-00682-9.
[35] van L. T, B. R, The need for non-oral therapy in Parkinson's disease; a potential role for apomorphine, Parkinsonism Relat. Disord. 33 Suppl 1 (2016) S22-S27. https://doi.org/10.1016/J.PARKRELDIS.2016.11.019.
[36] M. G. Zizzo, A. Bellanca, A. Amato, R. Serio, Opposite effects of dopamine on the mechanical activity of circular and longitudinal muscle of human colon, Neurogastroenterol. Motil. 32 (2020). https://doi.org/10.1111/nmo.13811.
[37] H. Tateno, R. Sakakibara, S. Shiina, H. Doi, F. Tateno, M. Sato, T. Masaka, M. Kishi, Y. Tsuyusaki, Y. Aiba, T. Ogata, Y. Suzuki, Transdermal Dopamine Agonist Ameliorates Gastric Emptying in Parkinson's Disease, J. Am. Geriatr. Soc. 63 (2015) 2416-2418. https://doi.org/10.1111/jgs.13800.
[38] T. Lüscher Dias, V. Schuch, P. C. B. Beltrão-Braga, D Martins-de-Souza, H. P. Brentani, G. R. Franco, H. I. Nakaya, Drug repositioning for psychiatric and neurological disorders through a network medicine approach, Transl. Psychiatry. 10 (2020) 141. https://doi.org/10.1038/s41398-020-0827-5.
[39] F. Gentile, V. Agrawal, M. Hsing, A. T. Ton, F. Ban, U. Norinder, M. E. Gleave, A. Cherkasov, Deep Docking: A Deep Learning Platform for Augmentation of Structure Based Drug Discovery, ACS Cent. Sci. 6 (2020) 939-949. https://doi.org/10.1021/acscentsci.0c00229.
[40] H. Li, Z. Gao, L. Kang, H. Zhang, K. Yang, K. Yul, X. Luo, W. Zhu, K. Chen, J. Shen, X. Wang, H. Jiang, TarFisDock: A web server for identifying drug targets with docking approach, Nucleic Acids Res. 34 (2006) W219-W224. https://doi.org/10.1093/nar/gkl114.
[41] K. Sachdev, M. K. Gupta, A comprehensive review of computational techniques for the prediction of drug side effects, Drug Dev. Res. 81 (2020) 650-670. https://doi.org/10.1002/DDR.21669.
[42] Liu J. Y. H., Rudd J. A., Du P.* (2021) A pipeline for phase-based analysis of in vitro micro-electrode array recordings of gastrointestinal slow waves. IEEE Engineering in Medicine and Biology Society. [PMID: 34891286]
[43] Vo A H, VanVleet T R, Gupta R R, Liguori M J, Rao M S. An Overview of Machine Learning and Big Data for Drug Toxicity Evaluation. Chem Res Toxicol 2020; 33:20-37. https://doi.org/10.1021/ACS.CHEMRESTOX.9B00227.
[44] Lee C Y, Chen Y P P. Prediction of drug adverse events using deep learning in pharmaceutical discovery. Brief Bioinform 2021; 22:1884-901. https://doi.org/10.1093/BIB/BBAA040.
[45] Petra A I, Panagiotidou S, Hatziagelaki E, Stewart J M, Conti P, Theoharides T C. Gut-microbiota-brain axis and effect on neuropsychiatric disorders with suspected immune dysregulation. Clin Ther 2015; 37:984. https://doi.org/10.1016/J.CLINTHERA 2015.04.002.
[46] Kuhn M, Letunic I, Jensen L J, Bork P. The SIDER database of drugs and side effects. Nucleic Acids Res 2016; 44:D1075-9. https://doi.org/10.1093/NAR/GKV1075.
[47] Radu B M, Banciu A, Banciu D D, Radu M, Cretoiu D, Cretoiu S M. Calcium Signaling in Interstitial Cells: Focus on Telocytes. Int J Mol Sci 2017; 18. https://doi.org/10.3390/IJMS18020397.
[48] Tyers M B, Bunce K T, Humphrey P P A. Pharmacological and anti-emetic properties of ondansetron. Eur J Cancer Clin Oncol 1989; 25 Suppl 1.
[49] Stern H. Serotonin, and its effects on the pituitary-adrenal axis in depression. Chic Med Sch Q 1970; 29:24-30.
Keren D F. Intestinal mucosal immune defense mechanisms. Am J Surg Pathol 1988; 12 Suppl 1:100-5.

We claim:

1. A method of testing effects of one or more substances on pacemaker activity on gastrointestinal tissues using a recording platform to determine whether the one or more substances belong to one or more classes, the method comprising:
applying a substance for testing on at least one sub-segment of freshly isolated gastrointestinal tissue from a living organism;
maintaining the tissue in oxygenated medium to maintain a viability of the tissue;
recording electrical signals from a surface of the tissue using the recording platform to create a recorded digital signal;
storing the recorded digital signal in a data storage device;
generating a plurality of test results by analyzing the recorded digital signal using a set of machine-readable instructions that allow a computer to extract at least one feature from the recorded digital signal;
storing the plurality of test results into a database;
training one or more machine learning models based on the plurality of test results stored in the database, to create a trained model;
applying the trained model for classifying, predicting, or comparing the substance; and
reporting a result of the classifying, predicting, or comparing.

2. The method according to claim 1, wherein the substance comprises one or more of drugs, pharmacological agents, chemical compounds, synthesized substances, food, remedies, herbs, extracts, and any combination thereof.

3. The method according to claim 1, wherein the recording platform comprises a signal receiver, an amplifier, an internal filter, a grounding electrode, and a microelectrode array chip; the microelectrode array chip comprising a multiplicity of microelectrodes embedded on a rigid substrate.

4. The method according to claim 1, comprising predicting and classifying between agonist and antagonist actions of the one or more substances, or predicting and classifying between high-risk and low-risk in a set of selected side effects of the substance.

5. The method according to claim 4, the set of selected side effects comprising one or more of vomiting, emesis, nausea, diarrhea, constipation, abdominal discomfort, and dysrhythmia.

6. The method according to claim 1, wherein the sub-segment of freshly isolated gastrointestinal tissue comprises tissue from an esophagus, stomach, duodenum, jejunum, ileum, rectum, caecum, or colon.

7. The method according to claim 1, wherein the living organism is an organism having functional gastrointestinal organs.

8. The method according to claim 1, wherein the living organism is human, mammalian, reptilian, or aquatic.

9. The method according to claim 1, wherein the living organism is healthy; or is diagnosed with a disease, genetic condition, or alteration; or is pre-treated with the substance prior to the applying the substance for testing.

10. The method according to claim 1, further comprising the step of removing contents from within the freshly isolated gastrointestinal tissue.

11. The method according to claim 1, further comprising maintaining the temperature of the freshly isolated gastrointestinal tissue within a range of twenty to forty degrees Celsius.

12. The method according to claim 1, further comprising recording a baseline signal for at least five minutes prior to the applying the substance for testing.

13. The method according to claim 12, the applying the substance for testing comprising delivering a specified quantity of the substance onto the sub-segment of freshly isolated gastrointestinal tissue at a specified time after the recording of the baseline signal.

14. The method according to claim 13, wherein the delivering comprises either direct delivery using a handheld pipette or machine-controlled delivery using a machine-controlled perfusion system.

15. The method according to claim 13, wherein the recording electrical signals occurs after the delivering of the specified quantity of the substance onto the sub-segment of freshly isolated gastrointestinal tissue at the specified time, and wherein the recorded digital signal is a post-substance delivery signal.

16. The method according to claim 15, further comprising comparing the baseline signal to the post-substance delivery signal.

17. The method according to claim 1, wherein the recorded digital signal is created within less than one hour after the applying one or more substances for testing.

18. The method according to claim 1, wherein the at least one feature from the recorded digital signal comprises one or more of:
the determination of a number of dominant propagation patterns using a factor of respective activation times found at each electrode within a baseline period and a post-substance delivery period, respectively, into a time interval between ten to sixty seconds;
the percentage of the dominant propagation patterns found in the baseline period and the post-substance delivery period, respectively; and
the change in the percentage of a first, second, or third propagation pattern based on a comparison between the baseline period and the post-substance delivery period.

19. The method according to claim 1, the substance being a first substance and the database comprising (i) a first unique individual database section configured to store the at least one feature from the recorded digital signal for the first substance and (i) a second unique individual database section configured to store at least one feature from a recorded digital signal for a second substance.

20. The method according to claim 19, comprising:
building a trained machine learning model based on the first unique individual database section and the second unique individual database section; and
integrating the first unique individual database section and the second unique individual database section with at least one other database or training model.
